(19) United States
(12) Patent Application Publication
(10) Pub. No.: US 2025/0289828 A1
(43) Pub. Date: Sep. 18, 2025

(54) MEDIUM- OR MACRO-CYCLIC BENZYL-SUBSTITUTED HETEROCYCLE DERIVATIVES AND RELATED USES

(71) Applicant: CENTESSA PHARMACEUTICALS (UK) LIMITED, Altrincham (GB)

(72) Inventors: Gregory R. OTT, Media, PA (US); Karl GIBSON, Sandwich (GB); Bruce LEFKER, Swampscott, MA (US); Paul HUMPHRIES, Santa Clara, CA (US); Matthew SPENDIFF, Cambridge (GB)

(21) Appl. No.: 19/177,396

(22) Filed: Apr. 11, 2025

Related U.S. Application Data
(63) Continuation of application No. 18/817,943, filed on Aug. 28, 2024.
Application No. 18/817,943 is a continuation of application No. PCT/US2023/014275, filed on Mar. 1, 2023.
(60) Provisional application No. 63/315,438, filed on Mar. 1, 2022.
(60) Provisional application No. 63/439,477, filed on Jan. 17, 2023.

(51) Int. Cl. C07D 498/04; A61K 31/407; A61K 31/4353; A61P 25/26

(52) U.S. Cl. CPC C07D 498/04; A61K 31/407; A61K 31/4353; A61P 25/26

(57) ABSTRACT

The present disclosure relates to compounds of Formula (I′):

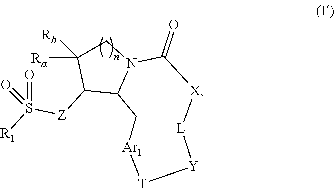

and to their prodrugs, pharmaceutically acceptable salts, pharmaceutical compositions, methods of use, and methods for their preparation. The compounds disclosed herein are useful for modulating orexin-2 receptor activity and may be used in the treatment of disorders in which orexin-2 receptor activity is implicated, such as narcolepsy, a hypersomnia disorder, a neurodegenerative disorder, a symptom of a rare genetic disorder, a mental health disorder, a metabolic syndrome, osteoporosis, cardiac failure, coma, or facilitating emergence from anaesthesia.

### RELATED APPLICATIONS

This application is a continuation application of U.S. application Ser. No. 18/817,943, filed on Aug. 28, 2024, which is a continuation application of International Application No. PCT/US2023/014275, filed on Mar. 1, 2023, which claims the benefit of U.S. Provisional Application No. 63/315,438, filed Mar. 1, 2022 and U.S. Provisional Application No. 63/439,477, filed Jan. 17, 2023, the entire contents of each of which are incorporated herein by reference.

### BACKGROUND

The present disclosure relates to small molecule, potent agonists of the orexin-2 receptor (OX2R), designed for the treatment of narcolepsy and other disorders associated with orexin insufficiency and/or excessive sleepiness. Narcolepsy afflicts 1 in 2000 individuals worldwide. Onset may occur during adolescence for a lifelong duration and debilitating impact on quality of life. Narcolepsy Type 1 (NT1) is caused by the loss of neurons in the brain which produce orexin neuropeptides. There is no known cure, and currently approved treatments are symptomatic. Thus, development of pharmacotherapeutics to restore lost orexin signaling is critically important for treatment of the root cause of NT1.

In narcolepsy Type 1 (NT1), the sole population of neurons that produce orexin A and B (also known as hypocretin-1 and 2) peptides are destroyed by an immune mechanism which causes arousal state boundary dysfunction. Mouse models of narcolepsy type 1 recapitulate the loss of orexin neurons and the two cardinal symptoms observed in NT1 patients, specifically excessive daytime sleepiness and cataplexy. Common symptoms of narcolepsy type 1 and type 2 may include excessive daytime sleepiness, disturbed nighttime sleep, and inappropriately timed rapid-eye-movement (REM) sleep, as well as sleep paralysis and hypnopompic/hypnogogic hallucinations. Cataplexy is the intrusion of sudden, reversible loss of muscle tone (the atonia of REM sleep) into wakefulness in response to emotional stimuli and is pathognomonic of NT1.

The two predominant symptoms of narcolepsy type 1, excessive daytime sleepiness and cataplexy, can be reduced by re-activation of orexin neurotransmission at OX2R in mouse models. Reversal of cataplexy-like events and sleep/wake fragmentation has been achieved by genetic, focal restoration of OX2R signaling in the dorsal raphe nucleus of the pons and the tuberomammillary nucleus of the hypothalamus, respectively, in mice that otherwise lack orexin receptors in those regions. Intracerebroventricular (ICV) administration of orexin A (OXA) has been shown to increase time spent awake and decreases cataplexy-like behavior in orexin-neuron ablated mice. Selective OX2R agonist, YNT-185 administered intraperitoneally or ICV, modestly increases wakefulness in wild type (WT) and orexin ligand-deficient mice, and decrease sleep-onset REM periods and cataplexy-like events in an NT1 mouse model. Subcutaneous administration of the selective OX2R agonist TAK-925 modestly increased wakefulness in WT mice, but not in OX2R-knockout mice. Brain penetrant and stable OX2R agonists that are bioavailable after alternative routes of administration (including but not limited to oral, intranasal, transmucosal, and transdermal) and that bind with high affinity for potent excitation of arousal-state regulating neurons will provide an improvement to current therapeutics for patients with NT1. In fact, initial clinical studies reported with TAK-925 showed both substantial levels of increased wakefulness and trends for decreasing cataplexy in individuals with NT1. Activation of the OX1R is implicated in regulation of mood and reward behaviors, and may also contribute to arousal.

Orexin receptor agonists may also be useful in other indications marked by some degree of orexin neurodegeneration and excessive daytime sleepiness, such as Parkinson's disease, Alzheimer's disease, Huntington's disease, multiple sclerosis, and traumatic brain injury. Because stimulation of OX2R promotes wakefulness in orexin-intact animals, orexin receptor agonists may treat excessive daytime sleepiness in patients with normal levels of orexin, including narcolepsy type 2, idiopathic hypersomnia, or sleep apnea. Similarly, orexin receptor agonists may confer wake-promoting benefits in disorders of recurrent hypersomnia, such as Klein-Levin syndrome, or inappropriately timed sleep (i.e., circadian rhythm sleep disorders), such as delayed- or advanced-sleep phase disorder, shift work disorder, and jet lag disorder. The abnormal daytime sleepiness, sleep onset REM periods, and cataplexy-like symptoms of rare genetic disorders (e.g., ADCA-DN, Coffin-Lowry syndrome, Moebius syndrome, Norrie disease, Niemann-Pick disease type C, and Prader-Willi syndrome) could be alleviated with orexin receptor agonists. Other indications in which orexin receptor agonists have been suggested to confer benefits include attention deficit hyperactivity disorder, age-related cognitive dysfunction, metabolic syndrome and obesity, osteoporosis, cardiac failure, coma, and emergence from anesthesia.

The disclosure arises from a need to provide further compounds for the modulation of orexin receptor activity in the brain, including activation of the orexin-2 receptor, with improved therapeutic potential. In particular, compounds with improved physicochemical, pharmacological and pharmaceutical properties to existing compounds are desirable.

### SUMMARY

In some aspects, the present disclosure provides a compound of Formula (I):

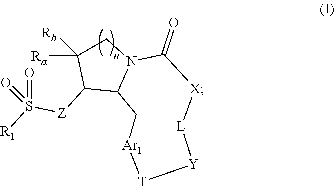

or a pharmaceutically acceptable salt thereof, wherein:
X is —N(CH₃)— or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (I′):

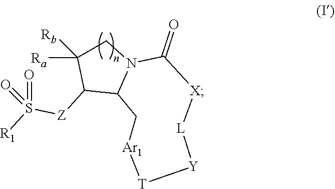

or a pharmaceutically acceptable salt thereof, wherein:
X is —N(CH₃)— or C₂-C₆ alkyl;
L is absent or C₂-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (I″):

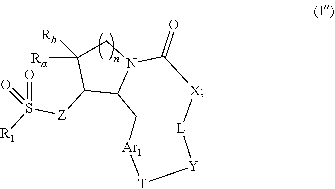

or a pharmaceutically acceptable salt thereof, wherein:
X is —N(CH₃)— or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (I″′):

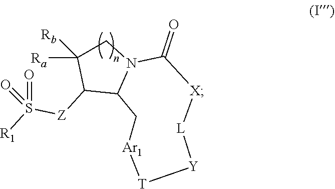

or a pharmaceutically acceptable salt thereof, wherein:
X is —N(CH₃)— or C₂-C₆ alkyl;
L is absent or C₂-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (II):

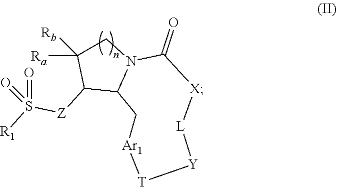

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (II′):

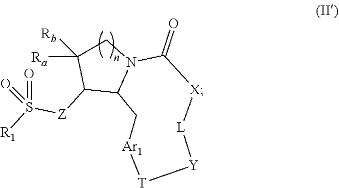

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (III):

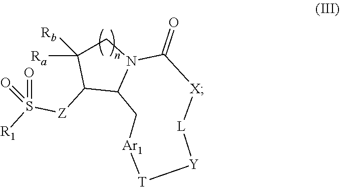

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (III′):

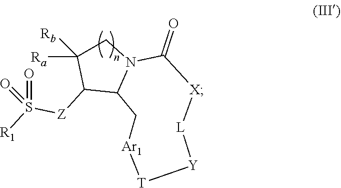

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₂-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound obtainable by, or obtained by, a method for preparing a compound as described herein (e.g., a method comprising one or more steps described in Schemes 1-9).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Examples 1-50).

In some aspects, the present disclosure provides a method of modulating orexin-2 receptor activity (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating orexin-2 receptor activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for modulating orexin-2 receptor activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to macrocyclic ([1,1′-biphenyl]-3-ylmethyl)-substituted heterocycle derivatives, prodrugs, and pharmaceutically acceptable salts thereof, which may modulate orexin-2 receptor activity and are accordingly useful in methods of treatment of the human or animal body. The present disclosure also relates to processes for the preparation of these compounds, to pharmaceutical compositions comprising them and to their use in the treatment of disorders in which the orexin-2 receptor is implicated, such as narcolepsy, a neurodegenerative disorder, a symptom of a rare genetic disorder, a mental health disorder, a metabolic syndrome, osteoporosis, cardiac failure, coma, or emergence from anesthesia.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options. For example, while various options for variables X, L, and Y are described herein, the disclosure may be interpreted as excluding structures for non-operable compound caused by certain combinations of variables X, L, and Y (e.g., when each of X, L, and Y is —O—).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, Cs or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, Cs and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

It is understood that when a variable has two attachments to the rest of the formula of the compound, the two attachments could be at the same atom or different atoms of the variable. For example, when a variable (e.g., variable X) is cycloalkyl or heterocycloalkyl, and has two attachments to the rest of the formula of the compound, the two attachments could be at the same atom or different atoms of the cycloalkyl or heterocycloalkyl.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for organic Synthesis, John Wiley and Sons (1994); and L. Paquette, ed., Encyclopedia of Reagents for organic Synthesis, John Wiley and Sons (1995), incorporated by reference herein, are useful and recognised reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), cyclodextrins and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, capsules or sachets. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, powders or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides a compound of Formula (I):

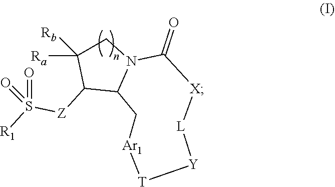

or a pharmaceutically acceptable salt thereof, wherein:
X is —N(CH₃)— or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (I′):

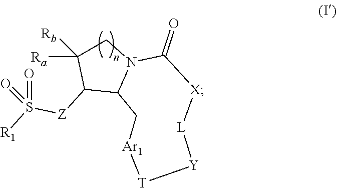

or a pharmaceutically acceptable salt thereof, wherein:
X is —N(CH₃)— or C₂-C₆ alkyl;
L is absent or C₂-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (I″):

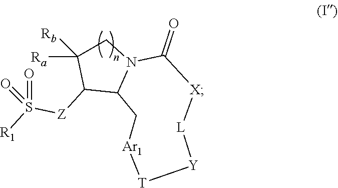

or a pharmaceutically acceptable salt thereof, wherein:
X is —N(CH₃)— or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (I″′):

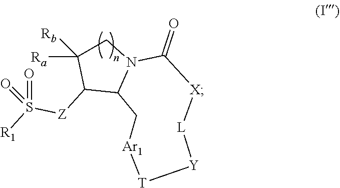

or a pharmaceutically acceptable salt thereof, wherein:
X is —N(CH₃)— or C₂-C₆ alkyl;
L is absent or C₂-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (II):

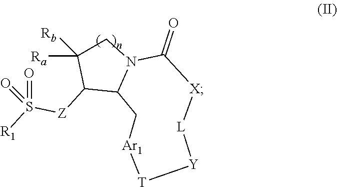

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (II′):

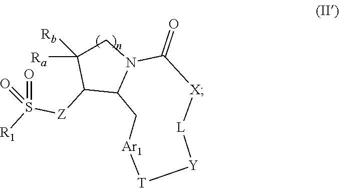

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

In some aspects, the present disclosure provides a compound of Formula (III):

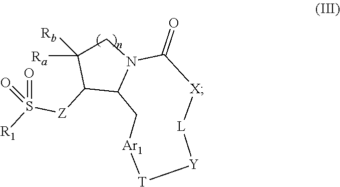

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (III′):

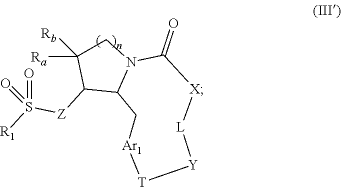

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₂-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (III″):

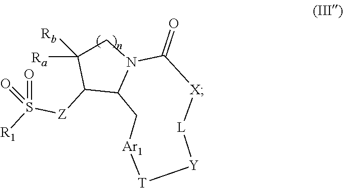

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl optionally substituted with one or more halogen or C₁-C₆ alkyl;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (III″′):

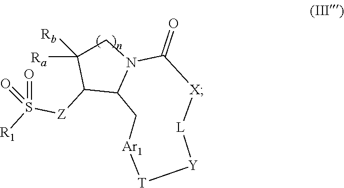

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₂-C₆ alkyl, or C₃ cycloalkyl optionally substituted with one or more halogen or C₁-C₆ alkyl;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (IV):

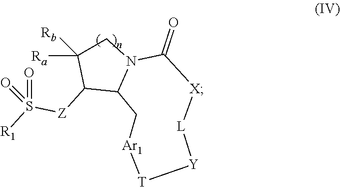

or a pharmaceutically acceptable salt thereof, wherein:
X is —O—, —N(C₁-C₆ alkyl)-, —N(C₁-C₆ haloalkyl)-, or C₁-C₆ alkyl optionally substituted with one or more C₁-C₆ alkyl or —OH;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (IV′):

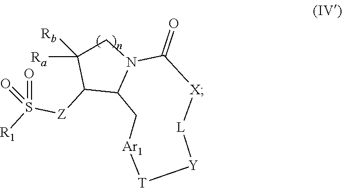

or a pharmaceutically acceptable salt thereof, wherein:
X is —O—, —N(C₁-C₆ alkyl)-, —N(C₁-C₆ haloalkyl)-, or C₁-C₆ alkyl optionally substituted with one or more C₁-C₆ alkyl or —OH;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₂-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (IV″):

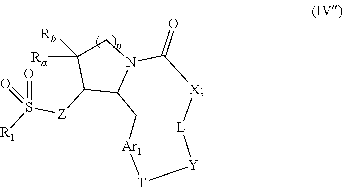

or a pharmaceutically acceptable salt thereof, wherein:
X is —O—, —N(C₁-C₆ alkyl)-, —N(C₁-C₆ haloalkyl)-, or C₁-C₆ alkyl optionally substituted with one or more C₁-C₆ alkyl or —OH;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl optionally substituted with one or more halogen or C₁-C₆ alkyl;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

In some aspects, the present disclosure provides a compound of Formula (IV″′):

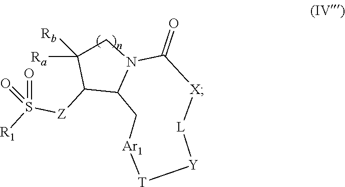

or a pharmaceutically acceptable salt thereof, wherein:
X is —O—, —N(C₁-C₆ alkyl)-, —N(C₁-C₆ haloalkyl)-, or C₁-C₆ alkyl optionally substituted with one or more C₁-C₆ alkyl or —OH;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₂-C₆ alkyl, or C₃ cycloalkyl optionally substituted with one or more halogen or C₁-C₆ alkyl;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

It is understood that, for a compound of the present disclosure, variables X, L, Y, n, Rₐ, R_{b}, Z, R₁, Ar₁, and T can each be, where applicable, selected from the groups described herein, and any group described herein for any of variables X, L, Y, n, Rₐ, R_{b}, Z, R₁, Ar₁, and T can be combined, where applicable, with any group described herein for one or more of the remainder of variables X, L, Y, n, Rₐ, R_{b}, Z, R₁, Ar₁, and T.

In some embodiments, X is —O—, —N(C₁-C₆ alkyl)-, —N(C₁-C₆ haloalkyl)-, or C₁-C₆ alkyl optionally substituted with one or more C₁-C₆ alkyl or —OH.

In some embodiments, X is —O—.

In some embodiments, X is —N(CH₃)—, —N(C₁-C₆ haloalkyl)-, or C₁-C₆ alkyl.

In some embodiments, X is —N(CH₃)— or C₁-C₆ alkyl.

In some embodiments, X is —N(CH₃)—.

In some embodiments, X is C₁-C₆ alkyl.

In some embodiments, X is C₁-C₆ alkyl optionally substituted with one or more C₁-C₆ alkyl or —OH.

In some embodiments, X is C₁-C₆ alkyl substituted with one or more C₁-C₆ alkyl or —OH.

In some embodiments, X is C₁-C₆ alkyl optionally substituted with one or more C₁-C₆ alkyl.

In some embodiments, X is C₁-C₆ alkyl substituted with one or more C₁-C₆ alkyl.

In some embodiments, X is C₁-C₆ alkyl optionally substituted with one or more —OH.

In some embodiments, X is C₁-C₆ alkyl substituted with one or more —OH.

In some embodiments, X is methyl. In some embodiments, X is ethyl. In some embodiments, X is propyl. In some embodiments, X is butyl. In some embodiments, X is pentyl. In some embodiments, X is hexyl. In some embodiments, X is isopropyl. In some embodiments, X is isobutyl. In some embodiments, X is isopentyl. In some embodiments, X is isohexyl. In some embodiments, X is secbutyl. In some embodiments, X is secpentyl. In some embodiments, X is sechexyl. In some embodiments, X is tertbutyl.

In some embodiments, X is —N(C₁-C₆ haloalkyl)-.

In some embodiments, X is —N(halomethyl)-. In some embodiments, X is —N(haloethyl)-. In some embodiments, X is —N(halopropyl)-. In some embodiments, X is —N(halobutyl)-. In some embodiments, X is —N(halopentyl)-. In some embodiments, X is —N(halohexyl)-.

In some embodiments, L is absent or C₁-C₆ alkyl.

In some embodiments, L is absent or C₂-C₆ alkyl.

In some embodiments, L is absent.

In some embodiments, L is C₁-C₆ alkyl.

In some embodiments, L is C₂-C₆ alkyl.

In some embodiments, L is methyl. In some embodiments, L is ethyl. In some embodiments, L is propyl. In some embodiments, L is butyl. In some embodiments, L is pentyl. In some embodiments, L is hexyl. In some embodiments, L is isopropyl. In some embodiments, L is isobutyl. In some embodiments, L is isopentyl. In some embodiments, L is isohexyl. In some embodiments, L is secbutyl. In some embodiments, L is secpentyl. In some embodiments, L is sechexyl. In some embodiments, L is tertbutyl.

In some embodiments, Y is —O—.

In some embodiments, n is 1 or 2.

In some embodiments, n is 1. In some embodiments, n is 2.

In some embodiments, it is H or halogen.

In some embodiments, it is H.

In some embodiments, it is halogen.

In some embodiments, it is F, Cl, Br, or I. In some embodiments, Rₐ is F, Cl, or Br. In some embodiments, Rₐ is F or Cl.

In some embodiments, Rₐ is F. In some embodiments, it is Cl. In some embodiments, Rₐ is Br. In some embodiments, Rₐ is I.

In some embodiments, R_{b} is H, halogen, or C₁-C₆ alkyl.

In some embodiments, R_{b} is H.

In some embodiments, R_{b} is halogen.

In some embodiments, R_{b} is F, Cl, Br, or I. In some embodiments, R_{b} is F, Cl, or Br. In some embodiments, R_{b} is F or Cl.

In some embodiments, R_{b} is F. In some embodiments, R_{b} is Cl. In some embodiments, R_{b} is Br. In some embodiments, R_{b} is I.

In some embodiments, R_{b} is C₁-C₆ alkyl.

In some embodiments, R_{b} is methyl. In some embodiments, R_{b} is ethyl. In some embodiments, R_{b} is propyl. In some embodiments, R_{b} is butyl. In some embodiments, R_{b} is pentyl. In some embodiments, R_{b} is hexyl. In some embodiments, R_{b} is isopropyl. In some embodiments, R_{b} is isobutyl. In some embodiments, R_{b} is isopentyl. In some embodiments, R_{b} is isohexyl. In some embodiments, R_{b} is secbutyl. In some embodiments, R_{b} is secpentyl. In some embodiments, R_{b} is sechexyl. In some embodiments, R_{b} is tertbutyl.

In some embodiments, Rₐ and R_{b}, together with the atom they attach to, form C₃ cycloalkyl.

In some embodiments, Z is —NH—.

In some embodiments, R₁ is C₁-C₆ haloalkyl or C₂-C₆ alkyl.

In some embodiments, R₁ is C₁-C₆ haloalkyl, C₁-C₆ alkyl, or C₃ cycloalkyl substituted with halogen.

In some embodiments, R₁ is C₁-C₆ haloalkyl, C₂-C₆ alkyl, or C₃ cycloalkyl substituted with halogen.

In some embodiments, R₁ is C₁-C₆ haloalkyl, C₁-C₆ alkyl, or C₃ cycloalkyl optionally substituted with one or more halogen.

In some embodiments, R₁ is C₁-C₆ haloalkyl, C₂-C₆ alkyl, or C₃ cycloalkyl optionally substituted with one or more halogen.

In some embodiments, R₁ is C₁-C₆ haloalkyl.

In some embodiments, R₁ is C₁-C₆ haloalkyl.

In some embodiments, R₁ is halomethyl. In some embodiments, R₁ is haloethyl. In some embodiments, R₁ is halopropyl. In some embodiments, R₁ is halobutyl. In some embodiments, R₁ is halopentyl. In some embodiments, R₁ is halohexyl.

In some embodiments, R₁ is C₂-C₆ alkyl.

In some embodiments, R₁ is C₁-C₆ alkyl.

In some embodiments, R₁ is methyl. In some embodiments, R₁ is ethyl. In some embodiments, R₁ is propyl. In some embodiments, R₁ is butyl. In some embodiments, R₁ is pentyl. In some embodiments, R₁ is hexyl. In some embodiments, R₁ is isopropyl. In some embodiments, R₁ is isobutyl. In some embodiments, R₁ is isopentyl. In some embodiments, R₁ is isohexyl. In some embodiments, R₁ is secbutyl. In some embodiments, R₁ is secpentyl. In some embodiments, R₁ is sechexyl. In some embodiments, R₁ is tertbutyl.

In some embodiments, R₁ is C₃ cycloalkyl.

In some embodiments, R₁ is C₃ cycloalkyl optionally substituted with one or more halogen or C₁-C₆ alkyl.

In some embodiments, R₁ is C₃ cycloalkyl optionally substituted with one or more halogen.

In some embodiments, R₁ is C₃ cycloalkyl substituted with halogen.

In some embodiments, R₁ is C₃ cycloalkyl optionally substituted with one or more C₁-C₆ alkyl.

In some embodiments, R₁ is C₃ cycloalkyl substituted with one or more C₁-C₆ alkyl.

In some embodiments, Ar₁ is C₆-C₁₀ aryl optionally substituted with one or more halogen.

In some embodiments, Ar₁ is C₆-C₁₀ aryl.

In some embodiments, Ar₁ is C₆-C₁₀ aryl substituted with one or more halogen.

In some embodiments, Ar₁ is phenyl optionally substituted with one or more halogen.

In some embodiments, Ar₁ is phenyl.

In some embodiments, Ar₁ is phenyl substituted with one or more halogen.

In some embodiments, T is C₆-C₁₀ aryl optionally substituted with one or more halogen or C₁-C₆ alkyl.

In some embodiments, T is C₆-C₁₀ aryl optionally substituted with one or more halogen.

In some embodiments, T is C₆-C₁₀ aryl.

In some embodiments, T is C₆-C₁₀ aryl substituted with one or more halogen or C₁-C₆ alkyl.

In some embodiments, T is C₆-C₁₀ aryl substituted with one or more halogen.

In some embodiments, T is phenyl optionally substituted with one or more halogen or C₁-C₆ alkyl.

In some embodiments, T is phenyl optionally substituted with one or more halogen.

In some embodiments, T is phenyl.

In some embodiments, T is phenyl substituted with one or more halogen or C₁-C₆ alkyl.

In some embodiments, T is phenyl substituted with one or more halogen.

In some embodiments, when X is alkyl, L is absent.

In some embodiments, the compound is of Formula (I′-a) or (I′-b):

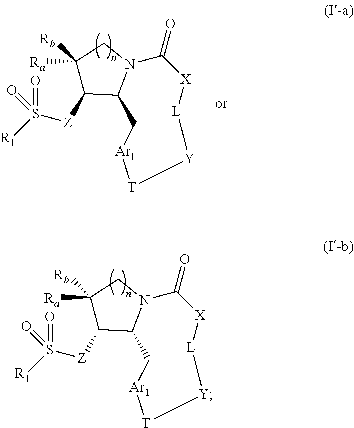

or a pharmaceutically acceptable salt thereof.

In some embodiments, the compound is of Formula (II′-a), (II′-b), or (II′-c):

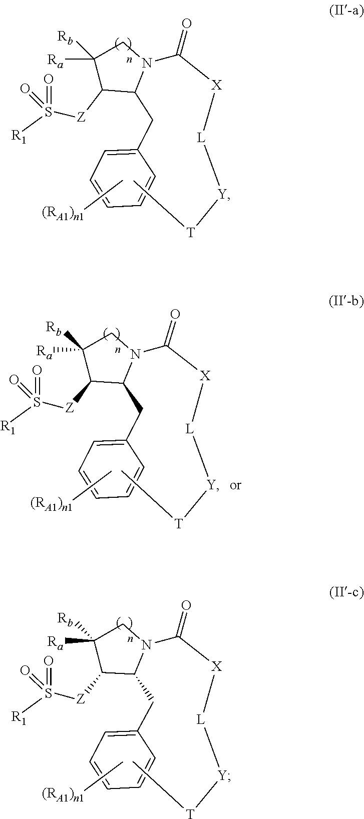

or a pharmaceutically acceptable salt thereof, wherein:
n1 is an integer ranging from 0 to 4.

In some embodiments, the compound is of Formula (IIIA′), (IIIA′-a), or (IIIA′-b):

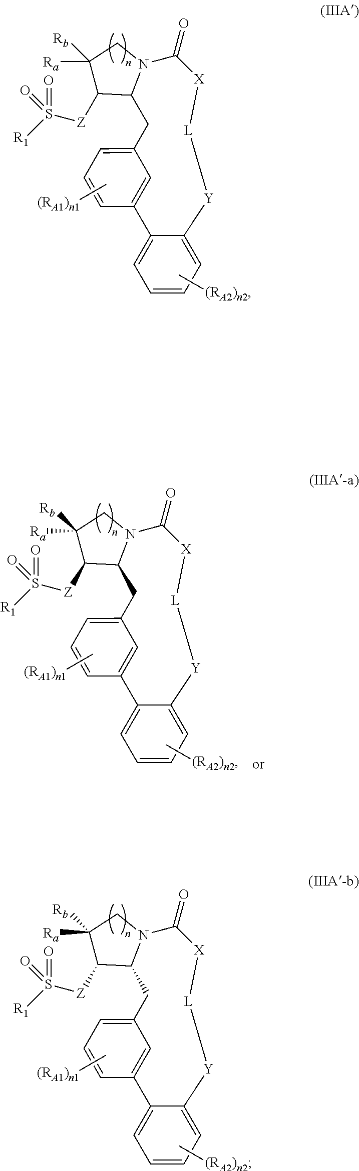

or a pharmaceutically acceptable salt thereof, wherein:
n1 is an integer ranging from 0 to 4; and
n2 is an integer ranging from 0 to 4.

In some embodiments, the compound is of Formula (IVA′), (IVA′-a), or (IVA′-b):

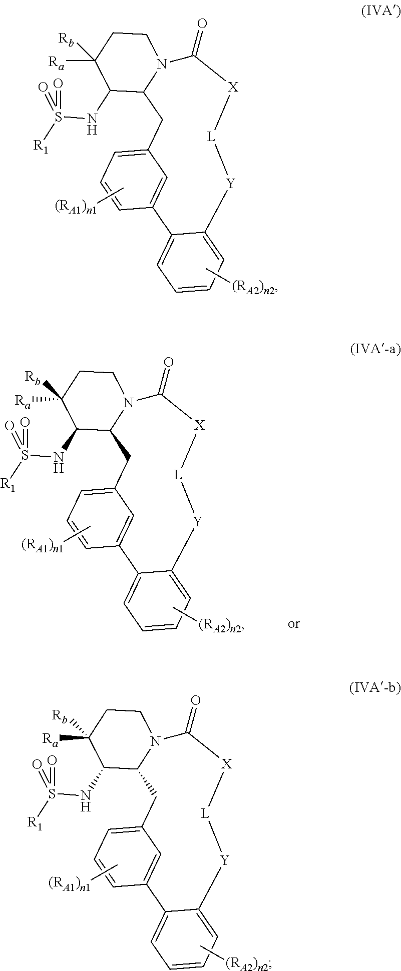

or a pharmaceutically acceptable salt thereof, wherein:
n1 is an integer ranging from 0 to 4; and
n2 is an integer ranging from 0 to 4.

In some embodiments, the compound is of Formula (I-a) or (I-b):

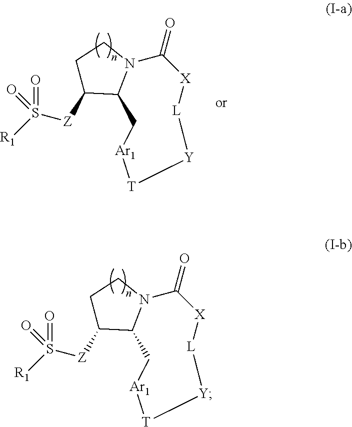

or a pharmaceutically acceptable salt thereof.

In some embodiments, the compound is of Formula (II-a), (II-b), or (II-c):

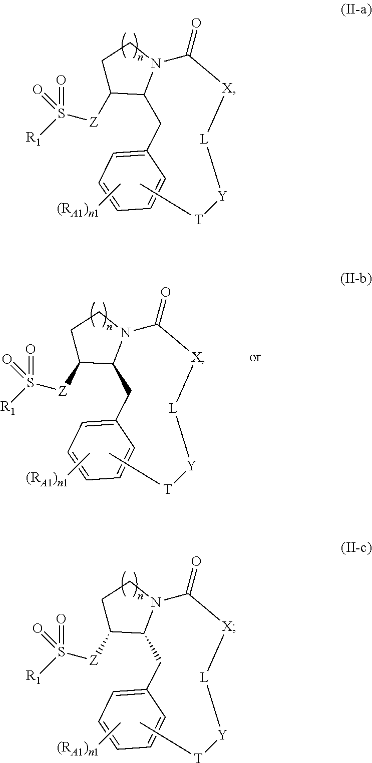

or a pharmaceutically acceptable salt thereof, wherein:
n1 is an integer ranging from 0 to 4.

In some embodiments, the compound is of Formula (IIIA), (IIIA-a), or (IIIA-b):

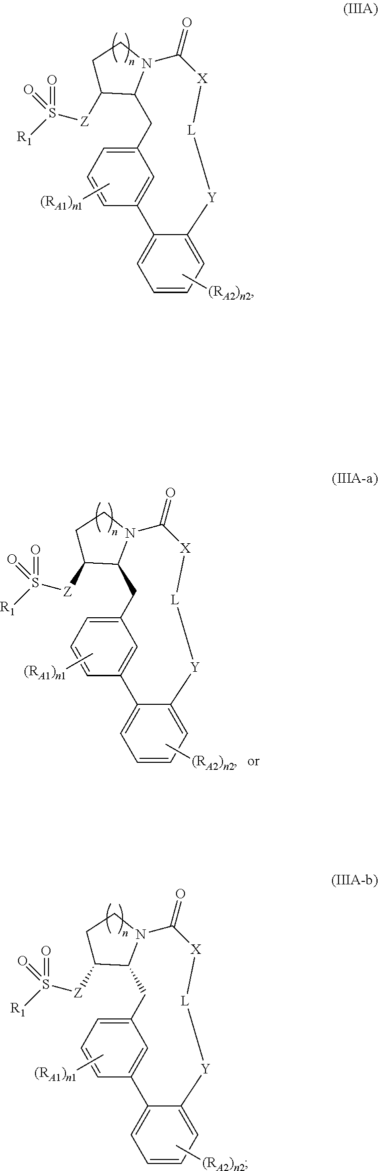

or a pharmaceutically acceptable salt thereof, wherein:
n1 is an integer ranging from 0 to 4; and
n2 is an integer ranging from 0 to 4.

In some embodiments, the compound is of Formula (IIIA′-1), (IIIA′-la), or (IIA′-1b):

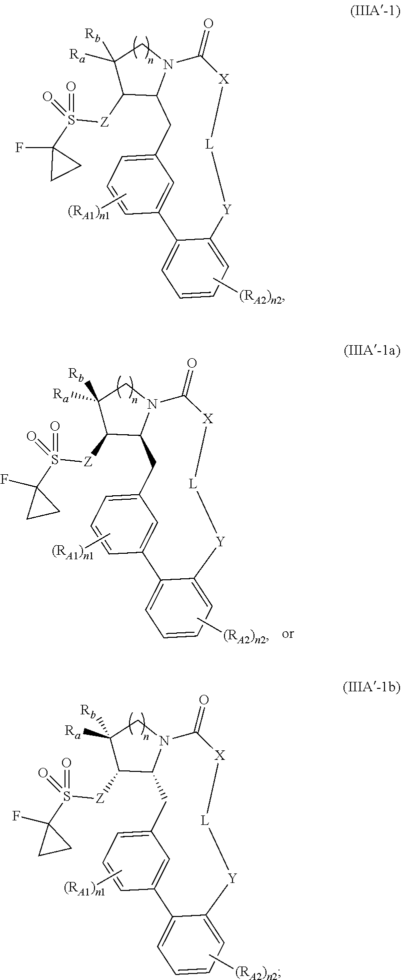

or a pharmaceutically acceptable salt thereof, wherein:
n1 is an integer ranging from 0 to 4; and
n2 is an integer ranging from 0 to 4.

In some embodiments, the compound is of Formula (IIIA′-2), (IIIA′-2a), or (IIIA′-2b):

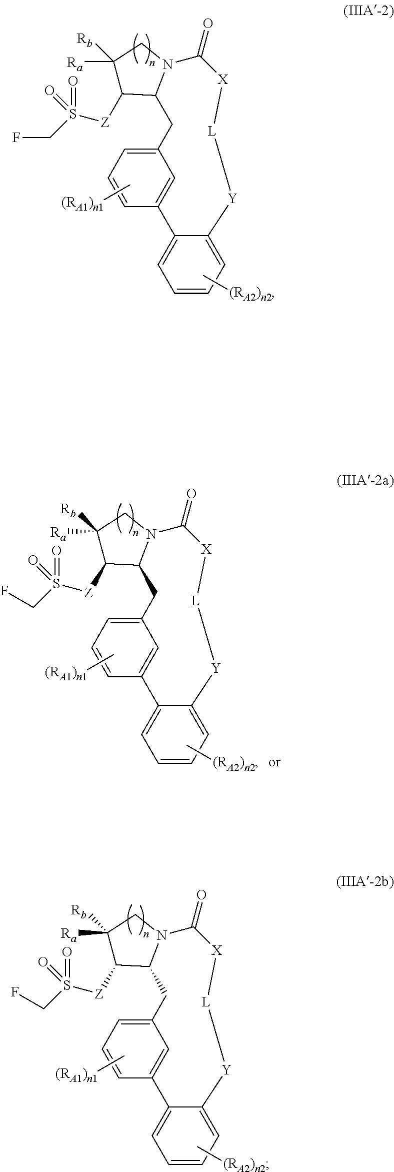

or a pharmaceutically acceptable salt thereof, wherein:
n1 is an integer ranging from 0 to 4; and
n2 is an integer ranging from 0 to 4.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

In some embodiments, the compound is selected from the compounds described in Table 1.

In some embodiments, the compound is selected from the compounds described in Table 2 and prodrugs and pharmaceutically acceptable salts thereof.

In some embodiments, the compound is selected from the compounds described in Table 2 and pharmaceutically acceptable salts thereof.

In some embodiments, the compound is selected from the prodrugs of compounds described in Table 2 and pharmaceutically acceptable salts thereof.

In some embodiments, the compound is selected from the compounds described in Table 2.

In some embodiments, the compound is selected from the compounds described in Table 3 and prodrugs and pharmaceutically acceptable salts thereof.

In some embodiments, the compound is selected from the compounds described in Table 3 and pharmaceutically acceptable salts thereof.

In some embodiments, the compound is selected from the prodrugs of compounds described in Table 3 and pharmaceutically acceptable salts thereof.

In some embodiments, the compound is selected from the compounds described in Table 3.

In some embodiments, the compound is selected from the compounds described in Table 4 and prodrugs and pharmaceutically acceptable salts thereof.

In some embodiments, the compound is selected from the compounds described in Table 4 and pharmaceutically acceptable salts thereof.

In some embodiments, the compound is selected from the prodrugs of compounds described in Table 4 and pharmaceutically acceptable salts thereof.

In some embodiments, the compound is selected from the compounds described in Table 4.

In some embodiments, the compound is selected from the compounds described in Table 5 and prodrugs and pharmaceutically acceptable salts thereof.

In some embodiments, the compound is selected from the compounds described in Table 5 and pharmaceutically acceptable salts thereof.

In some embodiments, the compound is selected from the prodrugs of compounds described in Table 5 and pharmaceutically acceptable salts thereof.

In some embodiments, the compound is selected from the compounds described in Table 5.

TABLE 1

| Compound No. | Name | Structure |
|---|---|---|
| 1 | 1,1-difluoro-N-((4²S,4³R)- 1³,4⁴,4⁴-trifluoro-6-methyl-5- oxo-9-oxa-6-aza-4(2,1)- pyrrolidina-1(1,2),2(1,3)- dibenzenacyclononaphane-4³- yl)methanesulfonamide | 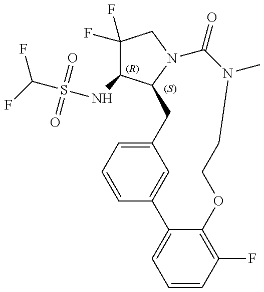 |
| 2 | 1-fluoro-N-((4²S,4³S,4⁴S)-2²- fluoro-4⁴,6-dimethyl-5-oxo-9- oxa-6-aza-4(2,1)-pyrrolidina- 1(1,2),2(1,3)- dibenzenacyclononaphane-4³- yl)methanesulfonamide | 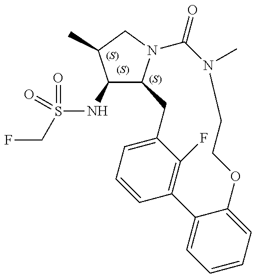 |
| 3 | N-((4²S,4³S)-2²-fluoro-5-oxo- 8-oxa-4(2,1)-piperidina- 1(1,2),2(1,3)- dibenzenacyclooctaphane-4³- yl)propane-2-sulfonamide | 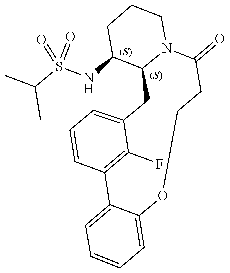 |
| 4 | 1,1-difluoro-N-((2′S,3′S)-2′- fluoro-6′-methyl-5′- oxospiro[cyclopropane-1,4′-9- oxa-6-aza-4(2,1)-pyrrolidina- 1(1,2),2(1,3)- dibenzenacyclononaphan]-3′- yl)methanesulfonamide | 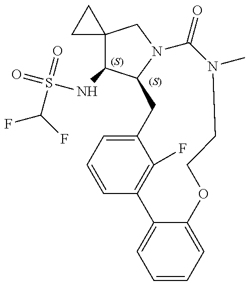 |

TABLE 2

| Compound No. | Name | Structure |
|---|---|---|
| 5 | 1,1-difluoro-N-((2′S,3′S)-2′,3′,5′- trifluoro-6′-methyl-5′- oxospiro[cyclopropane-1,4′-9- oxa-6-aza-4(2,1)-pyrrolidina- 1(1,2),2(1,3)- dibenzenacyclononaphan]-3′- yl)methanesulfonamide | 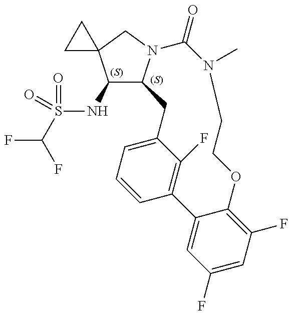 |
| 6 | 1-fluoro-N-((2′S,3′S)-2′,3′,5′- trifluoro-6′-methyl-5′- oxospiro[cyclopropane-1,4′-9- oxa-6-aza-4(2,1)-pyrrolidina- 1(1,2),2(1,3)- dibenzenacyclononaphan]-3′- yl)methanesulfonamide | 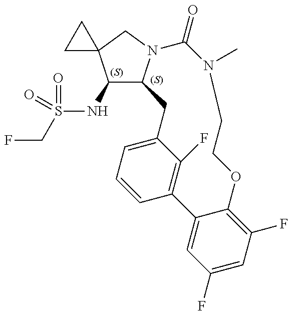 |
| 7 | 1-fluoro-N-((4²S,4³R)-1³,4⁴,4⁴- trifluoro-6-methyl-5-oxo-9-oxa- 6-aza-4(2,1)-pyrrolidina- 1(1,2),2(1,3)- dibenzenacyclononaphane-4³- yl)methanesulfonamide | 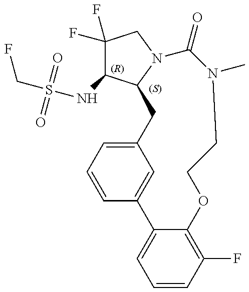 |
| 8 | N-((2′S,3′S)-2′,3′-difluoro-6′- methyl-5′- oxospiro[cyclopropane-1,4′-9- oxa-6-aza-4(2,1)-pyrrolidina- 1(1,2),2(1,3)- dibenzenacyclononaphan]-3′-yl)- 1-fluoromethanesulfonamide | 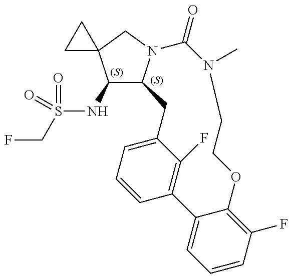 |
| 9 | N-((2′S,3′S)-2′,3′-difluoro-6′- methyl-5′- oxospiro[cyclopropane-1,4′-9- oxa-6-aza-4(2,1)-pyrrolidina- 1(1,2),2(1,3)- dibenzenacyclononaphan]-3′- yl)ethanesulfonamide | 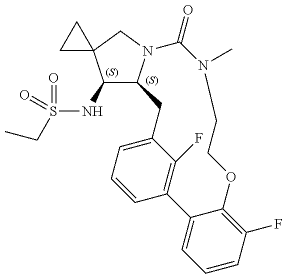 |
| 10 | N-((2′S,3′S)-6′-ethyl-5′- oxospiro[cyclopropane-1,4′-9- oxa-6-aza-4(2,1)-pyrrolidina- 1(1,2),2(1,3)- dibenzenacyclononaphan]-3′-yl)- 1,1-difluoromethanesulfonamide | 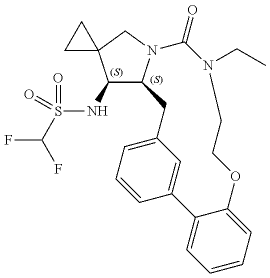 |
| 11 | 1,1-difluoro-N-((4²S,4³R,4⁴S)- 1³,1⁵,2²,4⁴-tetrafluoro-6-methyl- 5-ox0-9-oxa-6-aza-4(2,1)- pyrrolidina-1(1,2),2(1,3)- dibenzenacyclononaphane-4³- yl)methanesulfonamide | 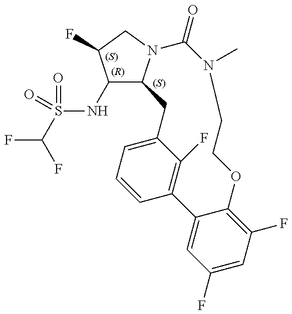 |
| 12 | 1-fluoro-N-((4²S,4³R,4⁴S)- 1³,2²,4⁴-trifluoro-6-methyl-5- oxo-9-oxa-6-aza-4(2,1)- pyrrolidina-1(1,2),2(1,3)- dibenzenacyclononaphane-4³- yl)methanesulfonamide | 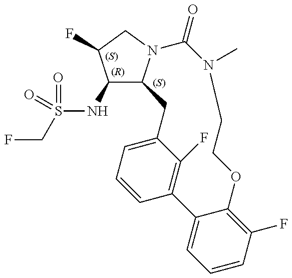 |
| 13 | N-((4²S,4³S)-1³,1⁵-difluoro-5- oxo-8-oxa-4(2,1)-piperidina- 1(1,2),2(1,3)- dibenzenacyclooctaphane-4³-yl)- 1-fluoromethanesulfonamide | 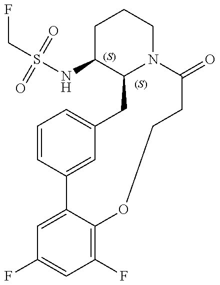 |
| 14 | N-((4²S,4³S)-1³,1⁵-difluoro-5- oxo-8-oxa-4(2,1)-piperidina- 1(1,2),2(1,3)- dibenzenacyclooctaphane-4³-yl)- 1,1-difluoromethanesulfonamide | 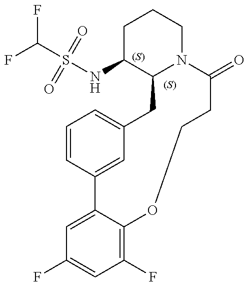 |
| 15 | N-((4²S,4³R,4⁴S)-4⁴-fluoro-6- methyl-5-oxo-9-oxa-6-aza- 4(2,1)-pyrrolidina-1(1,2),2(1,3)- dibenzenacyclononaphane-4³- yl)ethanesulfonamide | 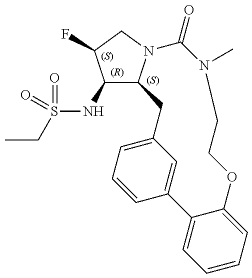 |
| 16 | N-((4²S,4³R,4⁴S)-1³,4⁴-difluoro- 6-methyl-5-oxo-9-oxa-6-aza- 4(2,1)-pyrrolidina-1(1,2),2(1,3)- dibenzenacyclononaphane-4³- yl)ethanesulfonamide | 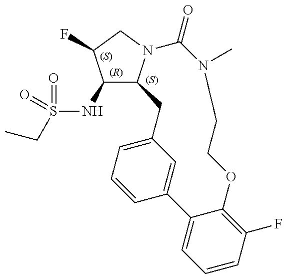 |
| 17 | N-((4²S,4³R,4⁴S)-2²,4⁴-difluoro- 6-methyl-5-oxo-9-oxa-6-aza- 4(2,1)-pyrrolidina-1(1,2),2(1,3)- dibenzenacyclononaphane-4³- yl)ethanesulfonamide | 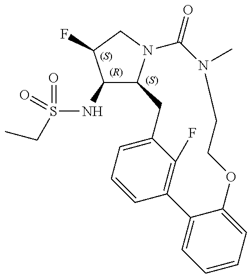 |
| 18 | N-((4²S,4³R,4⁴S)-1³,2²,4⁴- trifluoro-6-methyl-5-oxo-9-oxa- 6-aza-4(2,1)-pyrrolidina- 1(1,2),2(1,3)- dibenzenacyclononaphane-4³- yl)ethanesulfonamide | 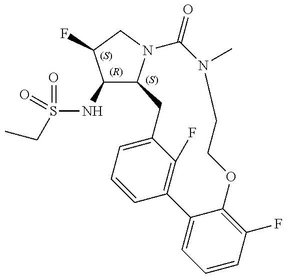 |
| 19 | N-((2′S,3′S)-3′-fluoro-6′-methyl- 5′-oxospiro[cyclopropane-1,4′-9- oxa-6-aza-4(2,1)-pyrrolidina- 1(1,2),2(1,3)- dibenzenacyclononaphan]-3′- yl)ethanesulfonamide | 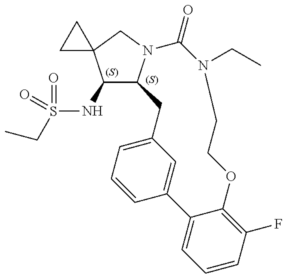 |
| 20 | N-((4²S,4³R,4⁴S)-1³,4⁴-difluoro- 6-methyl-5-oxo-9-oxa-6-aza- 4(2,1)-pyrrolidina-1(1,2),2(1,3)- dibenzenacyclononaphane-4³- yl)-1,1- difluoromethanesulfonamide | 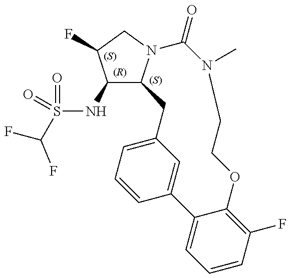 |
| 21 | N-((4²S,4³R,4⁴S)-1³,4⁴-difluoro- 6-methyl-5-oxo-9-oxa-6-aza- 4(2,1)-pyrrolidina-1(1,2),2(1,3)- dibenzenacyclononaphane-4³- yl)-1-fluoromethanesulfonamide | 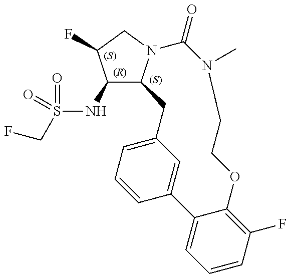 |
| 22 | N-((2′S,3′S)-6′-(2,2- difluoroethyl)-3′-fluoro-5′- oxospiro[cyclopropane-1,4′-9- oxa-6-aza-4(2,1)-pyrrolidina- 1(1,2),2(1,3)- dibenzenacyclononaphan]-3′-yl)- 1-fluoromethanesulfonamide | 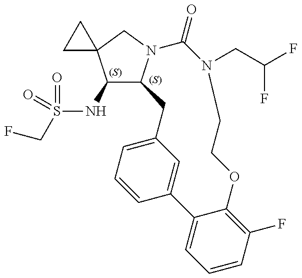 |
| 23 | N-((4²S,4³R)-4⁴,4⁴-difluoro-6- methyl-5-oxo-9-oxa-6-aza- 4(2,1)-pyrrolidina-1(1,2),2(1,3)- dibenzenacyclononaphane-4³- yl)ethanesulfonamide | 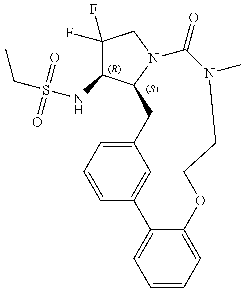 |
| 24 | 1,1-difluoro-N-((4²S,4³S)-1³- fluoro-5-oxo-8-oxa-4(2,1)- piperidina-1(1,2),2(1,3)- dibenzenacyclooctaphane-4³- yl)methanesulfonamide | 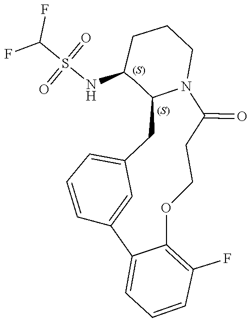 |
| 25 | N-((4²S,4³R,4⁴S)-4⁴-fluoro-6- methyl-5-oxo-9-oxa-6-aza- 4(2,1)-pyrrolidina-1(1,2),2(1,3)- dibenzenacyclononaphane-4³- yl)-N′,N′-dimethylsulfamide | 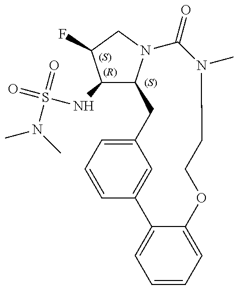 |
| 26 | 1-fluoro-N-((2′S,3′S)-2′-fluoro- 6′-methyl-5′- oxospiro[cyclopropane-1,4′-9- oxa-6-aza-4(2,1)-pyrrolidina- 1(1,2),2(1,3)- dibenzenacyclononaphan]-3′- yl)methanesulfonamide | 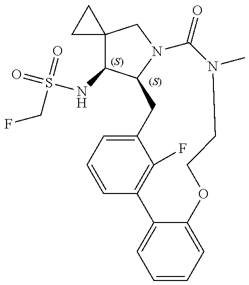 |
| 27 | 1,1-difluoro-N-((4²S,4³S)-5-oxo- 8-oxa-4(2,1)-piperidina- 1(1,2),2(1,3)- dibenzenacyclooctaphane-4³- yl)methanesulfonamide | 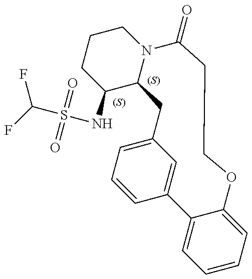 |
| 28 | N-((4²S,4³S)-2²-fluoro-5-oxo-8- oxa-4(2,1)-piperidina- 1(1,2),2(1,3)- dibenzenacyclooctaphane-4³- yl)ethanesulfonamide | 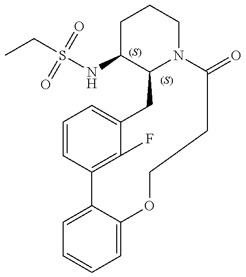 |

TABLE 3

| Compound | | |
|---|---|---|
| No. | Name | Structure |
| 29 | 2-fluoro-N-((2′S,3′S)-6′-methyl- 5′-oxospiro[cyclopropane-1,4′-9- oxa-6-aza-4(2,1)-pyrrolidina- 1(1,2),2(1,3)- dibenzenacyclononaphan]-3′- yl)-propane-2-sulfonamide | 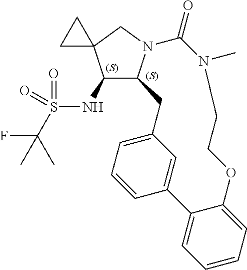 |
| 30 | N-((4²S,4³R)-13,44,44-trifluoro- 6-methyl-5-oxo-9-oxa-6-aza- 4(2,1)-pyrrolidina-1(1,2),2(1,3)- dibenzenacyclononaphane-4³- yl)ethanesulfonamide | 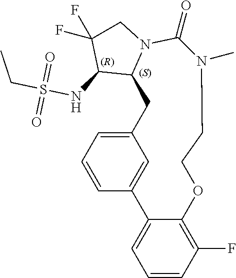 |
| 31 | N-((2′S,3′S)-2′-fluoro-6′-methyl- 5′-oxospiro[cyclopropane-1,4′-9- oxa-6-aza-4(2,1)-pyrrolidina- 1(1,2),2(1,3)- dibenzenacyclononaphan]-3′- yl)ethanesulfonamide | 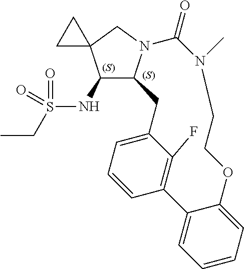 |
| 32 | 1-fluoro-N-((4²S,4³S)-2²-fluoro- 1³-methyl-5-oxo-8-oxa-4(2,1)- piperidina-1(1,2),2(1,3)- dibenzenacyclooctaphane-4³- yl)methanesulfonamide | 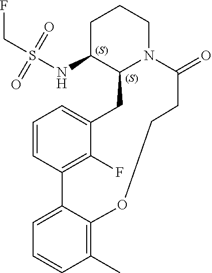 |
| 33 | N-((2′S,3′S)-6′-methyl-5′- oxospiro[cyclopropane-1,4′-9- oxa-6-aza-4(2,1)-pyrrolidina- 1(1,2),2(1,3)- dibenzenacyclononaphan]-3′- yl)ethanesulfonamide | 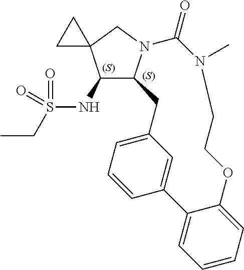 |
| 34 | N-((2′S,3′S)-6′-(2,2- difluoroethyl)-5′- oxospiro[cyclopropane-1,4′-9- oxa-6-aza-4(2,1)-pyrrolidina- 1(1,2),2(1,3)- dibenzenayclononaphan]-3′-yl)- 1-fluoromethanesulfonamide | 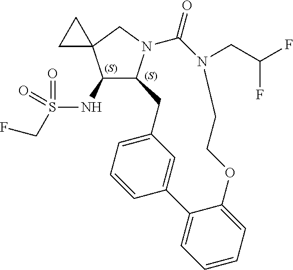 |
| 35 | 1,1-difluoro-N-((4²S,4³S)-2²- fluoro-1³-methyl-5-oxo-8-oxa- 4(2,1)-piperidina-1(1,2),2(1,3)- dibenzenacyclooctaphane-4³- yl)methanesulfonamide | 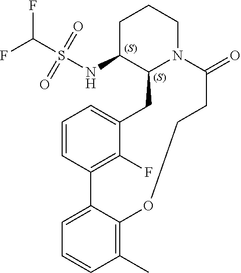 |
| 36 | 1,1-difluoro-N-((2′S,3′S)-6′- fluoro-6′-methyl-5′- oxospiro[cyclopropane-1,4′-9- oxa-6-aza-4(2,1)-pyrrolidina- 1(1,2),2(1,3)- dibenzenacyclononaphan]-3′- yl)methanesulfonamide | 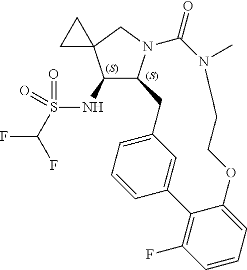 |
| 37 | 1,1-difluoro-N-((2′S,3′S)-5′- fluoro-6′-methyl-5′- oxospiro[cyclopropane-1,4′-9- oxa-6-aza-4(2,1)-pyrrolidina- 1(1,2),2(1,3)- dibenzenacyclononaphan]-3′- yl)methanesulfonamide | 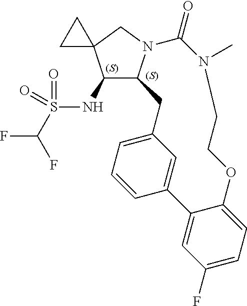 |
| 38 | 1-fluoro-N-((2′S,3′S)-6′-methyl- 5′-oxospiro[cyclopropane-1,4′-9- oxa-6-aza-4(2,1)-pyrrolidina- 1(1,2),2(1,3)- dibenzenacyclononaphan]-3′- yl)cyclopropane-1-sulfonamide | 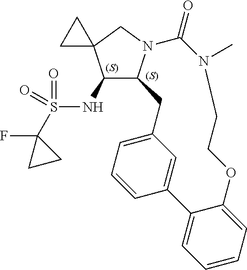 |
| 39 | N-((2′S,3′S)-2′-fluoro-6′-methyl- 5′-xospiro[cyclopropane-1,4′-9- oxa-6-aza-4(2,1)-pyrrolidina- 1(1,2),2(1,3)- dibenzenacyclononaphan]-3′- yl)propane-2-sulfonamide | 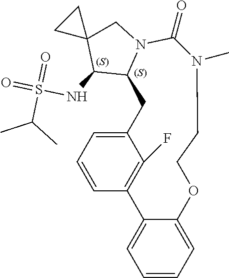 |

TABLE 4

| Compound | | |
|---|---|---|
| No. | Name | Structure |
| 40 | 1-fluoro-N-(5-oxo-8-oxa- 4(2,1)-piperidina- 1(1,2),2(1,3)- dibenzenacyclooctaphane- 4³-yl)ethane-1-sulfonamide | 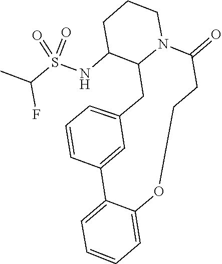 [SMILES: CC(F)S(=O)(=O)NC1CCCN2C(=O)CCOc3ccccc3-c3cccc(c3)CC12] |
| 41 | 1,1-difluoro-N-(6-methyl-5- oxo-9-oxa-6-aza-4(2,1)- pyrrolidina-1(1,2),2(1,3)- dibenzenacyclononaphane- 4³-yl)methanesulfonamide | 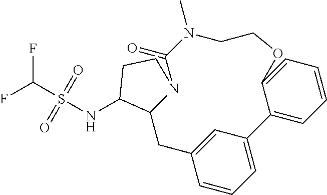 [SMILES: CN1CCOc2ccccc2-c2cccc(c2)CC2C(NS(=O)(=O)C(F)F)CCN2C1=O] |
| 42 | 1,1-difluoro-N-(1³-fluoro-6- methyl-5-oxo-9-oxa-6-aza- 4(2,1)-pyrrolidina- 1(1,2),2(1,3)- dibenzenacyclononaphane- 4³-yl)-methanesulfonamide | 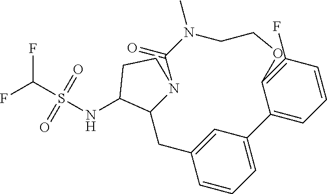 [SMILES: CN1CCOc2c(F)cccc2-c2cccc(c2)CC2C(NS(=O)(=O)C(F)F)CCN2C1=O] |
| 43 | N-(2²,4⁴-difluoro-5-oxo-9- oxa-4(2,1)-pyrrolidina- 1(1,2),2(1,3)- dibenzenacyclononaphane- 4³-yl)-ethanesulfonamide | 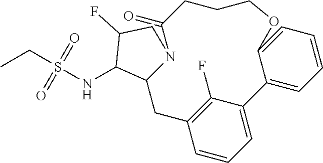 [SMILES: CCS(=O)(=O)NC1C(F)CN2C(=O)CCCOc3ccccc3-c3cccc(c3F)CC12] |
| 44 | N-(6′-(2,2-difluoroethyl)-5′- oxospiro[cyclopropane-1,4′- 9-oxa-6-aza-4(2,1)- pyrrolidina-1(1,2),2(1,3)- dibenzenacyclononaphan]- 3′-yl)-1,1- difluoromethanesulfonamide | 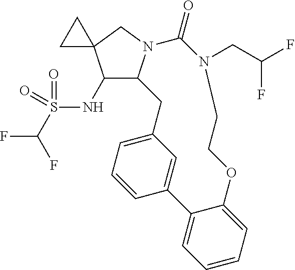 [SMILES: O=C1N(CC(F)F)CCOc2ccccc2-c2cccc(c2)CC2C(NS(=O)(=O)C(F)F)C3(CC3)CN12] |
| 45 | 1-fluoro-N-(2²-fluoro-4⁴- methyl-5-oxo-8-oxa-4(2,1)- piperidina-1(1,2),2(1,3)- dibenzenacyclooctaphane- 4³-yl)methanesulfonamide | 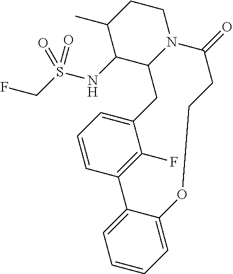 [SMILES: CC1CCN2C(=O)CCOc3ccccc3-c3cccc(c3F)CC2C1NS(=O)(=O)CF] |
| 46 | N-(2²-fluoro-5-oxo-8-oxa- 4(2,1)-piperidina- 1(1,2),2(1,3)- dibenzenacyclooctaphane- 4³-yl)-1- methylcyclopropane-1- sulfonamide | 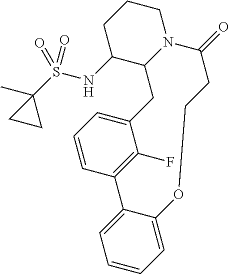 [SMILES: CC1(S(=O)(=O)NC2CCCN3C(=O)CCOc4ccccc4-c4cccc(c4F)CC23)CC1] |
| 47 | N-(2²-fluoro-5-oxo-8-oxa- 4(2,1)-piperidina- 1(1,2),2(1,3)- dibenzenacyclooctaphane- 4³-yl)-2-methylpropane-2- sulfonamide | 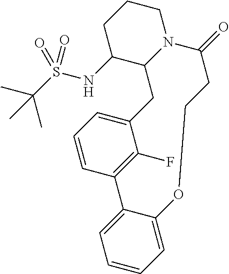 [SMILES: CC(C)(C)S(=O)(=O)NC1CCCN2C(=O)CCOc3ccccc3-c3cccc(c3F)CC12] |
| 48 | 1-fluoro-N-(1³,4⁴,4⁴- trifluoro-6-methyl-5-oxo-9- oxa-6-aza-4(2,1)- pyrrolidina-1(1,2),2(1,3)- dibenzenacyclononaphane- 4³-yl)ethane-1-sulfonamide | 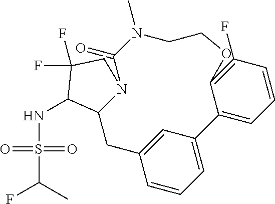 [SMILES: CC(F)S(=O)(=O)NC1C2Cc3cccc(c3)-c3cccc(F)c3OCCN(C)C(=O)N2CC1(F)F] |
| 49 | 1,1-difluoro-N-(1³,2²,4⁴- trifluoro-6-methyl-5-oxo-9- oxa-6-aza-4(2,1)- pyrrolidina-1(1,2),2(1,3)- dibenzenacyclononaphane- 4³-yl)ethane-1-sulfonamide | 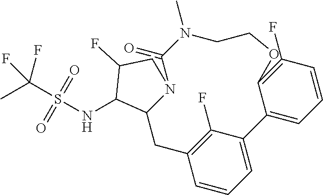 [SMILES: CN1CCOc2c(F)cccc2-c2cccc(c2F)CC2C(NS(=O)(=O)C(C)(F)F)C(F)CN2C1=O] |
| 50 | N-(1³,1⁵-difluoro-5-oxo-8- oxa-4(2,1)-piperidina- 1(1,2),2(1,3)- dibenzenacyclooctaphane- 4³-yl)-1,1-difluoroethane-1- sulfonamide | 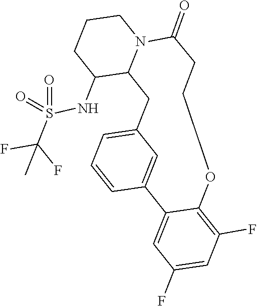 [SMILES: CC(F)(F)S(=O)(=O)NC1CCCN2C(=O)CCOc3c(F)cc(F)cc3-c3cccc(c3)CC12] |
| 51 | N-(1³,1⁵-difluoro-5-oxo-8- oxa-4(2,1)-piperidina- 1(1,2),2(1,3)- dibenzenacyclooctaphane- 4³-yl)-1-fluorocyclopropane- 1-sulfonamide | 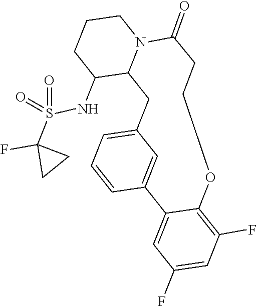 [SMILES: O=C1CCOc2c(F)cc(F)cc2-c2cccc(c2)CC2C(NS(=O)(=O)C3(F)CC3)CCCN12] |
| 52 | N-(1³,1⁵-difluoro-5-oxo-8- oxa-4(2,1)-piperidina- 1(1,2),2(1,3)- dibenzenacyclooctaphane- 4³-yl)-2-fluoropropane-2- sulfonamide | 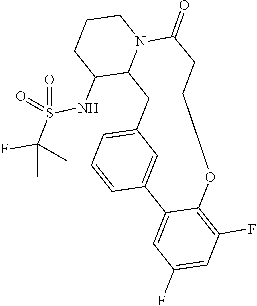 [SMILES: CC(C)(F)S(=O)(=O)NC1CCCN2C(=O)CCOc3c(F)cc(F)cc3-c3cccc(c3)CC12] |
| 53 | N-(1³,1⁵-difluoro-5-oxo-8- oxa-4(2,1)-piperidina- 1(1,2),2(1,3)- dibenzenacyclooctaphane- 4³-yl)-1-fluoroethane-1- sulfonamide | 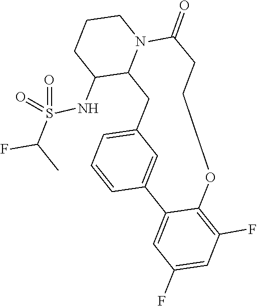 [SMILES: CC(F)S(=O)(=O)NC1CCCN2C(=O)CCOc3c(F)cc(F)cc3-c3cccc(c3)CC12] |
| 54 | 1,1-difluoro-N-(1³,2²,4⁴- trifluoro-5-oxo-8-oxa- 4(2,1)-piperidina- 1(1,2),2(1,3)- dibenzenacyclooctaphane- 4³-yl)methanesulfonamide | 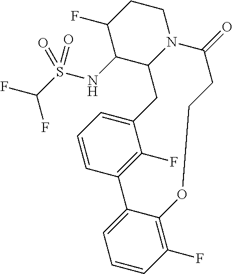 [SMILES: O=C1CCOc2c(F)cccc2-c2cccc(c2F)CC2C(NS(=O)(=O)C(F)F)C(F)CCN12] |
| 55 | N-(2²,4⁴-difluoro-6-methyl- 5-oxo-9-oxa-6-aza-4(2,1)- pyrrolidina-1(1,2),2(1,3)- dibenzenacyclononaphane- 4³-yl)-1,1-difluoroethane-1- sulfonamide | 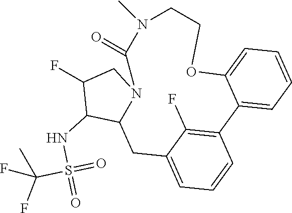 [SMILES: CN1CCOc2ccccc2-c2cccc(c2F)CC2C(NS(=O)(=O)C(C)(F)F)C(F)CN2C1=O] |
| 56 | 1,1-difluoro-N-(4⁴-fluoro-6- methyl-5-oxo-9-oxa-6-aza- 4(2,1)-pyrrolidina- 1(1,2),2(1,3)- dibenzenacyclononaphane- 4³-yl)ethane-1-sulfonamide | 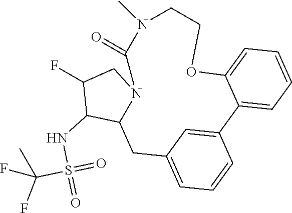 [SMILES: CN1CCOc2ccccc2-c2cccc(c2)CC2C(NS(=O)(=O)C(C)(F)F)C(F)CN2C1=O] |
| 57 | N-(8′,8′-difluoro-6′-methyl- 5′-oxospiro[cyclopropane- 1,4′-9-oxa-6-aza-4(2,1)- pyrrolidina-1(1,2),2(1,3)- dibenzenacyclononaphan]- 3′-yl)-1- fluoromethanesulfonamide | 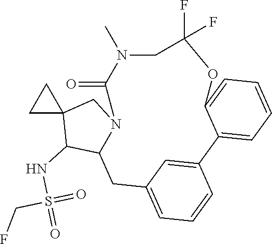 [SMILES: CN1CC(F)(F)Oc2ccccc2-c2cccc(c2)CC2C(NS(=O)(=O)CF)C3(CC3)CN2C1=O] |
| 58 | 1,1-difluoro-N-(1³-fluoro-5- oxo-8-oxa-4(2,1)-piperidina- 1(1,2),2(1,3)- dibenzenacyclooctaphane- 4³-yl)ethane-1-sulfonamide | 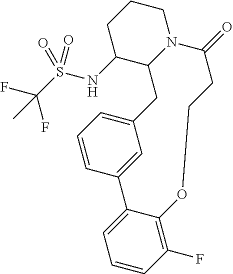 [SMILES: CC(F)(F)S(=O)(=O)NC1CCCN2C(=O)CCOc3c(F)cccc3-c3cccc(c3)CC12] |
| 59 | 1,1-difluoro-N-(6′-methyl- 5′-oxospiro[cyclopropane- 1,4′-9-oxa-6-aza-4(2,1)- pyrrolidina-1(1,2),2(1,3)- dibenzenacyclononaphan]- 3′-yl)ethane-1-sulfonamide | 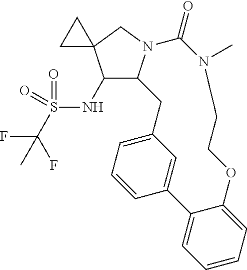 [SMILES: CN1CCOc2ccccc2-c2cccc(c2)CC2C(NS(=O)(=O)C(C)(F)F)C3(CC3)CN2C1=O] |
| 60 | N-(2²,4⁴-difluoro-6-methyl- 5-oxo-9-oxa-6-aza-4(2,1)- pyrrolidina-1(1,2),2(1,3)- dibenzenacyclononaphane- 4³-yl)-2-fluoropropane-2- sulfonamide | 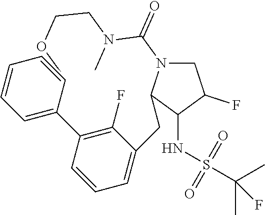 [SMILES: COCCN(C)C(=O)N1CC(F)C(NS(=O)(=O)C(C)(C)F)C1Cc1cccc(-c2ccccc2)c1F] |
| 61 | N-(2²,4⁴-difluoro-6-methyl- 5-oxo-9-oxa-6-aza-4(2,1)- pyrrolidina-1(1,2),2(1,3)- dibenzenacyclononaphane- 4³-yl)-1-fluorocyclopropane- 1-sulfonamide | 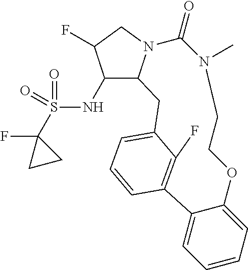 [SMILES: CN1CCOc2ccccc2-c2cccc(c2F)CC2C(NS(=O)(=O)C3(F)CC3)C(F)CN2C1=O] |
| 62 | 1-fluoro-N-(4⁴-fluoro-6- methyl-5-oxo-9-oxa-6-aza- 4(2,1)-pyrrolidina- 1(1,2),2(1,3)- dibenzenacyclononaphane- 4³-yl)cyclopropane-1- sulfonamide | 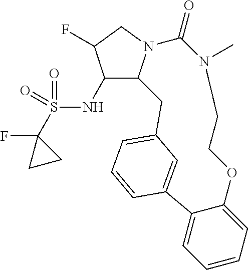 [SMILES: CN1CCOc2ccccc2-c2cccc(c2)CC2C(NS(=O)(=O)C3(F)CC3)C(F)CN2C1=O] |
| 63 | 1-fluoro-N-(1³,1⁵,2²- trifluoro-7-methyl-5-oxo-8- oxa-4(2,1)-piperidina- 1(1,2),2(1,3)- dibenzenacyclooctaphane- 4³-yl)methanesulfonamide | 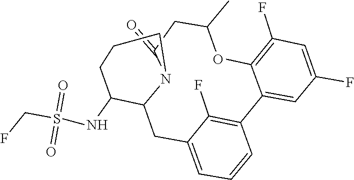 |
| 64 | 1-fluoro-N-(2²-fluoro-6- hydroxy-6-methyl-5-oxo-8- oxa-4(2,1)-piperidina- 1(1,2),2(1,3)- dibenzenacyclooctaphane- 4³-yl)methanesulfonamide | 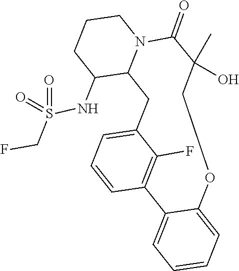 [SMILES: CC1(O)COc2ccccc2-c2cccc(c2F)CC2C(NS(=O)(=O)CF)CCCN2C1=O] |
| 65 | 1-fluoro-N-(2²-fluoro-6- methyl-5-oxo-6-aza-4(2,1)- piperidina-1(1,2),2(1,3)- dibenzenacyclooctaphane- 4³-yl)methanesulfonamide | 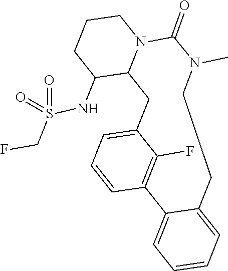 [SMILES: CN1CCc2ccccc2-c2cccc(c2F)CC2C(NS(=O)(=O)CF)CCCN2C1=O] |
| 66 | N-(1³,2²-difluoro-5-oxo-8- oxa-4(2,1)-piperidina- 1(1,2),2(1,3)- dibenzenacyclooctaphane- 4³-yl)-1-fluorocyclopropane- 1-sulfonamide | 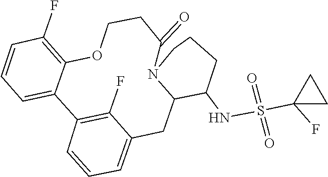 [SMILES: O=C1CCOc2c(F)cccc2-c2cccc(c2F)CC2C(NS(=O)(=O)C3(F)CC3)CCCN12] |
| 66A | N-[(4²S,4³S)-1³,2²-difluoro- 5-oxo-8-oxa-4(2,1)- piperidina-1(1,2),2(1,3)- dibenzenacyclooctaphane- 4³-yl]-1-fluoro- cyclopropane-1-sulfonamide | 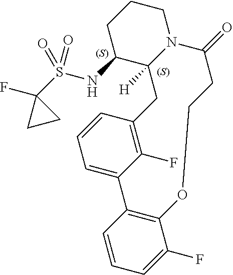 |
| 67 | 1-fluoro-N-(1³,1⁵,2²- trifluoro-5-oxo-8-oxa- 4(2,1)-piperidina- 1(1,2),2(1,3)- dibenzenacyclooctaphane- 4³-yl)cyclopropane-1- sulfonamide | 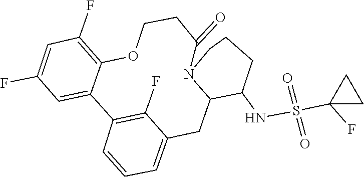 [SMILES: O=C1CCOc2c(F)cc(F)cc2-c2cccc(c2F)CC2C(NS(=O)(=O)C3(F)CC3)CCCN12] |
| 67A | 1-fluoro-N-[(4²S,4³S)- 1³,1⁵,2²--trifluoro-5-oxo-8- oxa-4(2,1)-piperidina- 1(1,2),2(1,3)- dibenzenacyclooctaphane- 4³-yl] cyclopropane-1- sulfonamide | 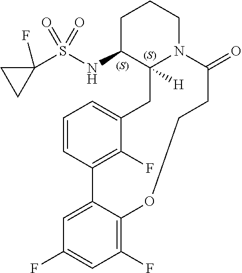 |
| 68 | 1,1-difluoro-N-(1³,4⁴,4⁴- trifluoro-5-oxo-8-oxa- 4(2,1)-piperidina- 1(1,2),2(1,3)- dibenzenacyclooctaphane- 4³-yl)ethanesulfonamide | 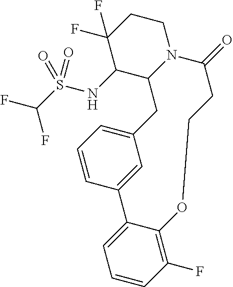 [SMILES: O=C1CCOc2c(F)cccc2-c2cccc(c2)CC2C(NS(=O)(=O)C(F)F)C(F)(F)CCN12] |
| 69 | N-(1³,4⁴-difluoro-5-oxo-8- oxa-4(2,1)-piperidina- 1(1,2),2(1,3)- dibenzenacyclooctaphane- 4³-yl)-1,1- difluoromethanesulfonamide | 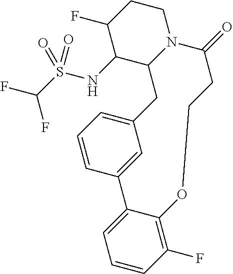 [SMILES: O=C1CCOc2c(F)cccc2-c2cccc(c2)CC2C(NS(=O)(=O)C(F)F)C(F)CCN12] |
| 70 | N-(2⁴,4⁴-difluoro-5-oxo-8- oxa-4(2,1)-piperidina- 1(1,2),2(1,3)- dibenzenacyclooctaphane- 4³-yl)-1- fluoromethanesulfonamide | 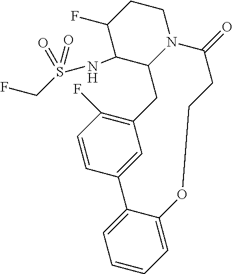 [SMILES: O=C1CCOc2ccccc2-c2ccc(F)c(c2)CC2C(NS(=O)(=O)CF)C(F)CCN12] |
| 71 | 1-fluoro-N-(1³,4⁴,4⁴- trifluoro-5-oxo-8-oxa- 4(2,1)-piperidina- 1(1,2),2(1,3)- dibenzenacyclooctaphane- 4³-yl)methanesulfonamide | 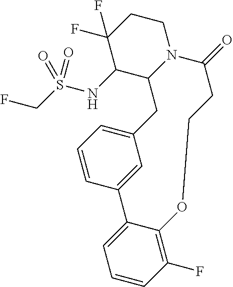 [SMILES: O=C1CCOc2c(F)cccc2-c2cccc(c2)CC2C(NS(=O)(=O)CF)C(F)(F)CCN12] |
| 72 | N-(1³,4⁴-difluoro-5-oxo-8- oxa-4(2,1)-piperidina- 1(1,2),2(1,3)- dibenzenacyclooctaphane- 4³-yl)-1- fluoromethanesulfonamide | 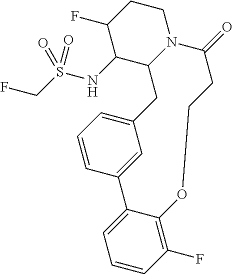 [SMILES: O=C1CCOc2c(F)cccc2-c2cccc(c2)CC2C(NS(=O)(=O)CF)C(F)CCN12] |
| 73 | N-(1³,1⁵,2²,4⁴,4⁴- pentafluoro-5-oxo-8-oxa- 4(2,1)-piperidina- 1(1,2),2(1,3)- dibenzenacyclooctaphane- 4³-yl)methanesulfonamide | 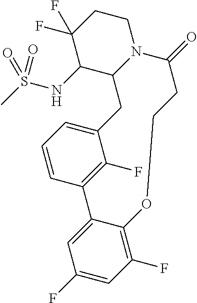 [SMILES: CS(=O)(=O)NC1C2Cc3cccc(c3F)-c3cc(F)cc(F)c3OCCC(=O)N2CCC1(F)F] |
| 74 | 1-fluoro-N-(1³,1⁵,2²,4⁴,4⁴- pentafluoro-5-oxo-8-oxa- 4(2,1)-piperidina- 1(1,2),2(1,3)- dibenzenacyclooctaphane- 43-yl)methanesulfonamide | 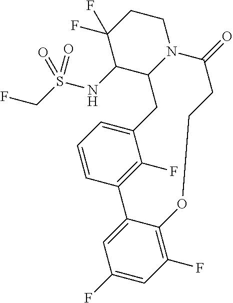 [SMILES: O=C1CCOc2c(F)cc(F)cc2-c2cccc(c2F)CC2C(NS(=O)(=O)CF)C(F)(F)CCN12] |
| 75 | 1,1-difluoro-N- (1³,1⁵,2²,4⁴,4⁴-pentafluoro-5- oxo-8-oxa-4(2,1)-piperidina- 1(1,2),2(1,3)- dibenzenacyclooctaphane- 4³-yl)methanesulfonamide | 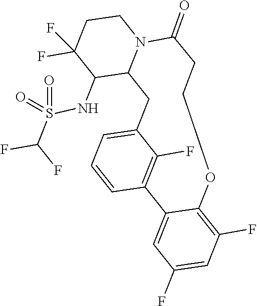 [SMILES: O=C1CCOc2c(F)cc(F)cc2-c2cccc(c2F)CC2C(NS(=O)(=O)C(F)F)C(F)(F)CCN12] |
| 76 | 1,1-difluoro-N-(1³,1⁵,2²,4⁴- tetrafluoro-5-oxo-8-oxa- 4(2,1)-piperidina- 1(1,2),2(1,3)- dibenzenacyclooctaphane- 4³-yl)methanesulfonamide | 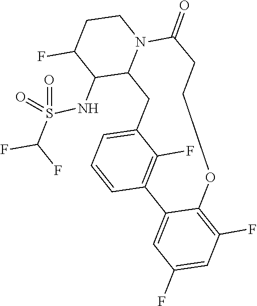 [SMILES: O=C1CCOc2c(F)cc(F)cc2-c2cccc(c2F)CC2C(NS(=O)(=O)C(F)F)C(F)CCN12] |
| 77 | 1-fluoro-N-(1³,2²,4⁴- trifluoro-5-oxo-8-oxa- 4(2,1)-piperidina- 1(1,2),2(1,3)- dibenzenacyclooctaphane- 4³-yl)methanesulfonamide | 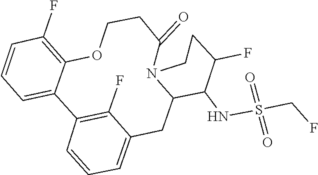 [SMILES: O=C1CCOc2c(F)cccc2-c2cccc(c2F)CC2C(NS(=O)(=O)CF)C(F)CCN12] |
| 78 | 1-fluoro-N-(1³,2²,4⁴,4⁴- tetrafluoro-5-oxo-8-oxa- 4(2,1)-piperidina- 1(1,2),2(1,3)- dibenzenacyclooctaphane- 4³-yl)methanesulfonamide | 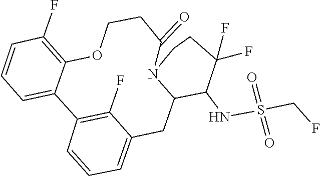 [SMILES: O=C1CCOc2c(F)cccc2-c2cccc(c2F)CC2C(NS(=O)(=O)CF)C(F)(F)CCN12] |
| 79 | 1,1-difluoro-N-(2²,4⁴,4⁴- trifluoro-5-oxo-8-oxa- 4(2,1)-piperidina- 1(1,2),2(1,3)- dibenzenacyclooctaphane- 4³-yl)methanesulfonamide | 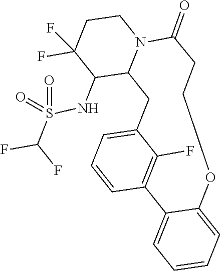 [SMILES: O=C1CCOc2ccccc2-c2cccc(c2F)CC2C(NS(=O)(=O)C(F)F)C(F)(F)CCN12] |
| 80 | N-(4⁴,4⁴-difluoro-5-oxo-8- oxa-4(2,1)-piperidina- 1(1,2),2(1,3)- dibenzenacyclooctaphane- 4³-yl)-1,1- difluoromethanesulfonamide | 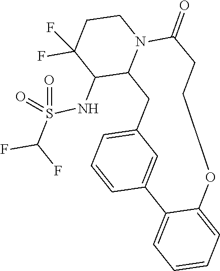 [SMILES: O=C1CCOc2ccccc2-c2cccc(c2)CC2C(NS(=O)(=O)C(F)F)C(F)(F)CCN12] |
| 81 | N-(4⁴,4⁴-difluoro-5-oxo-8- oxa-4(2,1)-piperidina- 1(1,2),2(1,3)- dibenzenacyclooctaphane- 4³-yl)-1- fluoromethanesulfonamide | 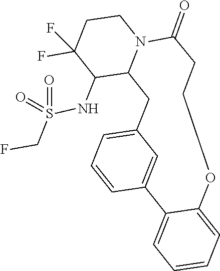 [SMILES: O=C1CCOc2ccccc2-c2cccc(c2)CC2C(NS(=O)(=O)CF)C(F)(F)CCN12] |
| 82 | N-(2²,4⁴-difluoro-5-oxo-8- oxa-4(2,1)-piperidina- 1(1,2),2(1,3)- dibenzenacyclooctaphane- 4³-yl)-1,1- difluoromethanesulfonamide | 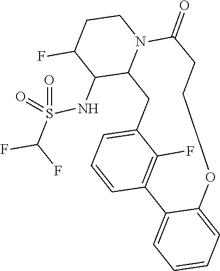 [SMILES: O=C1CCOc2ccccc2-c2cccc(c2F)CC2C(NS(=O)(=O)C(F)F)C(F)CCN12] |
| 83 | N-(2²,4⁴-difluoro-5-oxo-8- oxa-4(2,1)-piperidina- 1(1,2),2(1,3)- dibenzenacyclooctaphane- 4³-yl)-1- fluoromethanesulfonamide | 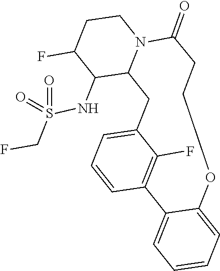 [SMILES: O=C1CCOc2ccccc2-c2cccc(c2F)CC2C(NS(=O)(=O)CF)C(F)CCN12] |
| 84 | 1,1-difluoro-N-(4⁴-fluoro-5- oxo-8-oxa-4(2,1)-piperidina- 1(1,2),2(1,3)- dibenzenacyclooctaphane- 4³-yl)methanesulfonamide | 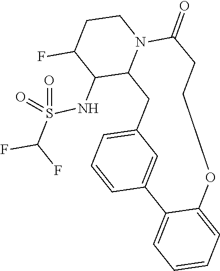 [SMILES: O=C1CCOc2ccccc2-c2cccc(c2)CC2C(NS(=O)(=O)C(F)F)C(F)CCN12] |
| 85 | N-(2²,4⁴-difluoro-6-methyl- 5-oxo-9-oxa-6-aza-4(2,1)- pyrrolidina-1(1,2),2(1,3)- dibenzenacyclononaphane- 4³-yl)-1-fluoroethane-1- sulfonamide | 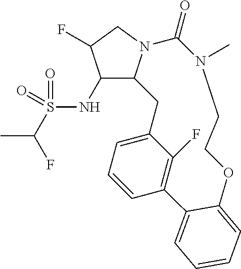 [SMILES: CC(F)S(=O)(=O)NC1C(F)CN2C(=O)N(C)CCOc3ccccc3-c3cccc(c3F)CC12] |
| 86 | 1-fluoro-N-(1³,2²,4⁴- trifluoro-6-methyl-5-oxo-9- oxa-6-aza-4(2,1)- pyrrolidina-1(1,2),2(1,3)- dibenzenacyclononaphane- 4³-yl)ethane-1-sulfonamide | 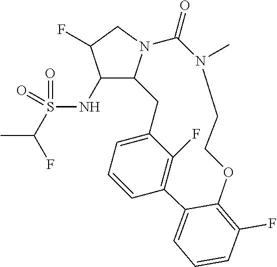 [SMILES: CC(F)S(=O)(=O)NC1C(F)CN2C(=O)N(C)CCOc3c(F)cccc3-c3cccc(c3F)CC12] |
| 87 | 1,1-difluoro-N- (1³,1⁵,2²,4⁴,4⁴-pentafluoro-6- methyl-5-oxo-9-oxa-6-aza- 4(2,1)-pyrrolidina- 1(1,2),2(1,3)- dibenzenacyclononaphane- 4³-yl)methanesulfonamide | 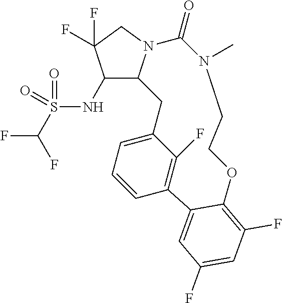 [SMILES: CN1CCOc2c(F)cc(F)cc2-c2cccc(c2F)CC2C(NS(=O)(=O)C(F)F)C(F)(F)CN2C1=O] |
| 88 | 1,1-difluoro-N-(2²,2⁵,4⁴- trifluoro-6-methyl-5-oxo-9- oxa-6-aza-4(2,1)- pyrrolidina-1(1,2),2(1,3)- dibenzenacyclononaphane- 4³-yl)methanesulfonamide | 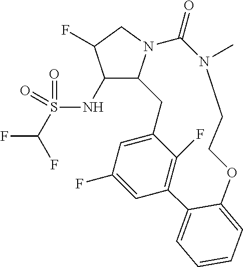 [SMILES: CN1CCOc2ccccc2-c2cc(F)cc(c2F)CC2C(NS(=O)(=O)C(F)F)C(F)CN2C1=O] |
| 89 | 1-fluoro-N-(2′-fluoro-6′- methyl-5′- oxospiro[cyclopropane-1,4′- 9-oxa-6-aza-4(2,1)- pyrrolidina-1(1,2),2(1,3)- dibenzenacyclononaphan]- 3′-yl)ethane-1-sulfonamide | 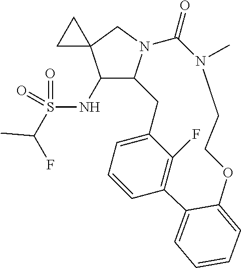 [SMILES: CC(F)S(=O)(=O)NC1C2Cc3cccc(c3F)-c3ccccc3OCCN(C)C(=O)N2CC12CC2] |
| 90 | 1-fluoro-N-(2²-fluoro-5-oxo- 4(2,1)-piperidina- 1(1,2),2(1,3)- dibenzenacyclooctaphane- 4³-yl)methanesulfonamide | 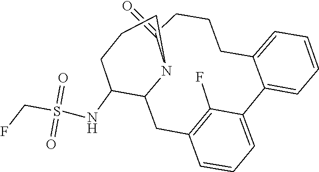 [SMILES: O=C1CCCc2ccccc2-c2cccc(c2F)CC2C(NS(=O)(=O)CF)CCCN12] |
| 91 | N-(4⁴,4⁴-difluoro-5-oxo-8- oxa-4(2,1)-pyrrolidina- 1(1,2),2(1,3)- dibenzenacyclooctaphane- 4³-yl)-1,1- difluoromethanesulfonamide | 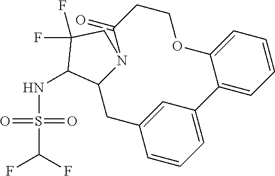 [SMILES: O=C1CCOc2ccccc2-c2cccc(c2)CC2C(NS(=O)(=O)C(F)F)C(F)(F)CN12] |
| 92 | 1,1-difluoro-N-(2²,4⁴,4⁴- trifluoro-5-oxo-8-oxa- 4(2,1)-pyrrolidina- 1(1,2),2(1,3)- dibenzenacyclooctaphane- 4³-yl)methanesulfonamide | 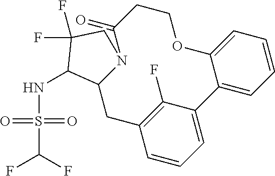 [SMILES: O=C1CCOc2ccccc2-c2cccc(c2F)CC2C(NS(=O)(=O)C(F)F)C(F)(F)CN12] |
| 93 | 1-fluoro-N-(2²,4⁴,4⁴- trifluoro-5-oxo-8-oxa- 4(2,1)-pyrrolidina- 1(1,2),2(1,3)- dibenzenacyclooctaphane- 4³-yl)methanesulfonamide | 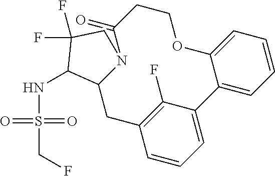 [SMILES: O=C1CCOc2ccccc2-c2cccc(c2F)CC2C(NS(=O)(=O)CF)C(F)(F)CN12] |
| 94 | N-(2²,4⁴,4⁴-trifluoro-5-oxo- 8-oxa-4(2,1)-pyrrolidina- 1(1,2),2(1,3)- dibenzenacyclooctaphane- 4³-yl)ethanesulfonamide | 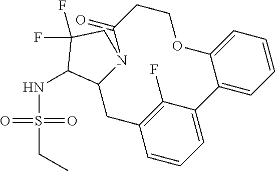 [SMILES: CCS(=O)(=O)NC1C2Cc3cccc(c3F)-c3ccccc3OCCC(=O)N2CC1(F)F] |
| 95 | 1,1-difluoro-N-(2²-fluoro-6- methyl-5-oxo-9-oxa-6-aza- 4(2,1)-pyrrolidina- 1(1,2),2(1,3)- dibenzenacyclononaphane- 4³-yl)methanesulfonamide | 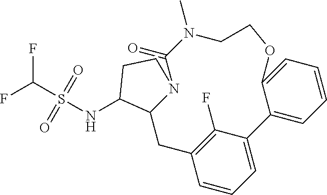 [SMILES: CN1CCOc2ccccc2-c2cccc(c2F)CC2C(NS(=O)(=O)C(F)F)CCN2C1=O] |
| 96 | N-(1³,2²-difluoro-6-methyl- 5-oxo-9-oxa-6-aza-4(2,1)- pyrrolidina-1(1,2),2(1,3)- dibenzenacyclononaphane- 4³-yl)-1,1- difluoromethanesulfonamide | 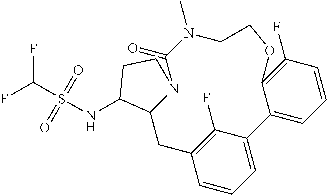 [SMILES: CN1CCOc2c(F)cccc2-c2cccc(c2F)CC2C(NS(=O)(=O)C(F)F)CCN2C1=O] |
| 97 | 1-fluoro-N-(6-methyl-5-oxo- 9-oxa-6-aza-4(2,1)- pyrrolidina-1(1,2),2(1,3)- dibenzenacyclononaphane- 4³-yl)methanesulfonamide | 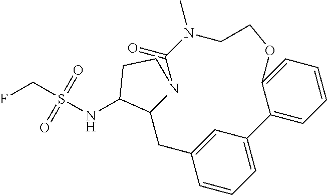 [SMILES: CN1CCOc2ccccc2-c2cccc(c2)CC2C(NS(=O)(=O)CF)CCN2C1=O] |
| 98 | 1-fluoro-N-(1³-fluoro-6- methyl-5-oxo-9-oxa-6-aza- 4(2,1)-pyrrolidina- 1(1,2),2(1,3)- dibenzenacyclononaphane- 4³-yl)methanesulfonamide | 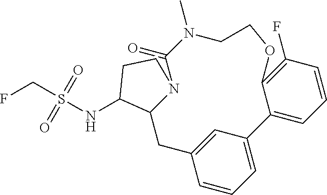 [SMILES: CN1CCOc2c(F)cccc2-c2cccc(c2)CC2C(NS(=O)(=O)CF)CCN2C1=O] |
| 99 | 1-fluoro-N-(2²-fluoro-6- methyl-5-oxo-9-oxa-6-aza- 4(2,1)-pyrrolidina- 1(1,2),2(1,3)- dibenzenacyclononaphane- 4³-yl)methanesulfonamide | 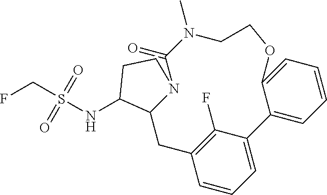 [SMILES: CN1CCOc2ccccc2-c2cccc(c2F)CC2C(NS(=O)(=O)CF)CCN2C1=O] |
| 100 | N-(1³,2²-difluoro-6-methyl- 5-oxo-9-oxa-6-aza-4(2,1)- pyrrolidina-1(1,2),2(1,3)- dibenzenacyclononaphane- 4³-yl)-1- fluoromethanesulfonamide | 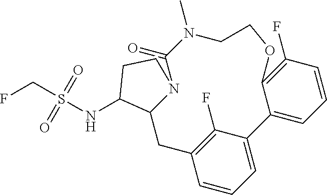 [SMILES: CN1CCOc2c(F)cccc2-c2cccc(c2F)CC2C(NS(=O)(=O)CF)CCN2C1=O] |
| 101 | N-(1³-fluoro-6-methyl-5- oxo-9-oxa-6-aza-4(2,1)- pyrrolidina-1(1,2),2(1,3)- dibenzenacyclononaphane- 4³-yl)ethanesulfonamide | 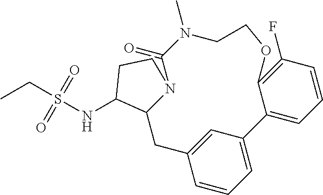 [SMILES: CCS(=O)(=O)NC1CCN2C(=O)N(C)CCOc3c(F)cccc3-c3cccc(c3)CC12] |
| 102 | N-(2²-fluoro-6-methyl-5- oxo-9-oxa-6-aza-4(2,1)- pyrrolidina-1(1,2),2(1,3)- dibenzenacyclononaphane- 4³-yl)ethanesulfonamide | 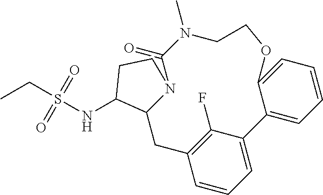 [SMILES: CCS(=O)(=O)NC1CCN2C(=O)N(C)CCOc3ccccc3-c3cccc(c3F)CC12] |
| 103 | N-(1³,2²-difluoro-6-methyl- 5-oxo-9-oxa-6-aza-4(2,1)- pyrrolidina-1(1,2),2(1,3)- dibenzenacyclononaphane- 4³-yl)ethanesulfonamide | 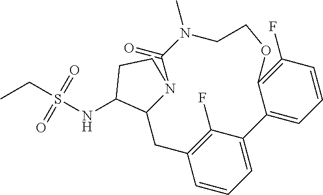 [SMILES: CCS(=O)(=O)NC1CCN2C(=O)N(C)CCOc3c(F)cccc3-c3cccc(c3F)CC12] |
| 104 | 1,1-difluoro-N-(2²-fluoro- 5-oxo-6,9-dioxa-4(2,1)- piperidina-1(1,2),2(1,3)- dibenzenacyclononaphane- 4³-yl)methanesulfonamide | 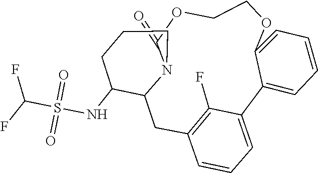 [SMILES: O=C1OCCOc2ccccc2-c2cccc(c2F)CC2C(NS(=O)(=O)C(F)F)CCCN12] |
| 105 | 1-fluoro-N-(2²-fluoro-5-oxo- 6,9-dioxa-4(2,1)-piperidina- 1(1,2),2(1,3)- dibenzenacyclononaphane- 4³-yl)methanesulfonamide | 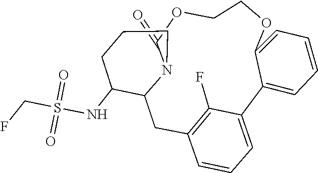 [SMILES: O=C1OCCOc2ccccc2-c2cccc(c2F)CC2C(NS(=O)(=O)CF)CCCN12] |
| 106 | 1,1-difluoro-N-(2²,4⁴,4⁴- trifluoro-6-methyl-5-oxo-9- oxa-6-aza-4(2,1)- pyrrolidina-1(1,2),2(1,3)- dibenzenacyclononaphane- 4³-yl)methanesulfonamide | 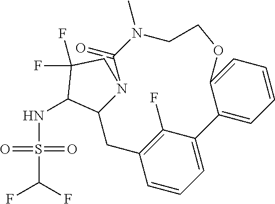 [SMILES: CN1CCOc2ccccc2-c2cccc(c2F)CC2C(NS(=O)(=O)C(F)F)C(F)(F)CN2C1=O] |
| 107 | 1,1-difluoro-N-(2²,4⁴,4⁴- trifluoro-5-oxo-9-oxa- 4(2,1)-pyrrolidina- 1(1,2),2(1,3)- dibenzenacyclononaphane- 4³-yl)methanesulfonamide | 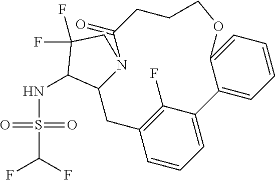 [SMILES: O=C1CCCOc2ccccc2-c2cccc(c2F)CC2C(NS(=O)(=O)C(F)F)C(F)(F)CN12] |
| 108 | 1,1-difluoro-N-(1³,2²,4⁴,4⁴- tetrafluoro-6-methyl-5-oxo- 9-oxa-6-aza-4(2,1)- pyrrolidina-1(1,2),2(1,3)- dibenzenacyclononaphane- 4³-yl)methanesulfonamide | 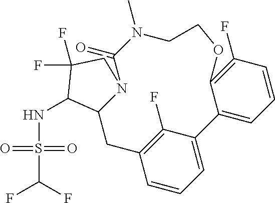 [SMILES: CN1CCOc2c(F)cccc2-c2cccc(c2F)CC2C(NS(=O)(=O)C(F)F)C(F)(F)CN2C1=O] |
| 109 | 1-fluoro-N-(2²,4⁴,4⁴- trifluoro-6-methyl-5-oxo-9- oxa-6-aza-4(2,1)- pyrrolidina-1(1,2),2(1,3)- dibenzenacyclononaphane- 4³-yl)methanesulfonamide | 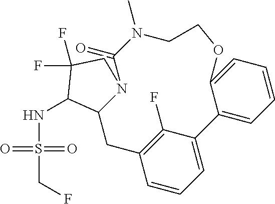 [SMILES: CN1CCOc2ccccc2-c2cccc(c2F)CC2C(NS(=O)(=O)CF)C(F)(F)CN2C1=O] |
| 110 | 1-fluoro-N-(1³,2²,4⁴,4⁴- tetrafluoro-6-methyl-5-oxo- 9-oxa-6-aza-4(2,1)- pyrrolidina-1(1,2),2(1,3)- dibenzenacyclononaphane- 4³-yl)methanesulfonamide | 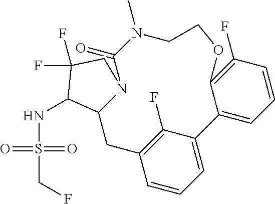 [SMILES: CN1CCOc2c(F)cccc2-c2cccc(c2F)CC2C(NS(=O)(=O)CF)C(F)(F)CN2C1=O] |
| 111 | N-(2²,4⁴,4⁴-trifluoro-6- methyl-5-oxo-9-oxa-6-aza- 4(2,1)-pyrrolidina- 1(1,2),2(1,3)- dibenzenacyclononaphane- 4³-yl)ethanesulfonamide | 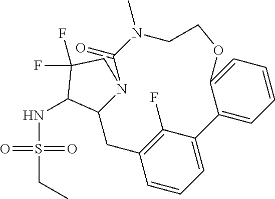 [SMILES: CCS(=O)(=O)NC1C2Cc3cccc(c3F)-c3ccccc3OCCN(C)C(=O)N2CC1(F)F] |
| 112 | N-(1³,2²,4⁴,4⁴-tetrafluoro-6- methyl-5-oxo-9-oxa-6-aza- 4(2,1)-pyrrolidina- 1(1,2),2(1,3)- dibenzenacyclononaphane- 4³-yl)ethanesulfonamide | 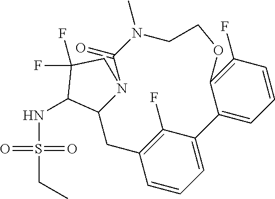 [SMILES: CCS(=O)(=O)NC1C2Cc3cccc(c3F)-c3cccc(F)c3OCCN(C)C(=O)N2CC1(F)F] |
| 113 | 1-fluoro-N-(6′-methyl-5′- oxospiro[cyclopropane-1,4′- 9-oxa-6-aza-4(2,1)- pyrrolidina-1(1,2),2(1,3)- dibenzenacyclononaphan]- 3′-yl)ethane-1-sulfonamide | 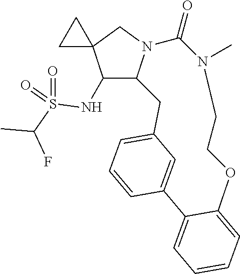 [SMILES: CC(F)S(=O)(=O)NC1C2Cc3cccc(c3)-c3ccccc3OCCN(C)C(=O)N2CC12CC2] |
| 114 | 1-fluoro-N-(2²,4⁴,4⁴- trifluoro-5-oxo-8-oxa- 4(2,1)-piperidina- 1(1,2),2(1,3)- dibenzenacyclooctaphane- 4³-yl)methanesulfonamide | 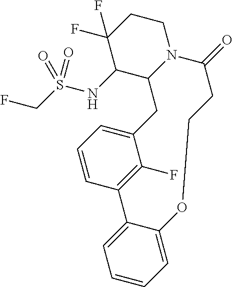 [SMILES: O=C1CCOc2ccccc2-c2cccc(c2F)CC2C(NS(=O)(=O)CF)C(F)(F)CCN12] |
| 115 | 1-fluoro-N-(2²-fluoro-4⁴- (fluoromethyl)-5-oxo-8-oxa- 4(2,1)-piperidina- 1(1,2),2(1,3)- dibenzenacyclooctaphane- 4³-yl)methanesulfonamide | 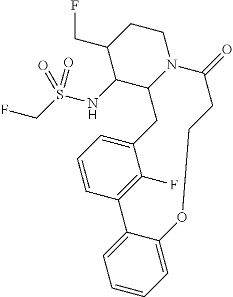 [SMILES: O=C1CCOc2ccccc2-c2cccc(c2F)CC2C(NS(=O)(=O)CF)C(CF)CCN12] |
| 116 | 1-fluoro-N-(2²-fluoro-4⁴- (fluoromethyl)-6-methyl-5- oxo-9-oxa-6-aza-4(2,1)- pyrrolidina-1(1,2),2(1,3)- dibenzenacyclononaphane- 4³-yl)methanesulfonamide | 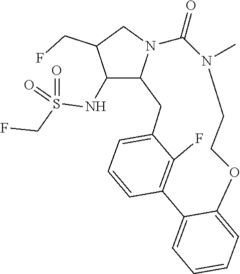 [SMILES: CN1CCOc2ccccc2-c2cccc(c2F)CC2C(NS(=O)(=O)CF)C(CF)CN2C1=O] |
| 117 | N-(4⁴,4⁴-difluoro-5-oxo-8- oxa-4(2,1)-pyrrolidina- 1(1,2),2(1,3)- dibenzenacyclooctaphane- 4³-yl)-1- fluoromethanesulfonamide | 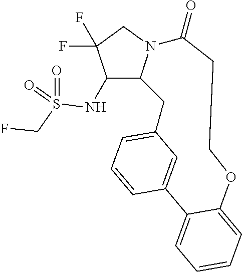 [SMILES: O=C1CCOc2ccccc2-c2cccc(c2)CC2C(NS(=O)(=O)CF)C(F)(F)CN12] |

TABLE 5

| Compound | | |
|---|---|---|
| No. | Name | Structure |
| 118 | N-((4²S,4³R,4⁴S)-1³,4⁴-difluoro- 5-oxo-8-oxa-4(2,1)-piperidina- 1(1,2),2(1,3)-dibenzenacyclo- octaphane-4³-yl)-1-fluoro- cyclopropane-1-sulfonamide | 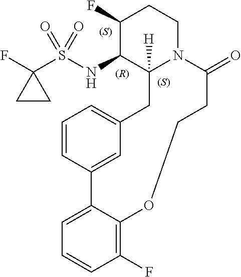 |
| 119 | 1-fluoro-N-[(4²S,4³R,4⁴S)- 1³,2²,4⁴-trifluoro-5-oxo-8-oxa- 4(2,1)-piperidina-1(1,2),2(1,3)- dibenzenacyclooctaphane-4³- yl]cyclopropanesulfonamide | 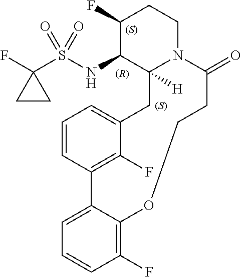 |
| 120 | 1-fluoro-N-[(4²S,4³R,4⁴S)- 1³,1⁵,2²,4⁴-tetrafluoro-5-oxo- 8-oxa-4(2,1)-piperidina- 1(1,2),2(1,3)-dibenzenacyclo- octaphane-4³-yl]cyclopropane- 1-sulfonamide | 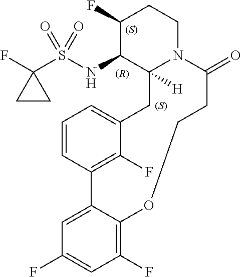 |
| 121 | 1-fluoro-N-((4²S,4³R,4⁴S)-4⁴- fluoro-5-oxo-8-oxa-4(2,1)- piperidina-1(1,2),2(1,3)- dibenzenacyclooctaphane-4³- yl)cyclopropane-1-sulfonamide | 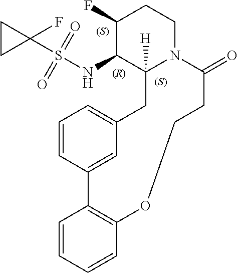 |
| 122 | 1-fluoro-N-[(4²S,4³S)-1³-fluoro- 5-oxo-8-oxa-4(2,1)-piperidina- 1(1,2),2(1,3)-dibenzenacyclo- octaphane-4³-yl]cyclopropane- 1-sulfonamide | 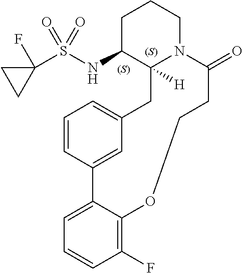 |
| 123 | 1-fluoro-N-[(4²S,4³S)-5-oxo- 8-oxa-4(2,1)-piperidina- 1(1,2),2(1,3)-dibenzenacyclo- octaphane-4³-yl]cyclopropane- 1-sulfonamide | 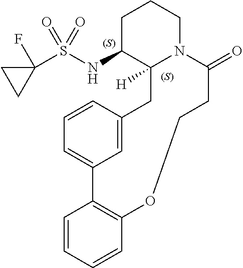 |
| 124 | 1-fluoro-N-[(4²S,4³R,4⁴S)- 1³,2^{2,}4⁴--trifluoro-5-oxo-8-oxa- 4(2,1)-piperidina-1(1,2),2(1,3)- dibenzenacyclooctaphane-4³- yl]methanesulfonamide | 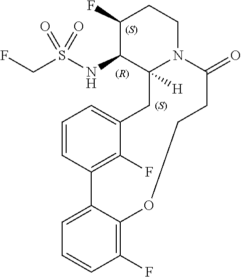 |
| 125 | 1-fluoro-N-[(4²S,4³R,4⁴S)- 1³,1⁵,2²,4⁴-tetrafluoro-5-oxo- 8-oxa-4(2,1)-piperidina- 1(1,2),2(1,3)-dibenzenacyclo- octaphane-4³-yl]methanesul- fonamide | 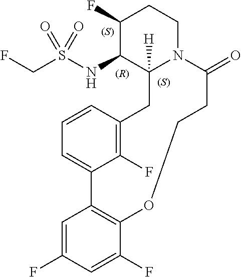 |

In some embodiments, the compound is not described in PCT/US2021/049003.

In some embodiments, the compounds is not selected from the compounds described in PCT/US2021/049003.

In some embodiments, the compound is not selected from the compounds described in Table 6.

TABLE 6
Structure

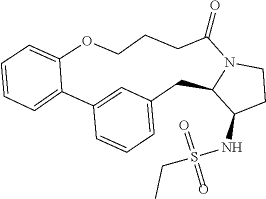

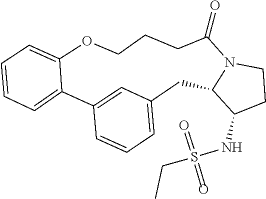

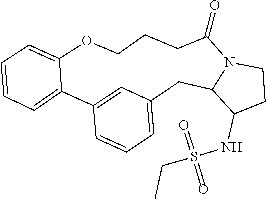

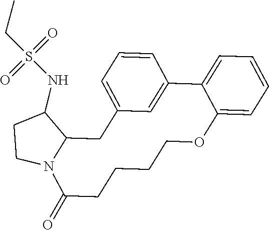 [SMILES: CCS(=O)(=O)NC1CCN2C(=O)CCCCOc3ccccc3-c3cccc(c3)CC12]

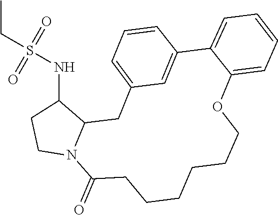 [SMILES: CCS(=O)(=O)NC1CCN2C(=O)CCCCCCOc3ccccc3-c3cccc(c3)CC12]

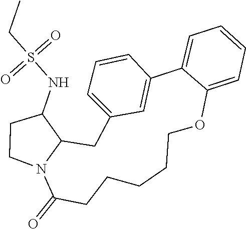 [SMILES: CCS(=O)(=O)NC1CCN2C(=O)CCCCCOc3ccccc3-c3cccc(c3)CC12]

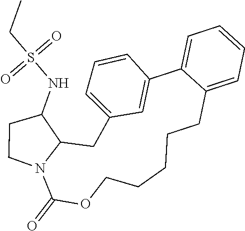 [SMILES: CCS(=O)(=O)NC1CCN2C(=O)OCCCCCc3ccccc3-c3cccc(c3)CC12]

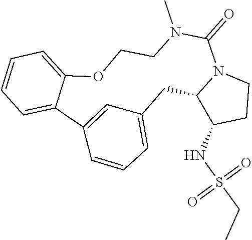 [SMILES: CCS(=O)(=O)N[C@H]1CCN2C(=O)N(C)CCOc3ccccc3-c3cccc(c3)C[C@@H]12]

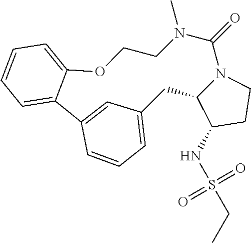 [SMILES: CCS(=O)(=O)N[C@H]1CCN2C(=O)N(C)CCOc3ccccc3-c3cccc(c3)C[C@@H]12]

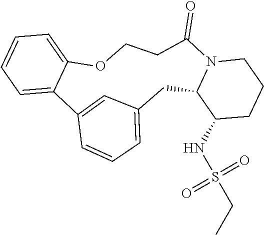 [SMILES: CCS(=O)(=O)N[C@H]1CCCN2C(=O)CCOc3ccccc3-c3cccc(c3)C[C@@H]12]

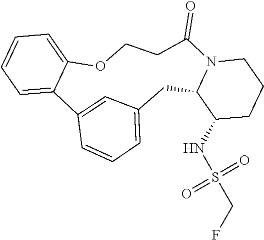 [SMILES: O=C1CCOc2ccccc2-c2cccc(c2)C[C@H]2[C@@H](NS(=O)(=O)CF)CCCN12]

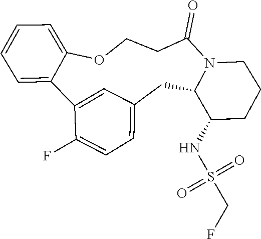 [SMILES: O=C1CCOc2ccccc2-c2cc(ccc2F)C[C@H]2[C@@H](NS(=O)(=O)CF)CCCN12]

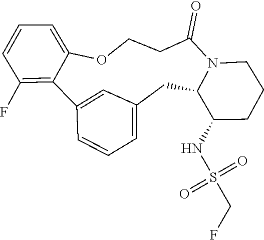 [SMILES: O=C1CCOc2cccc(F)c2-c2cccc(c2)C[C@H]2[C@@H](NS(=O)(=O)CF)CCCN12]

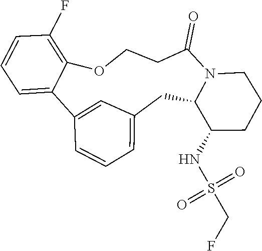 [SMILES: O=C1CCOc2c(F)cccc2-c2cccc(c2)C[C@H]2[C@@H](NS(=O)(=O)CF)CCCN12]

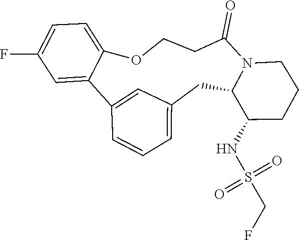 [SMILES: O=C1CCOc2ccc(F)cc2-c2cccc(c2)C[C@H]2[C@@H](NS(=O)(=O)CF)CCCN12]

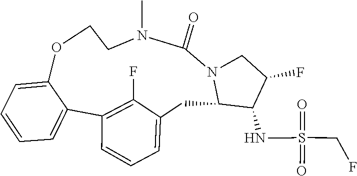 [SMILES: CN1CCOc2ccccc2-c2cccc(c2F)C[C@H]2[C@@H](NS(=O)(=O)CF)[C@@H](F)CN2C1=O]

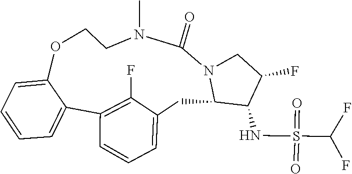 [SMILES: CN1CCOc2ccccc2-c2cccc(c2F)C[C@H]2[C@@H](NS(=O)(=O)C(F)F)[C@@H](F)CN2C1=O]

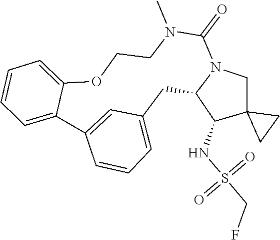 [SMILES: CN1CCOc2ccccc2-c2cccc(c2)C[C@H]2[C@@H](NS(=O)(=O)CF)C3(CC3)CN2C1=O]

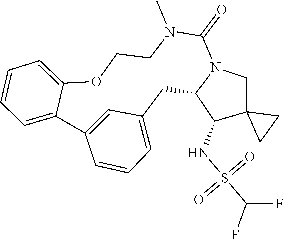 [SMILES: CN1CCOc2ccccc2-c2cccc(c2)C[C@H]2[C@@H](NS(=O)(=O)C(F)F)C3(CC3)CN2C1=O]

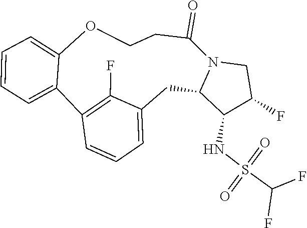 [SMILES: O=C1CCOc2ccccc2-c2cccc(c2F)C[C@H]2[C@@H](NS(=O)(=O)C(F)F)[C@@H](F)CN12]

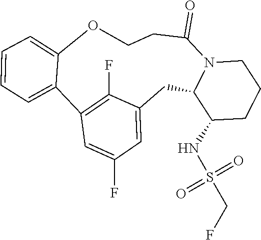 [SMILES: O=C1CCOc2ccccc2-c2cc(F)cc(c2F)C[C@H]2[C@@H](NS(=O)(=O)CF)CCCN12]

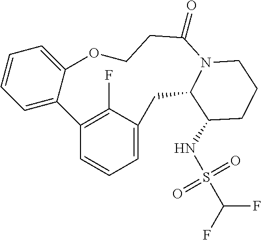 [SMILES: O=C1CCOc2ccccc2-c2cccc(c2F)C[C@H]2[C@@H](NS(=O)(=O)C(F)F)CCCN12]

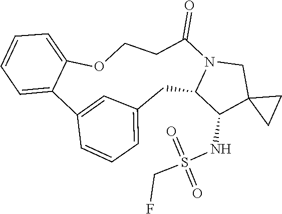 [SMILES: O=C1CCOc2ccccc2-c2cccc(c2)C[C@H]2[C@@H](NS(=O)(=O)CF)C3(CC3)CN12]

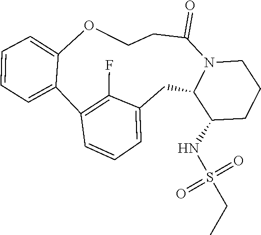 [SMILES: CCS(=O)(=O)N[C@H]1CCCN2C(=O)CCOc3ccccc3-c3cccc(c3F)C[C@@H]12]

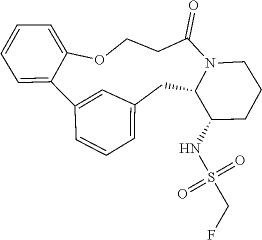 [SMILES: O=C1CCOc2ccccc2-c2cccc(c2)C[C@H]2[C@@H](NS(=O)(=O)CF)CCCN12]

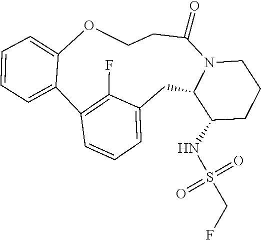 [SMILES: O=C1CCOc2ccccc2-c2cccc(c2F)C[C@H]2[C@@H](NS(=O)(=O)CF)CCCN12]

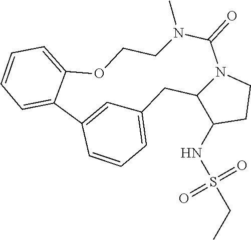 [SMILES: CCS(=O)(=O)NC1CCN2C(=O)N(C)CCOc3ccccc3-c3cccc(c3)CC12]

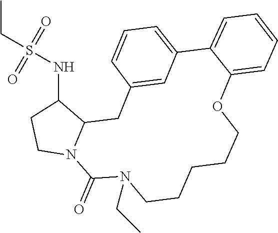 [SMILES: CCN1CCCCCOc2ccccc2-c2cccc(c2)CC2C(NS(=O)(=O)CC)CCN2C1=O]

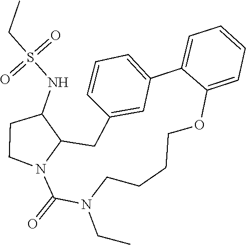 [SMILES: CCN1CCCCOc2ccccc2-c2cccc(c2)CC2C(NS(=O)(=O)CC)CCN2C1=O]

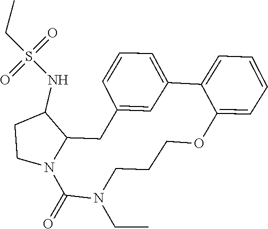 [SMILES: CCN1CCCOc2ccccc2-c2cccc(c2)CC2C(NS(=O)(=O)CC)CCN2C1=O]

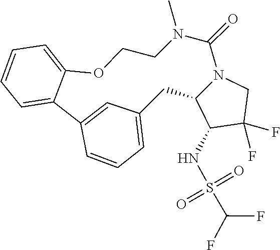 [SMILES: CN1CCOc2ccccc2-c2cccc(c2)C[C@H]2[C@@H](NS(=O)(=O)C(F)F)C(F)(F)CN2C1=O]

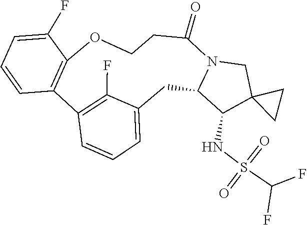 [SMILES: O=C1CCOc2c(F)cccc2-c2cccc(c2F)C[C@H]2[C@@H](NS(=O)(=O)C(F)F)C3(CC3)CN12]

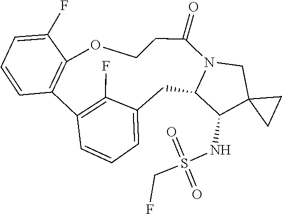 [SMILES: O=C1CCOc2c(F)cccc2-c2cccc(c2F)C[C@H]2[C@@H](NS(=O)(=O)CF)C3(CC3)CN12]

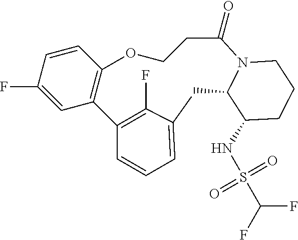 [SMILES: Cc1cccc(-c2cc(F)ccc2OCCC(=O)N2CCC[C@H](NS(=O)(=O)C(F)F)[C@@H]2C)c1F]

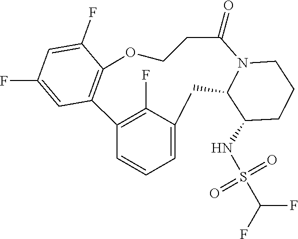 [SMILES: Cc1cccc(-c2cc(F)cc(F)c2OCCC(=O)N2CCC[C@H](NS(=O)(=O)C(F)F)[C@@H]2C)c1F]

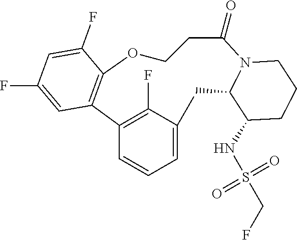 [SMILES: Cc1cccc(-c2cc(F)cc(F)c2OCCC(=O)N2CCC[C@H](NS(=O)(=O)CF)[C@@H]2C)c1F]

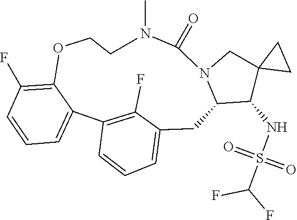 [SMILES: CN1CCOc2c(F)cccc2-c2cccc(c2F)C[C@H]2[C@@H](NS(=O)(=O)C(F)F)C3(CC3)CN2C1=O]

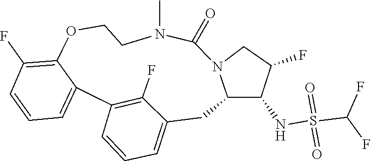 [SMILES: CN1CCOc2c(F)cccc2-c2cccc(c2F)C[C@H]2[C@@H](NS(=O)(=O)C(F)F)[C@@H](F)CN2C1=O]

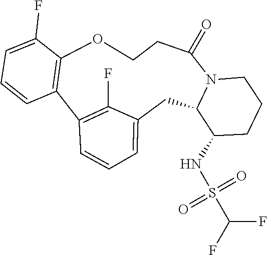 [SMILES: O=C1CCOc2c(F)cccc2-c2cccc(c2F)C[C@H]2[C@@H](NS(=O)(=O)C(F)F)CCCN12]

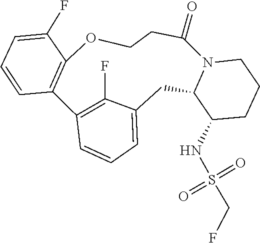 [SMILES: O=C1CCOc2c(F)cccc2-c2cccc(c2F)C[C@H]2[C@@H](NS(=O)(=O)CF)CCCN12]

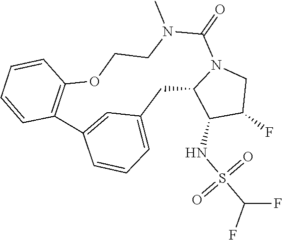 [SMILES: CN1CCOc2ccccc2-c2cccc(c2)C[C@H]2[C@@H](NS(=O)(=O)C(F)F)[C@@H](F)CN2C1=O]

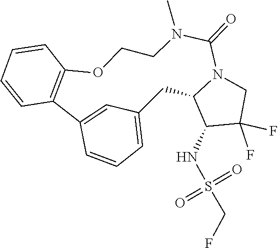 [SMILES: CN1CCOc2ccccc2-c2cccc(c2)C[C@H]2[C@@H](NS(=O)(=O)CF)C(F)(F)CN2C1=O]

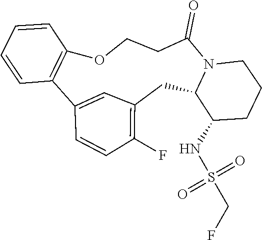 [SMILES: O=C1CCOc2ccccc2-c2ccc(F)c(c2)C[C@H]2[C@@H](NS(=O)(=O)CF)CCCN12]

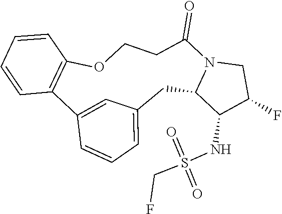 [SMILES: O=C1CCOc2ccccc2-c2cccc(c2)C[C@H]2[C@@H](NS(=O)(=O)CF)[C@@H](F)CN12]

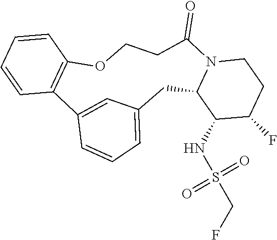 [SMILES: O=C1CCOc2ccccc2-c2cccc(c2)C[C@H]2[C@@H](NS(=O)(=O)CF)[C@@H](F)CCN12]

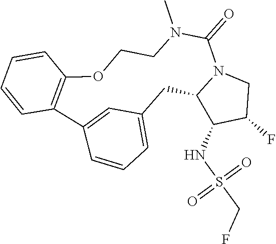 [SMILES: CN1CCOc2ccccc2-c2cccc(c2)C[C@H]2[C@@H](NS(=O)(=O)CF)[C@@H](F)CN2C1=O]

In some embodiments, the compound is a pharmaceutically acceptable salt of any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

In some embodiments, the compound is a pharmaceutically acceptable salt of any one of the compounds described in Table 2.

In some embodiments, the compound is an isotopic derivative of any one of the compounds described in Table 2 and prodrugs and pharmaceutically acceptable salts thereof.

In some embodiments, the compound is an isotopic derivative of any one of the compounds described in Table 2 and pharmaceutically acceptable salts thereof.

In some embodiments, the compound is an isotopic derivative of any one of prodrugs of the compounds described in Table 2 and pharmaceutically acceptable salts thereof.

In some embodiments, the compound is an isotopic derivative of any one of the compounds described in Table 2.

In some embodiments, the compound is a pharmaceutically acceptable salt of any one of the compounds described in Table 3.

In some embodiments, the compound is an isotopic derivative of any one of the compounds described in Table 3 and prodrugs and pharmaceutically acceptable salts thereof.

In some embodiments, the compound is an isotopic derivative of any one of the compounds described in Table 3 and pharmaceutically acceptable salts thereof.

In some embodiments, the compound is an isotopic derivative of any one of prodrugs of the compounds described in Table 3 and pharmaceutically acceptable salts thereof.

In some embodiments, the compound is an isotopic derivative of any one of the compounds described in Table 3.

In some embodiments, the compound is a pharmaceutically acceptable salt of any one of the compounds described in Table 4.

In some embodiments, the compound is an isotopic derivative of any one of the compounds described in Table 4 and prodrugs and pharmaceutically acceptable salts thereof.

In some embodiments, the compound is an isotopic derivative of any one of the compounds described in Table 4 and pharmaceutically acceptable salts thereof.

In some embodiments, the compound is an isotopic derivative of any one of prodrugs of the compounds described in Table 4 and pharmaceutically acceptable salts thereof.

In some embodiments, the compound is an isotopic derivative of any one of the compounds described in Table 4.

In some embodiments, the compound is a pharmaceutically acceptable salt of any one of the compounds described in Table 5.

In some embodiments, the compound is an isotopic derivative of any one of the compounds described in Table 5 and prodrugs and pharmaceutically acceptable salts thereof.

In some embodiments, the compound is an isotopic derivative of any one of the compounds described in Table 5 and pharmaceutically acceptable salts thereof.

In some embodiments, the compound is an isotopic derivative of any one of prodrugs of the compounds described in Table 5 and pharmaceutically acceptable salts thereof.

In some embodiments, the compound is an isotopic derivative of any one of the compounds described in Table 5.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I), Formula (I′), Formula (I″), Formula (I″′), Formula (II), Formula (II′), Formula (III), or Formula (III′) is isotopically enriched with regard to, or labelled with, one or more isotopes as compared to the corresponding compound of Formula (I), Formula (I′), Formula (I″), Formula (I″′), Formula (II), Formula (II′), Formula (III), or Formula (III′). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a ³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S ³⁵S, and/or ³⁶S labeled compound, can be prepared using any of a variety of art-recognised techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. More usually, the molecular weight of the compound will be less than 900, for example less than 800, or less than 750, or less than 700, or less than 650 daltons.

A suitable pharmaceutically acceptable salt of a compound of the disclosure is, for example, an acid-addition salt of a compound of the disclosure which is sufficiently basic, for example, an acid-addition salt with, for example, an inorganic organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

It will be understood that while compounds disclosed herein may be presented in one particular configuration. Such particular configuration is not to be construed as limiting the disclosure to one or another isomer, tautomer, regioisomer or stereoisomer, nor does it exclude mixtures of isomers, tautomers, regioisomers or stereoisomers. In some embodiments, the presentation of a compound herein in a particular configuration intends to encompass, and to refer to, each of the available isomers, tautomers, regioisomers, and stereoisomers of the compound, or any mixture thereof; while the presentation further intends to refer to the specific configuration of the compound. For example, when a compound is presented with a moiety of

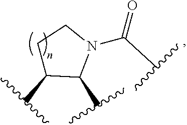

the presentation may intend to encompass, and to refer to, the compound with the moiety of

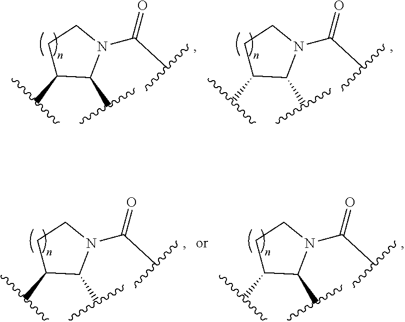

or any mixture thereof. Further, the presentation may intend to refer to the compound with the particular configuration of the moiety of

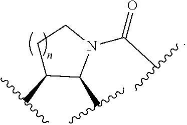

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof. For example, when a compound is presented with a moiety of

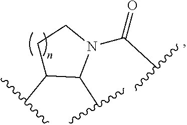

the presentation may intend to encompass, and to refer to, the compound with the moiety of

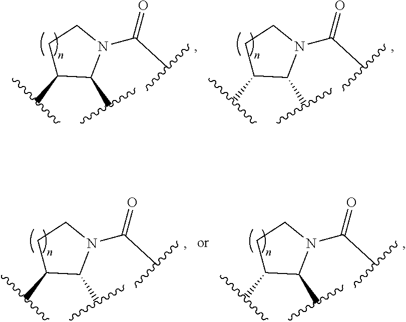

or, or any mixture thereof. Further, the presentation may intend to refer to the compound with a mixture of cis-isomers of the moiety, e.g., a mixture of the moieties of

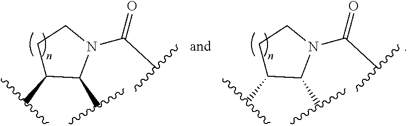

For another example, when a compound is presented with a moiety of

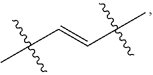 [SMILES: */C=C/*]

the presentation may intend to refer to the compound with the moiety of

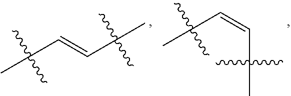

or a mixture thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NTR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

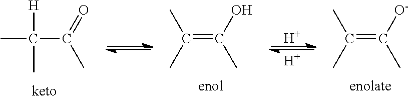

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I), Formula (I′), Formula (I″), Formula (I″′), Formula (II), Formula (II′), Formula (III), or Formula (III′) has been synthesised by any one of the processes defined herein, the processes may then further comprise the additional steps of: (i) removing any protecting groups present; (ii) converting the compound Formula (I), Formula (I′), Formula (I″), Formula (I″′), Formula (II), Formula (II′), Formula (III), or Formula (III′) into another compound of Formula (I), Formula (I′), Formula (I″), Formula (I″′), Formula (II), Formula (II′), Formula (III), or Formula (III′); (iii) forming a pharmaceutically acceptable salt, hydrate or solvate thereof; and/or (iv) forming a prodrug thereof.

The resultant compounds of Formula (I), Formula (I′), Formula (I″), Formula (I″′), Formula (II), Formula (II′), Formula (III), or Formula (III′) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes 1-9 herein.

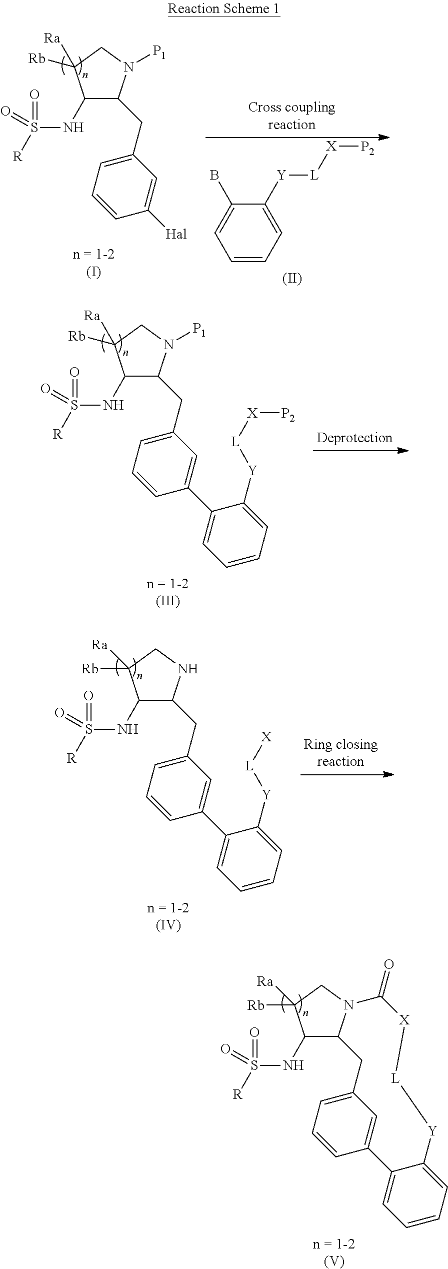

Compounds of Formula (V) can be prepared from compounds of Formula (I) according to the method shown in Reaction Scheme 1. Compounds of formula (I) can be produced according to the previously reported route in WO2019/027058 from commercially available materials or according to a method analogous thereto. As used herein, Hal is a halogen atom.

Examples of the protecting group represented by P₁ for an amino group include carbamate-type protecting groups such as tert-butyl carbamate and the like. Compound (II) when Y is oxygen may be commercially available or can be prepared by alkylation or Mitsunobu reaction from commercially available materials. B represents boronic acid, or boronic ester and the like.

Examples of the protecting group represented by P₂ when X is nitrogen include phthalimide-type protecting groups and the like. Examples of the protecting group represented by P₂ when X is carbon or oxygen include carboxyl protecting groups such as methyl, ethyl ester and the like.

Compound (III) can be produced by subjecting compound (I) and compound (II) to palladium mediated cross-coupling Suzuki type reaction. When the coupling reaction is carried out, examples of the metal catalyst to be used include palladium compounds such as palladium (II) acetate, tetrakis (triphenylphosphine)palladium(0), dichlorobis(triphenylphosphine)palladium (II), tris(dibenzylideneacetone)dipalladium(0), 1,1′-bis(diphenylphosphino)ferrocene palladium(II) chloride, (2-dicyclohexylphosphino-2′,4′,6′-triisopropyl-1,1′-biphenyl)[2-(2′-amino-1,1′-biphenyl)]palladium(II) methanesulfonate and the like. In addition, a base can be added to the reaction system, and examples thereof include inorganic bases and the like.

Compound (IV) can be produced by removing protecting groups represented by P₁ and P₂ according to a method known per se, for example, by employing a method using acid, base, or a nucleophile such hydrazine, and the like, a reduction method, and the like.

Compound (V) can be produced by subjecting compound (IV) to a ring closing reaction. When ring closing is carried out by amidation reaction, urea or carbamate formation, examples of the reagent to be used include activated carboxylic acids such as acid anhydrides, activated esters, activated carbamates and the like. Examples of the activating agent of the carboxylic acid include carbodiimide condensing agents such as N-(3-dimethylaminopropyl)-N′-ethylcarbodiimide hydrochloride (EDCI) and the like; carbonate condensing agents such as 1,1-carbonyldiimidazole (CDI), triphosgene and the like; O-(7-azabenzotriazol-1-yl)-N,N,N′,N′-tetramethyluronium hexafluorophosphorate (HATU); combinations thereof and the like. In addition, a base may be added to the reaction system. Examples of the base include inorganic bases, organic bases, and the like. When a carbodiimide condensing agent is used, an additive such as 1-hydroxybenzotriazole (HOBt), dimethylaminopyridine (DMAP) and the like may be further added to the reaction system.

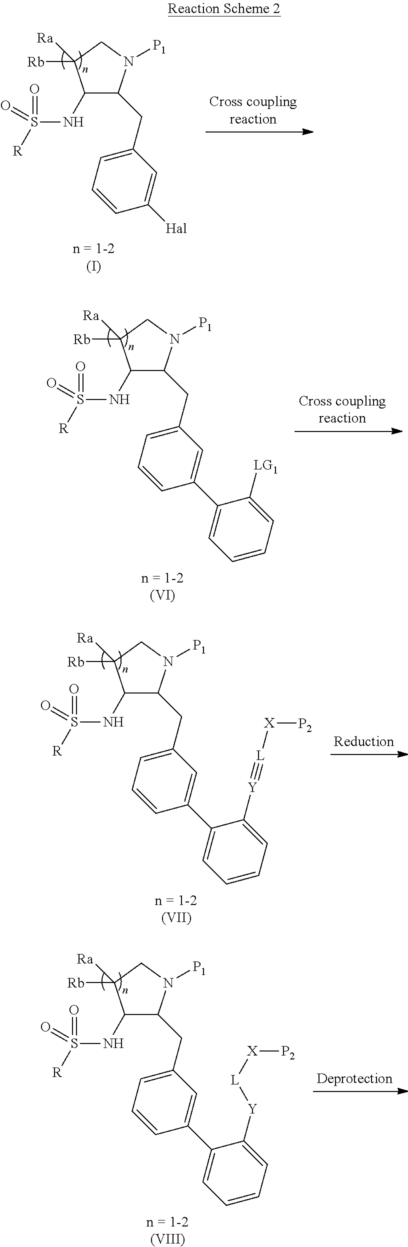

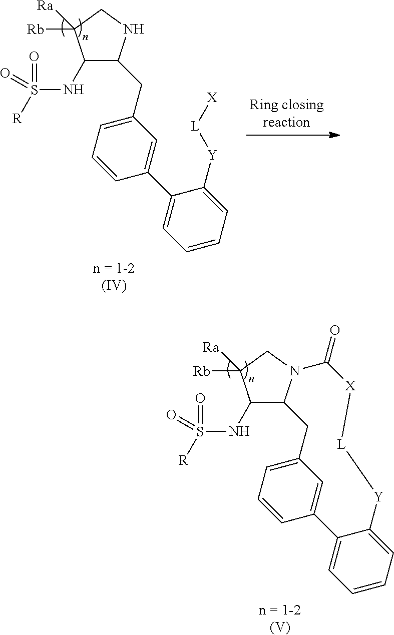

Alternatively, compounds of Formula (V) can be produced from compounds of Formula (I) according to the method shown in Reaction Scheme 2. Compound (VI) can be produced by subjecting a combination of compound (I), arylboronic acid or aryl boronic ester and the like to palladium mediated cross-coupling Suzuki type reaction. When coupling reaction is carried out in each step, examples of the metal catalyst to be used include palladium compounds such as palladium(II) acetate, tetrakis(triphenylphosphine)palladium(0), dichlorobis(triphenylphosphine)palladium (II), tris(dibenzylideneacetone)dipalladium(0), 1,1′-bis(diphenylphosphino)ferrocene palladium(II) chloride, (2-dicyclohexylphosphino-2′,4′,6′-triisopropyl-1,1′-biphenyl)[2-(2′-amino-1,1′-biphenyl)]palladium(II) methanesulfonate and the like. In addition, a base can be added to the reaction system, and examples thereof include inorganic bases and the like. Examples of the “leaving group” represented by LG₁ include halogen atoms, optionally halogenated C₁₋₆ alkylsulfonyloxy groups (e.g., methanesulfonyloxy, ethanesulfonyloxy, trifluoromethanesulfonyloxy) and the like.

Compound (VII) when Y is carbon can be produced by subjecting compound (VI) and an appropriate acetylene, available commercially or according to a known method, to Sonogashira type cross-coupling reaction using a metal catalyst. Examples of the metal catalyst to be used include palladium compounds such as palladium(II) acetate, tetrakis(triphenylphosphine)palladium(0), dichlorobis(triphenylphosphine)palladium (II), Bis(acetonitrile)dichloropalladium(II) and the like. A phosphine ligand can also be added to the reaction system such as 2-dicyclohexylphosphino-2′,4′,6′-triisopropylbiphenyl (XPhos), 2-dicyclohexylphosphino-2′,6′-dimethoxybiphenyl (SPhos) and the like. In addition, a base can be added to the reaction system, and examples thereof include inorganic bases and the like.

Compound (VIII) can be produced by reduction of compound (VII). When carbon-carbon double bond or triple bond is reduced, a method using a catalyst such as palladium-carbon, Lindlar's catalyst, and the like may be employed in conjunction with hydrogen gas.

Compound (VIII) when Y is oxygen and LG₁ represents hydroxy can be prepared directly from compound (VI) by Mitsunobu reaction from commercially available materials. When Mitsunobu reaction is carried out in each step, an azodicarboxylate (e.g., diethyl azodicarboxylate (DEAD), diisopropyl azodicarboxylate (DIAD) etc.) and triphenylphosphine are used as a reagent.

Compound (IV) can be produced by removing protecting groups represented by P₁ and P₂ according to a method known per se, for example, by employing a method using acid, base, hydrazine, and the like, a reduction method, and the like.

Compound (V) can be produced by the ring closing reaction as shown in Reaction Scheme 1.

Compounds of Formula (V) can also be prepared through ring-closing metathesis reaction approaches as outlined in Reaction Schemes 3, 4, and 5.

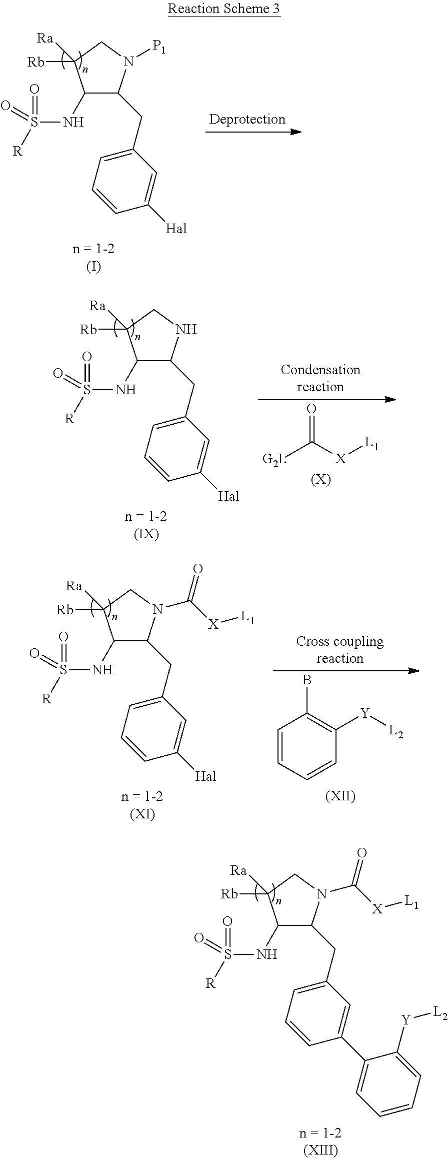

Compound (IX) can be produced by removing protecting groups from Compound (I) represented by P₁ according to an appropriate known method, for example, by using acid, base, hydrazine, and the like, or a reduction method, and the like.

Compound (XI) can be produced by subjecting compound (IX) and compound (X) to a condensation reaction. Where LG2 is an appropriate leaving group. When condensation reaction is carried out examples of the reagent to be used include activated carboxylic acids such as acid anhydrides, activated esters, activated carbonates, activated carbamates, isocyanates and the like. Examples of the activating agent of the carboxylic acid include carbodiimide condensing agents such as N-(3-Dimethylaminopropyl)-N′-ethylcarbodiimide hydrochloride (EDCI); carbonate condensing agents such as 1,1-carbonyldiimidazole (CDI), triphosgene and the like; O-(7-azabenzotriazol-1-yl)-N,N,N′,N′-tetramethyluronium hexafluorophosphorate (HATU); combinations thereof, and the like. In addition, a base may be added to the reaction system. Examples of the base include inorganic bases, organic bases and the like. When a carbodiimide condensing agent is used, an additive such as 1-hydroxybenzotriazole (HOBt) or dimethylaminopyridine (DMAP) may be further added to the reaction system. Compound (X) may be commercially available or produced from commercially available materials according to a method known per se or a method analogous thereto. L₁ represents C₁₋₅ allyl, allyloxy and the like. Examples of leaving group represented by LG₂ include halogen atoms, optionally halogenated C₁₋₆ alkylsulfonyloxy groups (e.g., methanesulfonyloxy, ethanesulfonyloxy, trifluoromethanesulfonyloxy), p-nitrophenol and the like.

Compound (XII) may be commercially available or produced from commercially available materials according to a method known per se or a method analogous thereto. B represents boronic acid, ester and the like. Li represents C₁₋₃ allyl and the like.

Compound (XIII) can be produced by subjecting a combination of compound (XI) and compound (XII) to the aforementioned palladium mediated cross-coupling Suzuki type as shown in Reaction Scheme 1.

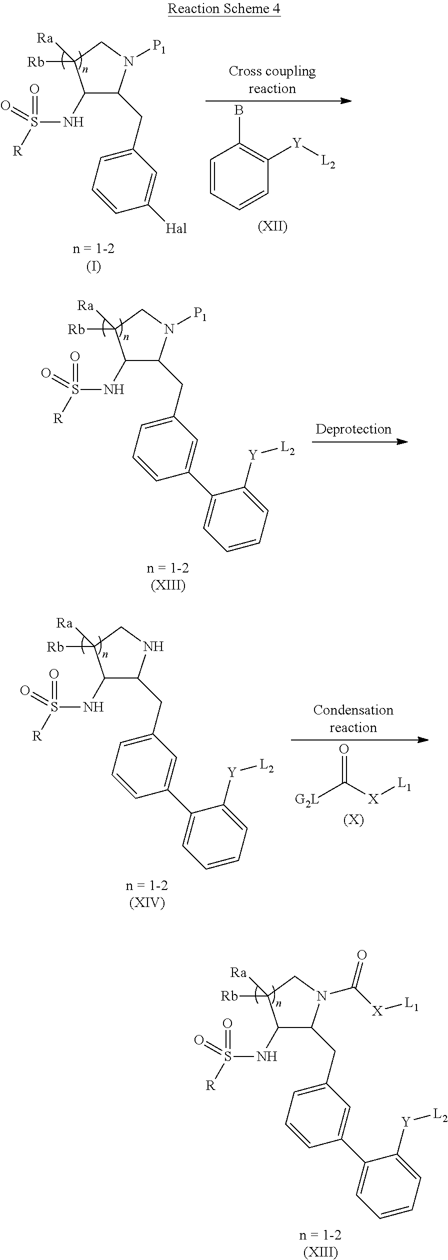

Compound (XIII) can be produced by subjecting a combination of compound (I) and compound (XII) to the aforementioned palladium mediated cross-coupling Suzuki type as shown in Reaction Scheme 1.

Compound (XIV) can be produced by removing protecting groups represented by P₁ according to a known method, for example, by employing an acid, base, hydrazine, and the like, or a reduction method, and the like.

Compound (XIII) can be produced by subjecting compound (XIV) and compound (X) to a condensation reaction as aforementioned in Reaction Scheme 3.

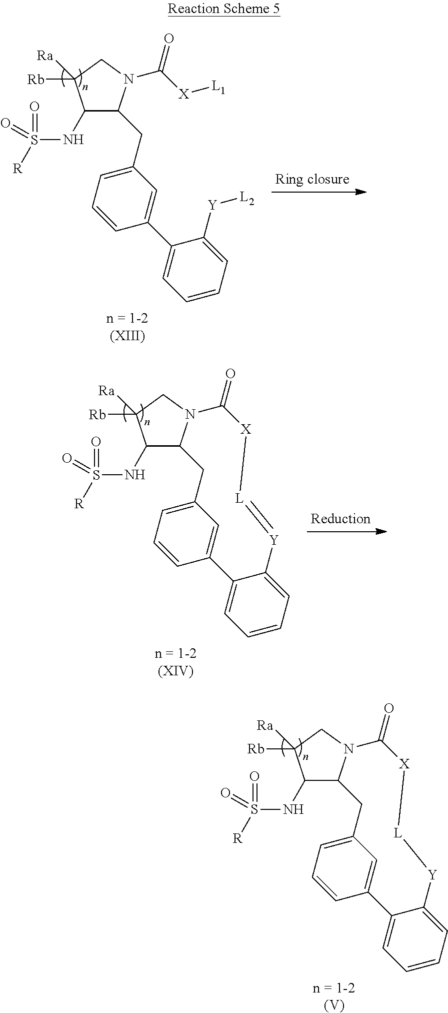

Compound (XIV) can be produced by subjecting compound (XIII) to ring closing reaction. When ring closing is carried out by ring closing metathesis reaction, examples of the catalyst to be used include Ruthenium compounds such as Grubbs I, Grubbs II, Hoveyda Grubbs and the like.

Compound (V) can be produced by reduction of compound (XIV). When carbon-carbon double bond is reduced, a method using a catalyst such as palladium-carbon, Lindlar's catalyst and the like may be employed with hydrogen gas.

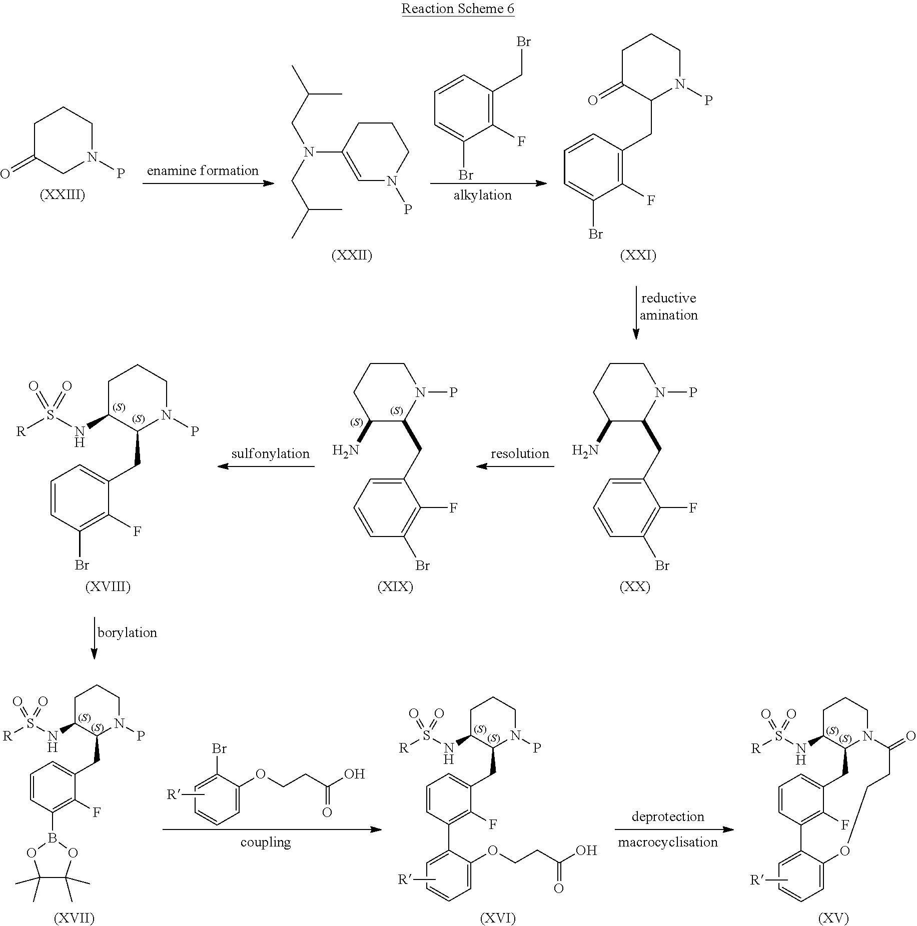

Compounds of formula (XV), formula V when R^{a}=R^{b}=H and n=2, can be prepared according to reaction scheme 6. Compound (XV) can be produced by deprotection and cyclisation of compound (XVI). When protecting group P is tert-butyloxy carbamate deprotection can be effected using an acid. Cyclisation can be achieved by activating the carboxylic acid using a reagent such as an imidazole-1-um chloride or phosphorous oxychloride.

Compound (XVI) can be produced can be produced by subjecting compound (XVII) to a palladium mediated cross-coupling with a substituted bromophenyl compound which can be prepared through known methods or a method analogous thereto. Suitable palladium catalysts include ditert-butyl(cyclopentyl)phosphane; dichloropalladium; iron.

Compounds of type (XVII) can be prepared by palladium mediated borylation of compound (XVIII) using a reagent such as bis-pinacolatodiborane and a catalyst such as palladium tricyclohexyl phosphine complex.

Compound (XVIII) was produced by sulfonylation of compound (XIX). Sulfonylation can be achieved in one step using an appropriate sulfonyl chloride, or in two steps by sulfinylation using a sulfinyl chloride and a subsequent oxidation step using an oxidant such as mCPBA.

Compound (XIX) can be prepared as single enantiomer by a resolution of compound (XX) by chiral salt formation using a chiral acid, for example a tartaric acid derivative. If this resolution is not conducted compounds of formula (XV) can still be prepared as single enantiomers by chiral chromatography of racemic compound (XV).

Compound (XX) can be prepared from compound (XXI) by a reductive amination using a source of ammonia and an appropriate catalyst. Ammonium formate is one such ammonia source and catalysts such as those detailed in J. Org. Chem. 2019, 84, 10962-10977 are suitable for this transformation.

Compound (XXI) is prepared by alkylation of commercially available ketone (XXIII), this can be achieved through the intermediacy of an enamine, such as compound (XXII) which will react with an appropriate benzylic halide, such as 2-fluoro-3-bromobenzyl bromide. The enamine can be prepared by treatment of (XXIII) with a suitable secondary amine and a Lewis acid such as titanium tetra-isopropoxide.

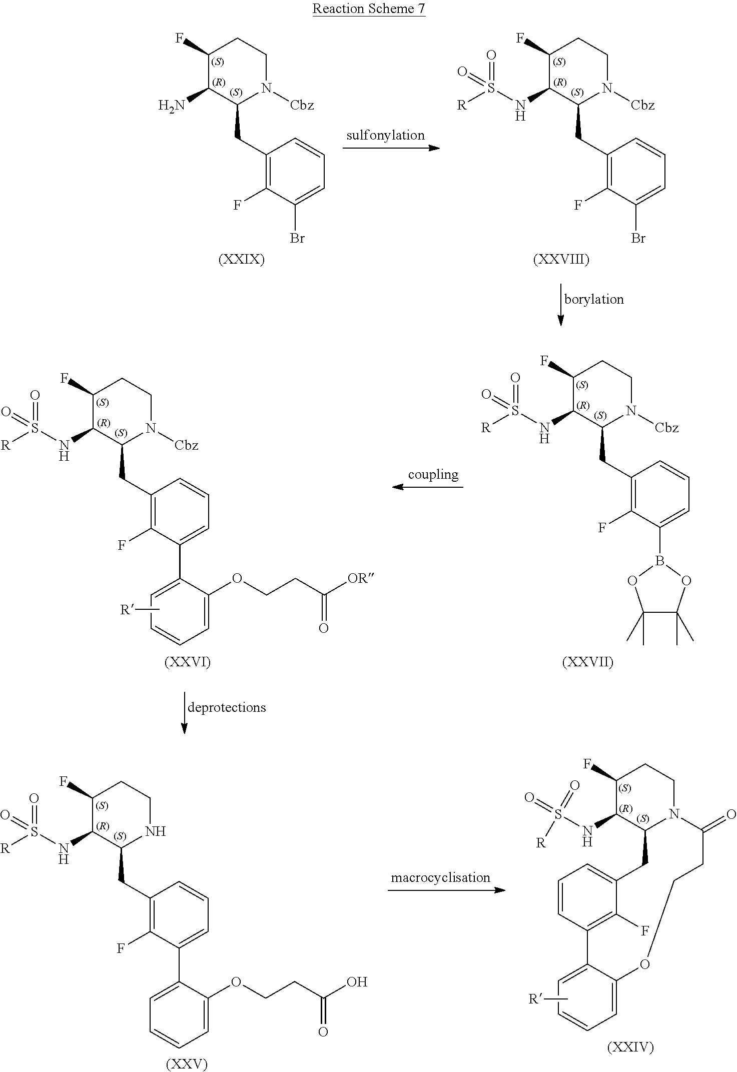

Compound (XXIV), compound (V) where one of R^{a} or R^{b} is fluorine, can prepared through a similar macrocyclization of (XXV). Compound (XXV) can be prepared through sequential deprotections of compound (XXVI), where the N protecting group is benzyloxy carbamate this deprotection can be achieved through hydrogenolysis using hydrogen and an appropriate palladium catalyst such as palladium on carbon. Where the ester protecting group is tert-butylester then the deprotection can be achieved through acid.

Compound (XXVI) can be prepared from compound (XXVII) in an analogous fashion to that carried out in Reaction Scheme 6.

Compound (XXVII) can be prepared from compound (XXVIII) through a borylation reaction analogous to that in Reaction Scheme 6.

Compound (XXVIII) can be prepared from compound (XXIX) by sulfonylation with a sulfonyl chloride or a two step sequence using a sulfinyl chloride and subsequent oxidation with an oxidant such as mCPBA.

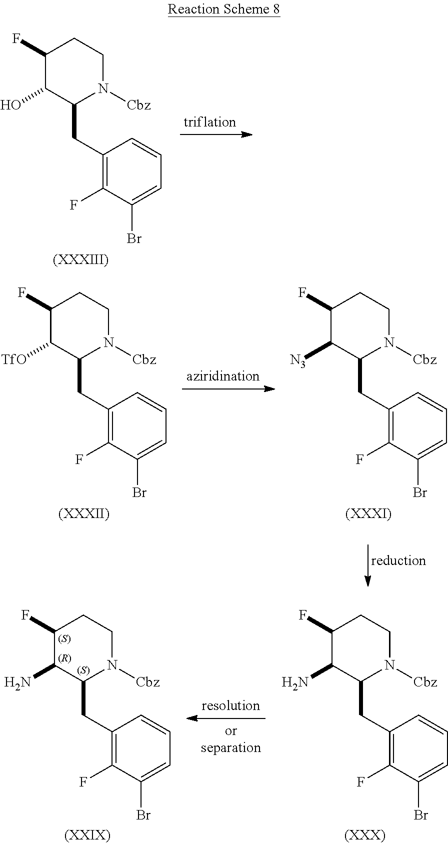

Enantiomerically pure compounds (XXIX) can be prepared through chiral chromatography or a resolution using a chiral salt of compound (XXX). Where a chiral salt is used a chiral acid such as a tartaric acid derivative may be employed.

Compound (XXX) can be prepared through reduction of an azide such as (XXXI). The reduction can be achieved with a phosphorous based reducing agent such as 3-[bis(2-carboxythyl)phosphanyl]propanoic acid hydrochloride.

Compound (XXXI) can be prepared by an azide displacement of a sulfonate ester (XXXII), trifluoromethanesulfonates are suitable for this reaction. Sodium azide can be a suitable azide source for this.

Sulfonate esters such as (XXXII) can be prepared by reaction of compound (XXXIII) with a sulonylating agent such as trifluoromethanesulfonic anhydride.

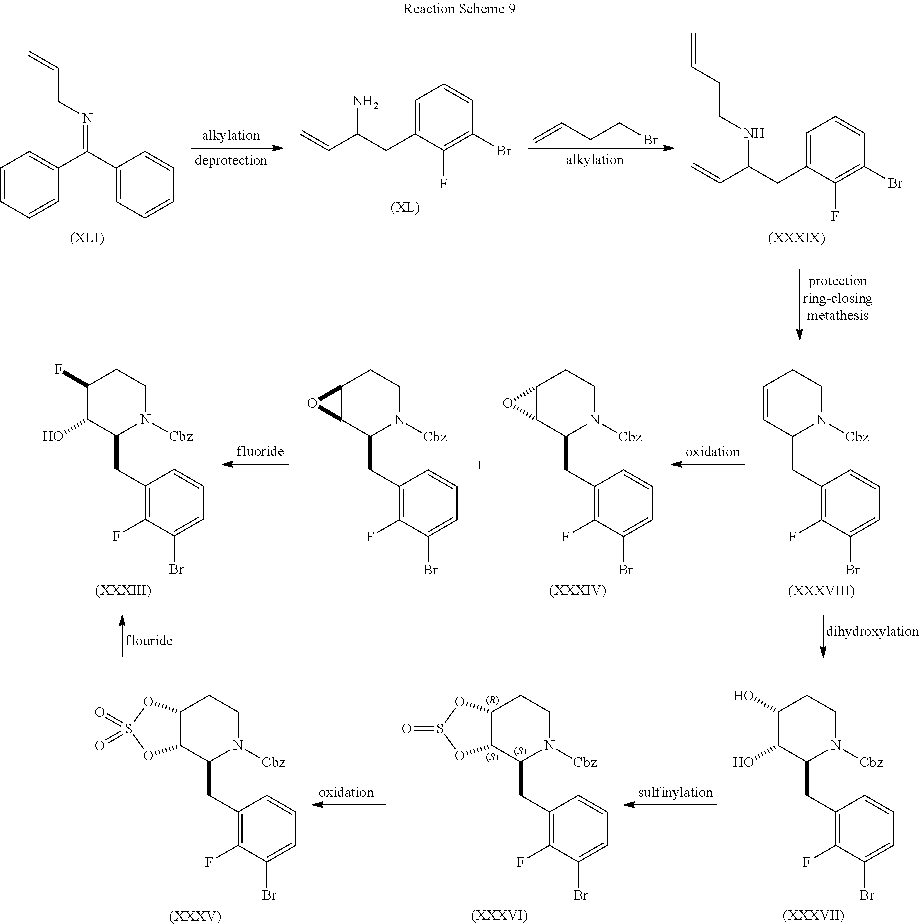

Compound (XXXIII) can be prepared by treatment of either epoxide compounds of formula (XXXIV) or cyclic sulfonates of formula (XXXV) with fluoride. Where epoxides are used hydrogen fluoride salts such as triethylamine·hydrofluoride is a suitable fluoride source. Where cyclic sulfonates are used then tetra-alkyl ammonium salts such as tetraethylammonium fluoride is suitable.

Cyclic sulfonates (XXXV) can be prepared by oxidation of cyclic sulfinates (XXXVI) with an oxidising agent such as sodium periodate, ruthenium trichloride.

Compound (XXXVI) is prepared by sulfination of diol (XXXVII). Sulfination may be effected with sulfonyl chloride.

Diol (XXVII) is prepared by a dihydroxylation reaction of alkene (XXXVIII). Dihydroxylation can be achieved using an oxidising system consisting of a catalytic osmium source and a reoxidant. Potassium osmate and N-methylmorpholine N-oxide is one such system.

Epoxide (XXXIV) can be prepared from alkene (XXXVIII) using an oxidation reaction and then chromatographic separation to isolate the desired relative stereochemistry. Suitable oxidising agents include oxone and trifluoromethyl-methylketone.

Alkene (XXXVIII) may be prepared by ring closing metathesis reactions of dienes such as (XXXIX). Ring closing metathesis can be conducted with a ruthenium catalyst. Examples of the catalyst to be used include Grubbs I, Grubbs II, Hoveyda Grubbs and the like.

Alkene (XXXIX) can be prepared by alkylation of compound (XL). Alkylation can be achieved through reductive amination or treatment with an alkyl halide in the presence of a base.

Compound (XL) can be prepared by alkylation and deprotection of compound (XLI). The alkylation reaction may require use of a strong base such as an alkyllithium to deprotonate (XLI). n-Butyl lithium may be a suitable base when used at low temperature. Deprotection can be achieved using an acid under aqueous conditions.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

Despite orexin cell loss and decreased orexin peptides in cerebrospinal fluid in NT1, orexin receptors on post synaptic neurons remain intact as suitable targets for pharmacotherapeutic intervention. The orexin peptides A and B (OXA and OXB) may be cleaved from a single precursor molecule (prepro-orexin) that is produced exclusively in the lateral hypothalamus. Both orexin peptides bind with similar high affinity to OX2R, but the orexin-1 receptor (OX1R) may be preferentially bound by OXA. Postsynaptic excitation of these G-protein coupled orexin receptors may stimulate the release of monoaminergic and cholinergic neurotransmitters that promote wakefulness and inhibitory neurotransmitters that suppress REM sleep atonia.

In some embodiments, the biological assay is described in the Examples herein.

In some embodiments, the biological assay is an assay mearing the agonist activity of the compound toward cells expressing human orexin type 2 or human orexin type 1 receptor.

In some embodiments, the assay involves preparing Chinese hamster ovary (CHO) cells expressing human orexin type 2 receptor (hOX2R) or human orexin type 1 receptor (hOX1R).

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Tables A1, A2, B1, and B2.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent an inflammasome related condition referred to herein, slow its progression and/or reduce the symptoms associated with the condition.

An effective amount of a compound of the present disclosure for use in therapy is an amount sufficient to treat an inflammasome related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

In some aspects, the present disclosure provides a method of modulating orexin receptor activity (e.g., in vitro or in vivo), comprising contacting a cell with an effective amount of a compound of the present disclosure or a pharmaceutically acceptable salt thereof.

In some aspects, the present disclosure provides a method of modulating orexin receptor activity (e.g., in vitro or in vivo), comprising contacting a cell with a compound of the present disclosure or a pharmaceutically acceptable salt thereof.

In some aspects, the present disclosure provides a method of modulating orexin-2 receptor activity (e.g., in vitro or in vivo), comprising contacting a cell with an effective amount of a compound of the present disclosure or a pharmaceutically acceptable salt thereof.

In some aspects, the present disclosure provides a method of modulating orexin-2 receptor activity (e.g., in vitro or in vivo), comprising contacting a cell with a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof.

In some aspects, the present disclosure provides a method of treating a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated orexin receptor activity. In some embodiments, the disease or disorder is a disease or disorder in which orexin receptor activity is implicated.

In some embodiments, the disease or disorder is associated with an implicated orexin-2 receptor activity. In some embodiments, the disease or disorder is a disease or disorder in which orexin-2 receptor activity is implicated.

In some embodiments, the disease or disorder is narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia.

In some aspects, the present disclosure provides a method of treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof, comprising administering to the subject a compound of the present disclosure or a pharmaceutically acceptable salt thereof, or a pharmaceutical composition of the present disclosure.

In some aspects, the present disclosure provides a method of treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing narcolepsy in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a hypersomnia disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a neurodegenerative disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a symptom of a rare genetic disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a mental health disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a metabolic syndrome in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing osteoporosis in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cardiac failure in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing coma in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a complication in emergence from anesthesia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating narcolepsy in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a hypersomnia disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a neurodegenerative disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a symptom of a rare genetic disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a mental health disorder in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a metabolic syndrome in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating osteoporosis in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating cardiac failure in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating coma in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a complication in emergence from anesthesia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing narcolepsy in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a hypersomnia disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a neurodegenerative disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a symptom of a rare genetic disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a mental health disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a metabolic syndrome in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing osteoporosis in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing cardiac failure in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing coma in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing a complication in emergence from anesthesia in a subject in need thereof, comprising administering to the subject a compound of the present disclosure or a pharmaceutically acceptable salt thereof, or a pharmaceutical composition of the present disclosure.

In some aspects, the present disclosure provides a method of treating narcolepsy in a subject in need thereof, comprising administering to the subject a compound of the present disclosure or a pharmaceutically acceptable salt thereof, or a pharmaceutical composition of the present disclosure.

In some aspects, the present disclosure provides a method of treating a hypersomnia disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating a neurodegenerative disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating a symptom of a rare genetic disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating a mental health disorder in a subject in need thereof, comprising administering to the subject a compound of the present disclosure or a pharmaceutically acceptable salt thereof, or a pharmaceutical composition of the present disclosure.

In some aspects, the present disclosure provides a method of treating a metabolic syndrome in a subject in need thereof, comprising administering to the subject a compound of the present disclosure or a pharmaceutically acceptable salt thereof, or a pharmaceutical composition of the present disclosure.

In some aspects, the present disclosure provides a method of treating osteoporosis in a subject in need thereof, comprising administering to the subject a compound of the present disclosure or a pharmaceutically acceptable salt thereof, or a pharmaceutical composition of the present disclosure.

In some aspects, the present disclosure provides a method of treating cardiac failure in a subject in need thereof, comprising administering to the subject a compound of the present disclosure or a pharmaceutically acceptable salt thereof, or a pharmaceutical composition of the present disclosure.

In some aspects, the present disclosure provides a method of treating coma in a subject in need thereof, comprising administering to the subject a compound of the present disclosure or a pharmaceutically acceptable salt thereof, or a pharmaceutical composition of the present disclosure.

In some aspects, the present disclosure provides a method of treating a complication in emergence from anesthesia in a subject in need thereof, comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating orexin receptor activity (e.g., in vitro or in vivo).

In some aspects, the present disclosure provides a compound of the present disclosure or a pharmaceutically acceptable salt thereof for use in modulating orexin-2 receptor activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides a compound of the present disclosure or a pharmaceutically acceptable salt thereof for use in treating or preventing narcolepsy in a subject in need thereof.

In some aspects, the present disclosure provides a compound of the present disclosure or a pharmaceutically acceptable salt thereof for use in treating or preventing a hypersomnia disorder in a subject in need thereof.

In some aspects, the present disclosure provides a compound of the present disclosure or a pharmaceutically acceptable salt thereof for use in treating or preventing a neurodegenerative disorder in a subject in need thereof.

In some aspects, the present disclosure provides a compound of the present disclosure or a pharmaceutically acceptable salt thereof for use in treating or preventing a symptom of a rare genetic disorder in a subject in need thereof.

In some aspects, the present disclosure provides a compound of the present disclosure or a pharmaceutically acceptable salt thereof for use in treating or preventing a mental health disorder in a subject in need thereof.

In some aspects, the present disclosure provides a compound of the present disclosure or a pharmaceutically acceptable salt thereof for use in treating or preventing a metabolic syndrome in a subject in need thereof.

In some aspects, the present disclosure provides a compound of the present disclosure or a pharmaceutically acceptable salt thereof for use in treating or preventing osteoporosis in a subject in need thereof.

In some aspects, the present disclosure provides a compound of the present disclosure or a pharmaceutically acceptable salt thereof for use in treating or preventing cardiac failure in a subject in need thereof.

In some aspects, the present disclosure provides a compound of the present disclosure or a pharmaceutically acceptable salt thereof for use in treating or preventing coma in a subject in need thereof.

In some aspects, the present disclosure provides a compound of the present disclosure or a pharmaceutically acceptable salt thereof for use in treating or preventing a complication in emergence from anesthesia in a subject in need thereof.

In some aspects, the present disclosure provides a compound of the present disclosure or a pharmaceutically acceptable salt thereof for use in treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides a compound of the present disclosure or a pharmaceutically acceptable salt thereof for use in treating narcolepsy in a subject in need thereof.

In some aspects, the present disclosure provides a compound of the present disclosure or a pharmaceutically acceptable salt thereof for use in treating a hypersomnia disorder in a subject in need thereof.

In some aspects, the present disclosure provides a compound of the present disclosure or a pharmaceutically acceptable salt thereof for use in treating a neurodegenerative disorder in a subject in need thereof.

In some aspects, the present disclosure provides a compound of the present disclosure or a pharmaceutically acceptable salt thereof for use in treating a symptom of a rare genetic disorder in a subject in need thereof.

In some aspects, the present disclosure provides a compound of the present disclosure or a pharmaceutically acceptable salt thereof for use in treating a mental health disorder in a subject in need thereof.

In some aspects, the present disclosure provides a compound of the present disclosure or a pharmaceutically acceptable salt thereof for use in treating a metabolic syndrome in a subject in need thereof.

In some aspects, the present disclosure provides a compound of the present disclosure or a pharmaceutically acceptable salt thereof for use in treating osteoporosis in a subject in need thereof.

In some aspects, the present disclosure provides a compound of the present disclosure or a pharmaceutically acceptable salt thereof for use in treating cardiac failure in a subject in need thereof.

In some aspects, the present disclosure provides a compound of the present disclosure or a pharmaceutically acceptable salt thereof for use in treating coma in a subject in need thereof.

In some aspects, the present disclosure provides a compound of the present disclosure or a pharmaceutically acceptable salt thereof for use in treating a complication in emergence from anesthesia in a subject in need thereof.

In some aspects, the present disclosure provides use of a compound of the present disclosure or a pharmaceutically acceptable salt thereof in the manufacture of a medicament for modulating orexin activity (e.g., in vitro or in vivo).

In some aspects, the present disclosure provides use of a compound of the present disclosure or a pharmaceutically acceptable salt thereof in the manufacture of a medicament for modulating orexin-2 activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides use of a compound of the present disclosure or a pharmaceutically acceptable salt thereof in the manufacture of a medicament for treating or preventing narcolepsy in a subject in need thereof.

In some aspects, the present disclosure provides use of a compound of the present disclosure or a pharmaceutically acceptable salt thereof in the manufacture of a medicament for treating or preventing a hypersomnia disorder in a subject in need thereof.

In some aspects, the present disclosure provides use of a compound of the present disclosure or a pharmaceutically acceptable salt thereof in the manufacture of a medicament for treating or preventing a neurodegenerative disorder in a subject in need thereof.

In some aspects, the present disclosure provides use of a compound of the present disclosure or a pharmaceutically acceptable salt thereof in the manufacture of a medicament for treating or preventing a symptom of a rare genetic disorder in a subject in need thereof.

In some aspects, the present disclosure provides use of a compound of the present disclosure or a pharmaceutically acceptable salt thereof in the manufacture of a medicament for treating or preventing a mental health disorder in a subject in need thereof.

In some aspects, the present disclosure provides use of a compound of the present disclosure or a pharmaceutically acceptable salt thereof in the manufacture of a medicament for treating or preventing a metabolic syndrome in a subject in need thereof.

In some aspects, the present disclosure provides use of a compound of the present disclosure or a pharmaceutically acceptable salt thereof in the manufacture of a medicament for treating or preventing osteoporosis in a subject in need thereof.

In some aspects, the present disclosure provides use of a compound of the present disclosure or a pharmaceutically acceptable salt thereof in the manufacture of a medicament for treating or preventing cardiac failure in a subject in need thereof.

In some aspects, the present disclosure provides use of a compound of the present disclosure or a pharmaceutically acceptable salt thereof in the manufacture of a medicament for treating or preventing coma in a subject in need thereof.

In some aspects, the present disclosure provides use of a compound of the present disclosure or a pharmaceutically acceptable salt thereof in the manufacture of a medicament for treating or preventing a complication in emergence from anesthesia in a subject in need thereof.

In some aspects, the present disclosure provides use of a compound of the present disclosure or a pharmaceutically acceptable salt thereof in the manufacture of a medicament for treating narcolepsy, a hypersomnia disorder, a neurodegenerative disorder, a symptom of a rare genetic disorder, a mental health disorder, a metabolic syndrome, osteoporosis, cardiac failure, coma, or a complication in emergence from anesthesia in a subject in need thereof.

In some aspects, the present disclosure provides use of a compound of the present disclosure or a pharmaceutically acceptable salt thereof in the manufacture of a medicament for treating narcolepsy in a subject in need thereof.

In some aspects, the present disclosure provides use of a compound of the present disclosure or a pharmaceutically acceptable salt thereof in the manufacture of a medicament for treating a hypersomnia disorder in a subject in need thereof.

In some aspects, the present disclosure provides use of a compound of the present disclosure or a pharmaceutically acceptable salt thereof in the manufacture of a medicament for treating a neurodegenerative disorder in a subject in need thereof.

In some aspects, the present disclosure provides use of a compound of the present disclosure or a pharmaceutically acceptable salt thereof in the manufacture of a medicament for treating a symptom of a rare genetic disorder in a subject in need thereof.

In some aspects, the present disclosure provides use of a compound of the present disclosure or a pharmaceutically acceptable salt thereof in the manufacture of a medicament for treating a mental health disorder in a subject in need thereof.

In some aspects, the present disclosure provides use of a compound of the present disclosure or a pharmaceutically acceptable salt thereof in the manufacture of a medicament for treating a metabolic syndrome in a subject in need thereof.

In some aspects, the present disclosure provides use of a compound of the present disclosure or a pharmaceutically acceptable salt thereof in the manufacture of a medicament for treating osteoporosis in a subject in need thereof.

In some aspects, the present disclosure provides use of a compound of the present disclosure or a pharmaceutically acceptable salt thereof in the manufacture of a medicament for treating cardiac failure in a subject in need thereof.

In some aspects, the present disclosure provides use of a compound of the present disclosure or a pharmaceutically acceptable salt thereof in the manufacture of a medicament for treating coma in a subject in need thereof.

In some aspects, the present disclosure provides use of a compound of the present disclosure or a pharmaceutically acceptable salt thereof in the manufacture of a medicament for treating a complication in emergence from anesthesia in a subject in need thereof.

The present disclosure provides compounds that function as modulators of orexin receptor activity.

In some embodiments, the compounds of the present disclosure are agonists of the orexin receptor.

The present disclosure provides compounds that function as modulators of orexin-2 receptor activity.

In some embodiments, the compounds of the present disclosure are agonists of the orexin-2 receptor.

In some embodiments, the modulation of the orexin receptor is activation of the orexin receptor.

Effectiveness of compounds of the disclosure can be determined by industry-accepted assays/disease models according to standard practices of elucidating the same as described in the art and are found in the current general knowledge.

The present disclosure also provides a method of treating a disease or disorder in which orexin receptor activity is implicated in a patient in need of such treatment, said method comprising administering to said patient a therapeutically effective amount of a compound, or a pharmaceutically acceptable salt thereof, or a pharmaceutical composition as defined herein.

The present disclosure also provides a method of treating a disease or disorder in which orexin-2 receptor activity is implicated in a patient in need of such treatment, said method comprising administering to said patient a therapeutically effective amount of a compound, or a pharmaceutically acceptable salt thereof, or a pharmaceutical composition as defined herein.

The present disclosure also provides a method of treating a disease or disorder in which orexin receptor activity is implicated in a patient in need of such treatment, said method comprising administering to said patient a compound, or a pharmaceutically acceptable salt thereof, or a pharmaceutical composition as defined herein.

The present disclosure also provides a method of treating a disease or disorder in which orexin-2 receptor activity is implicated in a patient in need of such treatment, said method comprising administering to said patient a compound, or a pharmaceutically acceptable salt thereof, or a pharmaceutical composition as defined herein.

In some embodiments, the present disclosure also provides a method for treating a disease or disorder by decreasing excessive sleepiness and/or excessive daytime sleepiness.

In some embodiments, the present disclosure also provides a method for treating a disease or disorder by decreasing excessive sleepiness.

In some embodiments, the present disclosure also provides a method for treating a disease or disorder by decreasing excessive daytime sleepiness.

In some embodiments, the disease or disorder is associated with excessive sleepiness and/or excessive daytime sleepiness.

In some embodiments, the disease or disorder is a primary hypersomnia disorder, neurodegenerative disorder, a symptom of a hypersomnia/neurodegenerative disorder, a symptom of a rare genetic disorder, a mental health disorder, a metabolic syndrome, osteoporosis, cardiac failure, coma, or emergence from anesthesia.

In some embodiments, the disease or disorder is a primary hypersomnia disorder, neurodegenerative disorder, a symptom of a hypersomnia/neurodegenerative disorder, a symptom of a rare genetic disorder, a mental health disorder, a metabolic syndrome, osteoporosis, cardiac failure, coma, or a complication in emergence from anesthesia.

In some embodiments, the excessive daytime sleepiness is associated with a neurodegenerative disorder.

In some embodiments, the neurodegenerative disorder associated with excessive daytime sleepiness is Parkinson's disease, Alzheimer's disease, Huntington's disease, or multiple sclerosis.

In some embodiments, the disease or disorder is a recurrence of hypersomnia.

In some embodiments, the recurrence of hypersomnia is narcolepsy type 1, narcolepsy type 2, or idiopathic hypersomnia.

In some embodiments, the disease or disorder is sleep apnea, traumatic brain injury, age-related cognitive dysfunction, or excessive daytime sleepiness.

In some embodiments, excessive daytime sleepiness is associated with sleep apnea, traumatic brain injury, or age-related cognitive dysfunction.

In some embodiments, the disorder is narcolepsy. In some embodiments, narcolepsy is narcolepsy type 1. In some embodiments, the narcolepsy is narcolepsy type 2.

In some embodiments, the hypersomnia is a symptom of narcolepsy.

In some embodiments, the disease or disorder is a symptom of narcolepsy.

In some embodiments, a symptom of narcolepsy is excessive daytime sleepiness, cataplexy, sleep paralysis, hypnopompic and hynogogic hallucinations, disturbed nighttime sleep, or inappropriately timed rapid-eye-movement (REM) sleep.

In some embodiments, a symptom of narcolepsy is excessive daytime sleepiness.

In some embodiments, the symptom of narcolepsy is cataplexy. In some embodiments, cataplexy is pathognomonic of narcolepsy (e.g., narcolepsy type 1).

In some embodiments, a symptom of narcolepsy is sleep paralysis.

In some embodiments, a symptom of narcolepsy is hypnopompic and hynogogic hallucinations.

In some embodiments, a symptom of narcolepsy is disturbed nighttime sleep.

In some embodiments, a symptom of narcolepsy is inappropriately timed rapid-eye-movement (REM) sleep.

In some embodiments, the neurodegenerative disorder is characterized by cataplexy.

In some embodiments, the neurodegenerative disorder is characterized by excessive daytime sleepiness.

In some embodiments, the neurodegenerative disorder is Parkinson's disease.

In some embodiments, the neurodegenerative disorder is Alzheimer's disease.

In some embodiments, the neurodegenerative disorder is Huntington's disease.

In some embodiments, the neurodegenerative disorder is multiple sclerosis.

In some embodiments, the neurodegenerative disorder is a traumatic brain injury.

In some embodiments, the neurodegenerative disorder is sleep apnea.

In some embodiments, the neurodegenerative disorder is age-related cognitive dysfunction.

In some embodiments, the neurodegenerative disorder is a disorder of recurrent hypersomnia.

In some embodiments, a disorder of recurrent hypersomnia is Klein-Levin syndrome, inappropriately timed sleep, (e.g., delayed- or advanced-sleep phase disorder), shift work disorder, or jet lag disorder.

In some embodiments, the disease or disorder is a symptom of a rare genetic disorder.

In some embodiments, a symptom of a rare genetic disorder is abnormal daytime sleepiness.

In some embodiments, a symptom of a rare genetic disorder is excessive daytime sleepiness.

In some embodiments, a symptom of a rare genetic disorder is sleep onset REM periods.

In some embodiments, a symptom of a rare genetic disorder is characterized by cataplexy-like symptoms.

In some embodiments, a rare genetic disorder is ADCA-DN, Coffin-Lowry syndrome, Moebius syndrome, Norrie disease, Niemann-Pick disease type C, or Prader-Willi syndrome.

In some embodiments, the disease or disorder is a mental health disorder.

In some embodiments, the mental health disorder is attention deficit hyperactivity disorder.

In some embodiments, the mental health disorder is attention deficit disorder.

In some embodiments, the disease or disorder is a metabolic syndrome.

In some embodiments, the metabolic syndrome is obesity.

In some embodiments, the disease or disorder is osteoporosis.

In some embodiments, the disease or disorder is cardiac failure.

In some embodiments, the disease or disorder is a coma.

In some embodiments, the disease or disorder is emergence from anesthesia.

In some embodiments, the disease or disorder is a complication in emergence from anesthesia.

In some embodiments, the disease or disorder is narcolepsy, a hypersomnia disorder, a neurodegenerative disorder, a neurological disorder, a symptom of a rare genetic disorder, a psychiatric disorder, a mental health disorder, a circadian rhythm disorder, a metabolic syndrome, osteoporosis, cardiac failure, coma, or a complication in emergence from anesthesia.

In some embodiments, the disease or disorder is narcolepsy, idiopathic hypersomnia, or sleep apnea.

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I), Formula (I′), Formula (I″), Formula (I″′), Formula (II), Formula (II′), Formula (III), or Formula (III′) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which orexin activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I), Formula (I′), Formula (I″), Formula (I″′), Formula (II), Formula (II′), Formula (III), or Formula (III′) and pharmaceutically acceptable salts thereof are also useful as pharmacological tools in the development and standardisation of in vitro and in vivo test systems for the evaluation of the effects of modulators of orexin-2 receptor activity in laboratory animals such as dogs, rabbits, monkeys, mini-pigs, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray or powder); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Exemplary Embodiments

Exemplary Embodiment No. 1. A compound of Formula (I):

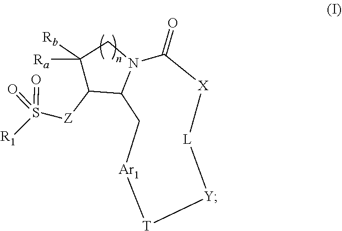

or a pharmaceutically acceptable salt thereof, wherein:
X is —N(CH₃)— or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

Exemplary Embodiment No. 2. A compound of Formula (I′):

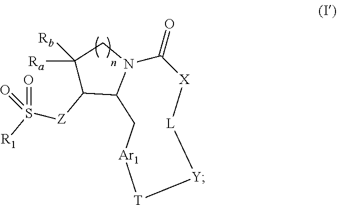

or a pharmaceutically acceptable salt thereof, wherein:
X is —N(CH₃)— or C₂-C₆ alkyl;
L is absent or C₂-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

Exemplary Embodiment No. 3. A compound of Formula (I″):

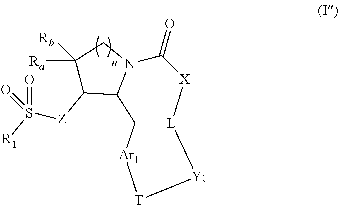

or a pharmaceutically acceptable salt thereof, wherein:
X is —N(CH₃)— or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

Exemplary Embodiment No. 4. A compound of Formula (I″′):

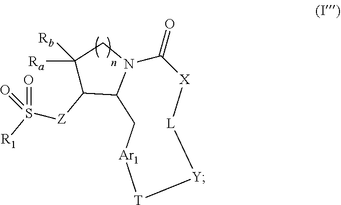

or a pharmaceutically acceptable salt thereof, wherein:
X is —N(CH₃)— or C₂-C₆ alkyl;
L is absent or C₂-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

Exemplary Embodiment No. 5. A compound of Formula (II):

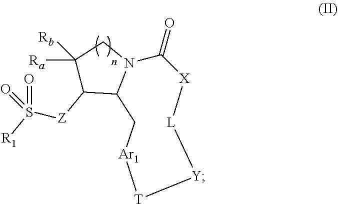

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₁-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

Exemplary Embodiment No. 6. A compound of Formula (II′):

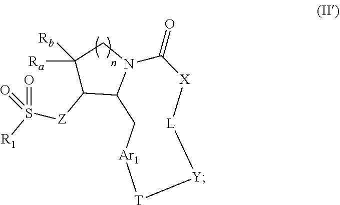

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl or C₂-C₆ alkyl;
Ar₁ is C₆-C₁₀ aryl optionally substituted with one or more halogen; and
T is C₆-C₁₀ aryl optionally substituted with one or more halogen,
provided that when X is alkyl, L is absent.

Exemplary Embodiment No. 7. A compound of Formula (III):

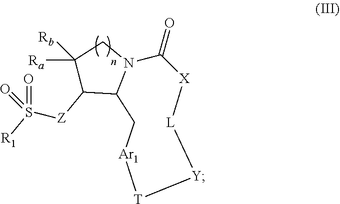

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

Exemplary Embodiment No. 8. A compound of Formula (III′):

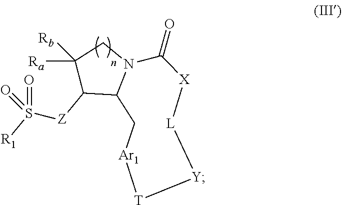

or a pharmaceutically acceptable salt thereof, wherein:
X is —N(C₁-C₆ alkyl)-, —N(C₁-C₆ haloalkyl)-, or C₁-C₆ alkyl;
L is absent or C₁-C₆ alkyl;
Y is —O—;
n is 1 or 2;
Rₐ is H or halogen;
R_{b} is H, halogen, or C₁-C₆ alkyl;
or Rₐ and R_{b}, together with the atom they attach to, form C₃ cycloalkyl;
Z is —NH—;
R₁ is C₁-C₆ haloalkyl, C₂-C₆ alkyl, or C₃ cycloalkyl substituted with halogen;
Ar₁ is C₆-C₁₀ aryl optionally substituted with one or more halogen; and
T is C₆-C₁₀ aryl optionally substituted with one or more halogen or C₁-C₆ alkyl,
provided that when X is alkyl, L is absent.

Exemplary Embodiment No. 9. The compound of any one of the preceding Exemplary Embodiments, wherein X is —N(CH₃)—.

Exemplary Embodiment No. 10. The compound of any one of Exemplary Embodiments 1-8, wherein X is —N(C₁-C₆ haloalkyl)- or C₁-C₆ alkyl.

Exemplary Embodiment No. 11. The compound of any one of the preceding Exemplary Embodiments, wherein L is absent.

Exemplary Embodiment No. 12. The compound of any one of Exemplary Embodiments 1-10, wherein L is C₁-C₆ alkyl.

Exemplary Embodiment No. 13. The compound of any one of the preceding Exemplary Embodiments, wherein n is 1.

Exemplary Embodiment No. 14. The compound of any one of Exemplary Embodiments 1-12, wherein n is 2.

Exemplary Embodiment No. 15. The compound of any one of the preceding Exemplary Embodiments, wherein Rₐ is H.

Exemplary Embodiment No. 16. The compound of any one of Exemplary Embodiments 1-14, wherein Rₐ is halogen.

Exemplary Embodiment No. 17. The compound of any one of the preceding Exemplary Embodiments, wherein R_{b} is H.

Exemplary Embodiment No. 18. The compound of any one of Exemplary Embodiments 1-16, wherein R_{b} is halogen.

Exemplary Embodiment No. 19. The compound of any one of Exemplary Embodiments 1-14, wherein Rₐ and R_{b}, together with the atom they attach to, form C₃ cycloalkyl.

Exemplary Embodiment No. 20. The compound of any one of the preceding Exemplary Embodiments, wherein R₁ is C₂-C₆ alkyl.

Exemplary Embodiment No. 21. The compound of any one of Exemplary Embodiments 1-19, wherein R₁ is C₃ cycloalkyl substituted with halogen.

Exemplary Embodiment No. 22. The compound of any one of the preceding Exemplary Embodiments, wherein Ar₁ is phenyl optionally substituted with one or more halogen.

Exemplary Embodiment No. 23. The compound of any one of the preceding Exemplary Embodiments, wherein T is phenyl optionally substituted with one or more halogen.

Exemplary Embodiment No. 24. The compound of any one of the preceding Exemplary Embodiments, wherein the compound is of Formula (I′-a) or (I′-b):

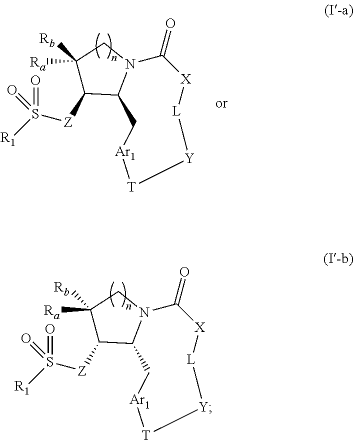

or a pharmaceutically acceptable salt thereof.

Exemplary Embodiment No. 25. The compound of any one of the preceding Exemplary Embodiments, wherein the compound is of Formula (II′-a), (II′-b), or (II′-c):

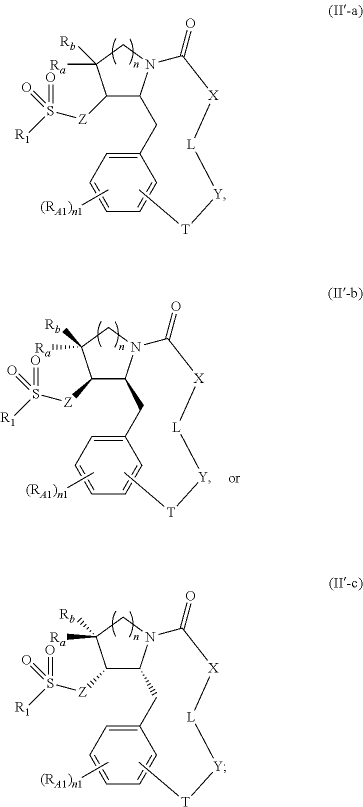

or a pharmaceutically acceptable salt thereof, wherein:
n1 is an integer ranging from 0 to 4.

Exemplary Embodiment No. 26. The compound of any one of the preceding Exemplary Embodiments, wherein the compound is of Formula (IIIA′), (IIIA′-a), or (IIIA′-b):

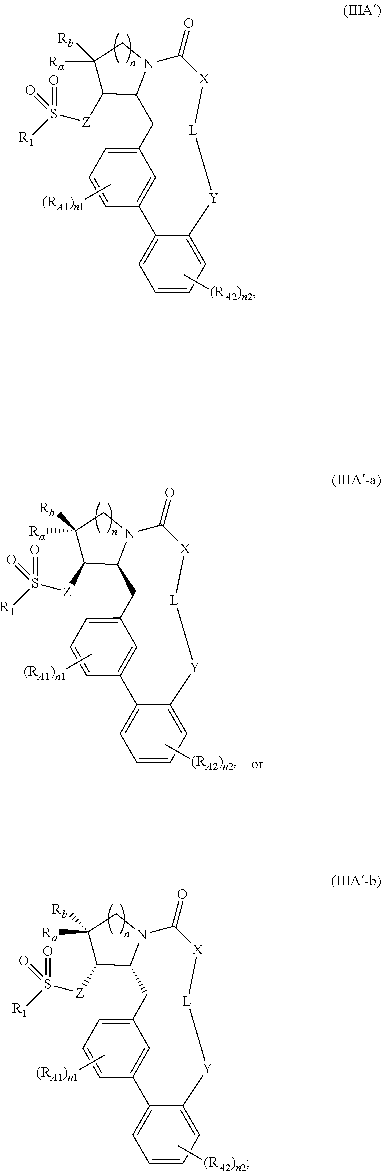

or a pharmaceutically acceptable salt thereof, wherein:
n1 is an integer ranging from 0 to 4; and
n2 is an integer ranging from 0 to 4.

Exemplary Embodiment No. 27. The compound of any one of the preceding Exemplary Embodiments, wherein the compound is of Formula (IVA′), (IVA′-a), or (IVA′-b):

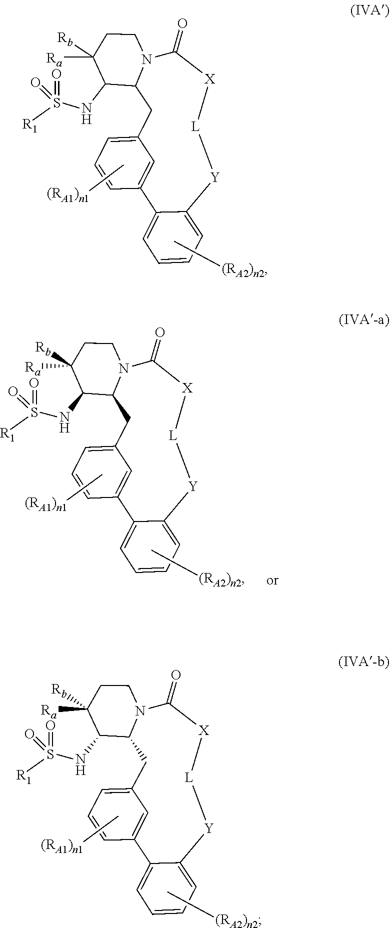

or a pharmaceutically acceptable salt thereof, wherein:
n1 is an integer ranging from 0 to 4; and
n2 is an integer ranging from 0 to 4.

Exemplary Embodiment No. 28. The compound of any one of Exemplary Embodiments 1-24, wherein the compound is of Formula (I-a) or (I-b):

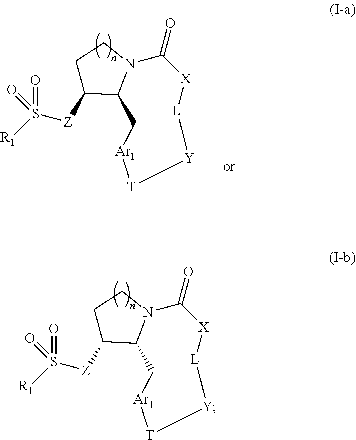

or a pharmaceutically acceptable salt thereof.

Exemplary Embodiment No. 29. The compound of any one of Exemplary Embodiments 1-24, wherein the compound is of Formula (II-a), (II-b), or (II-c):

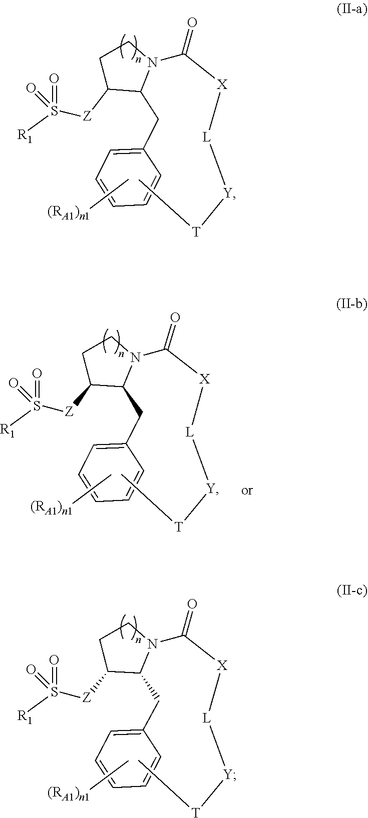

or a pharmaceutically acceptable salt thereof, wherein:
n1 is an integer ranging from 0 to 4.

Exemplary Embodiment No. 30. The compound of any one of Exemplary Embodiments 1-24, wherein the compound is of Formula (IIIA), (IIIA-a), or (IIIA-b):

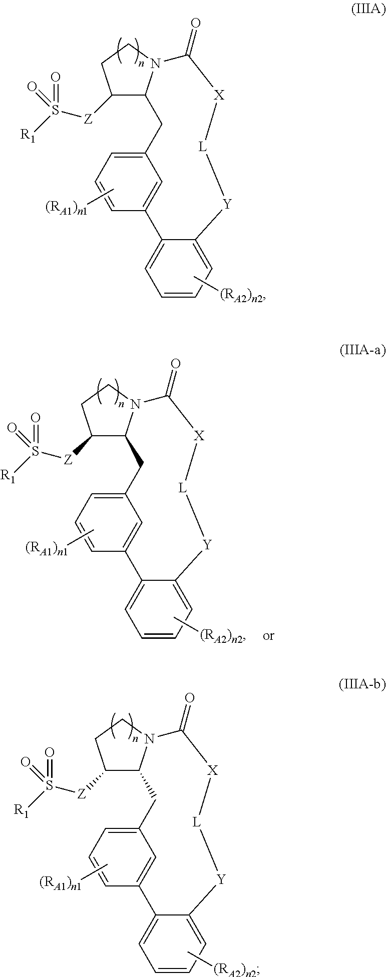

or a pharmaceutically acceptable salt thereof, wherein:
n1 is an integer ranging from 0 to 4; and
n2 is an integer ranging from 0 to 4.

Exemplary Embodiment No. 31. The compound of any one of Exemplary Embodiments 1-24, wherein the compound is of Formula (IIIA′-1), (IIIA′-la), or (IIIA′-1b):

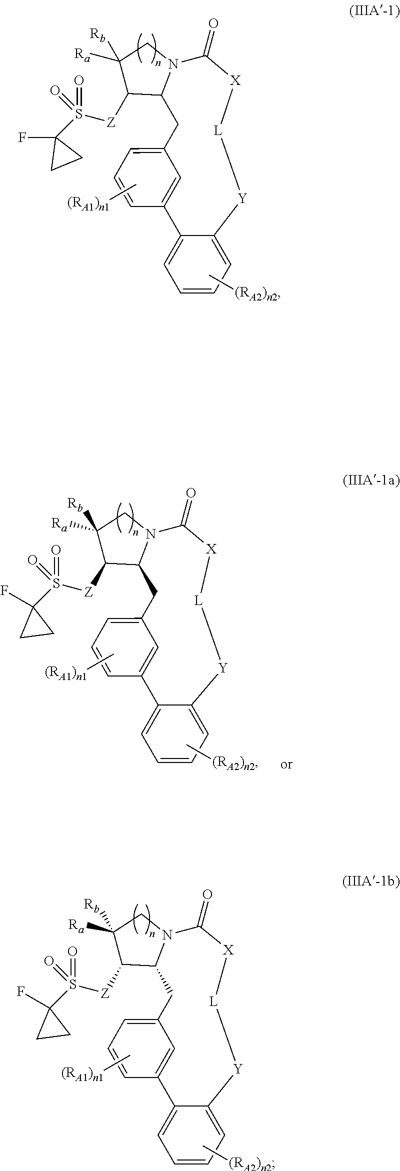

or a pharmaceutically acceptable salt thereof, wherein:
n1 is an integer ranging from 0 to 4; and
n2 is an integer ranging from 0 to 4.

Exemplary Embodiment No. 32. The compound of any one of Exemplary Embodiments 1-24, wherein the compound is of Formula (IIIA′-2), (IIIA′-2a), or (IIIA′-2b):

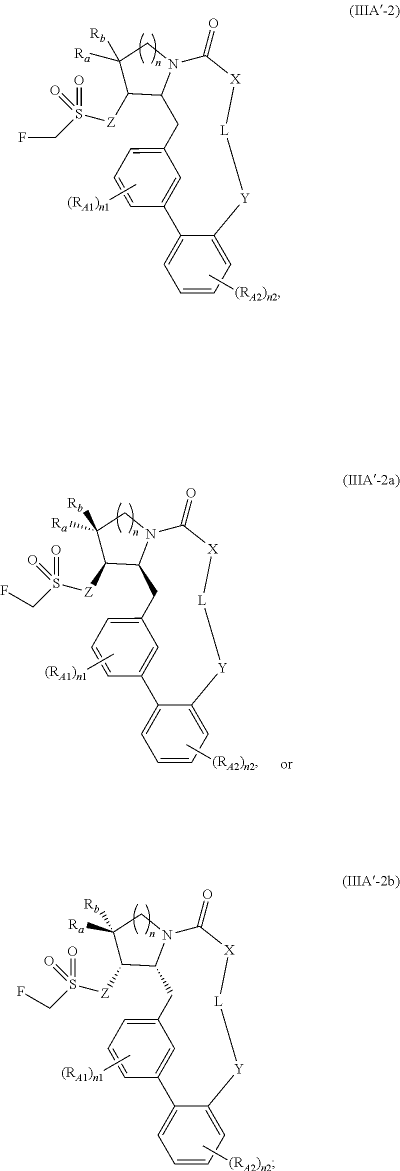

or a pharmaceutically acceptable salt thereof, wherein:
n1 is an integer ranging from 0 to 4; and
n2 is an integer ranging from 0 to 4.

Exemplary Embodiment No. 33. The compound of any one of the preceding Exemplary Embodiments, wherein the compound is selected from a compound described in Table 1, Table 2, Table 3, Table 4, or Table 5, or a pharmaceutically acceptable salt thereof.

Exemplary Embodiment No. 34. A compound obtainable by, or obtained by, a method described herein; optionally, the method comprises one or more steps described in Schemes 1-9.

Exemplary Embodiment No. 35. A pharmaceutical composition comprising the compound of any one of the preceding Exemplary Embodiments or a pharmaceutically acceptable salt thereof, and a pharmaceutically acceptable diluent or carrier.

Exemplary Embodiment No. 36. The pharmaceutical composition of Exemplary Embodiment 35, wherein the compound is selected from a compound described in Table 1, Table 2, Table 3, Table 4, or Table 5.

Exemplary Embodiment No. 37. A method of modulating orexin-2 receptor activity, comprising contacting a cell with an effective amount of the compound of any one of Exemplary Embodiments 1-34; optionally the activity is in vitro or in vivo.

Exemplary Embodiment No. 38. A method of treating or preventing a disease or disorder in a subject in need thereof, comprising administering to the subject a therapeutically effective amount of the compound of any one of Exemplary Embodiments 1-34 or pharmaceutical composition of Exemplary Embodiment 35 or Exemplary Embodiment 36.

Exemplary Embodiment No. 39. The compound of any one of Exemplary Embodiments 1-34 or pharmaceutical composition of Exemplary Embodiment 35 or Exemplary Embodiment 36 for use in modulating orexin-2 receptor activity; optionally, the activity is in vitro or in vivo.

Exemplary Embodiment No. 40. The compound of any one of Exemplary Embodiments 1-34 or pharmaceutical composition of Exemplary Embodiment 35 or Exemplary Embodiment 36 for use in treating or preventing a disease or disorder.

Exemplary Embodiment No. 41. Use of the compound of any one of Exemplary Embodiments 1-34 in the manufacture of a medicament for modulating orexin-2 receptor activity; optionally, the activity is in vitro or in vivo.

Exemplary Embodiment No. 42. Use of the compound of any one of Exemplary Embodiments 1-34 in the manufacture of a medicament for treating or preventing a disease or disorder.

Exemplary Embodiment No. 43. The method, compound, pharmaceutical composition, or use of any one of Exemplary Embodiments 37-42, wherein the disease or disorder is associated with an implicated orexin-2 receptor.

Exemplary Embodiment No. 44. The method, compound, pharmaceutical composition, or use of any one of Exemplary Embodiments 37-43, wherein the disease or disorder is narcolepsy, a hypersomnia disorder, a neurodegenerative disorder, a neurological disorder, a symptom of a rare genetic disorder, a psychiatric disorder, a mental health disorder, a circadian rhythm disorder, a metabolic syndrome, osteoporosis, cardiac failure, coma, or facilitating emergence from anesthesia.

Exemplary Embodiment No. 45. The method, compound, pharmaceutical composition, or use of any one of Exemplary Embodiments 37-44, wherein the disease or disorder is narcolepsy, idiopathic hypersomnia, sleep apnea, or insomnia.

### EXAMPLES

For exemplary purpose, neutral compounds of Formula (I), Formula (I′), Formula (I″), Formula (I″′), Formula (II), Formula (II′), Formula (III), or Formula (III′) are synthesized and tested in the examples. It is understood that the neutral compounds of Formula (I), Formula (I′), Formula (I″), Formula (I″′), Formula (II), Formula (II′), Formula (III), or Formula (III′) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

### Abbreviations:

ACN Acetonitrile
AIBN Azobisisobutyronitrile
BOC tert-butyl carbamate
BOP (benzotriazol-1-yloxy)tris(dimethylamino)phosphonium hexafluorophosphate
BTC bis(trichloromethyl) carbonate
CDI carbonyl diimidazole
DAD diode array detector
DCM Dichloromethane
DIEA/DIPEA N,N-diisopropylethylamine
DMF N,N-dimethylformamide
DMSO Dimethylsulfoxide
EA ethyl acetate
EDCI 1-ethyl-3-(3-dimethylaminopropyl) carbodiimide
ELSD evaporative light scattering detector
ES/ESI electrospray ionisation
HATU 1-[Bis(dimethylamino)methylene]-1H-1,2,3-triazolo[4,5-b]pyridinium 3-oxid hexafluorophosphate
HOAT 1-hydroxy-7-azabenzotriazole
HOBT hydroxy benzotriazole
HPLC high-performance liquid chromatography
IPA Isopropylalcohol
LC liquid chromatography
LiHMDS lithium hexamethyl disilazide
MS mass spectrometry
NMR nuclear magnetic resonance
Py Pyridine
RT retention time
SFC supercritical fluid chromatography
TBAI tetrabutyl ammonium iodide
TEA Triethylamine
TFA trifluoroacetic acid
TFAA trifluoroacetic anhydride
THF Tetrahydrofuran
TLC thin layer chromatography
TMS tetramethyl silane
UV Ultraviolet

NMR spectra were recorded on Bruker Avance III HD UltraShield 400 MHz with a 5 mm PABBO probe, Bruker DPX 300 MHz equipped with a 5 mm BBI probe, Bruker AV 400 MHz equipped with a 5 mm PABBO probe, Bruker DRX 500 MHz equipped with a 5 mm PABBI probe and Bruker Avance III 600 spectrometer equipped with a 5 mm RT BBI probe. The samples were recorded at 25° C. using DMSO-d₆, CDCl₃ or MeOH-d₄ as a solvent and TMS as the internal standard.

### Lcms Conditions

Method A: General UPLC method parameters: gradient mobile phase of 0.1% formic acid in H₂O and CH₃CN or 0.05% NH₃ in H₂O and CH₃CN. Column: Acquity BEH 2.1×50 mm, 1.7 μm and Acquity BEH 2.1×100 mm, 1.7 μm. PDA detector settings: wavelength: 210-400 nm, resolution: 1.2 nm, sampling rate: 1.0 points/sec, filter response: 1. MS detector settings: MS scan: centroid, ionization mode: ES+ and ES−, mass range: 100-1250, scan time: 0.225 s, capillary: 1.30 kV (ES+) and 0.80 kV (ES−), cone: 15 V, extractor: 3.00 V, RF Lens: 0.2 V, source temp.: 120° C., desolvation temp.: 600° C., LM 1 resolution: 0.02, HM 1 resolution: 0.11.

Method B: LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Luna C18 50×2.0 mm column (5 μm particles). Detection methods are diode array (DAD) and evaporative light scattering (ELSD) detection as well as positive electrospray ionization. MS range was 100-1000.)

Method C: 5_95AB_6 min-220-254-ELSD-MS2000: LC/MS (The gradient was 5% B in 0.40 min and 5-95% B in 2.60 min, hold on 95% B in 1.00 min, and then 95-5% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Kinetex C18 2.1*50 mm, 5 μm. Detection methods are diode array (DAD), and evaporative light scattering detection (ELSD). MS mode was positive electrospray ionization. MS range was 100-2000.

Method D: Hewlett Packard 1100 series with Masslynx software, Aqueous (C): Water (2.5 L) with 2.5 mL of 28% Ammonia in water solution Organic (D): Acetonitrile (2.5 L) with 125 mL Water and 2.5 mL of 28% Ammonia in water solution, System runs at a flow rate of 1.5 mL/min, Injection volume of 1 μL, Phenomenex Gemini-NX, 5 μm, C18, 30×2 mm. Column oven temp of 45° C. Hewlett Packard G1315A Diode Array Detector with UV detection from 230 to 400 nm and Waters micromass ZQ mass spectrometer. Gradients written in the following format: [Time (min)/% C: % D], Short Run: [0.00/98:2], [0.1/98:2], [2.5/5:95], [3.5/5:95].

Method E: 5-95 AB_2 min: LC/MS (The column used for chromatography was a Kinetex 5 m EVO C18 100 Å. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 2.20 min 0.5% B in 0.01 min, 5-95% B (0.01-1.00 min), 95-100% B (1.00-1.80 min), 5% B in 1.81 min with a hold at 5% B for 0.39 min. The flow rate was 1.0 mL/min (0.01-1.80) 1.2 mL/min (1.81-2.20).

Method F: 5-95CD_4.5 min: LC/MS (The column used for chromatography was Xbridge C18 2.1*50 mm, 5 μm). Detection methods are diode array (DAD). MS mode was positive electrospray ionization. MS range was 100-1000. Mobile phase A was 10 mM Ammonium bicarbonate in water, and mobile phase B was HPLC grade acetonitrile. The gradient was 5-95% B in 4.30 min. 5% B in 0.01 min, 5-95% B (0.01-3.00 min), and hold at 95% B within 0.5 min, 95-5% B (3.50-3.51 min), with a hold at 5% B for 0.79 min. The flow rate was 1.0 mL/min (0.01-4.30 min).

Method G: 10-80AB_10 min: LC/MS (The gradient was 10-80% B in 8.00 min with a hold at 80% B for 2.00 min, 80-10% B in 0.01 min, and then held at 10% for 2.99 min (0.5 mL/min flow rate). Mobile phase A was 0.04% Trifluoroacetic Acid in water, mobile phase B was 0.02% Trifluoroacetic Acid in acetonitrile. The column used for chromatography was a Halo C18 3.0*10 mm column (2.7 μm particles). Detection methods are diode array (DAD). MS mode was positive electrospray ionization. MS range was 100-1000.

Method H: 5_95CD_6 min-220-254: LC/MS The gradient was 5% B in 0.40 min and 5-95% B at 0.40-3.40 min, hold on 95% B for 0.45 min, and then 95-5% B in 0.01 min, the flow rate was 0.8 mL/min. Mobile phase A was H₂O+10 mM NH₄HCO₃, mobile phase B was Acetonitrile. The column used for chromatography was a Xbridge C18 2.1*50 mm column (5 μm particles). Detection methods are diode array (DAD) detection. MS mode was positive electrospray ionization. MS range was 100-1000.

Method I: 5_95AB_6 min-220-254: LC/MS The gradient was 5% B in 0.40 min and 5-95% B in 2.60 min, hold on 95% B in 1.00 min, and then 95-5% B in 0.01 min, the flow rate was 1.0 mL/min. Mobile phase A was 0.04% Trifluoroacetic Acid in water, mobile phase B was 0.02% Trifluoroacetic Acid in acetonitrile. The column used for chromatography was a Luna C18 50*2.0 mm column (5 μm particles). Detection methods are diode array (DAD) detection. MS mode was positive electrospray ionization. MS range was 100-1000.

Method J: 5-95AB_2 min: LC/MS The column used for chromatography was a Halo 5 m C18 90A, 30*3.0 mm. Detection methods are diode array (DAD). MS mode was positive electrospray ionization. MS range was 50-2000. Mobile phase A was 0.04% trifluoroacetic acid in water, and mobile phase B was 0.02% trifluoroacetic acid in HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

Method K: 10-100AB_2MIN: LC/MS The column used for chromatography was a Halo C18 5 m, 3.0*30 mm (5 μm particles). Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 10-100% B in 1.30 min 0.10% B in 0.01 min, 10-100% B (0.01-0.70 min) with a hold at 100% B for 0.60 min. The flow rate was 1.5 mL/min (0.00-1.30 min).

Method L: 10-80 AB_10 min: LC/MS (The gradient was 10-80% B in 8.00 min with a hold at 80% B for 2.00 min, 80-10% B in 0.01 min, and then held at 10% for 2.99 min (0.5 mL/min flow rate). Mobile phase A was 0.04% trifluoroacetic acid in water, mobile phase B was 0.02% trifluoroacetic acid in acetonitrile. The column used for chromatography was a Halo C18 3.0*100 mm column (2.7 μm particles). Detection methods are diode array (DAD). MS mode was positive electrospray ionization. MS range was 100-1000.

Method M: Column: CSH C18 1.7 m 2.1×50 mm; Run Time: 1.40 min; Solvents A) water B) acetonitrile D) 2% formic acid in water: the gradient runs with 5% D throughout. Gradient: 2-95% B with A and 5% D in 1.20 min, hold at 95% B 5% D to 1.40 min @0.8 mL/min, 40° C.; photodiode array detection at 215-350 nm.

Method N: column: Agilent Poroshell SB-C18 3.0×30 mm, 2.7 μm. Detection methods: diode array. Mobile phase A: 0.04% TFA in water, mobile phase B: 0.02% TFA in HPLC grade acetonitrile. Gradient: 10% B for 0.01 min, then 10-100% B (0.01-0.70 min) with a hold at 100% B for 0.60 min. The flow rate was 1.5 mL/min.

Method O: LC/MS (The column used for chromatography was a Kinetex 5 m EVO C18 100 Å 2.1*30 mm. Detection methods are diode array (DAD). MS mode was positive electrospray ionization. MS range was 100-1000. Mobile phase A was 0.04% TFA in water, and mobile phase B was 0.02% TFA in HPLC grade acetonitrile. The gradient was 5-95% B in 4.30 min 0.5% B in 0.01 min, 5-95% B (0.01-3.00 min), with a hold at 95% B for 0.5 mins, 95-5% B (3.50-3.51 min), with a hold at 5% B for 0.79 min. The flow rate was 1.0 mL/min.

### Synthesis of Intermediates

### Synthesis of tert-butyl (2S,3S)-3-amino-2-(3-bromo-2-fluorobenzyl)piperidine-1-carboxylate

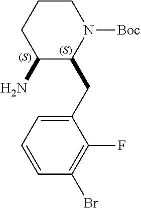

### Intermediate 1: tert-butyl 5-(diisobutylamino)-3,4-dihydropyridine-1(2H)-carboxylate

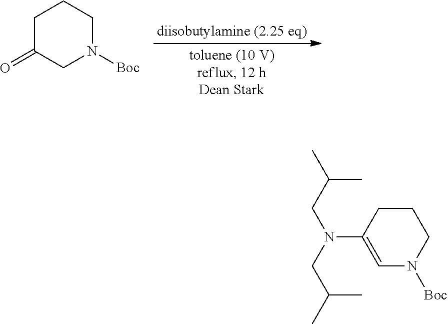 [SMILES: CC(C)(C)OC(=O)N1CCCC(=O)C1 ; CC(C)CN(CC(C)C)C1=CN(C(=O)OC(C)(C)C)CCC1]

Tert-butyl 3-oxopiperidine-1-carboxylate (1.5 kg, 6.62 mol, 1 eq) was added to toluene (15 L) under nitrogen at 20-30° C. and stirred 25° C. Diisobutylamine (1.95 kg, 14.91 mol, 2.25 eq) was added and the reaction mixture heated to reflux (110-113° C.) for 12 hours with water removal (100 mL) by Dean Stark. The reaction was cooled down to 20-30° C. for 60 min (1.5° C./min) to give a brown solution before concentration under reduced pressure (55° C., 0.01 mPa) to give crude Intermediate 1 (2.25 kg, QNMR purity: 59%, yield: 65%) as a brown viscous oil, which was used for next step directly without purification. ¹H NMR (400 MHz, CDCl₃) δ ppm 0.50-1.00 (m, 14H), 1.48 (s, 9H), 1.75-1.85 (m, 2H), 2.05-2.15 (m, 3H), 2.50-2.60 (m, 4H), 5.9-6.1 (m, 1H).

### Intermediate 2: tert-butyl 2-(3-bromo-2-fluorobenzyl)-3-oxopiperidine-1-carboxylate

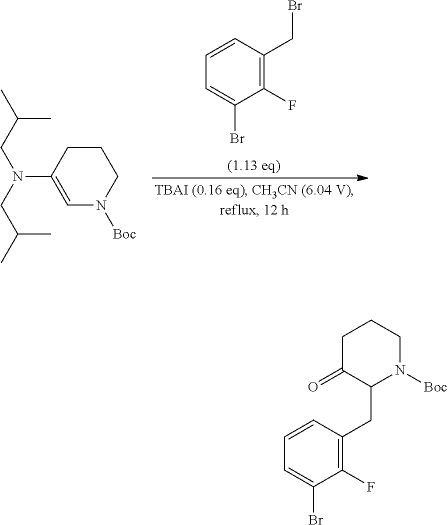 [SMILES: CC(C)CN(CC(C)C)C1=CN(C(=O)OC(C)(C)C)CCC1 ; Fc1c(Br)cccc1CBr ; CC(C)(C)OC(=O)N1CCCC(=O)C1Cc1cccc(Br)c1F]

A 50 L reactor was charged with CH₃CN (4.6 L) and a solution of Intermediate 1 (2.25 kg, 4.28 mol, 1 eq) in CH₃CN (3 L) was added at 20-30° C. A solution of 1-bromo-3-(bromomethyl)-2-fluorobenzene (1.36 kg, 4.82 mol, 1.13 eq) dissolved in CH₃CN (3 L) was added at 20-30° C. over 30 mins, followed by a solution of TBAI (260.46 g, 684 mmol, 0.16 eq) in CH₃CN (3 L) 20-30° C. over 10 mins. The reaction mixture was heated to 80° C. (internal temperature) for 30 min (1.84° C./min) and the temperature held at 80° C. for 12 h under N₂. The reaction was cooled down to 20-30° C. (internal) for 30 min (1.84° C./min) to give a brown solution.

Water (6.5 L) was added to the reaction mixture. Then the mixture was adjusted to pH=4-5 by AcOH (813 mL) at 20-30° C. The mixture was heated to 60° C. (internal) for 30 min (1.16° C./min) and then held at 50-60° C. (internal) for 1-2 h. The mixture was concentrated under reduced pressure (45° C., 0.09 mPa) to remove the CH₃CN (12 L) to give a brown solution. Ethyl acetate (6 L) was added and the mixture was stirred for 10 minutes and the aqueous layer was separated. This extraction was repeated 2 times. The organic layers were adjusted to pH=2-3 by slow addition of aqueous HCl (0.5 N, 6 L). The mixture was stirred for 10 minutes and the aqueous layer was separated. The organic layers were adjusted to pH=7-8 by slow addition of saturated NaHCO₃ solution (9 L). The mixture was stirred for 10 minutes and the aqueous layer was separated. The organic layer was washed with brine (3 L×2), dried over anhydrous Na₂SO₄ (1.5 kg) and filtered. To the organic layers was added silica gel (2 kg) and the mixture was stirred at 20-30° C. for 1 h before filtering. The filter cake was washed with ethyl acetate (3 L×3). The filtrate was concentrated under reduced pressure (45° C., 0.09 mPa) to give a crude product as a brown oil. The crude product was purified by re-crystallization from n-heptane/MTBE (10/1, 1 V) at 20-25° C. There was a gradual precipitation of yellow solid. The suspension was stirred at 20° C. for 2 h. The suspension was filtered and the filter cake was washed with n-heptane (500 mL×4). The solid was dried under reduced pressure (45° C., 0.01 mPa) to give Intermediate 2 (1.45 kg, purity: 99.4%, yield: 87.3%) as a yellow solid. ¹H NMR (400 MHz, CDCl₃) δ ppm 1.30-1.55 (m, 9H), 1.80-2.10 (m, 2H), 2.45-2.60 (m, 2H), 2.90-3.25 (m, 3H), 3.85-4.25 (m, 1H), 4.70-4.90 (m, 1H), 6.95 (t, 1H), 7.05-7.35 (m, 1H), 7.40-7.50 (m, 1H).

### Intermediate 3: (rac)-tert-butyl (2S,3S)-3-amino-2-(3-bromo-2-fluorobenzyl)piperidine-1-carboxylate

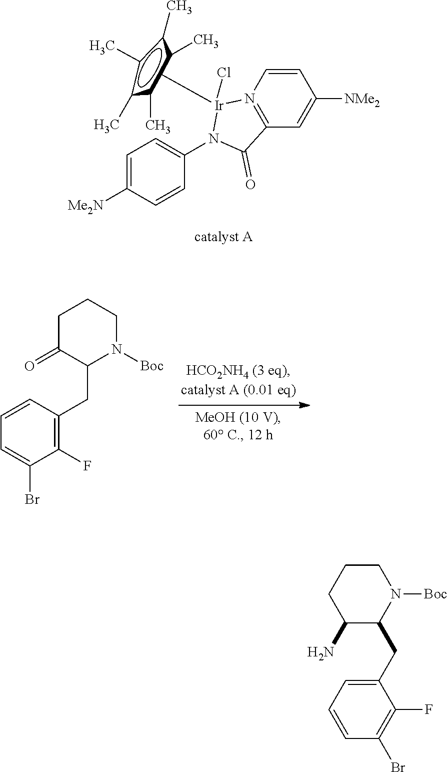

HCO₂NH₄ (470.01 g, 7.30 mol, 3 eq) was added to a solution of Intermediate 2 (950 g, 2.43 mol, 1 eq) in MeOH (9.5 L) at 20-30° C. (internal temperature) over 30 mins. Catalyst A (prepared according to J. Org. Chem. 2019, 84, 10962-10977, 16.03 g, 24.35 mmol, 0.01 eq) was added over 10 mins and the reaction mixture to then heated to 60° C. (internal temperature) for 30 min (1.84° C./min) before being held at 60° C. (internal) for 12 h under N₂. The reaction mixture was cooled to 20° C. adjusted to pH=7-8 by slow addition of aqueous NaHCO₃ solution (5 L). The reaction mixture was concentrated under reduced pressure (40° C., 0.01 mPa) to ⅓ of the original volume before extraction with ethyl acetate (2.5 L×3). The combined organic layers were dried over Na₂SO₄, filtered and concentrated under reduced pressure (45° C., 0.01 mPa) to give crude intermediate 3 (942 g). This batch was combined with another batch of intermediate 3 (1.45 Kg from intermediate 2), in total, 2.36 kg of intermediate 3 was obtained. The crude material was dissolved in ethyl acetate (5 L) and NH₃·H₂O (3 L, 7% w/w) was added to the reactor at 25° C. (pH=10-11). The mixture was stirred at 25° C. for 1 h before extraction with ethyl acetate (5 L×3). The combined organic layers were washed with saturated aqueous solution of sodium chloride (1 L), dried over Na₂SO₄, filtered and concentrated under reduced pressure (45° C., 0.09 mPa) to give intermediate 3 (2.17 Kg, QNMR: 91%, yield: 82.9% based on QNMR) as a yellow oil. ¹H NMR (400 MHz, d6-DMSO) δ ppm 0.90-1.20 (m, 9H), 1.25-1.50 (m, 3H), 1.55-1.75 (m, 3H), 2.75-2.95 (m, 3H), 3.10 (d, 1H), 3.65-3.95 (m, 1H), 4.25-4.45 (m, 1H), 7.00-7.10 (m, 1H), 7.25 (t, 1H), 7.50-7.60 (m, 1H).

### Intermediate 3: Purification by Salt Formation

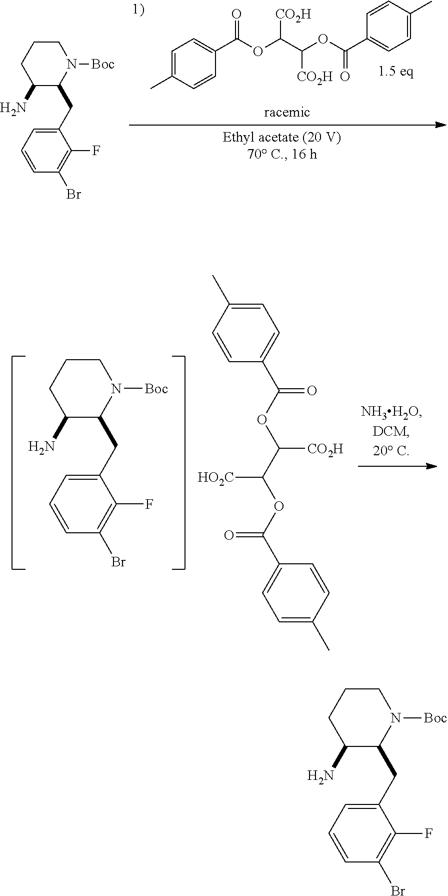

Racemic di-toluoyltartaric acid (149.64 g, 387.31 mmol, 1.5 eq) was dissolved as suspension in ethyl acetate (1 L) and added to a solution of intermediate 3 (100 g, 258.21 mmol, 1 eq) in EtOAc (1 L) at 70° C. The mixture was stirred at 70° C. for 16 h under N₂ to give a yellow slurry. The reaction mixture was cooled down to 20° C. gradually and the solid was isolated via filtration, washed with ethyl acetate (0.5 L) and dried in vacuum (40° C., 10 mbar) to give intermediate 3 salt (155 g, yield: 85%) as a white solid. The intermediate 3 salt (155 g, 200.88 mmol) was dissolved in dichloromethane (775 mL) and NH₃·H₂O (7.0% w/w %, 650 mL, 5 eq) was added until the pH=9-10. The mixture was stirred at 25° C. for 1 h under N₂. The resulting mixture was extracted with dichloromethane (0.5 L×3). The combined organic layers were washed with saturated brine (0.5 L), dried over Na₂SO₄, filtered and concentrated under reduced pressure (45° C., 0.01 mPa) to give racemic intermediate 3 (71 g, QNMR: 96.5%, yield: 76%) as a yellow solid.

### Synthesis of tert-butyl (2S,3S)-3-amino-2-(3-bromo-2-fluorobenzyl)piperidine-1-carboxylate

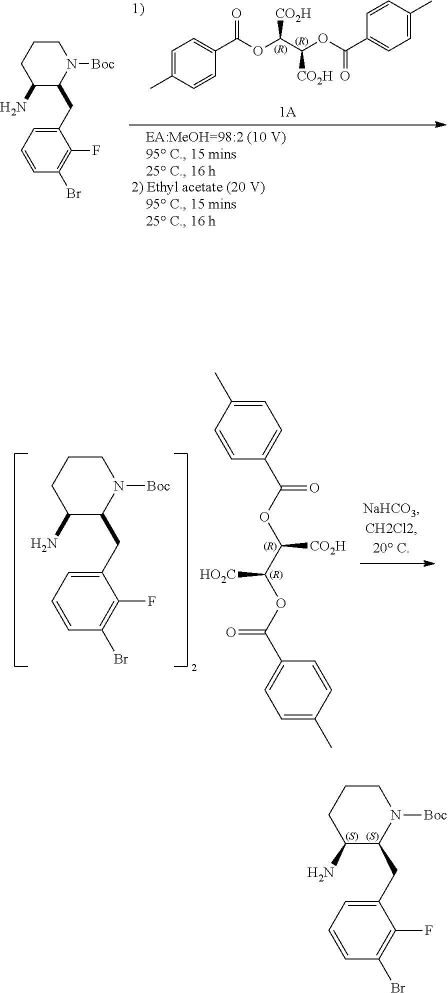

L-(−)-di-toluoyltartaric acid (3.99 g, 10.33 mmol, 0.4 eq) was added to a solution of intermediate 3 (10 g, 25.82 mol, 1 eq) in ethyl acetate (98 mL) and MeOH (2 mL) at 95° C. The suspension was left to stir at reflux temperature for 10 minutes after which the temperature was gradually lowered by switching off the heating and left to stir for 16 h. The temperature fell from 75° C. to 25° C. (internal temperature) during 2 hrs. The solids were isolated by filtration and washed with ethyl acetate (300 mL) and dried in vacuum (40° C., 0.01 MPa) to yield 5.7 g of the intermediate 3 salt with e.e %=92.5%. This was suspended in ethyl acetate (71 mL), and the temperature was increased to reflux temperature and held at this temperature for 15 mins. The temperature was gradually lowered by switching off the heating and the suspension left to stir for 16 h. The solid was isolated via filtration, washed with ethyl acetate (20 mL) and dried in vacuum to give intermediate 3 Salt (5.2 g, purity: 98.5%, e.e %=98.2%) as a white solid.

The salt was suspended in dichloromethane (100 mL) and washed with 0.5 M aqueous NaHCO₃ solution (150 mL). The organic phase was isolated, and the aqueous phase was extracted with dichloromethane (50 mL×3). The combined organic phases were dried with Na₂SO₄, filtered, and concentrated under reduce pressure to give tert-butyl (2S,3S)-3-amino-2-(3-bromo-2-fluorobenzyl)piperidine-1-carboxylate (3.4 g, yield 34%, 68% theoretical) as white solid.

### Synthesis of benzyl (2S,3R,4S)-3-amino-2-(3-bromo-2-fluorobenzyl)-4-fluoropiperidine-1-carboxylate

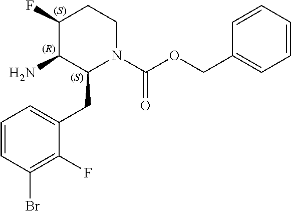

The synthesis of benzyl (2S,3R,4S)-3-amino-2-(3-bromo-2-fluorobenzyl)-4-fluoropiperidine-1-carboxylate is shown in Reaction Schemes 1-3.

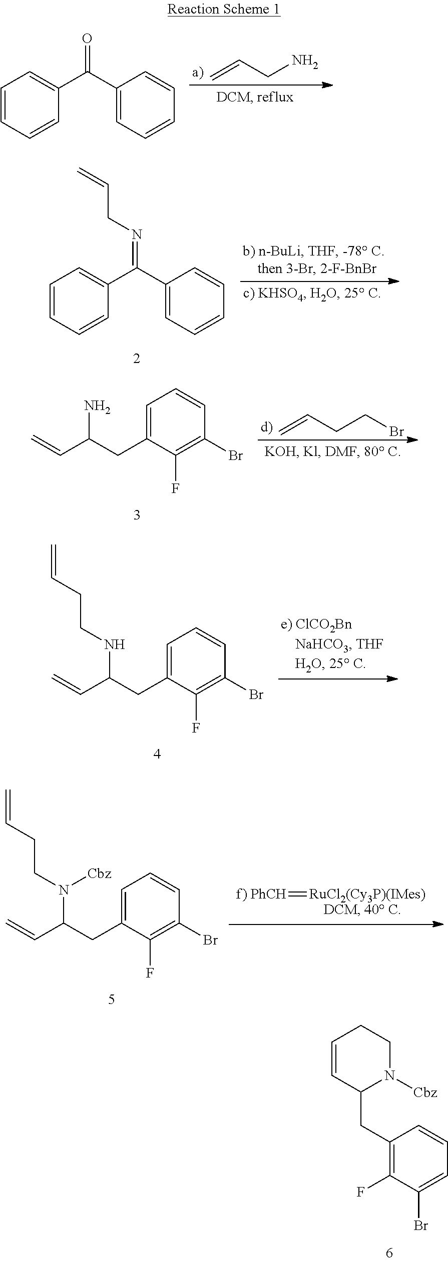

Step a) N-Allyl-1,1-diphenylmethanimine was prepared by the method of Li et al., (Organic Letters, 2017, 19, 4239) by heating a mixture of benzophenone imine with allylamine in dichloromethane under reflux for 18 h. Yield 95%.

UPLC-MS, method M: (CSH-C18, 2 to 95% MeCN) Rₜ=0.52 min, MS (ESI⁺): m/z=[M+H]⁺ 222. ¹H NMR (400 MHz, CDCl₃): δ [ppm]=4.03 (dt, 2H), 5.11 (dq, 1H), 5.17 (dq, 1H), 6.05 (ddt, 1H), 7.19-7.16 (m, 2H), 7.31-7.40 (m, 3H), 7.41-7.49 (m, 3H), 7.62-7.64 (m, 2H).

Step b) To a solution of N-allyl-1,1-diphenylmethanimine (2) (300 g, 1.36 mol) in tetrahydrofuran (2 L) was added dropwise n-butyllithium (2.5 M, 651 mL) over 30 min at −78° C. After the addition was complete, the mixture was stirred at −78° C. for 2 h. Then a solution of 3-bromo-2-fluorobenzyl bromide (399.53 g, 1.49 mol) in tetrahydrofuran (1 L) was added dropwise to the reaction over 1 h at −78° C. The resulting mixture was stirred at −78° C. for 2 h. The reaction was quenched with ethanol (20 mL) to afford a solution of the benzophenone imine of 1-(3-bromo-2-fluorophenyl)but-3-en-2-amine (3), which was used directly for the step c). LCMS (ESI⁺): m/z 407.9 (M+H)⁺

Step c) The solution of the benzophenone imine of 1-(3-bromo-2-fluorophenyl)but-3-en-2-amine (3) was added to a solution of potassium hydrogen sulfate (300.00 g, 2.20 mol) in water (1 L) at −30° C. and the mixture was stirred at 25° C. for 12 h. The organic layer was separated, and the aqueous layer containing the product was washed with methyl tert-butyl ether (3.5 L) followed by dichloromethane (2×3.5 L). The aqueous layer was cooled to 0° C. and basified with 50% NaOH solution (400 mL) to pH=9-10. The product was extracted into dichloromethane (1 L). The extract was washed with brine (2×500 mL), dried (Na₂SO₄) and concentrated under reduced pressure.

The product from five reactions (steps b) and c) starting with N-allyl-1,1-diphenylmethanimine (2) (300 g) were combined to give 1-(3-bromo-2-fluorophenyl)but-3-en-2-amine (3) (870 g, 52% over two steps) as a brown oil.

UPLC, method M: (CSH-C18 2 to 95% MeCN) Rₜ=0.46 min (99.2%), MS (ESI⁺): m/z=244.0/246.0 [M+H]⁺, Br isotopes. 1H NMR (400 MHz, CDCl₃) δ ppm 2.66-2.92 (m, 2H), 3.58-3.67 (m, 1H), 5.02-5.15 (m, 2H), 5.87 (ddd, 1H), 6.93-6.99 (m, 1H), 7.12-7.18 (m, 1H), 7.39-7.45 (m, 1H). ¹⁹F NMR (376 MHz, CDCl₃): δ [ppm]=−110.9 (t, 1F).

Step d) To a mixture of 1-(3-bromo-2-fluorophenyl)but-3-en-2-amine (3) (150 g, 614 mmol) and 4-bromobut-1-ene (124.4 g, 922 mmol) in N,N-dimethylformamide (1.5 L) was added potassium hydroxide (68.96 g, 1.23 mol) and potassium iodide (204 g, 1.23 mol) in one portion at 25° C. under nitrogen. The mixture was stirred at 80° C. for 12 h, cooled to 25° C. and concentrated under reduced pressure. The residue was poured into iced water (1 L) and stirred for 3 min. The aqueous phase was extracted with ethyl acetate (3×1 L). The combined organic phases were dried (Na₂SO₄), filtered and concentrated under reduced pressure.

N-(1-(3-bromo-2-fluorophenyl)but-3-en-2-yl)but-3-en-i-amine (4) (995 g, crude weight of a yellow oil) was obtained from six reactions carried out in parallel and used in step e) without further purification. LCMS (ESI⁺): m/z 298.0 [M+H]⁺.

Step e) Benzyl chloroformate (171.63 g, 1.01 mol) was added dropwise to a mixture of N-(1-(3-bromo-2-fluorophenyl)but-3-en-2-yl)but-3-en-i-amine (4) (200 g, 671 mmol) and sodium bicarbonate (112.69 g, 1.34 mol) in a mixture of tetrahydrofuran (1 L) and water (1 L) under nitrogen at 25° C., and the mixture was stirred at 25° C. for 12 h. The mixture was diluted with water (2 L) and extracted with ethyl acetate (3×1 L). The combined organic phases were dried (Na₂SO₄), filtered and concentrated under reduced pressure. The product from five reactions on this scale were combined and purified by column chromatography on silica gel, eluting with a gradient of petroleum ether:ethyl acetate=1:0 to 5:1, to afford benzyl (1-(3-bromo-2-fluorophenyl)but-3-en-2-yl)(but-3-en-1-yl)carbamate (5) (650 g, yield 41% over two steps) as a yellow oil. ¹H NMR (400 MHz, CDCl₃) δ ppm 2.08-2.39 (m, 2H), 2.94-3.31 (m, 4H), 4.38-4.57 (m, 1H), 4.99 (br d, 2H), 5.06-5.14 (m, 2H), 5.15-5.22 (m, 2H), 5.57-5.80 (m, 1H), 5.88-6.15 (m, 1H), 6.82-6.90 (m, 1H), 6.91-7.13 (m, 1H), 7.29-7.45 (m, 6H).

Step f) Benzylidene[1,3-bis(2,4,6-trimethylphenyl)-2-imidazolidinylidene]dichloro (tricyclohexylphosphine) ruthenium (Grubbs' generation II catalyst, 19.64 g, 23.13 mmol) was added to a solution of benzyl (1-(3-bromo-2-fluorophenyl)but-3-en-2-yl)(but-3-en-1-yl)carbamate (5) (100 g, 231 mmol) in dichloromethane (2 L) at 25° C., under nitrogen and with protection from light. The mixture was heated at 40° C. for 12 h, and then concentrated under reduced pressure. The crude product from six reactions carried out on this scale was purified by column chromatography on silica gel, eluting with a gradient of petroleum ether:ethyl acetate=1:0 to 5:1, to afford benzyl 6-(3-bromo-2-fluorobenzyl)-3,6-dihydropyridine-1(2H)-carboxylate (6) (477 g, yield 82%), as a yellow oil.

UPLC, method M: (column CSH-C18, 2 to 95% MeCN) Rₜ=3.17 min (100%), MS (ESI⁺): m/z 404/406 [M+H]⁺, Br isotopes. ¹H NMR (400 MHz, DMSO-d₆, 80° C.): δ [ppm]=1.95-1.99 (m, 1H), 2.06-2.14 (m, 1H), 2.86-3.02 (m, 3H), 4.04 (dd, 1H), 4.64-5.02 (m, 3H), 5.68 (d, 1H), 5.86-5.90 (m, 1H), 7.02 (t, 1H), 7.21-7.41 (m, 6H), 7.50 (t, J 7.3 Hz, 1H). ¹⁹F NMR (376 MHz, DMSO-d₆, 80° C.): δ [ppm]=−112.2 (1F).

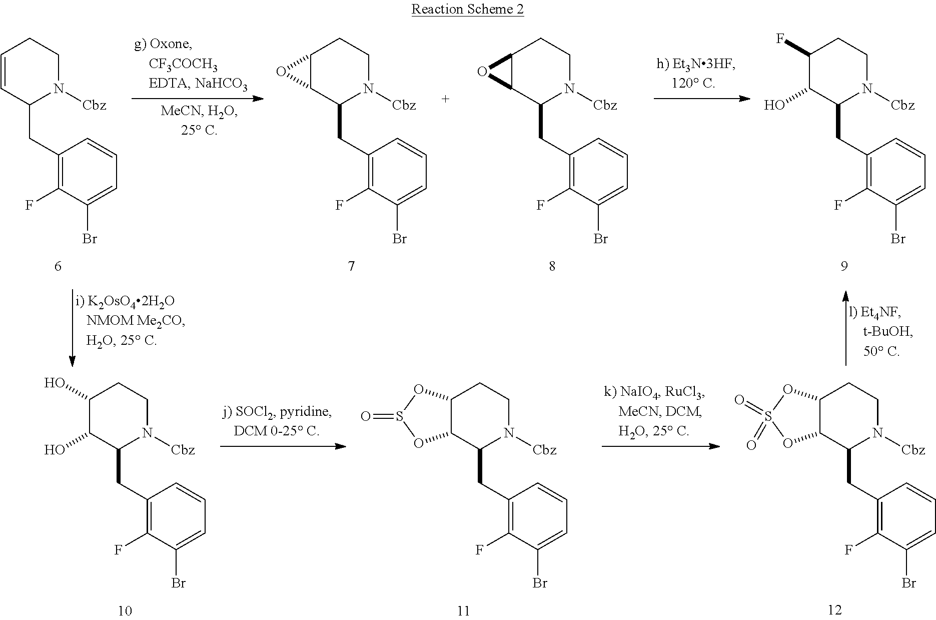

Step g) To a mixture of benzyl 6-(3-bromo-2-fluorobenzyl)-3,6-dihydropyridine-1(2H)-carboxylate (6) (200 g, 498 mmol) and 1,1,1-trifluoropropan-2-one (355 mL, 3.96 mol) in a mixture of acetonitrile (1 L) and water (1 L) was added ethylenediamine tetraacetic acid (EDTA, 14.46 g, 49.5 mmol), Oxone® (CAS 70693-62-8, containing potassium persulfate 3.26 mmol/g, 912 g, 1.48 mol) and solid sodium bicarbonate (623 g, 7.42 mol) at 0° C. under nitrogen. The mixture was stirred at 0° C. for 3 min, then heated to 25° C. and stirred for 12 h. The mixture was poured into water (2 L) and stirred for 3 min. The aqueous phase was extracted with ethyl acetate (3×1 L). The combined organic phases were dried (Na₂SO₄), filtered and concentrated under reduced pressure.

The crude product was combined with another reaction performed using 177 g of compound 6.

Separation of the isomeric epoxides 7 and 8 was achieved by column chromatography on silica gel, eluting with a gradient of petroleum ether:ethyl acetate=1:0 to 3:1). (rac)-Benzyl (1RS,2RS,6SR)-2-(3-bromo-2-fluorobenzyl)-7-oxa-3-azabicyclo[4.1.0]heptane-3-carboxylate (7) (150 g, 38%) was obtained as a yellow oil. ¹H NMR (400 MHz, CDCl₃) δ ppm 1.64-1.87 (m, 1H), 1.94-2.15 (m, 1H), 3.06 (br s, 4H), 3.21-3.42 (m, 1H), 3.56-3.96 (m, 1H), 4.71-4.89 (m, 1H), 4.91-5.16 (m, 2H), 6.82-7.07 (m, 1.5H), 7.27 (s, 2H), 7.28-7.47 (m, 4.5H).

Step h) A mixture of (rac)-benzyl (1RS,2RS,6SR)-2-(3-bromo-2-fluorobenzyl)-7-oxa-3-azabicyclo[4.1.0]heptane-3-carboxylate (7) (20.00 g, 47.59 mmol) and triethylamine trihydrofluoride (80 mL) under nitrogen in a teflon-lined autoclave was heated at 120° C. for 12 h. The mixture was poured into water (2 L), stirred for 3 min and extracted with ethyl acetate (3×1 L). The combined organic phases were dried (Na₂SO₄), filtered and concentrated under reduced pressure.

The crude products from seven reactions were combined. Purification by column chromatography on silica gel, eluting with a gradient of petroleum ether:ethyl acetate=1:0 to 2:1 gave (rac)-benzyl (2RS,3RS,4RS)-2-(3-bromo-2-fluorobenzyl)-4-fluoro-3-hydroxypiperidine-1-carboxylate (9)(130 g, 89%), as a yellow oil.

UPLC, method M: Rₜ=2.55 min (96.0% by UV-TAC), MS (ESI*): m/z=440.0/442.0, [M+H]⁺, Br isotopes. 1H NMR (396 MHz, DMSO-d₆, 80° C.) δ [ppm]1.76-1.83 (m, 1H), 1.91-2.11 (m, 1H), 2.97 (dd, 1H), 3.03-3.09 (m, 1H), 3.26 (dt, 1H), 3.77 (m, 1H), 3.92 (dd, 1H), 4.44 (dd, 1H), 4.68 (dq, 1H), 4.77-4.80 (m, 1H), 4.92 (d, 1H), 5.23 (d, 1H), 7.02 (t, 1H), 7.16 (d, 1H), 7.21-7.25 (m, 1H), 7.26-7.34 (m, 2H), 7.49-7.52 (m, 1H). ¹⁹F NMR (373 MHz, DMSO-d₆, 80° C.) δ [ppm] −112.3 (s, 1F), −185.8 (br t, 1F).

Step i) To a solution of (rac)-benzyl 6-(3-bromo-2-fluorobenzyl)-3,6-dihydropyridine-1(2H)-carboxylate (6) (30.0 g, 80% purity, 59.4 mmol) in a mixture of acetone (600 mL) and water (150 mL) was added potassium osmate dihydrate (547 mg, 1.49 mmol) and N-methylmorpholine-N-oxide (20.9 g, 178 mmol). The mixture was stirred at 25° C. for 3 h. The acetone was removed under reduced pressure and the residue was stirred with the mixture of ethyl acetate (200 mL) and a solution of sodium bisulfite (30.8 g, 297 mmol) in water (600 mL) for 30 min. After being filtered, the aqueous phase was separated, and the organic phase was extracted with ethyl acetate (3×200 mL). The combined organic layers were washed with brine (200 mL), dried (Na₂SO₄), filtered and concentrated under reduced pressure. The residue was triturated with petroleum ether:ethyl acetate=5:1 (1 V) at 25° C. for 120 min to give benzyl (2RS,3RS,4SR)-2-(3-bromo-2-fluorobenzyl)-3,4-dihydroxypiperidine-1-carboxylate (10) (21 g, 75%), as an off-white solid.

UPLC method M: Rₜ=2.07 min (97.7% by UV-TAC), MS (ESI⁺): m/z=438/440 [M+H]⁺, Br isotopes, MS (ESI⁻): m/z=436/438 [M−H]⁻, Br isotopes. 1H NMR (396 MHz, DMSO-d₆, 80° C.) δ [ppm]1.46-1.50 (m, 1H), 1.69 (qd, 1H), 2.86 (d, 2H), 2.98-3.11 (m, 1H), 3.59 (s, 1H), 3.83 (d, 1H), 3.96 (dd, 1H), 4.19-4.33 (m, 2H), 4.46 (t, 1H), 4.69-4.91 (m, 2H), 6.98 (t, 1H), 7.08-7.32 (m, 6H), 7.46 (td, 1H). ¹⁹F NMR (373 MHz, DMSO-d₆, 80° C.) 6 [ppm] −112.2 (s, 1F).

Step j) (rac)-benzyl (2RS,3RS,4SR)-2-(3-bromo-2-fluorobenzyl)-3,4-dihydroxypiperidine-1-carboxylate (10)(21.0 g, 47.9 mmol) was suspended in dichloromethane (600 mL) under nitrogen. Anhydrous pyridine (15.16 g, 192 mmol) was added, and the resulting solution was cooled in an ice-water bath. Thionyl chloride (7.11 g, 71.9 mmol) was added dropwise, and the mixture was removed from the ice-water bath and stirred at room temperature for 1 h. The mixture was evaporated under reduced pressure. The residue was partitioned between ethyl acetate (200 mL) and water (100 mL). The organic phase was washed with brine (100 mL), dried (MgSO₄), filtered and evaporated under reduced pressure to give (rac)-benzyl (3aR,4R,7aS)-4-(3-bromo-2-fluorobenzyl)tetrahydro-[1,3,2]dioxathiolo[4,5-c]pyridine-5(4H)-carboxylate 2-oxide (11)(21 g, 88%, 4:1 mixture of epimers at sulfur), as a white solid, which was used in step k) without any purification.

UPLC method M: Rₜ=2.16 min (92% by UV-254 nM), MS (ESI⁺): m/z=484/486.0 [M+H]⁺, Br isotopes. ¹H NMR (396 MHz, DMSO-d₆, 80° C.) δ 1.62 (td, 0.8H), 2.08 (qd, 0.2H), 2.13-2.25 (m, 1H), 2.95-3.02 (m, 1H), 3.09-3.23 (m, 2H), 3.63-3.70 (dt, 0.8H), 3.87 (dt, 0.2H), 4.70-5.00 (m, 4H), 5.11-5.16 (m, 0.2H), 5.39 (dd, 0.8H), 7.00 (t, 1H), 7.05-7.17 (m, 2H), 7.21-7.32 (m, 4H), 7.48-7.52 (m, 1H). ¹⁹F NMR (373 MHz, DMSO-d₆, 80° C.) 6 [ppm] −112.0 (s, 1F).

Step k) To a solution of (rac)-benzyl (3aR,4R,7aS)-4-(3-bromo-2-fluorobenzyl)tetrahydro-[1,3,2]dioxathiolo[4,5-c]pyridine-5(4H)-carboxylate 2-oxide (11) (21.0 g, 39.4 mmol) in acetonitrile (84 mL), dichloromethane (84 mL) and water (168 mL) was added sodium metaperiodate (9.26 g, 43.3 mmol) and ruthenium trichloride (81.7 mg, 0.394 mmol). The mixture was stirred at 25° C. for 1 h. The mixture was filtered and the solid was washed with ethyl acetate (50 mL). The filtrate was evaporated under reduced pressure and the residue partitioned between ethyl acetate (150 mL) and water (50 mL). The organic layer was separated, dried (MgSO₄), filtered and evaporated under reduced pressure to give benzyl (rac)-(3aR,4R,7aS)-4-(3-bromo-2-fluorobenzyl)tetrahydro-[1,3,2]dioxathiolo[4,5-c]pyridine-5(4H)-carboxylate 2,2-dioxide (12) (19.3 g, purity 90%, yield 88%) as a white solid which was used to next step without further purification.

UPLC, method M: Rₜ=3.66 min (96.0% by UV-254 nm), MS (ESI⁻): m/z=498/500 [M−H]⁻, Br isotopes. 1H NMR (396 MHz, DMSO-d₆, 80° C.) δ [ppm]1.95-2.06 (m, 1H), 2.27-2.34 (m, 1H), 2.94-3.12 (m, 1H), 3.17-3.24 (m, 1H), 3.87 (dt, 1H), 4.72-4.83 (m, 1H), 4.86-4.94 (m, 2H), 5.27-5.32 (m, 0.04H), 5.32-5.37 (m, 0.96H), 5.59-5.70 (m, 1H), 7.00 (t, 1H), 7.06-7.13 (m, 2H), 7.22-7.32 (m, 4H), 7.48-7.53 (m, 1H). ¹⁹F NMR (373 MHz, DMSO-d₆, 80° C.) δ [ppm] −111.9 (s, 1F).

Step l) To a solution of benzyl (rac)-(3aR,4R,7aS)-4-(3-bromo-2-fluorobenzyl)tetrahydro-[1,3,2]dioxathiolo [4,5-c]pyridine-5(4H)-carboxylate 2,2-dioxide (12) (18 g, 36 mmol) in tert-butanol (360 mL) was added tetraethylammonium fluoride hydrate (12.0 g, 72 mmol). The mixture was stirred at 50° C. for 3 h. The mixture was filtered, and the solid was washed with ethyl acetate (50 mL). The filtrate was evaporated under reduced pressure and the residue partitioned between ethyl acetate (200 mL) and water (80 mL). The organic layer was separated, dried (MgSO₄), filtered and evaporated under reduced pressure. The residue was dissolved in methyl tert-butyl ether (900 mL) and aqueous sulfuric acid (2 M, 900 mL) was added. The mixture was stirred at 25° C. for 1 h. The mixture was diluted with water (500 mL) at 0° C. and extracted with ethyl acetate (3×500 mL). The combined organic extracts were washed with brine (500 mL), dried (Na₂SO₄), filtered and concentrated under reduced pressure. The residue was purified by column chromatography on silica gel, eluting with petroleum ether:ethyl acetate=3:1. Product containing fractions were combined, concentrated under reduced pressure and the resulting solid was triturated with petroleum ether:methyl tert-butyl ether=10:1 (3 V) at 0° C. for 120 min to give (rac)-benzyl (2RS,3RS,4RS)-2-(3-bromo-2-fluorobenzyl)-4-fluoro-3-hydroxypiperidine-1-carboxylate (9) (9.5 g, 73%), as a colourless solid.

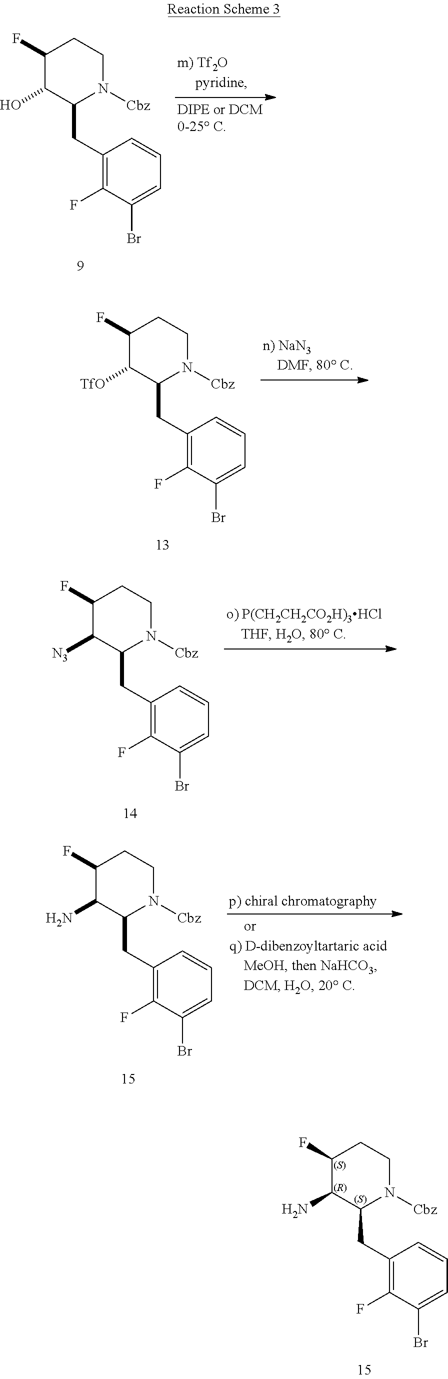

Step m) To a mixture of (rac)-benzyl (2R,3R,4R)-2-(3-bromo-2-fluorobenzyl)-4-fluoro-3-hydroxypiperidine-1-carboxylate (9)(130 g, 295 mmol) and pyridine (477 mL, 5.91 mol) in diisopropyl ether (1.9 L) was added dropwise triflic anhydride (244 mL, 1.48 mol) at 0° C. under nitrogen. The mixture was stirred at 0° C. for 3 min, then allowed to warm to 25° C. and stirred for 12 h. The mixture was poured into hydrochloric acid (0.25 M, 2 L). The aqueous phase was extracted with ethyl acetate (3×1 L). The combined organic phases were dried (Na₂SO₄), filtered and concentrated under reduced pressure. The residue was purified by column chromatography on silica gel, eluting with petroleum ether:ethyl acetate=1:0 to 3:1) to afford (rac)-benzyl (2R,3R,4R)-2-(3-bromo-2-fluorobenzyl)-4-fluoro-3-(((trifluoromethyl)sulfonyl)oxy)piperidine-1-carboxylate (13)(110 g, 60% yield) as a yellow oil.

UPLC, method M: (CSH-C18 50 to 95% MeCN) Rₜ=0.92 min (100%), MS (ESI⁺): m/z=572.1, 574.1 [M+H]⁺, Br isotopes. 1H NMR (400 MHz, CDCl₃): δ [ppm]1.97-2.14 (m, 2H), 3.06-3.01 (m, 1H), 3.12-3.18 (m, 1H), 3.30 (m, 1H), 4.1 (br s, 1H minor rotamer), 4.24 (d, 1H major rotamer), 4.88-5.09 (m, 5H), 6.89 (t, J=7 Hz, 1H), 7.02 (t, 1H), 7.11-7.21 (m, 1H), 7.28-7.38 (m, 3H), 7.42 (t, 1H). ¹⁹F NMR (376 MHz, CDCl₃): δ [ppm] −187.94 (s, 1F major rotamer), −187.85 (s, 1F minor rotamer), −111.6 (s, 1F major rotamer), −111.2 (s, 1F minor rotamer), −75.0 (s, 3F, major rotamer), −74.8 (s, 3F minor rotamer).

Step m) alternative procedure: A solution of (rac)-benzyl (2R,3R,4R)-2-(3-bromo-2-fluorobenzyl)-4-fluoro-3-hydroxypiperidine-1-carboxylate (9) (0.5 g, 0.91 mmol) and pyridine (0.36 g, 4.54 mmol) in dichloromethane (10 mL) was degassed and purged with nitrogen three times. The mixture was cooled to 0° C. A solution of triflic anhydride (0.77 g, 2.72 mmol) in dichloromethane (2 mL) was added dropwise at 0° C. The mixture was stirred at 0° C. for 2 h and poured into aqueous copper sulfate (0.65 g in 30 mL water) at 0° C. The resulting mixture was partitioned between dichloromethane and water. The organic phase was washed twice with aqueous copper sulfate (0.65 g in 30 mL water), saturated aqueous sodium bicarbonate (2.4 g in 25 mL water), and finally aqueous potassium dihydrogen phosphate (1 M, 20 mL) to remove any alkaline traces from the triflate. Then the organic phase was dried (Na₂SO₄) and concentrated under reduced pressure. The crude triflate was co-evaporated with tetrahydrofuran (2×10 mL) to give (rac)-benzyl (2R,3R,4R)-2-(3-bromo-2-fluorobenzyl)-4-fluoro-3-(((trifluoromethyl) sulfonyl)oxy)piperidine-1-carboxylate (13)(510 g, 98%), as an almost colourless oil.

Step n) To a mixture of (rac)-benzyl (2R,3R,4R)-2-(3-bromo-2-fluorobenzyl)-4-fluoro-3-(((trifluoromethyl) sulfonyl)oxy)piperidine-1-carboxylate (13)(100 g, 174 mmol) in N,N-dimethylformamide (1.0 L) was added sodium azide (34.1 g, 524 mmol) in one portion at 25° C. under nitrogen. The mixture was heated to 80° C. and stirred for 1 h. The mixture was cooled to 25° C., poured into water (2 L) and stirred for 3 min. The aqueous phase was extracted with ethyl acetate (3×1 L). The combined organic phases were washed with brine (3×800 mL), dried (Na₂SO₄), filtered and concentrated under reduced pressure. The residue was purified by column chromatography on silica gel, eluting with petroleum ether:ethyl acetate=1:0 to 3:1, to afford (rac)-benzyl (2R,3S,4R)-3-azido-2-(3-bromo-2-fluorobenzyl)-4-fluoropiperidine-1-carboxylate (14) (56 g, yield 69%) as a light yellow oil.

LCMS method E (ESI⁺): Rₜ=0.775 min, m/z 465.0 (M+H)⁺. ¹H NMR (400 MHz, DMSO-d₆) 1.73-1.86 (m, 1H), 2.02-2.08 (m, 1H), 2.97-3.11 (m, 2H), 3.30 (m, 1H), 3.91 (m, 1H), 4.17 (dd, 1H), 4.55-4.84 (br d, 3H), 5.13 (d, 1H), 7.00 (t, 2H), 7.35-7.25 (m, 5H), 7.53 (t, 1H).

Step o) To a mixture of (rac)-benzyl (2R,3S,4R)-3-azido-2-(3-bromo-2-fluorobenzyl)-4-fluoropiperidine-1-carboxylate (14) (56 g, 120 mmol) in tetrahydrofuran (560 mL) and water (560 mL) was added 3-[bis(2-carboxyethyl)phosphanyl]propanoic acid hydrochloride (69.0 g, 241 mmol) in one portion at 25° C. under nitrogen. The mixture was stirred at 25° C. for 3 min, then heated to 80° C. and stirred for 12 h. The mixture was cooled to 25° C. The residue was poured into iced-water (1 L) and stirred for 3 min. The aqueous phase was extracted with ethyl acetate (3×1 L). The combined organic phases were dried (Na₂SO₄), filtered and concentrated under reduced pressure. The residue was purified by column chromatography on silica gel, eluting with a gradient of petroleum ether:ethyl acetate=1:0 to 0:1) to afford (rac)-(2R,3S,4R)-3-amino-2-(3-bromo-2-fluorobenzyl)-4-fluoropiperidine-1-carboxylate (15) (35 g, yield 66%), as a colourless oil. LCMS (ESI⁺): m/z 439.1 (M+H)⁺, Rₜ=0.60 min

Step p) The enantiomers of (rac)-(2R,3S,4R)-3-amino-2-(3-bromo-2-fluorobenzyl)-4-fluoropiperidine-1-carboxylate (15) (35 g, 79.7 mmol) were separated by chiral supercritical fluid chromatography.

For analytical purposes, the enantiomers were separated using a Daicel Chiralpak IC-3 (50 mm×4.6 mm, 3 μm); flow rate 3.4 mL/min, 35° C. Mobile phase A=scCO₂; Mobile phase B=0.1% isopropylamine in ethanol). Gradient: 0-0.2 min 5% B; 0.2-1.2 min 5-50% B, 1.2-2.2 min 50% B. The (+)-enantiomer eluted first (Rₜ=1.11 min), followed by the desired (−)-enantiomer (Rₜ=1.28 min).

For a preparative separation, the enantiomers were separated using a Daicel Chiralpak IC (250 mm×50 mm, 10 μm); mobile phase: 30% (isocratic) of 0.1% aqueous ammonia in ethanol with 70% scCO₂, 4.3 min). The (+)-enantiomer eluted first, followed by the desired (−)-enantiomer.

Benzyl (2S,3R,4S)-3-amino-2-(3-bromo-2-fluorobenzyl)-4-fluoropiperidine-1-carboxylate was obtained as a colourless oil (14.7 g, yield 39%, ⁹5.9% purity, e.e. =99.2%, [α]_{D}=−2.11°, c=1.04, MeOH, 20° C.).

LCMS method E: Rₜ=2.14 min; (ESI): m/z 439.0 (M+H)⁺. ¹H NMR (400 MHz, CD₃CN) δ ppm 1.65-1.88 (m, 1H+NH₂), 1.98-2.06 (m, 1H), 2.87-3.01 (m, 2H), 3.14-3.31 (m, 2H), 3.95 (br d, 1H), 4.35-4.60 (m, 2H), 4.81 (dq, 1H), 4.82 (br s, 1H), 6.94 (t, 1H), 7.17 (br s, 2H), 7.18 (m, 1H), 7.24-7.35 (m, 3H), 7.41 (t, 1H). ¹⁹F NMR (376 MHz, DMSO-d₆) δ ppm −113.5 (s, 1F), −198.3 (s, 1F).

Step q) Alternative method: A 100 mL round-bottom-flask was charged with (rac)-(2R,3S,4R)-3-amino-2-(3-bromo-2-fluorobenzyl)-4-fluoropiperidine-1-carboxylate (15) (565.50 mg, 1.29 mmol), methanol (5.65 mL), and D-(+)-di-benzoyltartaric acid (461 mg, 1.29 mmol). The solution was stirred at room temperature for 16 h during which time a white precipitate formed. The solid was collected by filtration, washed with methanol (50 mL), and dried at 45° C. for 2 h to yield the D-(+)-di-benzoyltartaric acid salt of compound 1 (1:1 mol/mol ratio, 373 mg, 36%, 99% ee), as a colourless powder.

The salt (373 mg) was suspended in dichloromethane (50 mL) and washed with aqueous sodium bicarbonate (0.5 M, 50 mL). The organic phase was isolated, and the aqueous phase was extracted with dichloromethane (2×10 mL). The combined organic phases were dried (Na₂SO₄), filtered, and concentrated in vacuo to yield benzyl (2S,3R,4S)-3-amino-2-(3-bromo-2-fluorobenzyl)-4-fluoropiperidine-1-carboxylate (1) (191 mg, 34%, 98% ee), as a colourless oil.

### Example 1. Procedure for the Preparation of Example 1

### Step 1: tert-Butyl-3-amino-2-(3-bromo-benzyl)-4,4-difluoropyrrolidine-1-carboxylate_cis racemic

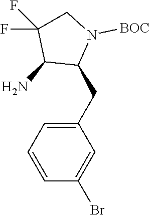 [SMILES: CC(C)(C)OC(=O)N1CC(F)(F)[C@H](N)[C@@H]1Cc1cccc(Br)c1]

Compound was synthesised according to the procedure reported in WO2020/158958 A1 for tert-Butyl-3-amino-2-(3-chloro-benzyl)-4,4-difluoropyrrolidine-1-carboxylate_cis racemic.

### Step 2: tert-butyl 3-amino-2-[[3-[2-[2-[tert-butoxycarbonyl(methyl)amino]ethoxy]-3-fluoro-phenyl]phenyl]methyl]-4,4-difluoro-pyrrolidine-1-carboxylate_cis racemic

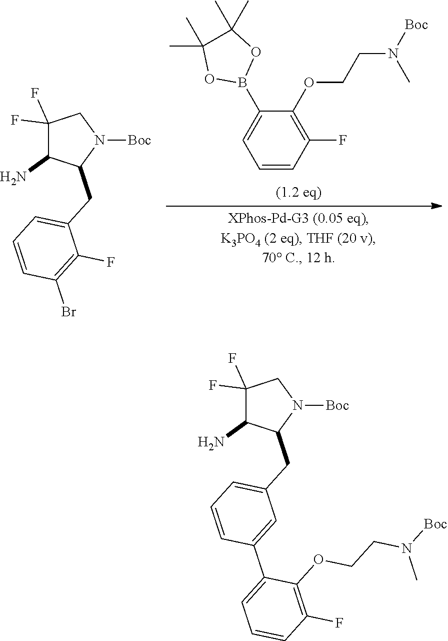 [SMILES: CC(C)(C)OC(=O)N1CC(F)(F)[C@H](N)[C@@H]1Cc1cccc(Br)c1F ; CN(CCOc1c(F)cccc1B1OC(C)(C)C(C)(C)O1)C(=O)OC(C)(C)C ; CN(CCOc1c(F)cccc1-c1cccc(C[C@H]2[C@@H](N)C(F)(F)CN2C(=O)OC(C)(C)C)c1)C(=O)OC(C)(C)C]

To a solution of tert-Butyl-3-amino-2-(3-bromo-benzyl)-4,4-difluoropyrrolidine-1-carboxylate_cis racemic (3 g, 7.7 mmol, 1 eq) in THE (60 mL) was added tert-butyl N-methyl-N-[2-[2-(4,4,5,5-tetramethyl-1,3,2-dioxaborolan-2-yl)phenoxy]ethyl]carbamate (3.6 g, 9.2 mmol, 1.2 eq), K₃PO₄ (3.27 g, 15.33 mmol, 2 eq) and XPhos-Pd-G3 (325 mg, 128 μmol, 0.05 eq). The mixture was stirred at 70° C. for 12 hrs under N₂. The mixture was poured into water (50 mL) and extracted with ethyl acetate (3×30 mL). The organic layer was washed with brine (50 mL) and dried over sodium sulfate, concentrated under reduced pressure to give crude product. The residue was purified by column chromatography (SiO₂, Petroleum ether/Ethyl acetate=100:1 to 1:1) to afford tert-butyl 3-amino-2-[[3-[2-[2-[tert-butoxycarbonyl(methyl)amino]ethoxy]-3-fluoro-phenyl]phenyl]methyl]-4,4-difluoro-pyrrolidine-1-carboxylate_cis racemic (3 g, 68% yield) as white solid. LCMS, Method E: (ESI⁺): m/z 580 (M+H)⁺, RT: 0.77 min.

### Step 3: tert-Butyl 2-[[3-[2-[2-[tert-butoxycarbonyl(methyl)amino]ethoxy]-3-fluoro-phenyl]phenyl]methyl]-3-(difluoromethylsulfonylamino)-4,4-difluoro-pyrrolidine-1-carboxylate_cis racemic

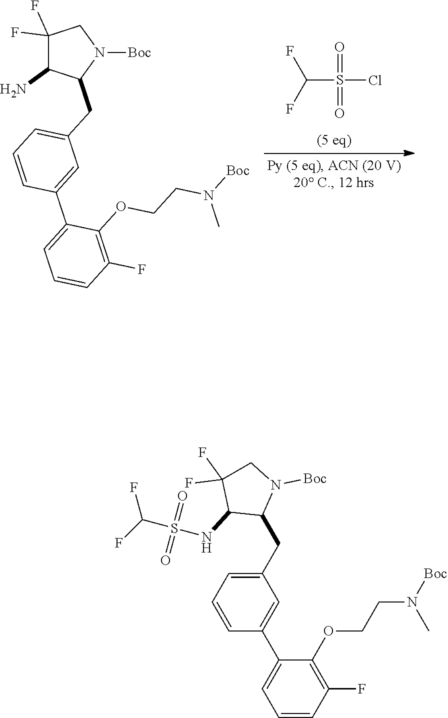 [SMILES: CN(CCOc1c(F)cccc1-c1cccc(C[C@H]2[C@@H](N)C(F)(F)CN2C(=O)OC(C)(C)C)c1)C(=O)OC(C)(C)C ; O=S(=O)(Cl)C(F)F ; CN(CCOc1c(F)cccc1-c1cccc(C[C@H]2[C@@H](NS(=O)(=O)C(F)F)C(F)(F)CN2C(=O)OC(C)(C)C)c1)C(=O)OC(C)(C)C]

To a solution of tert-butyl3-amino-2-[[3-[2-[2-[tert-butoxycarbonyl(methyl)amino]ethoxy]-3-fluoro-phenyl]phenyl]methyl]-4,4-difluoro-pyrrolidine-1-carboxylate_cis racemic (1.3 g, 1.6 mmol, 1 eq) in acetonitrile (26 mL) was added pyridine (890 mg, 11.25 mmol, 910 μL, 5 eq). Then the reaction mixture was cooled to 0° C. and difluoromethanesulfonyl chloride (1.69 g, 11.25 mmol, 5 eq) was added. The mixture was stirred at 20° C. for 12 hrs. The mixture was poured into water (20 mL), and extracted with ethyl acetate (3×20 mL). The organic layer was washed with brine (20 mL) and dried over sodium sulfate, concentrated under reduced pressure to give crude product. The residue was purified by column chromatography (SiO₂, Petroleum ether/Ethyl acetate=100:1 to 1:1) to afford tert-butyl 2-[[3-[2-[2-[tert-butoxycarbonyl(methyl)amino]ethoxy]-3-fluoro-phenyl]phenyl]methyl]-3-(difluoromethylsulfonylamino)-4,4-difluoro-pyrrolidine-1-carboxylate_cis racemic (850 mg, 55% yield) as white solid. LCMS, Method E: (ESI⁺): m/z 594 (M+H−100)⁺, RT: 0.93 min.

### Step 4: N-[4,4-difluoro-2-[[3-[3-fluoro-2-[2-(methylamino)ethoxy]phenyl]phenyl]meth yl]pyrrolidin-3-yl]-1,1-difluoro-methanesulfonamide_cis racemic

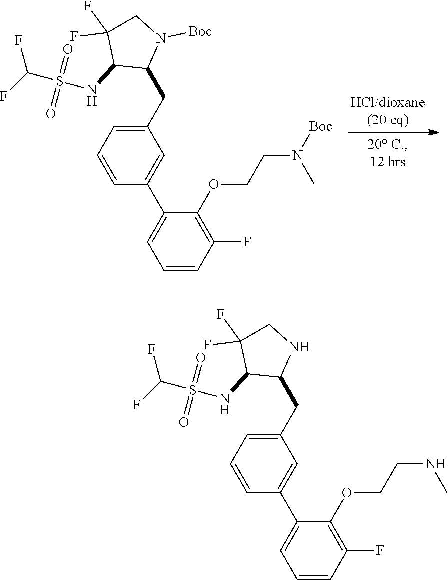 [SMILES: CN(CCOc1c(F)cccc1-c1cccc(C[C@H]2[C@@H](NS(=O)(=O)C(F)F)C(F)(F)CN2C(=O)OC(C)(C)C)c1)C(=O)OC(C)(C)C ; CNCCOc1c(F)cccc1-c1cccc(C[C@@H]2NCC(F)(F)[C@@H]2NS(=O)(=O)C(F)F)c1]

To a solution of tert-butyl 2-[[3-[2-[2-[tert-butoxycarbonyl(methyl)amino]ethoxy]-3-fluoro-phenyl]phenyl]methyl]-3-(difluoromethylsulfonylamino)-4,4-difluoro-pyrrolidine-1-carboxylate_cis racemic (850 mg, 1.23 mmol, 1 eq) in dioxane (10 mL) was added HCl/dioxane (4 M, 6.13 mL, 20 eq). The mixture was stirred at 20° C. for 12 hours. The resulting mixture was concentrated under reduced pressure to give N-[4,4-difluoro-2-[[3-[3-fluoro-2-[2-(methylamino)ethoxy]phenyl]phenyl]methyl]pyrrolidin-3-yl]-1,1-difluoro-methanesulfonamide (500 mg, 83% yield) as a white solid. The crude product was used for next step directly without purification. LCMS, Method E: (ESI⁺): m/z 494 (M+H)⁺, RT: 0.57 min.

### Step 5: 1,1-difluoro-N-((4²S, 4³R)-1³,4⁴, 4⁴-trifluoro-6-methyl-5-oxo-9-oxa-6-aza-4(2,1)-pyrrolidina-1(1,2),2(1,3)-dibenzenacyclononaphane-4³-yl)methanesulfonamide_cis racemic

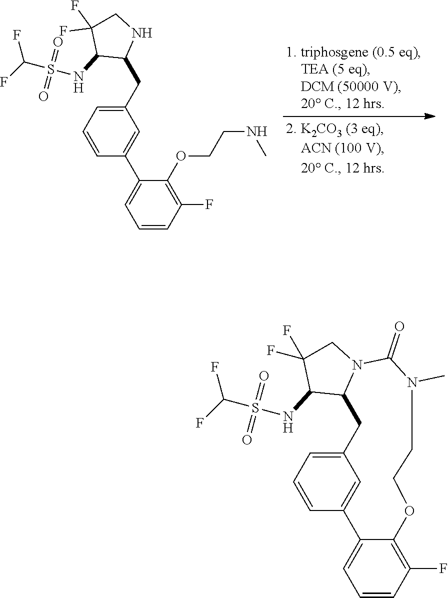 [SMILES: CNCCOc1c(F)cccc1-c1cccc(C[C@@H]2NCC(F)(F)[C@@H]2NS(=O)(=O)C(F)F)c1 ; CN1CCOc2c(F)cccc2-c2cccc(c2)C[C@H]2[C@@H](NS(=O)(=O)C(F)F)C(F)(F)CN2C1=O]

To a solution of N-[4,4-difluoro-2-[[3-[3-fluoro-2-[2-(methylamino)ethoxy]phenyl]phenyl]methyl]pyrrolidin-3-yl]-1,1-difluoro-methanesulfonamide_cis racemic (500 mg, 1.0 mmol, 1 eq) in dichloromethane (2.5 L) was added triethylamine (510 mg, 5.1 mmol, 700 μL, 5 eq) and triphosgene (150 mg, 500 μmol, 0.5 eq). The mixture was stirred at 20° C. for 12 hours. Then the resulting mixture was concentrated under reduced pressure to give a residue, which was dissolved in acetonitrile (50 mL). To the solution was added K₂CO₃ (420 mg, 3.0 mmol, 3 eq) at 20° C. The reaction was stirred at 20° C. for 12 hrs. The mixture was poured into water (30 mL) and extracted with ethyl acetate (3×30 mL). The organic layer was washed with brine (30 mL) and dried over sodium sulfate, concentrated under reduced pressure to give crude product. The residue was purified by column chromatography (SiO₂, Petroleum ether/Ethyl acetate=100:1 to 1:1) to afford 1,1-difluoro-N-((4²S,4³R)-1³,4⁴,4⁴-trifluoro-6-methyl-5-oxo-9-oxa-6-aza-4(2,1)-pyrrolidina-1(1,2),2(1,3)-dibenzenacyclononaphane-4³-yl) methanesulfonamide_cis racemic (150 mg, 28.50% yield) as a white solid. LCMS, Method E: (ESI⁺): m/z 520.2 (M+H)⁺, RT: 2.441 min.

### Step 6: 1,1-difluoro-N-((4²S, 4³R)-1³, 4⁴, 4⁴-trifluoro-6-methyl-S-oxo-9-oxa-6-aza-4(2,1)-pyrrolidina-1(1,2),2(1,3)-dibenzenacyclononaphane-4³-yl)methanesulfonamide and 1,1-difluoro-N-((4²R,4³S)-1³,4⁴, 4⁴-trifluoro-6-methyl-S-oxo-9-oxa-6-aza-4(2,1)-pyrrolidina-1(1,2),2(1,3)-dibenzenacyclononaphane-4³-yl)methanesulfonamide

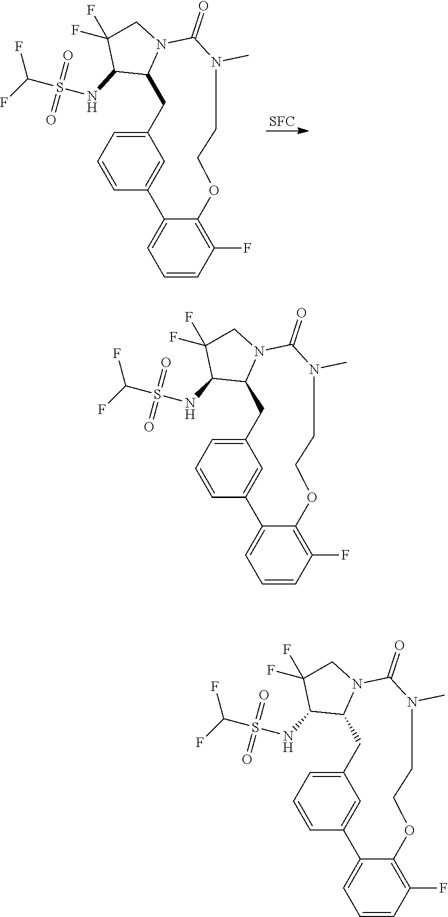

1,1-difluoro-N-((4²S,4³R)-1³,4⁴,4⁴-trifluoro-6-methyl-5-oxo-9-oxa-6-aza-4(2,1)-pyrrolidina-1(1,2),2(1,3)-dibenzenacyclononaphane-4³-yl) methanesulfonamide_cis racemic (150 mg) was subjected to super-fluid CO₂ chromatographic separation to give 1,1-difluoro-N-((4²S,4³R)-1³,4⁴,4⁴-trifluoro-6-methyl-5-oxo-9-oxa-6-aza-4(2,1)-pyrrolidina-1(1,2),2(1,3)-dibenzenacyclononaphane-4³-yl)methanesulfonamide (36 mg, 43% yield) and 1,1-difluoro-N-((4²R,4³S)-1³,4⁴,4⁴-trifluoro-6-methyl-5-oxo-9-oxa-6-aza-4(2,1)-pyrrolidina-1(1,2),2(1,3)-dibenzenacyclononaphane-4³-yl)methanesulfonamide (36 mg, 43% yield) as white solid.

### SFC Separation Method:

Instrument: Waters SFC150AP preparative SFC; Column: DAICEL CHIRALPAK AD (250 mm*30 mm, 10 μm); Mobile phase: A for CO₂ and B for EtOH (0.1% NH₃ in H₂O); Gradient: B % from 10% to 35% in 12 min isocratic elution mode; Flow rate: 35 g/min; Wavelength: 220 nm; Column temperature: 35° C.; System back pressure: 120 bar.

### Example 2. Procedure for the Preparation of Example 2

### Step 1: 2-(2-(3-bromo-2-fluorobenzyl)-2,5-dihydro-1H-pyrrol-1-yl)-1-phenyl-2λ2-ethan-1-one

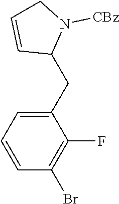

Compound was synthesised according to the procedure reported in WO2020/158958 A1 for 2-(2-(3-chloro-2-fluorobenzyl)-2,5-dihydro-1H-pyrrol-1-yl)-1-phenyl-2λ2-ethan-1-one.

### Step 2: Benzyl 2-[(3-bromo-2-fluoro-phenyl)methyl]-6-oxa-3-azabicyclo[3.1.0]hexane-3-carboxylate

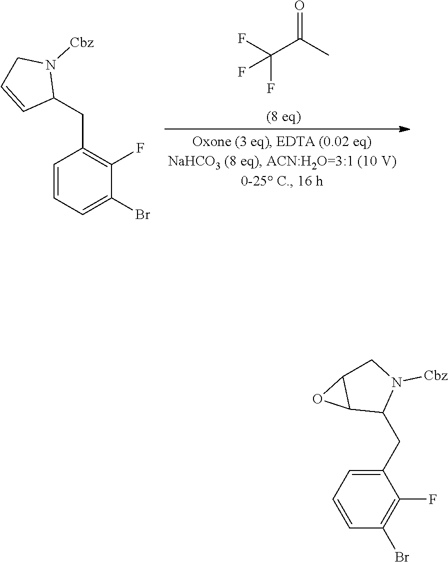

To a solution of 2-(2-(3-bromo-2-fluorobenzyl)-2,5-dihydro-1H-pyrrol-1-yl)-1-phenyl-2λ2-ethan-1-one (15 g, 38.45 mmol, 1 eq) in acetonitrile (90 mL) and water (60 mL) was added EDTA (225 mg, 770 μmol, 470 μL, 0.02 eq) and trifluoroacetone (34.45 g, 308 mmol, 27.55 mL, 8 eq) at 0° C. The mixture was stirred at 0° C. for 4 h. Then oxone (70.9 g, 115 mmol, 3 eq) and sodium carbonate (25.85 g, 308 mmol, 8 eq) were added at 0° C. The mixture was stirred at 25° C. for 12 h. Aqueous solution of sodium sulfite (100 ml) was added into the reaction mixture and the mixture was extracted with ethyl acetate (150 mL). The aqueous phase was extracted with ethyl acetate (3×150 mL). The combined organic phase was dried with anhydrous sodium sulfate, filtered and concentrated to give a residue. The residue was purified by column chromatography on silica gel eluted with petroleum ether/ethyl acetate=(71:29 to 0:100) to afford benzyl 2-[(3-bromo-2-fluoro-phenyl)methyl]-6-oxa-3-azabicyclo[3.1.0]hexane-3-carboxylate (10 g, yield 80%) as yellow oil.

LCMS, Method E: (ESI⁺): m/z 406 (M+H)⁺, RT: 0.84 min. ¹H NMR (400 MHz, methanol-d₄) δ ppm 1.29-1.39 (m, 1H), 1.53 (br s, 2H), 1.97-2.11 (m, 2H), 2.13-2.24 (m, 1H), 2.72-2.82 (m, 1H), 3.36-3.63 (m, 2H), 5.37-5.46 (m, 1H), 5.52-5.64 (m, 1H), 5.71-5.85 (m, 5H), 5.92 (dddd, 1H).

### Step 3: Benzyl 2-[(3-bromo-2-fluoro-phenyl)methyl]-3-hydroxy-4-methyl-pyrrolidine-1-carboxylate

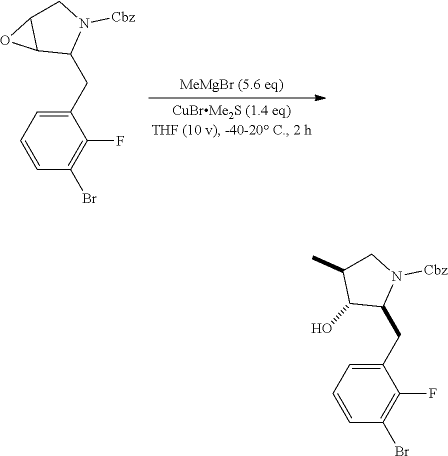

To a mixture of 2-[(3-bromo-2-fluoro-phenyl)methyl]-6-oxa-3-azabicyclo[3.1.0]hexane-3-carboxylate (10 g, 24.6 mmol, 1 eq), CuBr·Me₂S (7.08 g, 34.5 mmol, 1.4 eq) in THE (140 mL) −40° C. was added MeMgBr (3 M in THF, 45.9 mL, 5.6 eq). The mixture was then stirred at −20° C. for 2 h. Saturated aqueous ammonium chloride solution (200 mL) was added into the mixture and the mixture was allowed to warm to room temperature before extracting with ethyl acetate (3×300 mL). The organic layer was dried over anhydrous sodium sulfate and concentrated under reduced pressure to give a residue. The residue was purified by column chromatography (SiO₂, petroleum ether/ethyl acetate=15:85 to 15:85) to afford benzyl 2-[(3-bromo-2-fluoro-phenyl)methyl]-3-hydroxy-4-methyl-pyrrolidine-1-carboxylate (6 g, 58% yield) as yellow oil.

LCMS, Method E: (ESI⁺): m/z 422 (M+H)⁺, RT: 0.83 min. ¹H NMR (400 MHz, chloroform-d) δ ppm 0.92-0.92 (m, 1H), 1.82-1.92 (m, 1H), 2.66 (dd, 1H), 2.70-2.81 (m, 1H,), 2.85-2.96 (m, 1H), 3.10-3.18 (m, 1H), 3.27-3.34 (m, 1H), 3.42-3.53 (m, 1H), 3.69-3.76 (m, 2H), 3.76-3.86 (m, 1H), 4.99-5.10 (m, 2H), 6.76-6.88 (2H, m), 7.01-7.09 (m, 1H), 7.15 (s, 1H), 7.16-7.27 (m, 5H), 7.28-7.34 (m, 1H).

### Step 4: Benzyl 2-[(3-bromo-2-fluoro-phenyl)methyl]-4-methyl-3-oxo-pyrrolidine-1-carboxylate_cis racemic

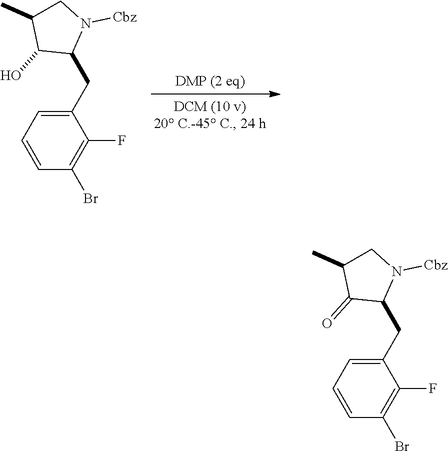

To a mixture of benzyl 2-[(3-bromo-2-fluoro-phenyl)methyl]-3-hydroxy-4-methyl-pyrrolidine-1-carboxylate (6 g, 14.2 mmol, 1 eq) in dichloromethane (60 mL) was added Dess-Martin periodinane (12.06 g, 28.4 mmol, 2 eq) at 25° C. Then the mixture was stirred at 45° C. for 12 h. The reaction mixture was poured into water (70 mL) and extracted with ethyl acetate (75 mL). The aqueous phase was extracted with ethyl acetate (3×75 mL). The combined organic phase was dried with sodium sulfate, filtered and concentrated to give a residue. The combined residue was purified by column chromatography on silica gel eluted with petroleum ether/ethyl acetate=(92:8 to 92:8) to afford benzyl 2-[(3-bromo-2-fluoro-phenyl)methyl]-4-methyl-3-oxo-pyrrolidine-1-carboxylate_cis racemic (4.63 g, yield 78%) as light yellow oil.

LCMS, Method E: (ESI⁺): m/z 420 (M+H)⁺, RT: 1.09 min. ¹H NMR (400 MHz, acetonitrile-d₃) δ ppm 0.76-0.90 (m, 3H), 2.44-2.51 (m, 1H), 2.54-2.69 (m, 1H), 2.92-3.09 (m, 1H), 3.21-3.33 (m, 1H), 4.12-4.24 (m, 1H), 4.30-4.43 (m, 1H), 4.96 (br d, 1H), 4.92-5.24 (m, 1H), 6.94-7.05 (m, 2H), 7.33-7.41 (m, 5H), 7.43-7.53 (m, 1H).

### Step 5: Benzyl 3-amino-2-[(3-bromo-2-fluoro-phenyl)methyl]-4-methyl-pyrrolidine-1-carboxylate_cis racemic

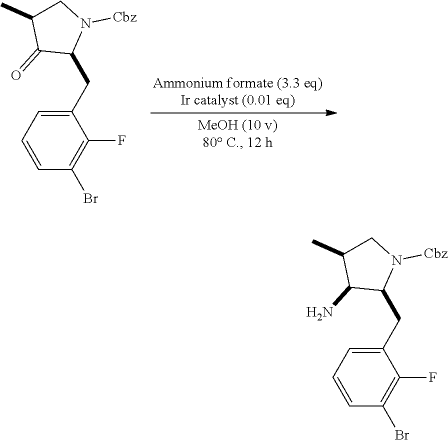

To a mixture of benzyl 2-[(3-bromo-2-fluoro-phenyl)methyl]-4-methyl-3-oxo-pyrrolidine-1-carboxylate_cis racemic (1.5 g, 3.57 mmol, 1 eq) in MeOH (15 mL) was added ammonium formate (675 mg, 10.7 mmol, 3 eq). Nitrogen was bubbled into the reaction mixture for 2 min. Bis[2-(2-pyridyl)phenyl]iridium(1+); 2-(2-pyridyl)pyridine; hexafluorophosphate (52 mg, 65 μmol, 0.01 eq) was added into the mixture above under nitrogen. The mixture was stirred at 80° C. for 12 h. The reaction mixture was poured into water (30 mL) and extracted with ethyl acetate (40 mL). The aqueous phase was extracted with ethyl acetate (3×40 mL). The organic phase was dried with sodium sulfate, filtered and concentrated to give a residue. Three vials were set up as described above, all four residues were combined together. The combined residue was purified by column chromatography on silica gel eluted with petroleum ether/ethyl acetate=(45:65 to 5:95) to afford benzyl 3-amino-2-[(3-bromo-2-fluoro-phenyl)methyl]-4-methyl-pyrrolidine-1-carboxylate_cis racemic (2.3 g, yield 44%) as light yellow oil.

LCMS, Method E: (ESI⁺): m/z 421 (M+H)⁺, RT: 0.65 min. ¹H NMR (400 MHz, acetonitrile-d₃) δ ppm 0.90-1.12 (m, 5H), 2.06-2.22 (m, 2H), 2.93-3.19 (m, 3H), 3.63-3.73 (m, 1H), 3.95-4.08 (m, 1H), 4.80-5.13 (m, 2H), 6.88-7.06 (m, 1H), 7.13-7.60 (m, 7H).

### Step 6: Benzyl 2-[(3-bromo-2-fluoro-phenyl)methyl]-3-(fluoromethylsulfonylamino)-4-methyl-pyrrolidine-1-carboxylate_cis racemic

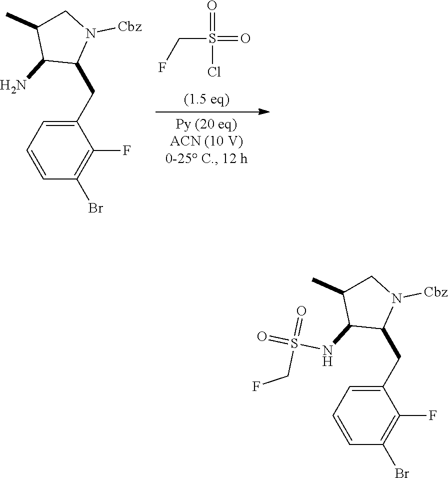

To a mixture of 3-amino-2-[(3-bromo-2-fluoro-phenyl)methyl]-4-methyl-pyrrolidine-1-carboxylate_cis racemic (1.4 g, 3.32 mmol, 1 eq) in acetonitrile (14 mL) was added pyridine (5.26 g, 66.5 mmol, 5.4 mL, 20 eq). Then fluoromethanesulfonyl chloride (1.10 g, 4.98 mmol, 60% purity, 1.5 eq) was added into the mixture at 0° C. The mixture was stirred at 25° C. for 12 h. The reaction mixture was poured into ice water (30 mL) and extracted with ethyl acetate (35 mL). The aqueous phase was further extracted with ethyl acetate (3×35 mL). The combined organic phase was dried with sodium sulfate, filtered and concentrated to give a residue. Two vials were set up as described above, all three residues were combined together. The combined residue was purified by column chromatography on silica gel eluted with petroleum ether/ethyl acetate (93:7 to 85:15) to afford benzyl 2-[(3-bromo-2-fluoro-phenyl)methyl]-3-(fluoromethylsulfonylamino)-4-methyl-pyrrolidine-1-carboxylate_cis racemic (1.3 g, yield 35.92%) as light yellow oil.

LCMS, Method E: (ESI⁺): m/z 519 (M+H)⁺, RT: 0.82 min. ¹H NMR (400 MHz, chloroform-d) δ ppm 1.14-1.22 (m, 3H), 2.06 (s, 1H), 2.43-2.57 (m, 1H), 3.02-3.10 (m, 1H), 3.74-3.90 (m, 1H), 4.08-4.24 (m, 1H), 4.15-4.23 (m, 1H), 4.33 (q, 1H), 4.64-5.02 (m, 2H), 4.69-4.81 (m, 2H), 5.05-5.12 (m, 1H), 5.09 (br d, 1H), 6.87-6.98 (m, 1H), 7.26-7.28 (m, 3H), 7.32-7.42 (m, 4H).

### Step 7: Benzyl 2-[[3-[2-[2-[tert-butoxycarbonyl(methyl)amino]ethoxy]phenyl]-2-fluoro-phenyl]methyl]-3-(fluoromethylsulfonylamino)-4-methyl-pyrrolidine-1-carboxylate_cis racemic

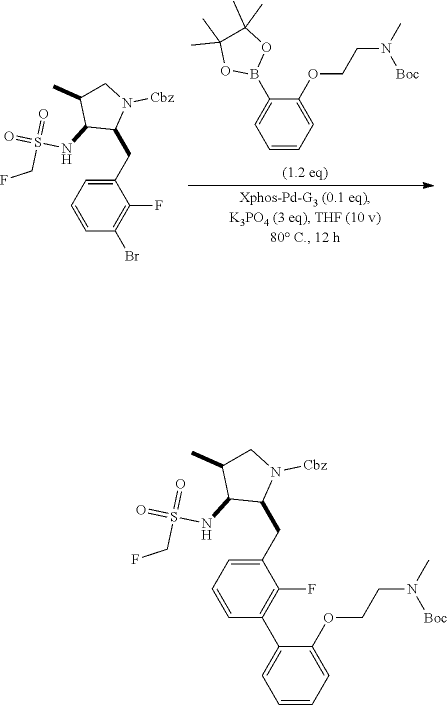

To a mixture of afford benzyl 2-[(3-bromo-2-fluoro-phenyl)methyl]-3-(fluoromethylsulfonylamino)-4-methyl-pyrrolidine-1-carboxylate_cis racemic (1.15 g, 2.22 mmol, 1 eq) in THE (12 mL) was added tert-butyl N-methyl-N-[2-[2-(4,4,5,5-tetramethyl-1,3,2-dioxaborolan-2-yl)phenoxy]ethyl]carbamate (1.82 g, 3.33 mmol, 69% purity, 1.2 eq), K₃PO₄ (1.42 g, 6.67 mmol, 3 eq) and Xphos-Pd-G3 (188 mg, 222 μmol, 0.1 eq) under nitrogen. The mixture was stirred at 80° C. for 12 h. The reaction mixture was poured into water (30 mL) and the mixture was extracted with ethyl acetate (25 mL). The aqueous phase was further extracted with ethyl acetate (3×25 mL). The organic phase was dried with sodium sulfate, filtered and concentrated to give a residue. The residue was dissolved with acetonitrile (4.5 mL). The filtrate was purified by prep-HPLC (column: Phenomenex C18 75×30 mm×3 μm; mobile phase: [water (NH₄HCO₃)-acetonitrile]; B %: 50%-70%, 8 min) to afford benzyl 2-[[3-[2-[2-[tert-butoxycarbonyl(methyl)amino]ethoxy]phenyl]-2-fluoro-phenyl]methyl]-3-(fluoromethylsulfonylamino)-4-methyl-pyrrolidine-1-carboxylate_cis racemic (460 mg, yield 30%) as a white solid. LCMS, Method E: (ESI⁺): m/z 588 (M+H−100)⁺, RT: 0.92 min.

### Step 8: tert-butyl N-[2-[2-[2-fluoro-3-[[3-(fluoromethylsulfonylamino)-4-methyl-pyrrolidin-2-yl]methyl]phenyl]phenoxy]ethyl]-N-methyl-carbamate_cis racemic

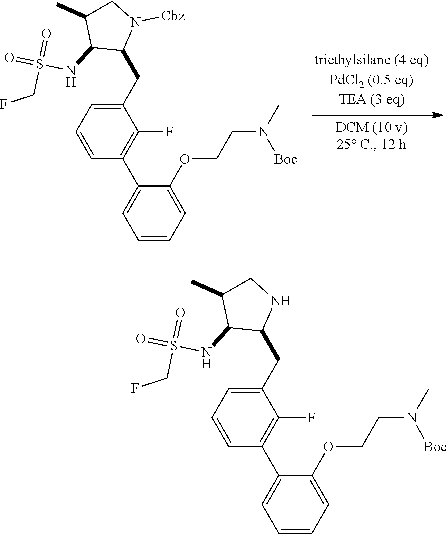 [SMILES: C[C@H]1CN(C(=O)OCc2ccccc2)[C@@H](Cc2cccc(-c3ccccc3OCCN(C)C(=O)OC(C)(C)C)c2F)[C@H]1NS(=O)(=O)CF ; C[C@H]1CN[C@@H](Cc2cccc(-c3ccccc3OCCN(C)C(=O)OC(C)(C)C)c2F)[C@H]1NS(=O)(=O)CF]

To a mixture of benzyl 2-[[3-[2-[2-[tert-butoxycarbonyl(methyl)amino]ethoxy]phenyl]-2-fluoro-phenyl]methyl]-3-(fluoromethylsulfonylamino)-4-methyl-pyrrolidine-1-carboxylate_cis racemic (450 mg, 654 μmol, 1 eq) and triethylsilane (300 mg, 2.6 mmol, 420 μL, 4 eq) in dichloromethane (4.5 mL) was added PdCl₂ (58 mg, 327 μmol, 0.5 eq) and triethylamine (200 mg, 1.96 mmol, 275 μL, 3 eq) at 25° C. N₂ was bubbled into the reaction mixture for 2 mins. Then the whole reaction mixture was stirred at 25° C. for 12 h. The reaction mixture was poured into water (20 mL) and the mixture was extracted with ethyl acetate (25 mL). The aqueous phase was further extracted with ethyl acetate (3×25 mL). The organic phase was dried with sodium sulfate, filtered and concentrated to afford tert-butyl N-[2-[2-[3-[[(2S,3R,4S)-3-(difluoromethylsulfonylamino)-4-fluoro-pyrrolidin-2-yl]methyl]-2-fluoro-phenyl]-4,6-difluoro-phenoxy]ethyl]-N-methyl-carbamate_cis racemic (300 mg, yield 83%) as yellow oil. LCMS, Method E: (ESI⁺): m/z 611 (M+AcOH)⁺, RT: 1.00 min.

### Step 9: 1-Fluoro-N-[2-[[2-fluoro-3-[2-[2-(methylamino)ethoxy]phenyl]phenyl]methyl]-4-methyl-pyrrolidin-3-yl]methanesulfonamide_cis racemic

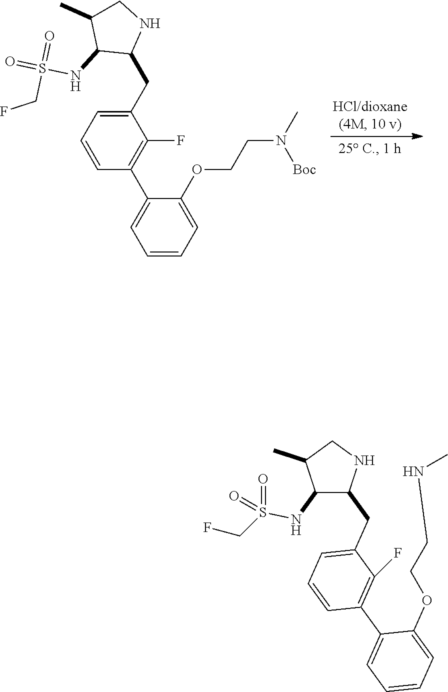 [SMILES: C[C@H]1CN[C@@H](Cc2cccc(-c3ccccc3OCCN(C)C(=O)OC(C)(C)C)c2F)[C@H]1NS(=O)(=O)CF ; CNCCOc1ccccc1-c1cccc(C[C@@H]2NC[C@H](C)[C@@H]2NS(=O)(=O)CF)c1F]

tert-Butyl-N-[2-[2-[3-[[(2S,3R,4S)-3-(difluoromethylsulfonylamino)-4-fluoro-pyrrolidin-2-yl]methyl]-2-fluoro-phenyl]-4,6-difluoro-phenoxy]ethyl]-N-methyl-carbamate_cis racemic (300 mg, 540 μmol, 1 eq) was dissolved in HCl/dioxane (3 mL) and stirred at 25° C. for 1 h. The reaction mixture was concentrated to give 1-fluoro-N-[2-[[2-fluoro-3-[2-[2-(methylamino) ethoxy]phenyl]phenyl]methyl]-4-methyl-pyrrolidin-3-yl]methanesulfonamide_cis racemic (240 mg, yield 98%) as orange oil, which was used directly without any purification. LCMS, Method E: (ESI⁺): m/z 454 (M+H)⁺, RT: 0.54 min.

### Step 10: 1-Fluoro-N-((4²S, 4³S,4⁴S)-2²-fluoro-4⁴,6-dimethyl-5-oxo-9-oxa-6-aza-4(2,1)-pyrrolidina-1(1,2),2(1,3)-dibenzenacyclononaphane-4³-yl)methanesulfonamide and 1-fluoro-N-((4²R,43R,4⁴R)-2²-fluoro-4⁴,6-dimethyl-5-oxo-9-oxa-6-aza-4(2,1)-pyrrolidina-1(1,2),2(1,3)-dibenzenacyclononaphane-4³-yl)methanesulfonamide

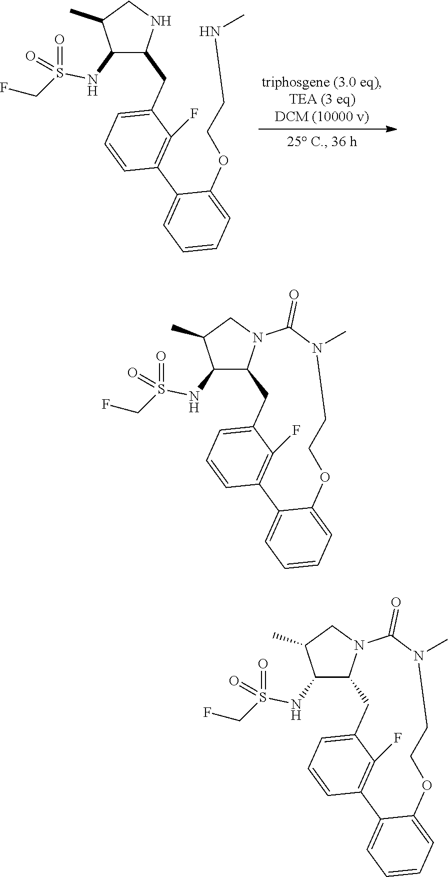 [SMILES: CNCCOc1ccccc1-c1cccc(C[C@@H]2NC[C@H](C)[C@@H]2NS(=O)(=O)CF)c1F ; C[C@H]1CN2C(=O)N(C)CCOc3ccccc3-c3cccc(c3F)C[C@H]2[C@H]1NS(=O)(=O)CF ; C[C@@H]1CN2C(=O)N(C)CCOc3ccccc3-c3cccc(c3F)C[C@@H]2[C@@H]1NS(=O)(=O)CF]

To a solution of 1-fluoro-N-[2-[[2-fluoro-3-[2-[2-(methylamino) ethoxy]phenyl]phenyl]methyl]-4-methyl-pyrrolidin-3-yl]methanesulfonamide_cis racemic (145 mg, 320 μmol, 1 eq) in dichloromethane (1.45 L) was added triethylamine (97 mg, 960 μmol, 135 μL, 3 eq). The mixture was stirred at 25° C. for 1 min. Then a solution of triphosgene (28 mg, 95.9 μmol, 0.3 eq) in dichloromethane (1 mL) was added dropwise and the whole reaction mixture was stirred at 25° C. for 36 h. Three reactions were set up as described above, all four reaction mixtures were combined together. The reaction mixture was concentrated and the residue was dissolved with dimethyl formamide (1.5 mL) and the solution was filtered. The filtrate was purified by prep-HPLC (column: Phenomenex C18 75×30 mm×3 μm; mobile phase: [water (NH₃·H₂O+NH₄HCO₃)-acetonitrile]; B %: 35%-65%, 8 min) to afford racemic desired compound (71 mg, purity 99%) as white solid, which was further separated by SFC (column: DAICEL CHIRALPAK AD (250 mm×30 mm, 10 μm); mobile phase: [Neu-IPA]; B %: 50%-50%, 10 min) to give 1-fluoro-N-((4²S,4³S,4⁴S)-2²-fluoro-4⁴,6-dimethyl-5-oxo-9-oxa-6-aza-4(2,1)-pyrrolidina-1(1,2),2(1,3)-dibenzenacyclononaphane-4³-yl)methanesulfonamide (26.9 mg, 37% yield) as white solid and 1-fluoro-N-((4²R,4³R,4⁴R)-2²-fluoro-4⁴,6-dimethyl-5-oxo-9-oxa-6-aza-4(2,1)-pyrrolidina-1(1,2),2(1,3)-dibenzenacyclononaphane-4³-yl)methanesulfonamide (21.3 mg, yield 30%) as white solid.

### Example 3. Procedure for the Preparation of Example 3

### Step 1: tert-butyl 2-[(3-bromo-2-fluoro-phenyl)methyl]-3-oxo-piperidine-1-carboxylate

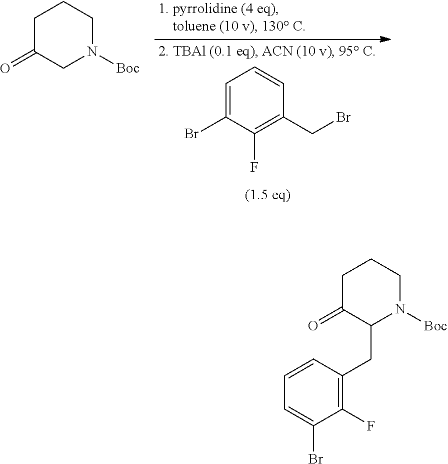 [SMILES: CC(C)(C)OC(=O)N1CCCC(=O)C1 ; Fc1c(Br)cccc1CBr ; CC(C)(C)OC(=O)N1CCCC(=O)C1Cc1cccc(Br)c1F]

To a solution of piperidine-3-one-N-tert-butyloxycarbamate (50 g, 250 mmol, 1 eq) in toluene (500 mL) was added pyrrolidine (71 g, 1.00 mol, 84 mL, 4 eq) at 25° C. The mixture was heated and stirred at 130° C. for 12 hrs under nitrogen atmosphere. The mixture was concentrated under reduced pressure. The residue was dissolved in acetonitrile (500 mL) and then cooled to 0° C. Tetrabutylammonium iodide (3.68 g, 9.95 mmol, 0.1 eq) and 1-bromo-3-(bromomethyl)-2-fluoro-benzene (40 g, 149.30 mmol, 1.5 eq) were added at 0° C. and the mixture was then heated to 95° C. and stirred for 12 hrs under nitrogen atmosphere. The mixture was poured into ice water (500 mL), and extracted with ethyl acetate (3×100 mL). The organic layer was separated and washed with saturated aqueous NaHCO₃ solution (100 mL), dried over anhydrous Na₂SO₄, filtered and concentrated under reduced pressure. The residue was purified by column chromatography on silica gel (eluting with petroleum ether/ethyl acetate=2/1) to give tert-butyl 2-[(3-bromo-2-fluoro-phenyl)methyl]-3-oxo-piperidine-1-carboxylate (25 g, yield 26.1%) as a white solid. LCMS, Method E: (ESI⁺): m/z 332 (M-tBu)⁺, RT: 0.88 min.

### Step 2: tert-Butyl 2-[[2-fluoro-3-(2-hydroxyphenyl)phenyl]methyl]-3-oxo-piperidine-1-carboxylate

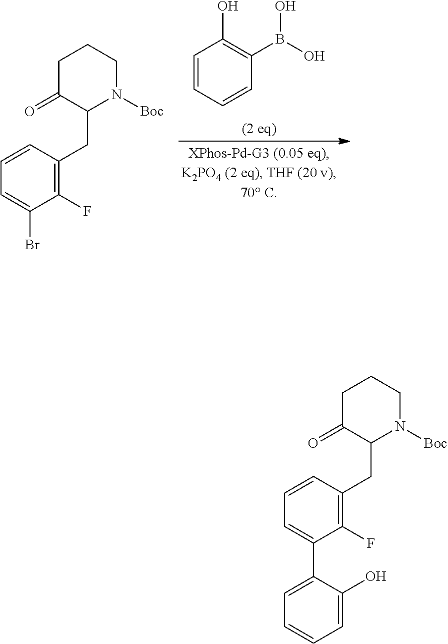 [SMILES: CC(C)(C)OC(=O)N1CCCC(=O)C1Cc1cccc(Br)c1F ; OB(O)c1ccccc1O ; CC(C)(C)OC(=O)N1CCCC(=O)C1Cc1cccc(-c2ccccc2O)c1F]

To a solution of tert-butyl 2-[(3-bromo-2-fluoro-phenyl)methyl]-3-oxo-piperidine-1-carboxylate (10 g, 25.9 mmol, 1 eq) and (2-hydroxyphenyl)boronic acid (7.14 g, 51.8 mmol, 2 eq) in THE (200 mL) were added Xphos-Pd-G3 (1.10 g, 1.29 mmol, 0.05 eq) and K₃PO₄ (10.99 g, 51.8 mmol, 2 eq) at 25° C. under N₂. The reaction mixture was stirred at 70° C. for 8 hrs. The reaction mixture was poured into water (100 mL) and extracted with ethyl acetate (3×50 mL). The combined organic layers were washed with brine (100 mL), dried over anhydrous sodium sulfate, filtered and concentrated under reduced pressure. The crude product was purified by column chromatography on silica gel (eluting with petroleum ether/ethyl acetate=1/1)) to afford tert-butyl 2-[[2-fluoro-3-(2-hydroxyphenyl)phenyl]methyl]-3-oxo-piperidine-1-carboxylate (10 g, yield, 87%) as a white solid. LCMS, Method E: (ESI⁺): m/z 300 (M−100+H)⁺, RT: 0.82 min.

### Step 3: tert-butyl 2-[[3-[2-[(E)-3-tert-butoxy-3-oxo-prop-1-enoxy]phenyl]-2-fluoro-phenyl]methyl]-3-oxo-piperidine-1-carboxylate

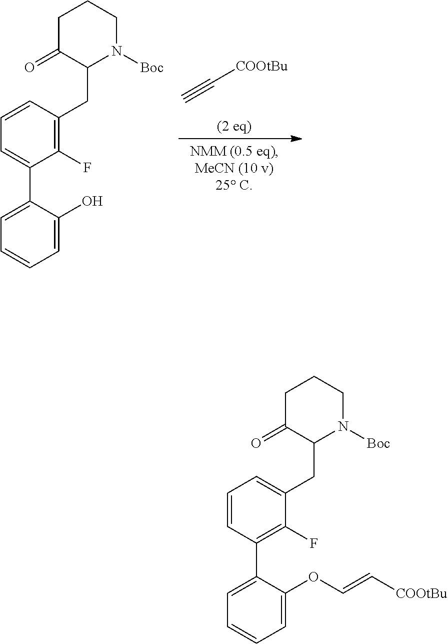

To solution of tert-butyl 2-[[2-fluoro-3-(2-hydroxyphenyl)phenyl]methyl]-3-oxo-piperidine-1-carboxylate (9.5 g, 20.2 mmol, 85% purity, 1 eq) and tert-butyl prop-2-ynoate (5.10 g, 40.4 mmol, 5.55 mL, 2 eq) in MeCN (95 mL) was added N-methyl-morpholine (1.02 g, 10.11 mmol, 1.11 mL, 0.5 eq) at 25° C. under N₂. The reaction mixture was stirred at 25° C. for 12 hrs. The reaction mixture was quenched by addition water (200 mL), and then extracted with ethyl acetate (3×100 mL). The combined organic layers were washed with brine (100 mL), dried over anhydrous magnesium sulfate, filtered and concentrated under reduced pressure. The crude product was purified by column chromatography on silica gel (eluting with petroleum ether/ethyl acetate=2/1)) to afford tert-butyl 2-[[3-[2-[(E)-3-tert-butoxy-3-oxo-prop-1-enoxy]phenyl]-2-fluoro-phenyl]methyl]-3-oxo-piperidine-1-carboxylate (9 g, yield, 76%) as a white solid. LCMS, Method E: (ESI⁺): m/z 370 (M−155)⁺ (M-Boc-tBu)⁺, RT: 0.95 min.

### Step 4: tert-butyl 2-[[3-[2-(3-tert-butoxy-3-oxo-propoxy)phenyl]-2-fluoro-phenyl]methyl]-3-oxo-piperidine-1-carboxylate

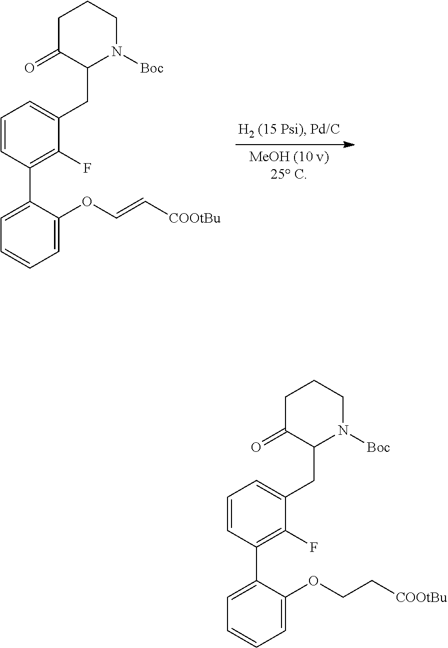

To a solution of tert-butyl 2-[[3-[2-[(E)-3-tert-butoxy-3-oxo-prop-1-enoxy]phenyl]-2-fluoro-phenyl]methyl]-3-oxo-piperidine-1-carboxylate (2.25 g, 4.28 mmol, 1 eq) in MeOH (22.5 mL) was added Pd/C (560 mg, 10% on charcoal). The suspension was degassed and purged with H₂ three times. The mixture was stirred under H₂ (15 psi) at 25° C. for 12 hrs. Three additional reactions were set up as detailed above. All four reaction mixtures were combined. The mixture was filtered and concentrated under reduced pressure. The residue was purified by column chromatography on silica gel (eluting with petroleum ether/ethyl acetate=30/1 to 10/1) to give tert-butyl 2-[[3-[2-(3-tert-butoxy-3-oxo-propoxy)phenyl]-2-fluoro-phenyl]methyl]-3-oxo-piperidine-1-carboxylate (5.5 g, yield, 55%) as white solid. LCMS, Method E: (ESI⁺): m/z 372 (M-Boc-t-Bu)⁺, RT: 0.88 min.

### Step 5: 3-[2-[2 fluoro-3-[(3-oxo-2-piperidyl)methyl]phenyl]phenoxy]propanoic acid

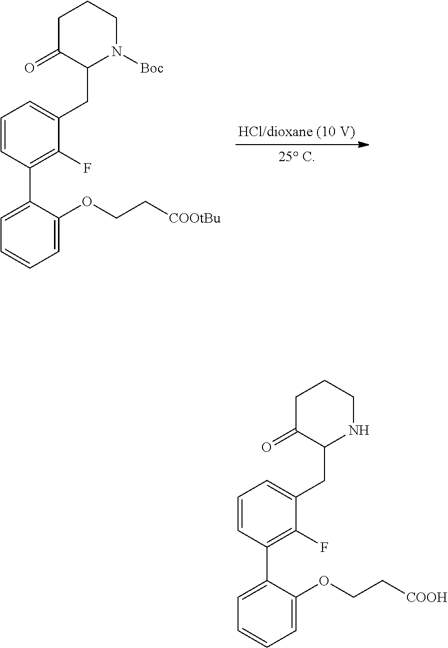

A solution of tert-butyl 2-[[3-[2-(3-tert-butoxy-3-oxo-propoxy)phenyl]-2-fluoro-phenyl]methyl]-3-oxo-piperidine-1-carboxylate (5.5 g, 10.42 mmol, 1 eq) in 2 N HCl/dioxane (55 mL) was stirred at 25° C. for 2 hrs. The mixture was concentrated under reduced pressure to give 3-[2-[2-fluoro-3-[(3-oxo-2-piperidyl)methyl]phenyl]phenoxy]propanoic acid (5 g, yield, 100%) as white solid, which was used in the next step without further purification. LCMS, Method E: (ESI⁺): m/z 404 (M+H)⁺, RT: 0.57 min.

### Step 6: 2²-fluoro-8-oxa-4(2,1)-piperidina-1(1,2),2(1,3)-dibenzenacyclooctaphane-4³, 5-dione

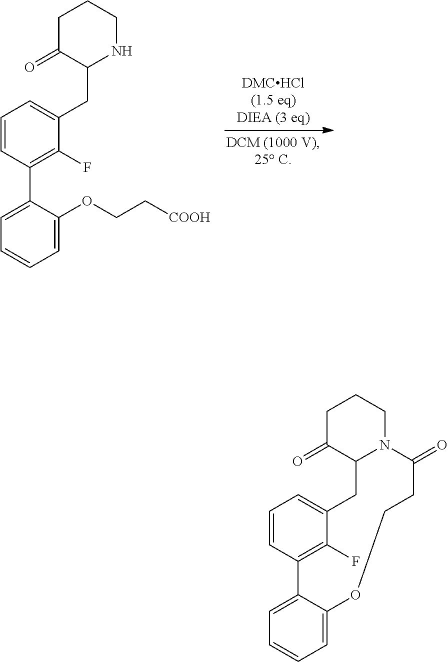

To a solution of 3-[2-[2-fluoro-3-[(3-oxo-2-piperidyl)methyl]phenyl]phenoxy]propanoic acid (5 g, 13.5 mmol, 1 eq) in dichloromethane (5 L) were added 2-chloro-1,3-dimethyl-4,5-dihydroimidazol-1-ium chloride (3.41 g, 20.2 mmol, 1.5 eq) and DIEA (5.2 g, 40.4 mmol, 7.0 mL, 3 eq). The mixture was stirred at 25° C. for 12 hrs. LCMS showed all of starting material was consumed. The mixture was concentrated under reduced pressure. The residue was purified by column chromatography on silica gel (eluting with petroleum ether/ethyl acetate=20/1 to 0/1) to give 2²-fluoro-8-oxa-4(2,1)-piperidina-1(1,2),2(1,3)-dibenzenacyclooctaphane-4³,5-dione (2.8 g, yield, 53%) as white solid. LCMS, Method E: (ESI⁺): m/z 354 (M+H)⁺, RT: 0.70 min

### Step 7: (4²S, 4³S)-4³-amino-2²-fluoro-8-oxa-4(2,1)-piperidina-1(1,2),2(1,3)-dibenzena cyclooctaphan-5-one_cis racemic

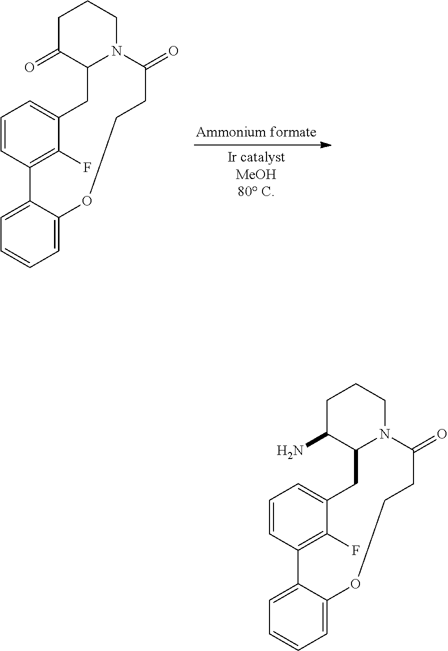

To a solution of 2²-fluoro-8-oxa-4(2,1)-piperidina-1(1,2),2(1,3)-dibenzenacyclooctaphane-4³,5-dione (200 mg, 570 μmol, 1 eq) in MeOH (20 mL) was added ammonium formate (107 mg, 1.70 mmol, 3 eq) and bis[2-(2-pyridyl)phenyl]iridium(1+); 2-(2-pyridyl)pyridine hexafluorophosphate (7.15 mg, 8.5 μmol, 0.015 eq). The mixture was stirred at 80° C. for 12 hrs under N₂ atmosphere. The reaction mixture was purified by prep-HPLC directly (column: Waters Xbridge BEH C18 100*30 mm*10 μm; mobile phase: [water(NH₄HCO₃)-acetonitrile]; B %: 25%-45%, 8 min) to give (4²S,4³S)-4³-amino-2²-fluoro-8-oxa-4(2,1)-piperidina-1(1,2),2(1,3)-dibenzena cyclooctaphan-5-one_cis racemic (180 mg, yield, 63%) as a white solid. LCMS, Method F: (ESI⁺): m/z 355 (M+H)⁺, RT: 1.54 min.

### Step 8: N-((4²S, 4³S)-2²-fluoro-5-oxo-8-oxa-4(2,1)-piperidina-1(1,2),2(1,3)-dibenzena cyclooctaphane-4³-yl)propane-2-sulfonamide_cis racemic

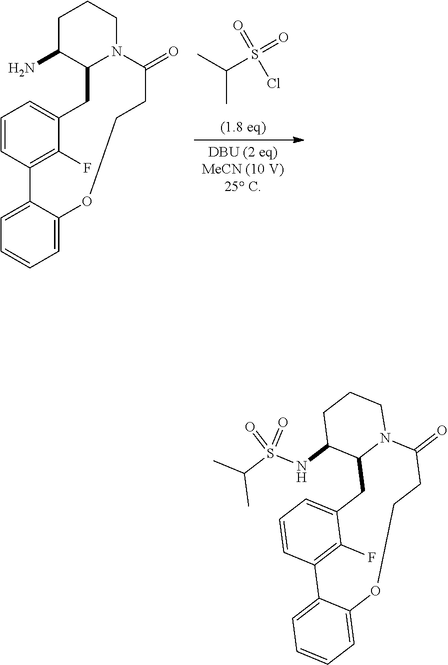

To a solution of (4²S,4³S)-4³-amino-2²-fluoro-8-oxa-4(2,1)-piperidina-1(1,2),2(1,3)-dibenzena cyclooctaphan-5-one_cis racemic (180 mg, 510 μmol, 1 eq) and DBU (155 mg, 1.02 mmol, 153 μL, 2 eq) in acetonitrile (1.8 mL) was added propane-2-sulfonyl chloride (130 mg, 914 μmol, 102 μL, 1.8 eq) at 0° C. The mixture was stirred at 25° C. for 12 hrs. The mixture was concentrated under reduced pressure and the residue was purified by prep-HPLC (column: Waters Xbridge Prep OBD C18 150*40 mm*10 μm; mobile phase: [water (NH₄HCO₃)-acetonitrile]; B %: 20%-50%, 8 min) to give N-((4²S,4³S)-2²-fluoro-5-oxo-8-oxa-4(2,1)-piperidina-1(1,2),2(1,3)-dibenzena cyclooctaphane-4³-yl)propane-2-sulfonamide_cis racemic (100 mg, yield, 43%) as white solid. LCMS, Method E: (ESI⁺): m/z 461 (M+H)⁺, RT: 0.71 min.

### Step 9: N-((4²S, 4³S)-2²-fluoro-5-oxo-8-oxa-4(2,1)-piperidina-1(1,2),2(1,3)-dibenzena cyclooctaphane-4³-yl)propane-2-sulfonamide

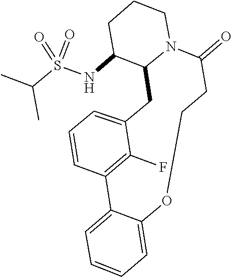

180 mg of N-((4²S,4³S)-2²-fluoro-5-oxo-8-oxa-4(2,1)-piperidina-1(1,2),2(1,3)-dibenzena cyclooctaphane-4³-yl)propane-2-sulfonamide_cis racemic was separated by SFC separation (column: DAICEL CHIRALPAK AD (250 mm*30 mm, 10 μm); mobile phase: [0.1% NH₃ in H₂O/ETOH]; B %: 40%-40%,13 min) to give N-((4²S,4³S)-2²-fluoro-5-oxo-8-oxa-4(2,1)-piperidina-1(1,2),2(1,3)-dibenzena cyclooctaphane-4³-yl)propane-2-sulfonamide (15 mg, yield, 6.4%) as white solid.

### Example 4. Procedure for the Preparation of Example 4

### Step 1: tert-butyl 6-(3-bromo-2-fluorobenzyl)-7-oxo-5-azaspiro[2.4]heptane-5-carboxylate

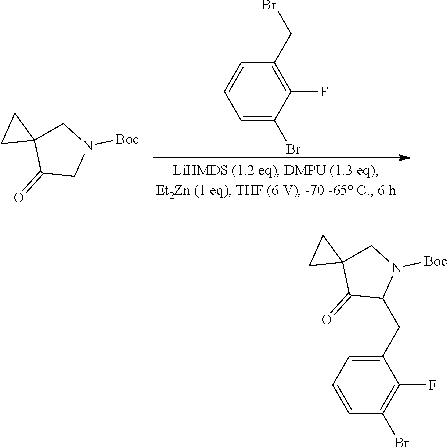 [SMILES: CC(C)(C)OC(=O)N1CC(=O)C2(CC2)C1 ; Fc1c(Br)cccc1CBr ; CC(C)(C)OC(=O)N1CC2(CC2)C(=O)C1Cc1cccc(Br)c1F]

Charge tetrahydrofuran (1.4 L) to a reactor under nitrogen and add LiHMDS (4.0 L, 1 M in THF, 1.2 eq). Cool the reaction mixture to −70° C. to −65° C. A solution of tert-butyl 7-oxo-5-azaspiro[2.4]heptane-5-carboxylate (700 g, 1.0 eq) in THE (1.4 L) was added dropwise to the reactor at −70° C. to −65° C. and stirred for 3 hrs. Then, Et₂Zn (3.3 L, 1 M in toluene, 1.0 eq) was added dropwise to the reactor at −70° C. to −65° C., following by DMPU (552 g, 1.3 eq) dropwise to the reactor at −70° C. to −65° C. Then, a solution of 1-bromo-3-(bromomethyl)-2-fluorobenzene (977 g, 1.1 eq) in THF (1.4 L) was added dropwise to the reactor and stirred at −70° C. to −65° C. for at least 3 h. Then the reaction mixture was poured into the ice-water (1.5 kg) at 0° C. and was extracted with ethyl acetate (14 L) twice. Separate and combine the organic phase and wash the organic phase with brine (3.5 L) twice and then dry the organic phases with Na₂SO₄ (500 g) and filter. Concentrate the organic phase under vacuum to give crude product which was purified by column chromatography (SiO₂, eluted with petroleum ether:ethyl acetate=1:0 to 13:1) to afford the title compound (969 g, yield 91.7%) as white solid. LCMS (method O) (ESI⁺): m/z 341.9 (M+H−55)⁺, RT: 1.97 min.

### Step 2: tert-butyl (E/Z)-6-(3-bromo-2-fluorobenzyl)-7-(((R)-tert-butylsulfinyl)imino)-5-azaspiro[2.4]heptane-5-carboxylate

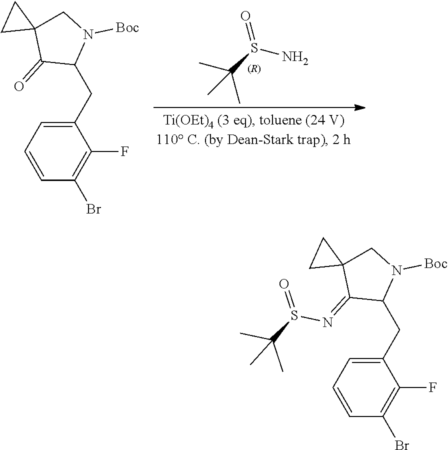

To a solution of tert-butyl 6-(3-bromo-2-fluorobenzyl)-7-oxo-5-azaspiro[2.4]heptane-5-carboxylate (100 g, 1.0 eq) in toluene (2.4 L) was added Ti(OEt)₄ (171.8 g, 3 eq) and (R)-2-methylpropane-2-sulfinamide (70 g, 2.3 eq). The mixture was heated to 110° C. and refluxed for 3-4 hrs. Totally 10 batches were set up, combined, cooled to 15-25° C. and poured into ice-water (1.5 kg) at 0° C. and the resulting white solid precipitate was filtered. The filtrate was extracted with ethyl acetate (3 L) and washed with brine (1 L) twice. The organic phases were dried over Na₂SO₄(500 g) and concentrated to afford crude product which was purified by column chromatography (SiO₂, eluted with petroleum ether:ethyl acetate=100:1 to 5:1) to afford the title compound (954 g, 75.8% yield) as yellow oil. LCMS (method O) (ESI⁺): m/z 445.1 (M+H−56)⁺, R.T=2.18 and 2.24.

### Step 3: tert-butyl (6S,7S)-6-(3-bromo-2-fluorobenzyl)-7-(((R)-tert-butylsulfinyl)amino)-5-azaspiro[2.4]heptane-5-carboxylate

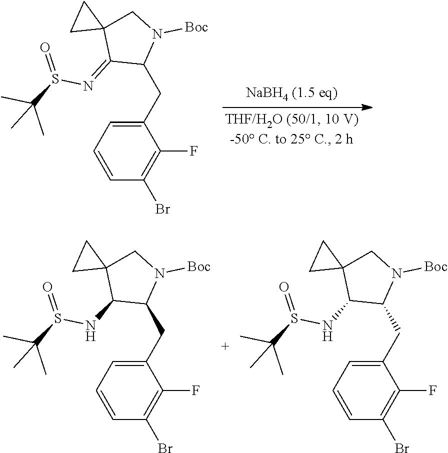

A solution of tert-butyl (E/Z)-6-(3-bromo-2-fluorobenzyl)-7-(((R)-tert-butylsulfinyl)imino)-5-azaspiro[2.4]heptane-5-carboxylate (150 g, 1.0 eq) in THE (1.5 L, 10 V) and H₂O (30 mL, 0.2 V) was cooled to −50° C. NaBH₄ (17 g, 1.5 eq) was added to the reactor under nitrogen at −50° C. The mixture was stirred for at least 2 h at 25±5° C. MeOH (0.9 L) was added to the reactor at 25-40° C. and stirred for at least 3 h at 25±5° C. Another seven reactions were set up as above, combined and concentrated under vacuum at 45° C. The residue was purified by column chromatography (SiO₂, eluted with petroleum ether:ethyl acetate=10:1 to 1:1) to obtained crude product which was triturated with petroleum ether/ethyl acetate=8/1 (6 V) for 8 h to afford the desired compound (318 g, 30.1% yield) as white solid. LCMS (method O) (ESI⁺): m/z 447.1 (M+H−56)⁺, R.T=2.03.

### Step 4: tert-butyl (6S,7S)-7-amino-6-(3-bromo-2-fluorobenzyl)-5-azaspiro[2.4]heptane-5-carboxylate

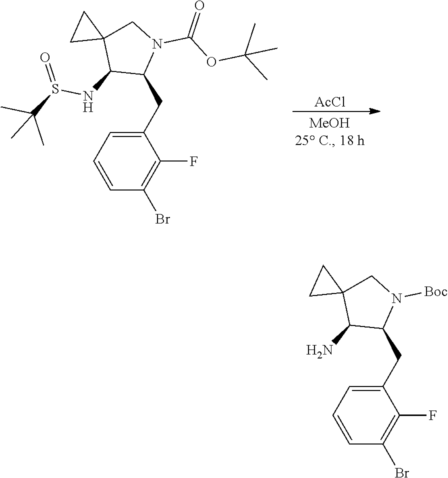 [SMILES: "AcCl" ; CC(C)(C)OC(=O)N1CC2(CC2)[C@H](N[S@](=O)C(C)(C)C)[C@@H]1Cc1cccc(Br)c1F ; CC(C)(C)OC(=O)N1CC2(CC2)[C@H](N)[C@@H]1Cc1cccc(Br)c1F]

A solution of tert-butyl (6S,7S)-6-(3-bromo-2-fluorobenzyl)-7-(((R)-tert-butylsulfinyl)amino)-5-azaspiro[2.4]heptane-5-carboxylate (110 g, 1.0 eq) in MeOH (2.2 L) was cooled to 0° C. Then Acetyl chloride (18.9 g, 1.1 eq) was added dropwise and stirred for at least 18 h at 25+5° C. under nitrogen. Totally 3 batches were set up, combined and transferred to a saturated NaHCO₃ solution (2.2 L) at 0-5° C. The pH was maintained between 7 to 8, brine (1.1 L) added and the product extracted with ethyl acetate (2.2 L) twice. The organic phases were separated, combined and dried over Na₂SO₄(300 g). The organic phase was concentrated under vacuum below 45° C. to give a residue, which was triturated with petroleum ether:ethyl acetate=10:1, 1 L for 8 h. The precipitated was collected and dried under vacuum <40° C. for 8 h to afford the title compound (178 g, 68.2% yield) as a yellow solid.

¹H NMR (400 MHz, methanol-d₄) δ 0.39-0.68 (m, 3H), 0.88-1.02 (m, 1H), 1.07-1.33 (m, 9H), 2.96-3.09 (m, 1H), 3.13 (m, 2H), 3.44-3.72 (m, 2H), 4.26 (ddd, 1H), 6.94-7.06 (m, 1H), 7.17 (br t, 1H), 7.48 (br t, 1H).

### Step 5: tert-butyl (6S,7S)-7-amino-6-[[3-[2-[2-[tert-butoxycarbonyl (methyl) amino]ethoxy]phenyl]-2-fluoro-phenyl]methyl]-5-azaspiro[2.4]heptane-5-carboxylate

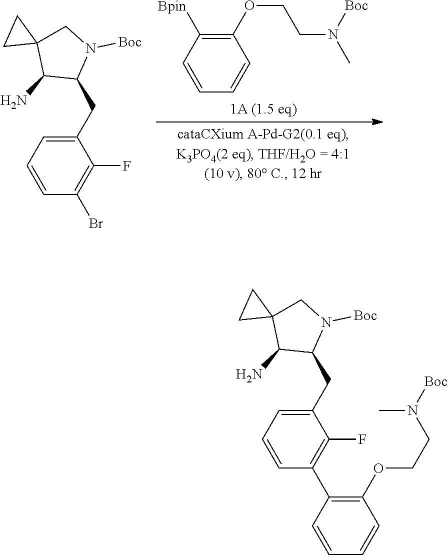

To a solution of tert-butyl (6S,7S)-7-amino-6-(3-bromo-2-fluorobenzyl)-5-azaspiro[2.4]heptane-5-carboxylate (900 mg, 2.25 mmol, 1 eq) in THE (8 mL) and H₂O (2 mL) was added K₃PO₄ (960 mg, 4.51 mmol, 2 eq), Cpd 1A (1.28 g, 3.38 mmol, 1.5 eq) and cataCXium A-Pd-G2 (151 mg, 225 μmol, 0.1 eq) under N₂ then the mixture was stirred at 80° C. for 12 h. Then the reaction mixture was poured into water (20 mL) and the mixture was extracted with ethyl acetate (10 mL). The aqueous phase was extracted with ethyl acetate (3×15 mL). The combined organic phase was dried with anhydrous sodium sulfate, filtered and concentrated under reduced pressure to give a residue (2.5 g). The residue was purified by column chromatography (SiO₂, Petroleum ether/Ethyl acetate=1/0 to 0/1) to give tert-butyl (6S,7S)-7-amino-6-[[3-[2-[2-[tert-butoxycarbonyl (methyl) amino]ethoxy]phenyl]-2-fluoro-phenyl]methyl]-5-azaspiro[2.4]heptane-5-carboxylate (1.2 g, yield 89%) as yellow oil. LCMS, Method E. (ESI⁺): m/z 570.3 (M+H)⁺, RT: 0.746 min.

### Step 6: tert-Butyl (6S,7S)-6-[[3-[2-[2-[tert-butoxycarbonyl (methyl) amino]ethoxy]phenyl]-2-fluoro-phenyl]methyl]-7-(difluoromethylsulfonylamino)-5-azaspiro[2.4]heptane-5-carboxylate

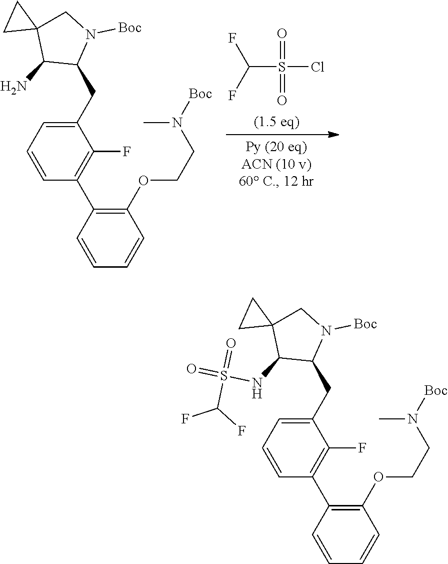

To a solution of tert-butyl (6S,7S)-7-amino-6-[[3-[2-[2-[tert-butoxycarbonyl (methyl) amino]ethoxy]phenyl]-2-fluoro-phenyl]methyl]-5-azaspiro[2.4]heptane-5-carboxylate (1.39 g, 2.44 mmol, 1 eq) in acetonitrile (14 mL) was added pyridine (3.87 g, 48.9 mmol, 3.94 mL, 20 eq), then difluoromethanesulfonyl chloride (551.75 mg, 3.67 mmol, 1.5 eq) was added at 0° C., then the mixture was heated and stirred at 60° C. for 12 h. The reaction mixture was poured into water (20 mL) and the mixture was extracted with ethyl acetate (15 mL). The aqueous phase was extracted with ethyl acetate (3×15 mL). The combined organic phase was dried with anhydrous sodium sulfate, filtered and concentrated under reduced pressure to give a residue (3 g). The residue was purified by column chromatography (SiO₂, Petroleum ether/Ethyl acetate=1/0 to 1/1) to give tert-butyl (6S,7S)-6-[[3-[2-[2-[tert-butoxycarbonyl (methyl) amino]ethoxy]phenyl]-2-fluoro-phenyl]methyl]-7-(difluoromethyl sulfonylamino)-5-azaspiro[2.4]heptane-5-carboxylate (700 mg, yield 41%) as colorless oil. LCMS, Method E: (ESI⁺): m/z 584.3 (M−100)⁺, RT: 0.922 min.

### Step 7: 1,1-difluoro-N-[(6S,7S)-6-[[2-fluoro-3-[2-[2-(methylamino) ethoxy]phenyl]phenyl]methyl]-5-azaspiro[2.4]heptan-7-yl]methanesulfonamide

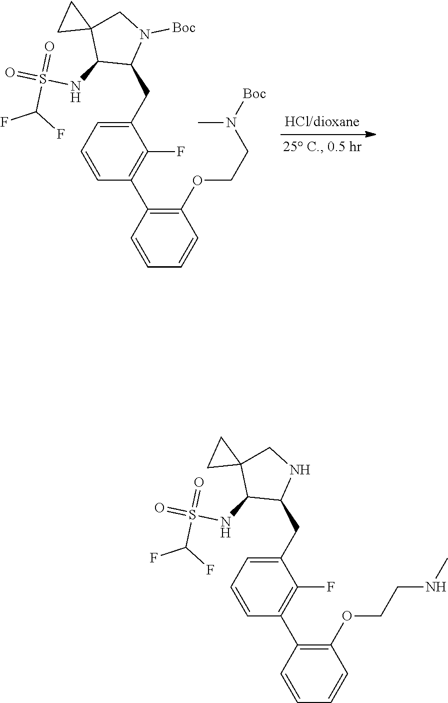 [SMILES: CN(CCOc1ccccc1-c1cccc(C[C@H]2[C@@H](NS(=O)(=O)C(F)F)C3(CC3)CN2C(=O)OC(C)(C)C)c1F)C(=O)OC(C)(C)C ; CNCCOc1ccccc1-c1cccc(C[C@@H]2NCC3(CC3)[C@@H]2NS(=O)(=O)C(F)F)c1F]

A solution of tert-butyl (6S,7S)-6-[[3-[2-[2-[tert-butoxycarbonyl (methyl) amino]ethoxy]phenyl]-2-fluoro-phenyl]methyl]-7-(difluoromethyl sulfonylamino)-5-azaspiro[2.4]heptane-5-carboxylate (558 mg, 816 μmol, 1 eq) in HCl/dioxane (30 mL) was stirred at 25° C. for 0.5 hr. The mixture was concentrated under reduced pressure to give 1,1-difluoro-N-[(6S,7S)-6-[[2-fluoro-3-[2-[2-(methylamino)ethoxy]phenyl]phenyl]methyl]-5-azaspiro[2.4]heptan-7-yl]methane sulfonamide (380 mg, yield 96%) as white solid, which was used directly without any purification. LCMS, Method E: (ESI⁺): m/z 484.2 (M+H)⁺, RT: 0.590 min.

### Step 8: 1,1-difluoro-N-((2'S, 3'S)-2′-fluoro-6′-methyl-5′-oxospiro[cyclopropane-1,4′-9-oxa-6-aza-4(2,1)-pyrrolidina-1(1,2), 2(1,3)-dibenzenacyclononaphan]-3′-yl)methanesulfonamide

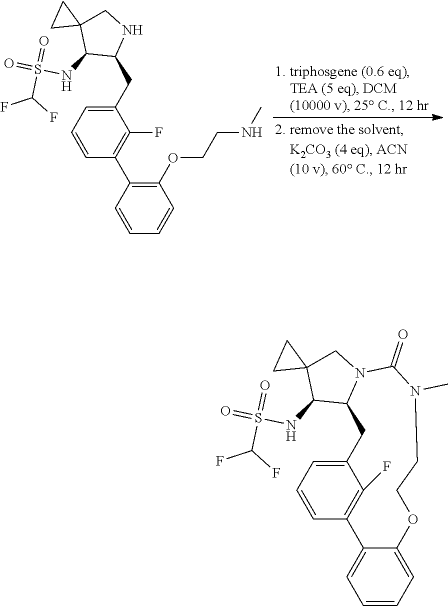 [SMILES: CNCCOc1ccccc1-c1cccc(C[C@@H]2NCC3(CC3)[C@@H]2NS(=O)(=O)C(F)F)c1F ; CN1CCOc2ccccc2-c2cccc(c2F)C[C@H]2[C@@H](NS(=O)(=O)C(F)F)C3(CC3)CN2C1=O]

To a solution of 1,1-difluoro-N-[(6S,7S)-6-[[2-fluoro-3-[2-[2-(methylamino)ethoxy]phenyl]phenyl]methyl]-5-azaspiro[2.4]heptan-7-yl]methane sulfonamide (213 mg, 440 μmol, 1 eq) in dichloromethane (2.2 L) was added TEA (223 mg, 2.20 mmol, 306 μL, 5 eq) and a solution of triphosgene (78 mg, 264 μmol, 0.6 eq) in dichloromethane (1 mL) was added dropwise into the mixture above. Then the mixture was stirred at 25° C. for 12 h. The reaction mixture was concentrated under reduced pressure to give a residue. Acetonitrile (2.2 mL) and K₂CO₃ (243 mg, 1.76 mmol, 4 eq) was added into the mixture then the mixture was stirred at 60° C. for 12 h. The mixture was concentrated under reduced pressure and give a residue and the residue was purified by prep-HPLC (column: Waters Xbridge Prep OBD C18 150*40 mm*10 μm; mobile phase: [water (NH₄HCO₃)-acetonitrile]; B %: 35%-65%, 8 min) to give 1,1-difluoro-N-((2'S,3'S)-2′-fluoro-6′-methyl-5′-oxospiro[cyclopropane-1,4′-9-oxa-6-aza-4(2,1)-pyrrolidina-1(1,2),2(1,3)-dibenzenacyclononaphan]-3′-yl)methanesulfonamide (66 mg, yield 16%) as a white solid.

TABLE A
Example Numbers 1-41 Experimental Details
The compounds of Table A were synthesized according to the methods described above with the appropriate
starting materials.

| Ex. | | | |
|---|---|---|---|
| No | Structure Name | Structure Data | Structure |
| 1 | 1,1-difluoro-N-((4²S,4³R)- 1³,4⁴,4⁴-trifluoro-6-methyl- 5-oxo-9-oxa-6-aza-4(2,1)- pyrrolidina-1(1,2),2(1,3)- dibenzenacyclononaphane- 4³-yl)methanesulfonamide | ¹H NMR (400 MHz, methanol- d4) δ ppm 2.65 (d, 1 H), 2.75 (s, 3 H), 2.90-3.00 (m, 1 H), 3.20- 3.30 (m, 1 H), 3.75 (dd, 1 H), 3.90-4.00 (m, 2 H), 4.05 (dd, 1 H), 4.30-4.45 (m, 2 H), 4.40- 4.50 (m, 1 H), 6.7 (t, 1H), 7.00- 7.25 (m, 5H), 7.35 (t, 1H), 7.50 (s, 1H). LCMS (method C) m/z 520 (ES+, M + H) at 2.64 min | 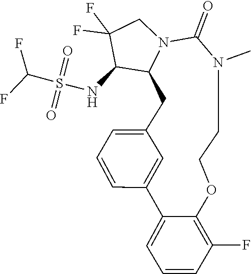 [SMILES: CN1CCOc2c(F)cccc2-c2cccc(c2)C[C@H]2[C@@H](NS(=O)(=O)C(F)F)C(F)(F)CN2C1=O] |
| 2 | 1-fluoro-N-((4²S,4³S,4⁴S)- 2²-fluoro-4⁴,6-dimethyl-5- oxo-9-oxa-6-aza-4(2,1)- pyrrolidina-1(1,2),2(1,3)- dibenzenacyclononaphane- 4³-yl)methanesulfonamide | ¹H NMR (400 MHz, aceto- nitrile-d3) δ ppm 1.00 (d, 3 H), 2.10-2.30 (m, 3H), 2.60 (d, 1H), 2.75 (br s, 3 H), 3.00 (t, 1 H), 3.20-3.30 (m, 2 H), 4.00-4.20 (m, 4 H), 4.40-4.50 (m, 1 H), 5.10-5.30 (m, 1 H), 5.80 (br s, 1 H), 6.90-7.15 (m, 4 H), 7.20- 7.25 (m, 2 H), 7.30 (td, 1H). LCMS (method B) m/z 480 (ES+, M + H) at 2.44 min | 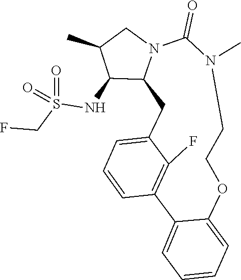 [SMILES: C[C@H]1CN2C(=O)N(C)CCOc3ccccc3-c3cccc(c3F)C[C@H]2[C@H]1NS(=O)(=O)CF] |
| 3 | N-((4²S,4³S)-2²-fluoro-5- oxo-8-oxa-4(2,1)-piperi- dina-1(1,2),2(1,3)- dibenzenacyclooctaphane- 4³-yl)propane-2-sulfon- amide | ¹H NMR (400 MHz, methanol- d4) δ ppm 1.33 (d, 3 H), 1.40 (d, 3 H), 1.50-1.70 (m, 1 H), 1.75-1.95 (m, 3 H), 2.40-2.50 (m, 1H), 2.80 (br d, 1 H), 3.10- 3.30 (m, 4 H), 3.40-3.50 (m, 1 H), 3.55 (t, 1 H), 3.80 (br d, 1 H), 4.05-4.15 (m, 1 H), 4.22- 4.28 (m, 1 H), 5.00-5.10 (m, 1 H), 7.10-7.25 (m, 4 H), 7.30 (t, 1 H), 7.32-7.40 (m, 2 H). LCMS (method B) m/z 461 (ES+, M + H) at 2.37 min | 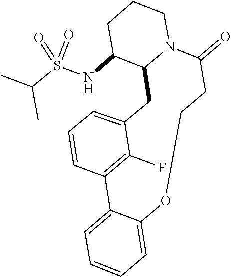 |
| 4 | 1,1-difluoro-N-((2′S,3′S)- 2′-fluoro-6′-methyl-5′- oxospiro[cyclopropane- 1,4′-9-oxa-6-aza-4(2,1)- pyrrolidina-1(1,2),2(1,3)- dibenzenacyclononaphan]- 3′-yl)methanesulfonamide | ¹H NMR (400 MHz, aceto- nitrile-d3) δ ppm 0.60-0.75 (m, 3 H), 0.80-0.90 (m, 1 H), 2.50- 2.60 (m, 1H), 2.70 (s, 3 H), 3.00-3.10 (m, 2 H), 3.50-3.75 (br m, 2 H), 3.77 (d, 1 H), 3.80 (d, 1 H), 3.95-4.05 (m, 1 H), 4.15-4.25 (m, 1 H), 4.60-4.70 (m, 1 H), 6.40 (br s, 1 H), 6.50 (t, 1 H) 6.90 (d, 1 H), 7.00-7.20 (m, 3 H), 7.25 (td, 1 H), 7.27 (dd, 1 H), 7.32 (td, 1 H). LCMS (method C) m/z 510 (ES+, M + H) at 2.94 min | 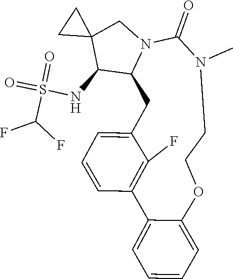 [SMILES: CN1CCOc2ccccc2-c2cccc(c2F)C[C@H]2[C@@H](NS(=O)(=O)C(F)F)C3(CC3)CN2C1=O] |
| 5 | 1,1-difluoro-N-((2′S,3′S)- 2′,3′,5′-trifluoro-6′-methyl- 5′-oxospiro[cyclopropane- 1,4′-9-oxa-6-aza-4(2,1)- pyrrolidina-1(1,2),2(1,3)- dibenzenacyclononaphan]- 3′-yl)methanesulfonamide | ¹H NMR (400 MHz, methanol- d4) δ ppm 0.55-0.70 (m, 3 H), 1.00-1.10 (m, 1 H), 2.75 (s, 3 H), 2.90-2.95 (m, 1 H), 3.10- 3.30 (m, 3 H), 3.75 (d, 1 H), 3.80-3.95 (m, 2 H), 3.98 (d, 1 H), 4.60-4.75 (m, 2 H), 6.70 (t, 1H), 6.80-7.00 (m, 1 H), 7.00- 7.10 (m, 2 H), 7.15 (t, 1H), 7.30 (t, 1H). LCMS (method B) m/z 546 (ES+, M + H) at 2.70 min | 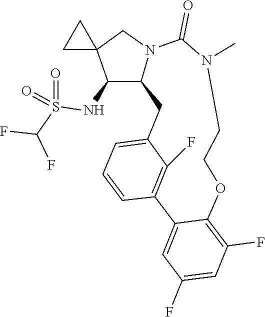 [SMILES: CN1CCOc2c(F)cc(F)cc2-c2cccc(c2F)C[C@H]2[C@@H](NS(=O)(=O)C(F)F)C3(CC3)CN2C1=O] |
| 6 | 1-fluoro-N-((2′S,3′S)- 2′,3′,5′-trifluoro-6′-methyl- 5′-oxospiro[cyclopropane- 1,4′-9-oxa-6-aza-4(2,1)- pyrrolidina-1(1,2),2(1,3)- dibenzenacyclononaphan]- 3′-yl)methanesulfonamide | ¹H NMR (400 MHz, methanol- d4) δ ppm 0.55-0.70 (m, 3 H), 1.00-1.10 (m, 1 H), 2.75 (s, 3 H), 2.90-2.95 (m, 1 H), 3.10 (t, 1 H), 3.15-3.25 (m, 2 H), 3.75 (d, 1 H), 3.80-3.95 (m, 3 H), 4.60-4.70 (m, 2 H), 5.76 (q, 1 H), 5.85 (q, 1 H), 6.80-7.00 (m, 1 H), 7.00-7.10 (m, 2 H), 7.20 (t, 1H), 7.30 (t, 1H). LCMS (method B) m/z 528 (ES+, M + H) at 2.61 min | 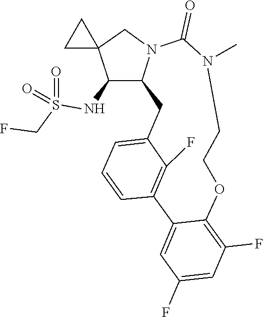 [SMILES: CN1CCOc2c(F)cc(F)cc2-c2cccc(c2F)C[C@H]2[C@@H](NS(=O)(=O)CF)C3(CC3)CN2C1=O] |
| 7 | 1-fluoro-N-((4²S,4³R)- 1³,4⁴,4⁴-trifluoro-6-methyl- 5-oxo-9-oxa-6-aza-4(2,1)- pyrrolidina-1(1,2),2(1,3)- dibenzenacyclononaphane- 4³-yl)methanesulfonamide | ¹H NMR (400 MHz, methanol- d4) δ ppm 2.60 (t, 1 H), 2.75 (s, 3 H), 3.00 (dd, 1 H), 3.20-3.30 (m, 1 H), 3.75 (q, 1 H), 3.95- 4.05 (m, 2 H), 4.10 (q, 1 H), 4.30-4.40 (m, 2 H), 4.70 (t, 1 H), 5.30 (d, 2 H), 7.00-7.15 (m, 4 H), 7.20 (d, 1 H), 7.30 (t, 1 H), 7.50 (s, 1 H). LCMS (method B) m/z 502 (ES+, M + H) at 2.55 min | 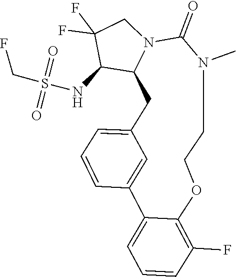 [SMILES: CN1CCOc2c(F)cccc2-c2cccc(c2)C[C@H]2[C@@H](NS(=O)(=O)CF)C(F)(F)CN2C1=O] |
| 8 | N-((2′S,3′S)-2′,3′-difluoro- 6′-methyl-5′-oxospiro [cyclopropane-1,4′-9-oxa- 6-aza-4(2,1)-pyrrolidina- 1(1,2),2(1,3)-dibenzena- cyclononaphan]-3′-yl)-1- fluoromethanesulfonamide | ¹H NMR (400 MHz, methanol- d4) δ ppm 0.55-0.70 (m, 3 H), 1.00-1.10 (m, 1 H), 2.75 (s, 3 H), 2.85-2.90 (m, 1 H), 3.10 (t, 1 H), 3.15-3.25 (m, 2 H), 3.30- 3.40 (m, 1 H), 3.75 (d, 1 H), 3.90-4.10 (m, 3 H), 4.60-4.75 (m, 2 H), 5.80 (dq, 2 H), 7.00- 7.25 (m, 5 H), 7.25 (t, 1H). LCMS (method B) m/z 510 (ES+, M + H) at 2.56 min | 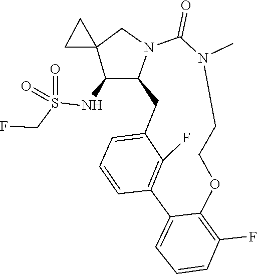 [SMILES: CN1CCOc2c(F)cccc2-c2cccc(c2F)C[C@H]2[C@@H](NS(=O)(=O)CF)C3(CC3)CN2C1=O] |
| 9 | N-((2′S,3′S)-2′,3′-difluoro- 6′-methyl-5′-oxospiro [cyclopropane-1,4′-9-oxa- 6-aza-4(2,1)-pyrrolidina- 1(1,2),2(1,3)-dibenzena- cyclononaphan]-3′-yl)- ethanesulfonamide | ¹H NMR (400 MHz, methanol- d4) δ ppm 0.55-0.75 (m, 3 H), 1.00-1.10 (m, 1 H), 1.40 (t, 3 H), 2.75 (s, 3 H), 2.80-2.90 (m, 1 H), 3.05-3.40 (m, 6 H), 3.75 (d, 1 H), 3.95-4.20 (m, 3 H), 4.60-4.75 (m, 2 H), 7.00-7.20 (m, 5 H), 7.30 (t, 1H). LCMS (method B) m/z 506 (ES+, M + H) at 2.53 min | 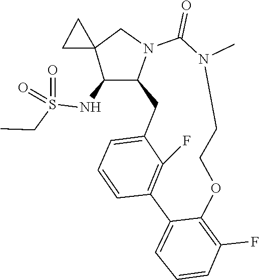 [SMILES: CCS(=O)(=O)N[C@@H]1[C@@H]2Cc3cccc(c3F)-c3cccc(F)c3OCCN(C)C(=O)N2CC12CC2] |
| 10 | N-((2′S,3′S)-6′-ethyl-5′- oxospiro[cyclopropane- 1,4′-9-oxa-6-aza-4(2,1)- pyrrolidina-1(1,2),2(1,3)- dibenzenacyclononaphan]- 3′-yl)-1,1-difluorometh- anesulfonamide | ¹H NMR (400 MHz, methanol- d4) δ ppm 0.60-0.75 (m, 3 H), 1.00-1.10 (m, 1 H), 1.10 (t, 3 H), 2.70 (t, 1 H), 2.80-2.90 (m, 2 H), 3.25 (d, 1 H), 3.40-3.50 (m, 2 H), 3.80 (d, 1 H), 3.90 (t, 2 H), 4.05-4.20 (m, 2 H), 4.70- 4.75 (m, 1 H), 6.65 (t, 1 H), 6.90-7.00 (m, 2 H), 7.10 (d, 1 H), 7.20 (d, 1 H), 7.25-7.35 (m, 2 H), 7.35 (dd, 1 H), 7.60 (s, 1 H). LCMS (method B) m/z 506 (ES+, M + H) at 2.70 min | 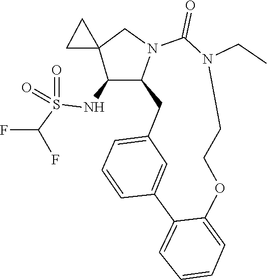 [SMILES: CCN1CCOc2ccccc2-c2cccc(c2)C[C@H]2[C@@H](NS(=O)(=O)C(F)F)C3(CC3)CN2C1=O] |
| 11 | 1,1-difluoro-N-((4²S,4³R, 4⁴S)-1³,1⁵,2²,4⁴-tetra- fluoro-6-methyl-5-oxo-9- oxa-6-aza-4(2,1)-pyrroli- dina-1(1,2),2(1,3)- dibenzenacyclononaphane- 4³-yl)methanesulfonamide | ¹H NMR (400 MHz, aceto- nitrile-d3) δ ppm 2.60 (s, 3 H), 2.70 (d, 1 H), 3.00 (t, 1 H), 3.10 (d, 1 H), 3.60 (dd, 1 H), 3.80 (dd, 1 H), 4.00-4.20 (m, 3 H), 4.50-4.60 (m, 1 H), 4.65-4.75 (m, 1 H), 5.20 (dt, 1 H), 6.55 (t, 1 H), 6.90-6.95 (m, 1 H), 7.00- 7.15 (m, 2 H), 7.20 (t, 1 H), 7.30 (t, 1 H). LCMS (method B) m/z 538 (ES+, M + H) at 2.61 min | 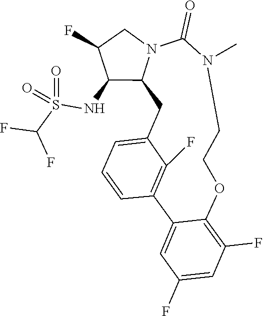 [SMILES: CN1CCOc2c(F)cc(F)cc2-c2cccc(c2F)C[C@H]2[C@@H](NS(=O)(=O)C(F)F)[C@@H](F)CN2C1=O] |
| 12 | 1-fluoro-N-((4²S,4³R,4⁴S)- 1³,2²,4⁴-trifluoro-6-methyl- 5-oxo-9-oxa-6-aza-4(2,1)- pyrrolidina-1(1,2),2(1,3)- dibenzenacyclononaphane- 4³-yl)methanesulfonamide | ¹H NMR (400 MHz, aceto- nitrile-d3) δ ppm 2.60 (s, 3 H), 2.70 (d, 1 H), 3.00 (t, 1 H), 3.10 (d, 1 H), 3.65 (dd, 1 H), 3.80 (dd, 1 H), 4.00-4.20 (m, 3 H), 4.50-4.60 (m, 1 H), 4.70-4.80 (m, 1 H), 5.10-5.40 (m, 3 H), 6.40 (br s, 1 H), 7.00-7.30 (m, 6 H). LCMS (method C) m/z 502 (ES+, M + H) at 2.86 min | 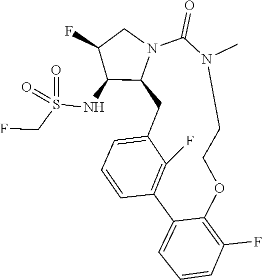 [SMILES: CN1CCOc2c(F)cccc2-c2cccc(c2F)C[C@H]2[C@@H](NS(=O)(=O)CF)[C@@H](F)CN2C1=O] |
| 13 | N-((4²S,4³S)-1³,1⁵-di- fluoro-5-oxo-8-oxa-4(2,1)- piperidina-1(1,2),2(1,3)- dibenzenacyclooctaphane- 4³-yl)-1-fluoromethanesul- fonamide | ¹H NMR (400 MHz, aceto- nitrile-d3) δ ppm 1.60-1.75 (m, 2 H), 1.80-1.90 (m, 2 H), 2.00- 2.05 (m, 2 H), 2.80-3.00 (m, 2 H), 3.20 (t, 1 H), 3.45 (t, 1 H), 3.50-3.65 (m, 2 H), 3.95 (d, 1 H), 4.10-4.20 (m, 1 H), 4.80- 4.90 (m, 1 H), 5.10-5.30 (m, 2 H), 6.10 (br s, 1 H), 7.00 (t, 1 H), 7.10 (d, 1 H), 7.25 (t, 1 H), 7.30-7.40 (m, 2 H). LCMS (method C) m/z 469 (ES+, M + H) at 2.89 min | 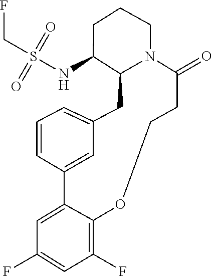 [SMILES: O=C1CCOc2c(F)cc(F)cc2-c2cccc(c2)C[C@H]2[C@@H](NS(=O)(=O)CF)CCCN12] |
| 14 | N-((4²S,4³S)-1³,1⁵-di- fluoro-5-oxo-8-oxa-4(2,1)- piperidina-1(1,2),2(1,3)- dibenzenacyclooctaphane- 4³-yl)-1,1-difluorometh- anesulfonamide | ¹H NMR (400 MHz, aceto- nitrile-d3) δ ppm 1.60-1.75 (m, 1 H), 1.75-1.90 (m, 3 H), 2.05 (ddd, 1 H), 2.80 (dd, 1 H), 2.95 (d, 1 H), 3.20-3.25 (m, 1 H), 3.45 (t, 1 H), 3.50-3.65 (m, 2 H), 4.00 (d, 1 H), 4.10-4.20 (m, 1 H), 4.80-4.90 (m, 1 H), 6.50 (t, 1 H), 7.00-7.15 (m, 2 H), 7.20-7.40 (m, 4 H). LCMS (method C) m/z 487 (ES+, M + H) at 2.97 min | 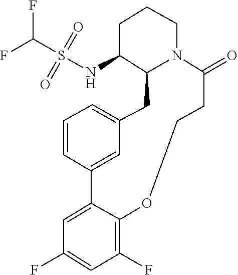 [SMILES: O=C1CCOc2c(F)cc(F)cc2-c2cccc(c2)C[C@H]2[C@@H](NS(=O)(=O)C(F)F)CCCN12] |
| 15 | N-((4²S,4³R,4⁴S)-4⁴-fluoro- 6-methyl-5-oxo-9-oxa-6- aza-4(2,1)-pyrrolidina- 1(1,2),2(1,3)-dibenzena- cyclononaphane-4³-yl)- ethanesulfonamide | ¹H NMR (400 MHz, chloro- form-d) δ ppm 1.50 (t, 3 H), 2.60 (d, 1 H), 2.70 (s, 3 H), 2.95 (d, 1 H), 3.20 (q, 2 H), 3.30 (d, 1 H), 3.60-3.80 (m, 1 H), 3.90- 4.25 (m, 5 H), 4.10-4.20 (m, 1 H), 4.75-4.90 (m, 2 H), 5.75 (dt, 1 H), 6.80 (d, 1 H), 7.00 (t, 1 H), 7.15 (d, 1 H), 7.20-7.30 (m, 1 H), 7.35 (t, 1 H), 7.40 (d, 1 H), 7.60 (s, 1 H). LCMS (method B) m/z 462 (ES+, M + H) at 2.48 min | 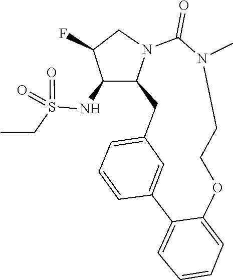 [SMILES: CCS(=O)(=O)N[C@H]1[C@@H](F)CN2C(=O)N(C)CCOc3ccccc3-c3cccc(c3)C[C@@H]12] |
| 16 | N-((4²S,4³R,4⁴S)-1³,4⁴- difluoro-6-methyl-5-oxo- 9-oxa-6-aza-4(2,1)-pyr- rolidina-1(1,2),2(1,3)- dibenzenacyclononaphane- 4³-yl)ethanesulfonamide | ¹H NMR (400 MHz, chloro- form-d) δ ppm 1.50 (t, 3 H), 2.60-2.70 (m, 1 H), 2.70 (s, 3 H), 3.05 (dd, 1 H), 3.20 (q, 2 H), 3.25-3.30 (m, 1 H), 3.65 (ddd, 1 H), 3.95 (dd, 2 H), 4.00-4.25 (m, 3 H), 4.70-4.80 (m, 1 H), 4.90 (d, 1 H), 5.20 (dt, 1 H), 7.00-7.15 (m, 3 H), 7.20- 7.25 (m, 2 H), 7.40 (t, 1 H), 7.50 (s, 1 H). LCMS (method C) m/z 478 (ES−, M − H) at 2.84 min | 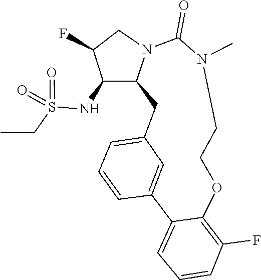 [SMILES: CCS(=O)(=O)N[C@H]1[C@@H](F)CN2C(=O)N(C)CCOc3c(F)cccc3-c3cccc(c3)C[C@@H]12] |
| 17 | N-((4²S,4³R,4⁴S)-2²,4⁴- difluoro-6-methyl-5-oxo- 9-oxa-6-aza-4(2,1)-pyr- rolidina-1(1,2),2(1,3)- dibenzenacyclononaphane- 4³-yl)ethanesulfonamide | ¹H NMR (400 MHz, chloro- form-d) δ ppm 1.50 (t, 3 H), 2.70 (s, 3 H), 2.70-2.80 (m, 1 H), 3.10 (t, 1 H), 3.20 (q, 2 H), 3.30 (d, 1 H), 3.70 (ddd, 1 H), 3.95-4.15 (m, 4 H), 4.30 (t, 1 H), 4.80-4.80 (m, 2 H), 5.20 (dt, 1 H), 6.80 (d, 1 H), 7.05 (t, 1 H), 7.10-7.25 (m, 3 H), 7.30- 7.40 (m, 2 H). LCMS (method B) m/z 480 (ES+, M + H) at 2.47 min | 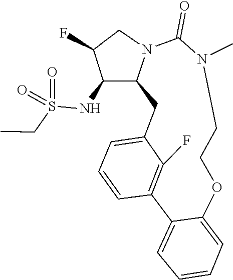 [SMILES: CCS(=O)(=O)N[C@H]1[C@@H](F)CN2C(=O)N(C)CCOc3ccccc3-c3cccc(c3F)C[C@@H]12] |
| 18 | N-((4²S,4³R,4⁴S)-1³,2²,4⁴- trifluoro-6-methyl-5-oxo- 9-oxa-6-aza-4(2,1)- pyrrolidina-1(1,2),2(1,3)- dibenzenacyclononaphane- 4³-yl)ethanesulfonamide | ¹H NMR (400 MHz, chloro- form-d) δ ppm 1.50 (t, 3 H), 2.70 (s, 3 H), 2.80 (d, 1 H), 3.00-3.25 (m, 4 H), 3.65 (ddd, 1 H), 3.90 (d, 1 H), 4.05-4.15 (m, 3 H), 4.70-5.00 (m, 3 H), 5.20 (dt, 1 H), 6.95-7.25 (m, 6 H). LCMS (method B) m/z 498 (ES+, M + H) at 2.53 min | 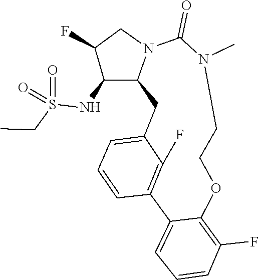 [SMILES: CCS(=O)(=O)N[C@H]1[C@@H](F)CN2C(=O)N(C)CCOc3c(F)cccc3-c3cccc(c3F)C[C@@H]12] |
| 19 | N-((2′S,3′S)-3′-fluoro-6′- methyl-5′-oxospiro[cyclo- propane-1,4′-9-oxa-6-aza- 4(2,1)-pyrrolidina- 1(1,2),2(1,3)-dibenzena- cyclononaphan]-3′-yl)- ethanesulfonamide | ¹H NMR (400 MHz, chloro- form-d) δ ppm 0.60-0.70 (m, 2 H), 0.75-0.80 (m, 1 H), 0.90- 1.00 (m, 1 H), 1.40 (t, 3 H), 2.70 (s, 3 H), 2.75 (dd, 1 H), 3.05 (dd, 1 H), 3.15-3.20 (m, 3 H), 3.25-3.30 (m, 1 H), 3.50 (d, 1 H), 3.70-3.80 (m, 1 H), 3.80- 3.90 (m, 1 H), 3.95-4.05 (m, 1 H), 4.20 (t, 1 H), 4.60-4.75 (m, 2 H), 7.00-7.15 (m, 4 H), 7.20 (d, 1 H), 7.20-7.25 (m, 1 H), 7.40 (t, 1 H), 7.50 (s, 1 H). LCMS (method B) m/z 488 (ES+, M + H) at 2.62 min | 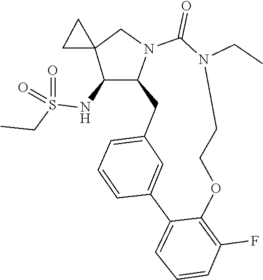 [SMILES: CCN1CCOc2c(F)cccc2-c2cccc(c2)C[C@H]2[C@@H](NS(=O)(=O)CC)C3(CC3)CN2C1=O] |
| 20 | N-((4²S,4³R,4⁴S)-1³,4⁴- difluoro-6-methyl-5-oxo- 9-oxa-6-aza-4(2,1)-pyr- rolidina-1(1,2),2(1,3)- dibenzenacyclononaphane- 4³-yl)-1,1-difluoromethane- sulfonamide | ¹H NMR (400 MHz, aceto- nitrile-d3) δ ppm 2.50-2.75 (m, 4 H), 2.90 (dd, 1 H), 3.05 (dd, 1 H), 3.25 (dd, 1 H), 3.70 (ddd, 1 H), 3.90 (dd, 1 H), 4.10-4.22 (m, 3 H), 4.45-4.60 (m, 2 H), 5.20 (dt, 1 H), 6.60 (t, 1 H), 7.00-7.25 (m, 5 H), 7.30 (t, 1 H), 7.60 (s, 1 H). LCMS (method C) m/z 502 (ES+, M + H) at 2.86 min | 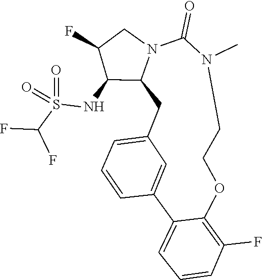 [SMILES: CN1CCOc2c(F)cccc2-c2cccc(c2)C[C@H]2[C@@H](NS(=O)(=O)C(F)F)[C@@H](F)CN2C1=O] |
| 21 | N-((4²S,4³R,4⁴S)-1³,4⁴- difluoro-6-methyl-5-oxo- 9-oxa-6-aza-4(2,1)-pyr- rolidina-1(1,2),2(1,3)- dibenzenacyclononaphane- 4³-yl)-1-fluoromethane- sulfonamide | ¹H NMR (400 MHz, aceto- nitrile-d3) δ ppm 2.50-2.60 (m, 2 H), 2.65 (s, 3 H), 2.90 (dd, 1 H), 3.20 (dt, 1 H), 3.70 (ddd, 1 H), 3.80 (dd, 1 H), 4.00-4.20 (m, 3 H), 4.45-4.60 (m, 2 H), 5.25-5.50 (m, 2 H), 6.30 (d, 1 H), 7.00-7.40 (m, 6 H), 7.60 (s, 1 H). LCMS (method C) m/z 484 (ES+, M + H) at 2.87 min | 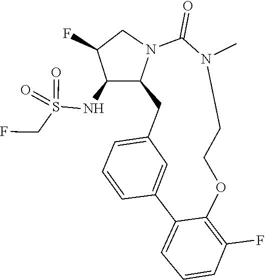 [SMILES: CN1CCOc2c(F)cccc2-c2cccc(c2)C[C@H]2[C@@H](NS(=O)(=O)CF)[C@@H](F)CN2C1=O] |
| 22 | N-((2′S,3′S)-6′-(2,2-di- fluoro-ethyl)-3′-fluoro-5′- oxospiro[cyclopropane- 1,4′-9-oxa-6-aza-4(2,1)- pyrrolidina-1(1,2),2(1,3)- dibenzenacyclononaphan]- 3′-yl)-1-fluoromethane- sulfonamide | ¹H NMR (400 MHz, DMSO-d6) δ ppm 0.50-1.00 (m, 4 H), 2.50- 2.60 (m, 2 H), 2.80 (d, 1 H), 3.10 (m, 1 H), 3.20-3.25 (m, 1 H), 3.60-3.85 (m, 3 H), 3.95- 4.15 (m, 2 H), 4.35 (t, 1 H), 4.40-4.50 (m, 1 H), 5.45 (dq, 2 H), 5.95 (tt, 1 H), 7.05-7.30 (m, 5 H), 7.35 (t, 1 H), 7.60 (s, 1 H), 8.1 (d, 1 H). LCMS (method C) m/z 542 (ES+, M + H) at 3.12 min | 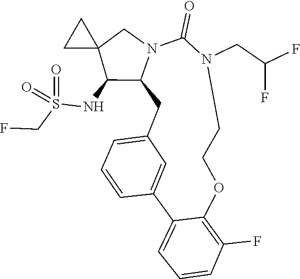 [SMILES: O=C1N(CC(F)F)CCOc2c(F)cccc2-c2cccc(c2)C[C@H]2[C@@H](NS(=O)(=O)CF)C3(CC3)CN12] |
| 23 | N-((4²S,4³R)-4⁴,4⁴-di- fluoro-6-methyl-5-oxo- 9-oxa-6-aza-4(2,1)-pyr- rolidina-1(1,2),2(1,3)- dibenzenacyclononaphane- 4³-yl)ethanesulfonamide | ¹H NMR (400 MHz, chloro- form-d) δ ppm 1.40 (t, 3 H), 2.60 (t, 1 H), 2.70 (s, 3 H), 3.05 (dd, 1 H), 3.15-3.25 (m, 2 H), 3.40 (d, 1 H), 3.80 (q, 1 H), 3.90-4.10 (m, 3 H), 4.20 (t, 1 H), 4.25-4.40 (m, 1 H), 4.80- 4.90 (m, 1 H), 5.00-5.10 (m, 1 H), 6.85 (d, 1 H), 7.05 (t, 1 H), 7.20-7.30 (m, 3 H), 7.35 (t, 1 H), 7.40 (d, 1 H), 7.60 (s, 1 H) LCMS (method B) m/z 480 (ES+, M + H) at 2.47 min | 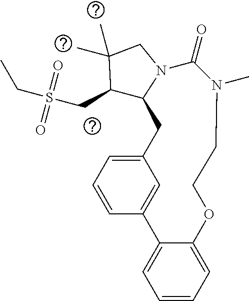 [SMILES: CCS(=O)(=O)C[C@@H]1[C@@H]2Cc3cccc(c3)-c3ccccc3OCCN(C)C(=O)N2CC1(C)C] |
| 24 | 1,1-difluoro-N-((4²S,4³S)- 1³-fluoro-5-oxo-8-oxa- 4(2,1)-piperidina- 1(1,2),2(1,3)-dibenzena- cyclooctaphane-4³-yl)- methanesulfonamide | ¹H NMR (400 MHz, DMSO- d6) δ ppm 1.45-1.55 (m, 1 H), 1.65-1.85 (m, 3 H), 2.00 (dd, 1 H), 2.75 (dd, 1 H), 2.90 (t, 1 H), 3.20-3.50 (m, 3 H), 3.55 (t, 1 H), 3.90-4.00 (m, 1 H), 4.10- 4.20 (m, 1 H), 4.75-4.85 (m, 1 H), 7.10 (t, 1 H), 7.15 (d, 1 H), 7.20-7.30 (m, 5 H), 7.35 (t, 1 H), 8.85 (d, 1 H). LCMS (method B) m/z 469 (ES+, M + H) at 2.59 min | 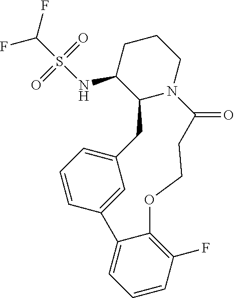 [SMILES: O=C1CCOc2c(F)cccc2-c2cccc(c2)C[C@H]2[C@@H](NS(=O)(=O)C(F)F)CCCN12] |
| 25 | 1-fluoro-N-((2′S,3′S)-2′- fluoro-6′-methyl-5′-oxo- spiro[cyclopropane-1,4′- 9-oxa-6-aza-4(2,1)-pyr- rolidina-1(1,2),2(1,3)- dibenzenacyclononaphan]- 3′-yl)methanesulfonamide | ¹H NMR (400 MHz, aceto- nitrile-d3) δ ppm 0.55-0.70 (m, 3 H), 0.80-0.90 (m, 1 H), 2.65 (d, 1 H), 2.70 (s, 3 H), 3.00 (t, 1 H), 3.10 (d, 1 H), 3.45-3.55 (m, 1 H), 3.60-3.65 (m, 1 H), 3.70 (d, 1 H), 3.80 (d, 1 H), 3.95-4.05 (m, 1 H), 4.15-4.25 (m, 1 H), 4.60-4.70 (m, 1 H), 5.25 (d, 2 H), 6.00 (br, 1 H), 6.95 (d, 1 H), 7.00-7.15 (m, 3 H), 7.20 (td, 1 H), 7.30 (dd, 1 H), 7.40 (td, 1 H). LCMS (method C) m/z 492 (ES+, M + H) at 2.87 min | 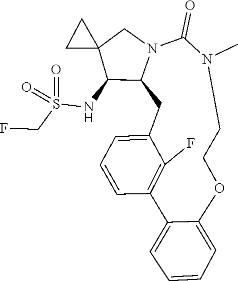 [SMILES: CN1CCOc2ccccc2-c2cccc(c2F)C[C@H]2[C@@H](NS(=O)(=O)CF)C3(CC3)CN2C1=O] |
| 26 | 1,1-difluoro-N-((4²S,4³S)- 5-oxo-8-oxa-4(2,1)- piperidina-1(1,2),2(1,3)- dibenzenacyclooctaphane- 4³-yl)methanesulfonamide | ¹H NMR (400 MHz, aceto- nitrile-d3) δ ppm 1.50-1.65 (m, 1 H), 1.75-1.90 (m, 3 H), 2.15- 2.25 (m, 2 H), 2.90 (dd, 1 H), 3.00-3.20 (m, 3 H), 3.65 (dt, 1 H), 3.80 (dd, 1 H), 4.00 (td, 1 H), 4.10-4.20 (m, 1 H), 5.15- 5.20 (m, 1 H), 6.5 (t, 1 H), 7.10-7.25 (m, 5 H), 7.30 (td, 1 H), 7.45 (dd, 1 H). LCMS (method C) m/z 451 (ES+, M + H) at 2.86 min | 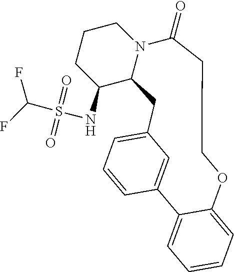 [SMILES: O=C1CCOc2ccccc2-c2cccc(c2)C[C@H]2[C@@H](NS(=O)(=O)C(F)F)CCCN12] |
| 27 | N-((4²S,4³S)-22-fluoro-5- oxo-8-oxa-4(2,1)- piperidina-1(1,2),2(1,3)- dibenzenacyclooctaphane- 4³-yl)ethanesulfonamide | ¹H NMR (400 MHz, methanol- d4) δ ppm 1.25-1.30 (m, 1 H), 1.40 (t, 3 H), 1.55-1.70 (m, 1 H), 1.75-1.95 (m, 3 H), 2.40- 2.50 (m, 1 H), 2.85 (d, 1 H), 3.20 (q, 2 H), 3.25 (t, 1 H), 3.40-3.50 (m, 1 H), 3.55 (t, 1 H), 3.85 (d, 1 H), 4.10 (td, 1 H), 4.15-4.25 (m, 1 H), 5.00-5.05 (m, 1 H), 7.05-7.20 (m, 4 H), 7.25-7.35 (m, 3 H). LCMS (method B) m/z 447 (ES+, M + H) at 2.34 min | 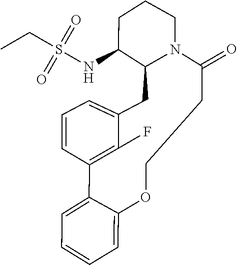 [SMILES: CCS(=O)(=O)N[C@H]1CCCN2C(=O)CCOc3ccccc3-c3cccc(c3F)C[C@@H]12] |
| 28 | (S)-1-fluoro-N-((4²S,4³S)- 5-oxo-8-oxa-4(2,1)- piperidina-1(1,2),2(1,3)- dibenzenacyclooctaphane- 4³-yl)ethane-1-sulfonamide | | 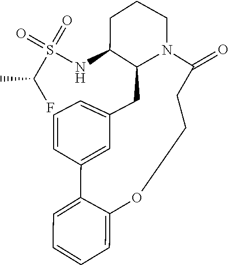 [SMILES: C[C@@H](F)S(=O)(=O)N[C@H]1CCCN2C(=O)CCOc3ccccc3-c3cccc(c3)C[C@@H]12] |
| 29 | N-((4²S,4³R,4⁴S)-2²,4⁴- difluoro-5-oxo-8-oxa- 4(2,1)-pyrrolidina- 1(1,2),2(1,3)-dibenzena- cyclooctaphane-4³-yl)- ethanesulfonamide | | 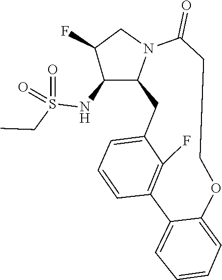 [SMILES: CCS(=O)(=O)N[C@H]1[C@@H](F)CN2C(=O)CCOc3ccccc3-c3cccc(c3F)C[C@@H]12] |
| 30 | 1-fluoro-N-((2′S,3′S)-3′- fluoro-6′-methyl-5′-oxo- spiro[cyclopropane-1,4′- 9-oxa-6-aza-4(2,1)-pyr- rolidina-1(1,2),2(1,3)- dibenzenacyclononaphan]- 3′-yl)methanesulfonamide | | 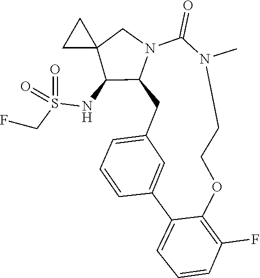 [SMILES: CN1CCOc2c(F)cccc2-c2cccc(c2)C[C@H]2[C@@H](NS(=O)(=O)CF)C3(CC3)CN2C1=O] |
| 31 | N-((2′S,3′S)-3′,5′-difluoro- 6′-methyl-5′-oxospiro [cyclopropane-1,4′-9-oxa- 6-aza-4(2,1)-pyrrolidina- 1(1,2),2(1,3)-dibenzena- cyclononaphan]-3′-yl)- 1,1-difluoromethanesul- fonamide | | 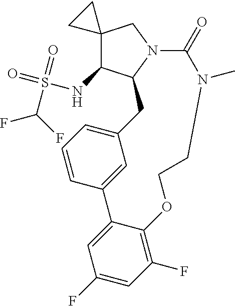 [SMILES: CN1CCOc2c(F)cc(F)cc2-c2cccc(c2)C[C@H]2[C@@H](NS(=O)(=O)C(F)F)C3(CC3)CN2C1=O] |
| 32 | 1-fluoro-N-((4²S,4³S)-2²- fluoro-6-methyl-5-oxo-9- oxa-6-aza-4(2,1)- piperidina-1(1,2),2(1,3)- dibenzenacyclononaphane- 4³-yl)methanesulfonamide | | 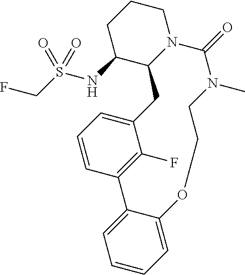 [SMILES: CN1CCOc2ccccc2-c2cccc(c2F)C[C@H]2[C@@H](NS(=O)(=O)CF)CCCN2C1=O] |
| 33 | 2-fluoro-N-((4²S,4³S)-2²- fluoro-5-oxo-8-oxa-4(2,1)- piperidina-1(1,2),2(1,3)- dibenzenacyclooctaphane- 4³-yl)propane-2-sulfon- amide | | 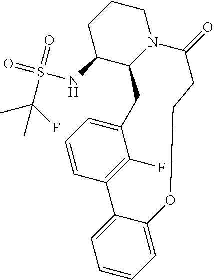 [SMILES: CC(C)(F)S(=O)(=O)N[C@H]1CCCN2C(=O)CCOc3ccccc3-c3cccc(c3F)C[C@@H]12] |
| 34 | 1,1-difluoro-N-((2′S,3′S)- 3′-fluoro-6′-methyl-5′- oxospiro[cyclopropane- 1,4′-9-oxa-6-aza-4(2,1)- pyrrolidina-1(1,2),2(1,3)- dibenzenacyclononaphan]- 3′-yl)methanesulfonamide | | 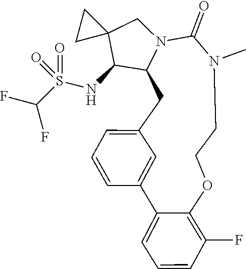 [SMILES: CN1CCOc2c(F)cccc2-c2cccc(c2)C[C@H]2[C@@H](NS(=O)(=O)C(F)F)C3(CC3)CN2C1=O] |
| 35 | N-((4²S,4³R,4⁴S)-2²,4⁴- difluoro-6-methyl-5-oxo- 9-oxa-6-aza-4(2,1)- pyrrolidina-1(1,2),2(1,3)- dibenzenacyclononaphane- 4³-yl)propane-2-sulfon- amide | | 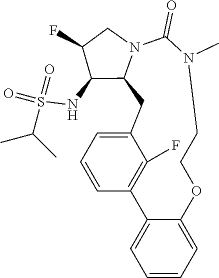 [SMILES: CC(C)S(=O)(=O)N[C@H]1[C@@H](F)CN2C(=O)N(C)CCOc3ccccc3-c3cccc(c3F)C[C@@H]12] |
| 36 | 1-fluoro-N-((4²S,4³S)-2²- fluoro-5-oxo-8-oxa-4(2,1)- piperidina-1(1,2),2(1,3)- dibenzenacyclooctaphane- 4³-yl)cyclopropane-1-sul- fonamide | | 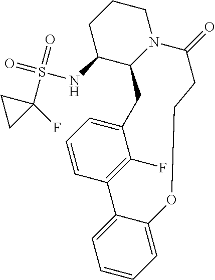 [SMILES: O=C1CCOc2ccccc2-c2cccc(c2F)C[C@H]2[C@@H](NS(=O)(=O)C3(F)CC3)CCCN12] |
| 37 | N-((2′S,3′S)-6′-ethyl-5′- oxospiro[cyclopropane- 1,4′-9-oxa-6-aza-4(2,1)- pyrrolidina-1(1,2),2(1,3)- dibenzenacyclononaphan]- 3′-yl)-1-fluoromethane- sulfonamide | | 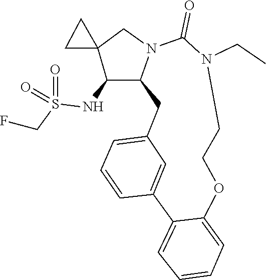 [SMILES: CCN1CCOc2ccccc2-c2cccc(c2)C[C@H]2[C@@H](NS(=O)(=O)CF)C3(CC3)CN2C1=O] |
| 38 | 2-fluoro-N-((2′S,3′S)-6′- methyl-5′-oxospiro[cyclo- propane-1,4′-9-oxa-6-aza- 4(2,1)-pyrrolidina- 1(1,2),2(1,3)-dibenzena- cyclononaphan]-3′-yl)- propane-2-sulfonamide | | 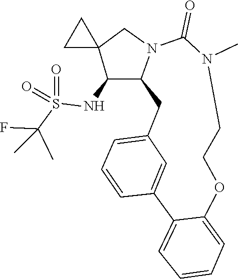 [SMILES: CN1CCOc2ccccc2-c2cccc(c2)C[C@H]2[C@@H](NS(=O)(=O)C(C)(C)F)C3(CC3)CN2C1=O] |
| 39 | 1,1-difluoro-N-((2′S,3′S)- 2′,3′,5′-trifluoro-6′-methyl- 5′-oxospiro[cyclopropane- 1,4′-9-oxa-6-aza-4(2,1)- pyrrolidina-1(1,2),2(1,3)- dibenzenacyclononaphan]- 3′-yl)methanesulfonamide | | 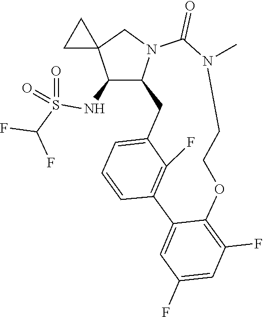 [SMILES: CN1CCOc2c(F)cc(F)cc2-c2cccc(c2F)C[C@H]2[C@@H](NS(=O)(=O)C(F)F)C3(CC3)CN2C1=O] |
| 40 | 1-fluoro-N-((2′S,3′S)- 2′,3′,5′-trifluoro-6′-methyl- 5′-oxospiro[cyclopropane- 1,4′-9-oxa-6-aza-4(2,1)- pyrrolidina-1(1,2),2(1,3)- dibenzenacyclononaphan]- 3′-yl)methanesulfonamide | | 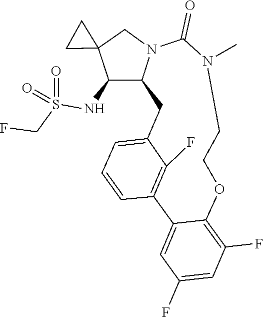 [SMILES: CN1CCOc2c(F)cc(F)cc2-c2cccc(c2F)C[C@H]2[C@@H](NS(=O)(=O)CF)C3(CC3)CN2C1=O] |

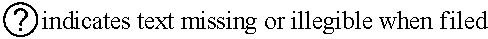

### Example 41. Synthesis of Compound 125, 1-fluoro-N-((4²S,4³R,4⁴S)-1³,1⁵,2²,4⁴-tetrafluoro-5-oxo-8-oxa-4(2,1)-piperidina-1(1,2),2(1,3)-dibenzenacyclooctaphane-4³-yl)methanesulfonamide

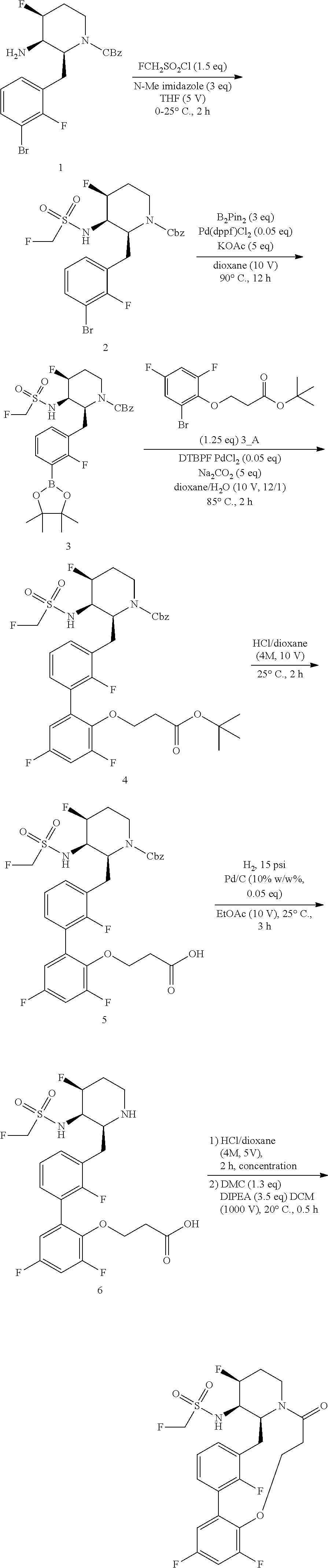

### Synthesis of intermediate 2, benzyl (2S,3R,4S)-2-[(3-bromo-2-fluoro-phenyl)methyl]-4-fluoro-3-(fluoromethylsulfonylamino)piperidine-1-carboxylate

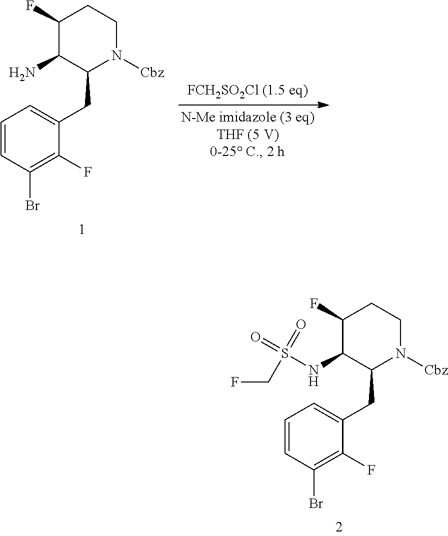 [SMILES: N[C@H]1[C@@H](F)CCN(C(=O)OCc2ccccc2)[C@H]1Cc1cccc(Br)c1F ; O=C(OCc1ccccc1)N1CC[C@H](F)[C@H](NS(=O)(=O)CF)[C@@H]1Cc1cccc(Br)c1F]

To a solution of benzyl (2S,3R,4S)-3-amino-2-(3-bromo-2-fluorobenzyl)-4-fluoropiperidine-1-carboxylate (1) (5.2 g, 11.8 mmol, 1 eq) and 1-methylimidazole (2.92 g, 35.5 mmol, 2.83 mL, 3 eq) in tetrahydrofuran (20 mL) was degassed and purged with nitrogen at 20° C. Fluoromethanesulfonyl chloride (2.35 g, 17.8 mmol, 1.5 eq) was dissolved in a solution of tetrahydrofuran (6 mL) and added dropwise at 0° C., the mixture was stirred at 0-25° C. for 2 h. The reaction mixture was diluted with water (30 mL) and extracted with ethyl acetate (3×30 mL). The combined organic phase was washed with brine (30 mL), dried over anhydrous sodium sulfate, filtered and the filtrate was concentrated under reduced pressure to give residue. The residue was triturated with petroleum ether:tert-butyl methyl ether (10:1, 20 mL). The resulting solid was collected by filtration, washed with petroleum ether:tert-butyl methyl ether (10:1, 5 mL) and dried under high vacuum to give benzyl (2S,3R,4S)-2-[(3-bromo-2-fluoro-phenyl)methyl]-4-fluoro-3-(fluoromethylsulfonylamino)piperidine-1-carboxylate (5.3 g, 84% yield) as a yellow solid. ¹H NMR: (400 MHz, DMSO-d₆) δ ppm 1.75-2.05 (m, 2H), 2.90-3.05 (br t, 1H), 3.30 (m, 2H), 3.65-4.00 (br m, 2H), 4.40-4.06 (br m, 2H), 4.80-5.05 (m, 2H), 5.20-5.60 (m, 2H), 6.80-7.15 (m, 3H), 7.15-7.40 (m, 4H), 7.50 (br t, 1H), 8.65 (br s, 1H)

### Synthesis of intermediate 3, benzyl (2S,3R,4S)-4-fluoro-3-(fluoromethylsulfonylamino)-2-[[2-fluoro-3-(4,4,5,5-tetramethyl-1,3,2-dioxaborolan-2-yl)phenyl]methyl]piperidine-1-carboxylate

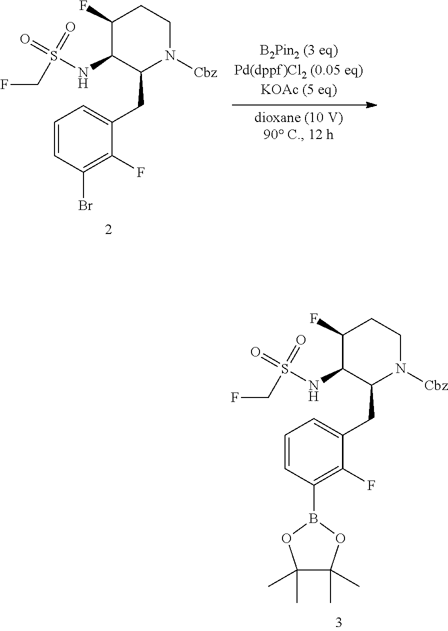 [SMILES: O=C(OCc1ccccc1)N1CC[C@H](F)[C@H](NS(=O)(=O)CF)[C@@H]1Cc1cccc(Br)c1F ; CC1(C)OB(c2cccc(C[C@H]3[C@@H](NS(=O)(=O)CF)[C@@H](F)CCN3C(=O)OCc3ccccc3)c2F)OC1(C)C]

To a solution of intermediate 2 (5.3 g, 9.90 mmol, 1 eq) in dioxane (53 mL) was added bispinacolatodiborane (7.54 g, 29.7 mmol, 3 eq), potassium acetate (4.86 g, 49.5 mmol, 5 eq) and Pd(dppf)Cl₂ (362 mg, 495 μmol, 0.05 eq) at 25° C. The mixture was stirred at 90° C. for 12 h under nitrogen atmosphere. The reaction was filtered and the filtrate was concentrated to give a residue. The residue was purified by column chromatography on silica gel eluted with petroleum ether:ethyl acetate (100:1 to 1:100) to give benzyl (2S,3R,4S)-4-fluoro-3-(fluoromethylsulfonylamino)-2-[[2-fluoro-3-(4,4,5,5-tetramethyl-1,3,2-dioxaborolan-2-yl)phenyl]methyl]piperidine-1-carboxylate (intermediate 3, 5.1 g, yield 87%) as a yellow solid.

LCMS, Method G (ESI⁺): m/z 501 (M−81)⁺, RT: 6.436 min, m/z 583 (M+H)⁺, RT: 8.821 min Synthesis of intermediate 4, benzyl (2S,3R,4S)-2-[[3-[2-(3-tert-butoxy-3-oxo-propoxy)-3,5-difluoro-phenyl]-2-fluoro-phenyl]methyl]-4-fluoro-3-(fluoromethylsulfonylamino)piperidine-1-carboxylate

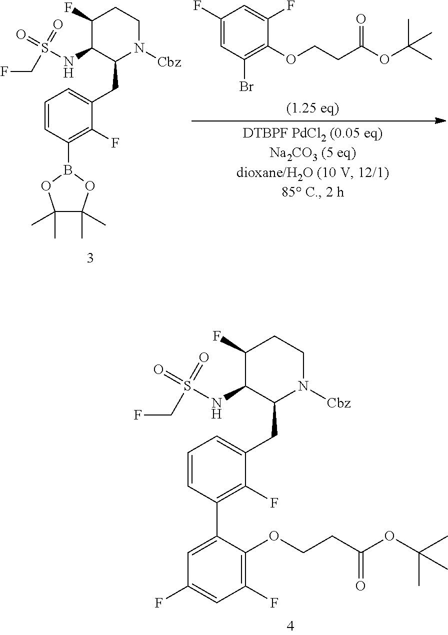 [SMILES: CC1(C)OB(c2cccc(C[C@H]3[C@@H](NS(=O)(=O)CF)[C@@H](F)CCN3C(=O)OCc3ccccc3)c2F)OC1(C)C ; CC(C)(C)OC(=O)CCOc1c(F)cc(F)cc1Br ; CC(C)(C)OC(=O)CCOc1c(F)cc(F)cc1-c1cccc(C[C@H]2[C@@H](NS(=O)(=O)CF)[C@@H](F)CCN2C(=O)OCc2ccccc2)c1F]

A mixture of tert-butyl 3-(2-bromo-4,6-difluorophenoxy)propanoate (1.81 g, 5.37 mmol, 1.25 eq), intermediate 3 (2.5 g, 4.29 mmol, 1 eq), ditert-butyl(cyclopentyl)phosphane; dichloropalladium; iron (140 mg, 215 μmol, 0.05 eq) and Na₂CO₃ (2.27 g, 21.5 mmol, 5 eq) in dioxane (23 mL) and water (1.92 mL) was degassed and purged with nitrogen 3 times, and then the mixture was stirred at 85° C. for 2 h. The reaction mixture was quenched by addition water (30 mL) at 20° C. and extracted with ethyl acetate (3×30 mL). The combined organic phase was washed with brine (30 mL), dried over anhydrous sodium sulfate, filtered and the filtrate was concentrated under reduced pressure to give residue. The residue was purified by prep-HPLC (trifluoroacetic acid condition, column: Phenomenex Luna 80*30 mm*3 μm; mobile phase: [water (trifluoroacetic acid)-acetonitrile]; gradient: 40%-70% B over 8 min) to give benzyl (2S,3R,4S)-2-[[3-[2-(3-tert-butoxy-3-oxo-propoxy)-3,5-difluoro-phenyl]-2-fluoro-phenyl]methyl]-4-fluoro-3-(fluoromethylsulfonylamino)piperidine-1-carboxylate (intermediate 4, 1.53 g, 50% yield) as a white solid. LCMS Method H: (ESI⁺): m/z 730 (M+18)⁺, RT: 3.618 min

### Synthesis of intermediate 5, 3-[2-[3-[[(2S,3R,4S)-1-benzyloxycarbonyl-4-fluoro-3-(fluoromethylsulfonylamino)-2-piperidyl]methyl]-2-fluoro-phenyl]-4,6-difluoro-phenoxy]propanoic acid

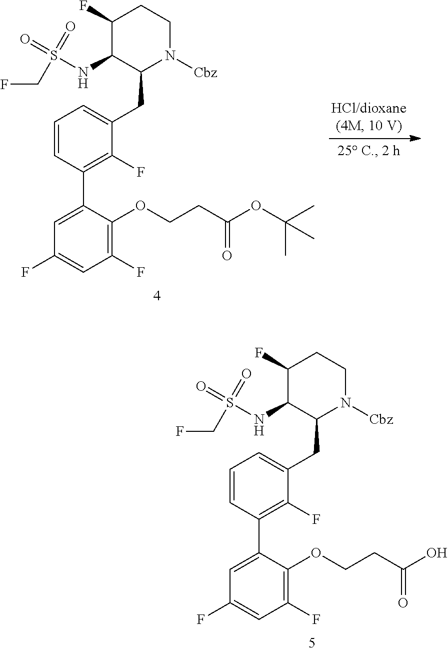 [SMILES: CC(C)(C)OC(=O)CCOc1c(F)cc(F)cc1-c1cccc(C[C@H]2[C@@H](NS(=O)(=O)CF)[C@@H](F)CCN2C(=O)OCc2ccccc2)c1F ; O=C(O)CCOc1c(F)cc(F)cc1-c1cccc(C[C@H]2[C@@H](NS(=O)(=O)CF)[C@@H](F)CCN2C(=O)OCc2ccccc2)c1F]

A solution of intermediate 4 (1.53 g, 2.15 mmol, 1 eq) in hydrochloric acid (15 mL, 4 M a solution of dioxane) was stirred at 25° C. for 2 h. The reaction was concentrated under reduced pressure to give 3-[2-[3-[[(2S,3R,4S)-1-benzyloxycarbonyl-4-fluoro-3-(fluoromethylsulfonylamino)-2-piperidyl]methyl]-2-fluoro-phenyl]-4,6-difluoro-phenoxy]propanoic acid (intermediate 5, 1.62 g, 97.69% yield) as a white solid, which was used directly in next step without purification. ¹H NMR (400 MHz, DMSO-d₆) δ ppm 1.60-2.5 (m, 2H), 2.35 (t, 1H), 2.95-3.35 (m, 3H), 3.70-3.95 (br m, 4H), 4.40-5.00 (m, 4H), 5.30-5.60 (m, 2H), 6.80-7.50 (m, 10H), 8.70 (br s, 1H), 12.20 (br s, 1H).

### Synthesis of intermediate 6, 3-[2,4-difluoro-6-[2-fluoro-3-[[(2S,3R,4S)-4-fluoro-3-(fluoromethylsulfonylamino)-2-piperidyl]methyl]phenyl]phenoxy]propanoic acid

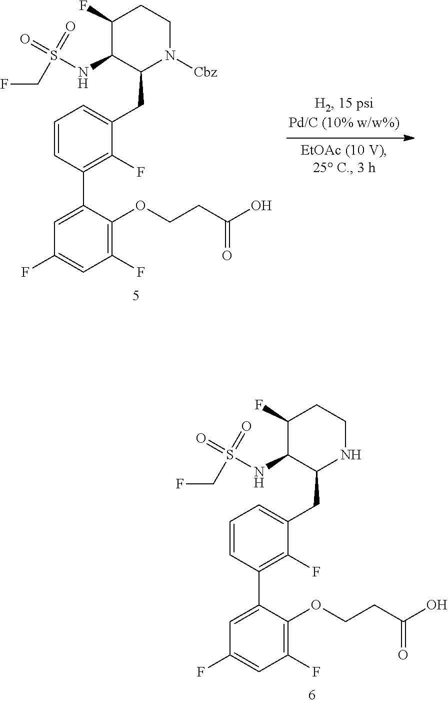 [SMILES: O=C(O)CCOc1c(F)cc(F)cc1-c1cccc(C[C@H]2[C@@H](NS(=O)(=O)CF)[C@@H](F)CCN2C(=O)OCc2ccccc2)c1F ; O=C(O)CCOc1c(F)cc(F)cc1-c1cccc(C[C@@H]2NCC[C@H](F)[C@@H]2NS(=O)(=O)CF)c1F]

A mixture of intermediate 5 (1.62 g, 2.47 mmol, 1 eq), Pd/C (162 mg, 10% purity) in ethyl acetate (16 mL) was degassed and purged with H₂ for 3 times, and then the mixture was stirred under H₂ (15 psi) at 25° C. for 3 h. The precipitate was filtered and the filter cake was washed with ethyl acetate (50 mL), and the filtrate was concentrated under high vacuum to give a residue. The residue was triturated with tert-butyl methyl ether (10 mL). The resulting solid was collected by filtration, washed with tert-butyl methyl ether (5 mL) and dried under high vacuum to give 3-[2,4-difluoro-6-[2-fluoro-3-[[(2S,3R,4S)-4-fluoro-3-(fluoromethylsulfonylamino)-2-piperidyl]methyl]phenyl]phenoxy]propanoic acid (intermediate 6, 0.792 g) as a white solid.

¹H NMR (400 MHz, DMSO-d₆) δ ppm 1.55-1.70 (m, 2H), 2.25-2.40 (m, 4H), 2.60-2.90 (m, 5H), 3.70-4.10 (m, 3H), 4.50-4.80 (m, 1H), 5.15-5.40 (m, 2H), 7.00-7.47 (m, 5H).

### Synthesis of Compound 125, 1-fluoro-N-((4²S, 4³R,4⁴S)-1³,1⁵,2²,4⁴-tetrafluoro-5-oxo-8-oxa-4(2,1)-piperidina-1(1,2),2(1,3)-dibenzenacyclooctaphane-4³-yl)methanesulfonamide

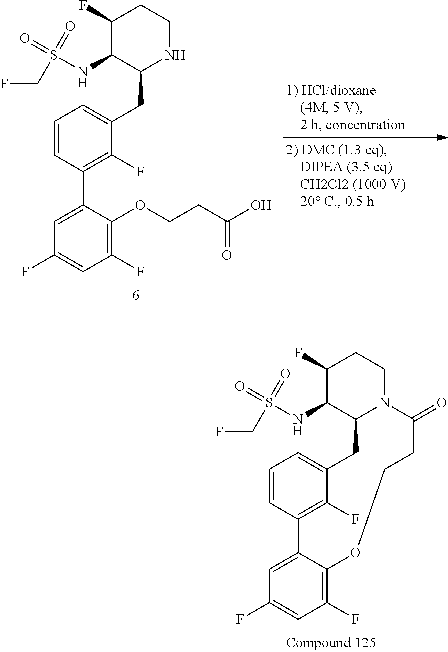 [SMILES: O=C(O)CCOc1c(F)cc(F)cc1-c1cccc(C[C@@H]2NCC[C@H](F)[C@@H]2NS(=O)(=O)CF)c1F ; O=C1CCOc2c(F)cc(F)cc2-c2cccc(c2F)C[C@H]2[C@@H](NS(=O)(=O)CF)[C@@H](F)CCN12]

A solution of compound 6 (600 mg) in hydrochloric acid (3 mL, 4 M a solution of dioxane) was stirred at 25° C. for 2 h. The reaction was concentrated under reduced pressure to give a hydrochloride, which was used directly in next step without purification. A mixture of compound 6 (HCl salt, 640 mg, 1.22 mmol, 1 eq), 2-Chloro-1,3-dimethylimidazolinium chloride (229 mg, 1.59 mmol, 1.3 eq) and N-ethyl-N-isopropylpropan-2-amine (554 mg, 4.29 mmol, 747 μL, 3.5 eq) in dichloromethane (640 mL), the mixture was stirred at 20° C. for 0.5 h. The reaction mixture was concentrated by vacuum at 35° C. to remove dichloromethane. The concentration was poured into ice water (20 mL) and brown gum was formed. And then diluted with dichloromethane (10 mL) and extracted with dichloromethane (10 mL) for three times. The combined organic layers were washed with brine (20 mL), dried over Na₂SO₄, filtered and concentrated under reduced pressure to give a residue. The residue was purified by column chromatography on silica gel eluted with dichloromethane:acetone (100:1 to 1:100) to afford an impure solid. The residue was triturated with tert-butyl methyl ether (3 mL). The resulting solid was collected by filtration, washed with tert-butyl methyl ether (1 mL) and dried under high vacuum to give 1-fluoro-N-((4²S,4³R,4⁴S)-1³,1⁵,2²,4⁴-tetrafluoro-5-oxo-8-oxa-4(2,1)-piperidina-1(1,2),2(1,3)-dibenzenacyclooctaphane-4³-yl)methanesulfonamide, Compound 125 (180 mg, 98% purity).

LCMS Method I: (ESI⁺): m/z 505 (M+H)⁺, RT: 2.511 min

¹H NMR (400 MHz, DMSO-d₆) δ ppm 1.80-2.05 (m, 2H), 2.10 (ddd, 1H), 2.95 (br d, 1H), 3.10-3.25 (m, 2H), 3.50-3.65 (m, 2H), 3.80 (br dd, 2H), 4.00-4.15 (m, 1H), 4.75-4.85 (m, 1H), 4.95 (d, 1H), 5.30-5.60 (m, 2H), 7.10-7.20 (m, 3H), 7.25-7.30 (m, 1H), 7.40 (ddd, 1H), 8.60 (br s, 1H).

### Example 42. Synthesis of Compound 124, 1-fluoro-N-((4²S,4³R,4⁴S)-1³,2²,4⁴-trifluoro-5-oxo-8-oxa-4(2,1)-piperidina-1(1,2),2(1,3)-dibenzenacyclooctaphane-4³-yl)methanesulfonamide

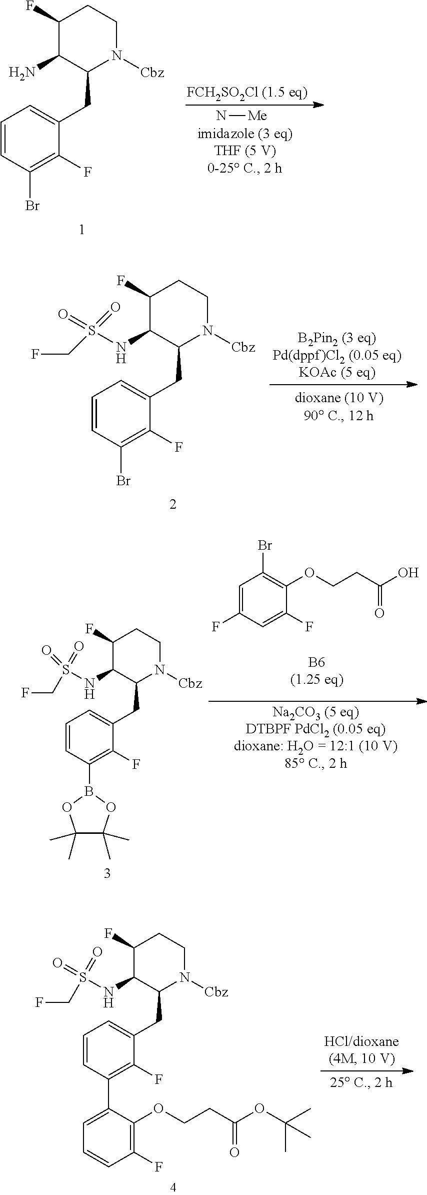 [SMILES: N[C@H]1[C@@H](F)CCN(C(=O)OCc2ccccc2)[C@H]1Cc1cccc(Br)c1F ; CN ; O=C(OCc1ccccc1)N1CC[C@H](F)[C@H](NS(=O)(=O)CF)[C@@H]1Cc1cccc(Br)c1F ; O=C(O)CCOc1c(F)cc(F)cc1Br ; CC1(C)OB(c2cccc(C[C@H]3[C@@H](NS(=O)(=O)CF)[C@@H](F)CCN3C(=O)OCc3ccccc3)c2F)OC1(C)C ; CC(C)(C)OC(=O)CCOc1c(F)cccc1-c1cccc(C[C@H]2[C@@H](NS(=O)(=O)CF)[C@@H](F)CCN2C(=O)OCc2ccccc2)c1F]

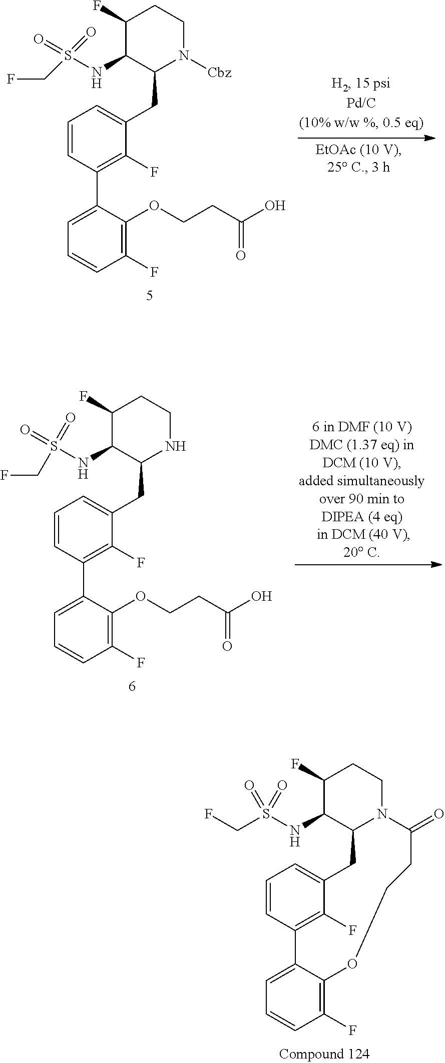 [SMILES: O=C(O)CCOc1c(F)cccc1-c1cccc(C[C@H]2[C@@H](NS(=O)(=O)CF)[C@@H](F)CCN2C(=O)OCc2ccccc2)c1F ; O=C(O)CCOc1c(F)cccc1-c1cccc(C[C@@H]2NCC[C@H](F)[C@@H]2NS(=O)(=O)CF)c1F ; O=C1CCOc2c(F)cccc2-c2cccc(c2F)C[C@H]2[C@@H](NS(=O)(=O)CF)[C@@H](F)CCN12]

### Synthesis of intermediate 4, tert-butyl 3-[2-[3-[[4-fluoro-3-(fluoromethylsulfonylamino)-2-piperidyl]methyl]phenyl]phenoxy]propanoate

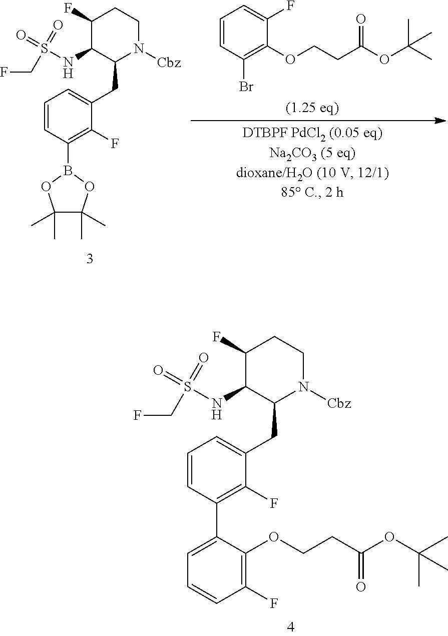 [SMILES: CC1(C)OB(c2cccc(C[C@H]3[C@@H](NS(=O)(=O)CF)[C@@H](F)CCN3C(=O)OCc3ccccc3)c2F)OC1(C)C ; CC(C)(C)OC(=O)CCOc1c(F)cccc1Br ; CC(C)(C)OC(=O)CCOc1c(F)cccc1-c1cccc(C[C@H]2[C@@H](NS(=O)(=O)CF)[C@@H](F)CCN2C(=O)OCc2ccccc2)c1F]

To a solution of intermediate 3 (5 g, 8.58 mmol, 1 eq) in 1,4-dioxane (46 mL) and H₂O (3.84 mL) was added tert-butyl 3-(2-bromo-6-fluorophenoxy)propanoate (3.42 g, 10.7 mmol, 1.25 eq), Na₂CO₃ (4.55 g, 42.9 mmol, 5 eq) and DTBPF PdCl₂ (280 mg, 429 μmol, 0.05 eq). The reaction mixture was stirred at 95° C. for 2 h under N₂ then cooled and filtered. The filtrate was diluted with H₂O (50 mL) and extracted with ethyl acetate (3×100 mL). The combined organic layers were washed with brine (50 mL), dried over Na₂SO₄, filtered and concentrated under reduced pressure to give a residue. The residue was purified by column chromatography on silica gel eluted with petroleum ether:ethyl acetate (9:1 to 3:1) to give intermediate 4, tert-butyl 3-[2-[3-[[4-fluoro-3-(fluoromethylsulfonylamino)-2-piperidyl]methyl]phenyl]phenoxy]propanoate (2.9 g, yield 49%) as yellow solid. LCMS Method I (ESI⁺): m/z 595.2 (M+H)⁺, RT: 3.146 min

### Synthesis of intermediate 5, 3-[2-[3-[[(2S,3R,4S)-1-benzyloxycarbonyl-4-fluoro-3-(fluoromethylsulfonylamino)-2-piperidyl]methyl]-2-fluoro-phenyl]-6-fluoro-phenoxy]propanoic acid

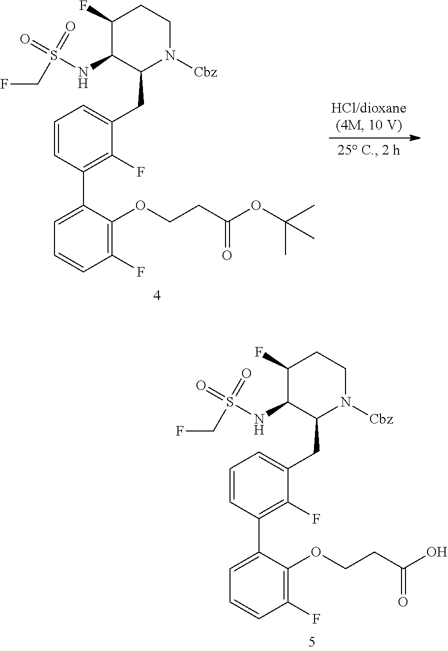 [SMILES: CC(C)(C)OC(=O)CCOc1c(F)cccc1-c1cccc(C[C@H]2[C@@H](NS(=O)(=O)CF)[C@@H](F)CCN2C(=O)OCc2ccccc2)c1F ; O=C(O)CCOc1c(F)cccc1-c1cccc(C[C@H]2[C@@H](NS(=O)(=O)CF)[C@@H](F)CCN2C(=O)OCc2ccccc2)c1F]

A mixture of Compound 4 (4 g, 5.76 mmol, 1 eq) in HCl/dioxane (4 M, 40.00 mL, 27.8 eq) was stirred at 25° C. for 2 h. The reaction mixture was concentrated under reduced pressure to give intermediate 5, 3-[2-[3-[[(2S,3R,4S)-1-benzyloxycarbonyl-4-fluoro-3-(fluoromethylsulfonylamino)-2-piperidyl]methyl]-2-fluoro-phenyl]-6-fluoro-phenoxy]propanoic acid (3.3 g, yield 90%) as white solid, which was used directly in next step without purification.

LCMS Method I (ESI⁺): m/z 595.2 (M+H−44)⁺, RT: 2.713 min

### Synthesis of intermediate 6, tert-butyl 3-[2-[3-[[4-fluoro-3-(fluoromethylsulfonylamino)-2-piperidyl]methyl]phenyl]phenoxy]propanoate

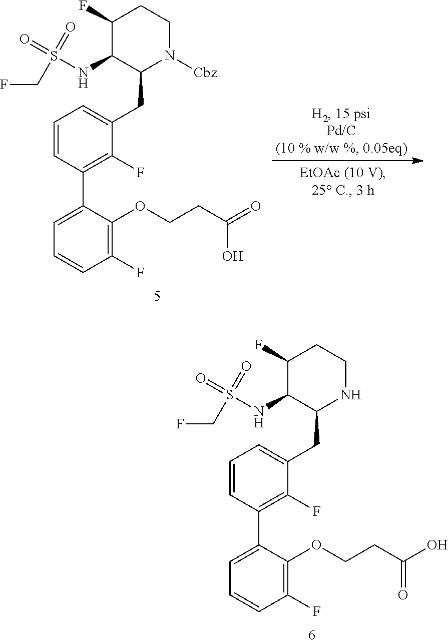 [SMILES: O=C(O)CCOc1c(F)cccc1-c1cccc(C[C@H]2[C@@H](NS(=O)(=O)CF)[C@@H](F)CCN2C(=O)OCc2ccccc2)c1F ; O=C(O)CCOc1c(F)cccc1-c1cccc(C[C@@H]2NCC[C@H](F)[C@@H]2NS(=O)(=O)CF)c1F]

To a solution of intermediate 5 (2.8 g, 4.38 mmol, 1 eq) in EtOAc (28 mL) was added Pd/C (233 mg, 219 μmol, 10% w/w, 0.05 eq). The mixture was stirred at 25° C. for 3 h under hydrogen atmosphere (15 psi). The reaction mixture was filtered and concentrated under reduced pressure to give a residue. The crude product was triturated with EA:PE=4:1 at 25° C. for 5 min to give intermediate 6, tert-butyl 3-[2-[3-[[4-fluoro-3-(fluoromethylsulfonylamino)-2-piperidyl]methyl]phenyl]phenoxy]propanoate (1.9 g, yield 86%) as light yellow oil.

LCMS Method I (ESI⁺): m/z 505.2 (M+H)⁺, RT: 1.980 min Synthesis of Compound 124, 1-fluoro-N-((4²S, 4³R,4⁴S)-1³,2²,4⁴-trifluoro-5-oxo-8-oxa-4(2,1)-piperidina-1(1,2),2(1,3)-dibenzenacyclooctaphane-4³-yl)methanesulfonamide

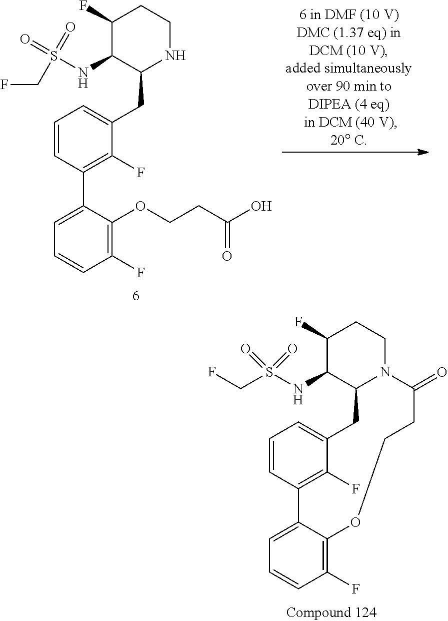 [SMILES: O=C(O)CCOc1c(F)cccc1-c1cccc(C[C@@H]2NCC[C@H](F)[C@@H]2NS(=O)(=O)CF)c1F ; O=C1CCOc2c(F)cccc2-c2cccc(c2F)C[C@H]2[C@@H](NS(=O)(=O)CF)[C@@H](F)CCN12]

A solution of intermediate 6 (53.6 mg, 99 μmol, 1 eq, HCl salt) in dimethylformamide (0.54 mL), a solution of 2-chloro-1,3-dimethyl-4,5-dihydroimidazol-1-ium chloride (23 mg, 138 μmol, 1.37 eq) in dichloromethane (0.54 mL) and diisopropylethylamine (256 mg, 1.98 mmol, 20 eq) were added to dichloromethane (107.2 mL) at 25° C. simultaneously every 30 sec. The same quantity of Compound 124 and 2-chloro-1,3-dimethyl-4,5-dihydroimidazol-1-ium chloride were added to the mixture for 49 times. And diisopropylethylamine (256 mg, 1.98 mmol, 20 eq) was added to the mixture per 5 batches. The reaction mixture was quenched by addition ice water (5 mL), and then diluted with dichloromethane (5 mL) and extracted with dichloromethane (10 mL×3). The combined organic layers were concentrated under reduced pressure to give a residue. The residue was purified by prep-HPLC (neutral condition) to give Compound 124, 1-fluoro-N-((4²S,4³R,4⁴S)-1³,2²,4⁴-trifluoro-5-oxo-8-oxa-4(2,1)-piperidina-1(1,2),2(1,3)-dibenzenacyclooctaphane-4³-yl)methanesulfonamide (780 mg purity: 98% and 120 mg purity: 95%, yield 34.6%) as a white solid.

LCMS Method I (ESI⁺): m/z 487.1 (M+H)⁺, RT: 2.584 min. ¹H NMR: (400 MHz, DMSO-d₆) δ ppm 1.80-2.00 (m, 2H), 2.05-2.20 (m, 1H), 2.95 (br d, 1H), 3.10-3.25 (m, 2H), 3.50-3.70 (m, 2H), 3.80-3.85 (m, 1H), 3.90-4.00 (m, 1H), 4.05-4.15 (m, 1H), 4.80 (br dd, 1H), 4.85-5.00 (m, 1H), 5.35-5.60 (m, 2H), 7.05-7.20 (m, 2H), 7.30-7.40 (m, 1H), 8.60 (br s, 1H).

### Example 43. Synthesis of Compound 120, 1-fluoro-N-((4²S,4³R,4⁴S)-1³,1⁵,2²,4⁴-tetrafluoro-5-oxo-8-oxa-4(2,1)-piperidina-1(1,2),2(1,3)-dibenzenacyclooctaphane-4³-yl)cyclopropane-1-sulfonamide

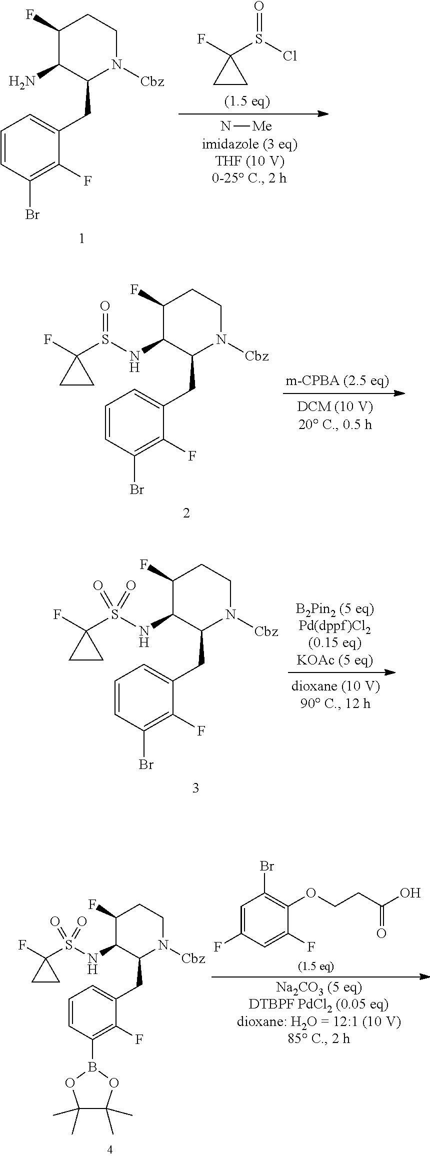

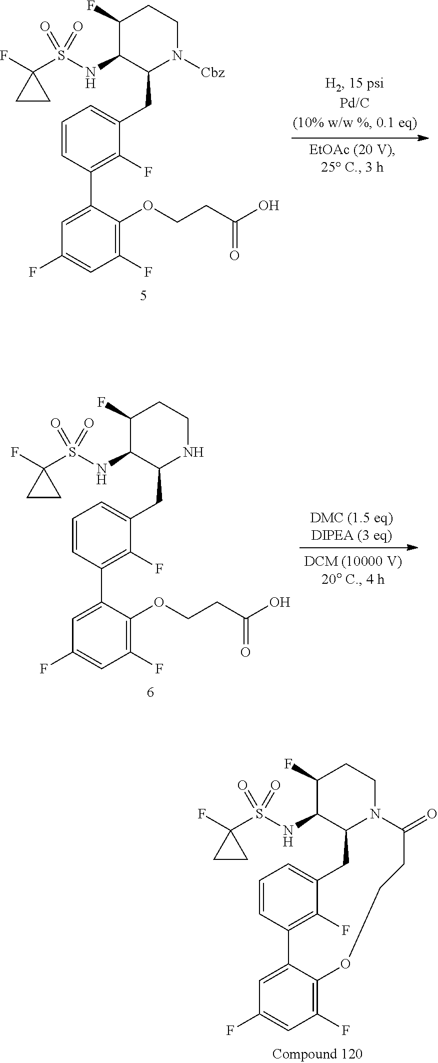 [SMILES: O=C(O)CCOc1c(F)cc(F)cc1-c1cccc(C[C@H]2[C@@H](NS(=O)(=O)C3(F)CC3)[C@@H](F)CCN2C(=O)OCc2ccccc2)c1F ; O=C(O)CCOc1c(F)cc(F)cc1-c1cccc(C[C@@H]2NCC[C@H](F)[C@@H]2NS(=O)(=O)C2(F)CC2)c1F ; O=C1CCOc2c(F)cc(F)cc2-c2cccc(c2F)C[C@H]2[C@@H](NS(=O)(=O)C3(F)CC3)[C@@H](F)CCN12]

### Synthesis of intermediate 2, benzyl (2S,3R,4S)-2-[(3-bromo-2-fluoro-phenyl)methyl]-4-fluoro-3-[(1-fluorocyclopropyl)sulfinylamino]piperidine-1-carboxylate

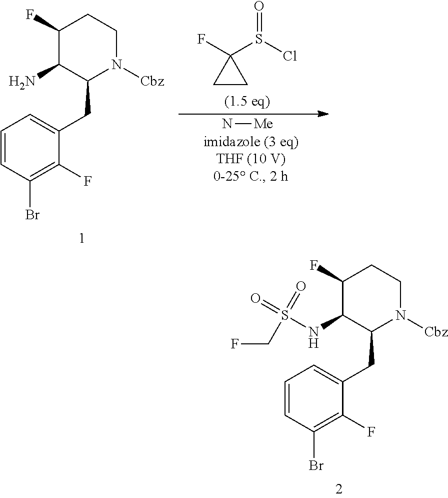

To a solution of Intermediate 1 (200 mg, 0.46 mmol, 1 eq) in tetrahydrofuran (2 mL) was added 2-methyl-1H-imidazole (112 mg, 1.37 mmol, 3 eq) and 1-fluorocyclopropanesulfinyl chloride (97 mg, 0.68 mmol, 1.5 eq) at 0° C. The mixture was stirred at 25° C. for 2 hours. The reaction mixture was quenched by addition aqueous citric acid solution (1 mL) at 25° C., then diluted with water (1 mL) and extracted with ethyl acetate (2 mL×3). The combined organic layers were washed with brine (5 mL×3), dried over Na₂SO₄, filtered and concentrated under reduced pressure to give a residue. The residue was used for next step reaction without purification. The crude product intermediate 2, benzyl (2S,3R,4S)-2-[(3-bromo-2-fluoro-phenyl)methyl]-4-fluoro-3-[(1-fluorocyclopropyl)sulfinylamino]piperidine-1-carboxylate (250 mg, 61% yield) was obtained as a yellow oil. LCMS Method J (ESI⁺): m/z 567.1 (M+Na)⁺, RT: 0.867 min

### Synthesis of intermediate 3, benzyl (2S,3R,4S)-2-[(3-bromo-2-fluoro-phenyl)methyl]-4-fluoro-3-[(1-fluorocyclopropyl)sulfonylamino]piperidine-1-carboxylate

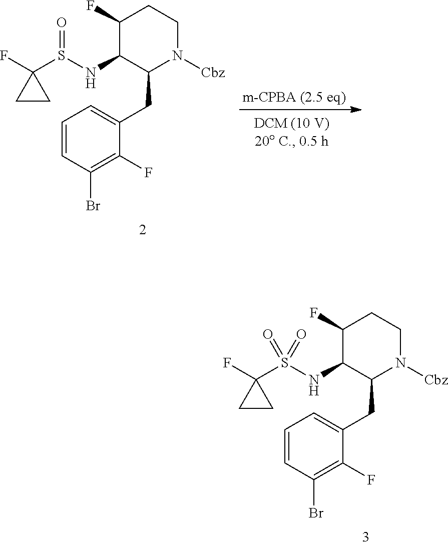 [SMILES: O=C(OCc1ccccc1)N1CC[C@H](F)[C@H](NS(=O)C2(F)CC2)[C@@H]1Cc1cccc(Br)c1F ; O=C(OCc1ccccc1)N1CC[C@H](F)[C@H](NS(=O)(=O)C2(F)CC2)[C@@H]1Cc1cccc(Br)c1F]

To a solution of intermediate 2 (237 mg, 0.27 mmol, 61% purity, 1 eq) in dichloromethane (2.5 mL) was added m-CPBA (117 mg, 0.66 mmol, 98% purity, 2.5 eq). The mixture was stirred at 20° C. for 0.5 hours. The reaction mixture was quenched by addition saturated sodium sulfite aqueous solution (2.5 mL) at 0° C., and then extracted with dichloromethane (5 mL×3). The combined organic layers were washed with brine (8 mL×3), dried over Na₂SO₄, filtered and concentrated under reduced pressure to give a residue. The residue was used for next step reaction without purification. The crude product intermediate 3, benzyl (2S,3R,4S)-2-[(3-bromo-2-fluoro-phenyl)methyl]-4-fluoro-3-[(1-fluorocyclopropyl) sulfonylamino]piperidine-1-carboxylate (238 mg, yield 99%) was obtained as a yellow oil. LCMS Method J (ESI⁺): m/z 561.1 (M+H)⁺, RT: 0.845 min

### Synthesis of intermediate 4, benzyl (2S,3R,4S)-4-fluoro-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate

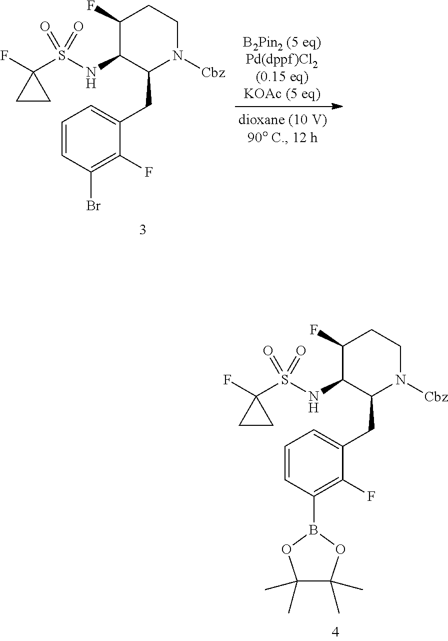 [SMILES: O=C(OCc1ccccc1)N1CC[C@H](F)[C@H](NS(=O)(=O)C2(F)CC2)[C@@H]1Cc1cccc(Br)c1F ; CC1(C)OB(c2cccc(C[C@H]3[C@@H](NS(=O)(=O)C4(F)CC4)[C@@H](F)CCN3C(=O)OCc3ccccc3)c2F)OC1(C)C]

To a solution of intermediate 3 (50 mg, 65 μmol, 73% purity, 1 eq), KOAc (32 mg, 325 μmol, 5 eq), bispinacolatodiborane (83 mg, 325 μmol, 5 eq) and Pd(dppf)Cl₂ (7 mg, 9.8 μmol, 0.15 eq) in dioxane (0.5 mL) was degassed and purged with N₂ for 3 times, and then the mixture was stirred at 90° C. for 12 hours under N₂ atmosphere. The reaction mixture was quenched by addition water (1 mL), and then extracted with ethyl acetate (1 mL×3). The combined organic layers were washed with brine (2 mL×3), dried over Na₂SO₄, filtered and concentrated under reduced pressure to give a residue. Another 3 reactions were set up. And all of four reactions were combined to give the residue. The residue was purified by prep-TLC (petroleum ether:ethyl acetate=3:2) to give intermediate 4, benzyl (2S,3R,4S)-4-fluoro-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate (114 mg, yield 53%) as a yellow oil. LCMS Method J (ESI⁺): m/z 631.1 (M+23)⁺, RT: 2.194 min

### Synthesis of intermediate 5, 3-[2-[3-[[(2S,3R,4S)-1-benzyloxycarbonyl-4-fluoro-3-[(1-fluorocyclopropyl)sulfonylamino]-2-piperidyl]methyl]-2-fluoro-phenyl]-4,6-difluoro-phenoxy]propanoic acid

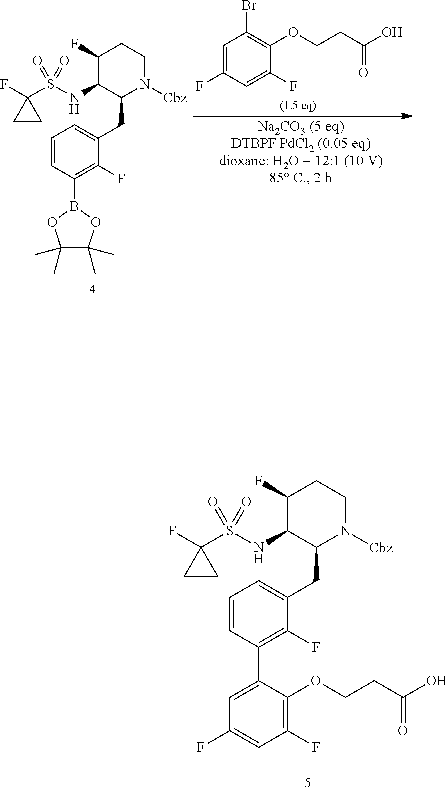

A mixture of intermediate 4 (50 mg, 60 μmol, 73% purity, 1 eq), 3-(2-bromo-4,6-difluoro-phenoxy)propanoic acid (29 mg, 90 μmol, 88% purity, 1.5 eq), ditert-butyl(cyclopentyl)phosphane:dichloropalladium:iron (2 mg, 3 μmol, 0.05 eq), Na₂CO₃ (32 mg, 0.3 mmol, 5 eq) in 1,4-dioxane (0.23 mL) and H₂O (0.02 mL) was degassed and purged with N₂ 3 times, and then the mixture was stirred at 85° C. for 2 hours under N₂ atmosphere. The reaction mixture was adjusted to pH 3 with 1N HCl, and then extracted with ethyl acetate (1 mL×3). The combined organic layers were washed with brine (3 mL×3), dried over Na₂SO₄, filtered and concentrated under reduced pressure to give a residue. Another identical reaction was conducted and both reactions were combined to give the residue. The residue was purified by prep-HPLC (neutral condition). Intermediate 5, 3-[2-[3-[[(2S,3R,4S)-1-benzyloxycarbonyl-4-fluoro-3-[(1-fluorocyclopropyl)sulfonylamino]-2-piperidyl]methyl]-2-fluoro-phenyl]-4,6-difluoro-phenoxy]propanoic acid (40 mg, yield 48%) was obtained as a white solid.

(column: Phenomenex C18 75*30 mm*3 μm; mobile phase: [water(NH₄HCO₃)-ACN]; B %: 25%-55%, 8 min). LCMS Method J (ESI⁺): m/z 705.2 (M+23)⁺, RT: 1.965 min

### Synthesis of intermediate 6, 3-[2,4-difluoro-6-[2-fluoro-3-[[(2S,3R,4S)-4-fluoro-3-[(1-fluorocyclopropyl)sulfonylamino]-2-piperidyl]methyl]phenyl]phenoxy]propanoic acid

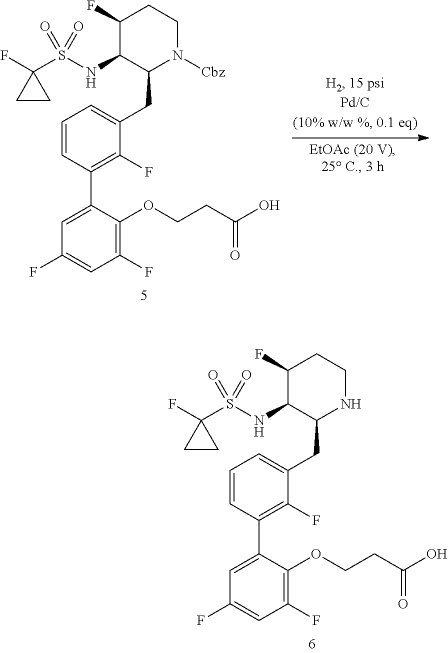 [SMILES: O=C(O)CCOc1c(F)cc(F)cc1-c1cccc(C[C@H]2[C@@H](NS(=O)(=O)C3(F)CC3)[C@@H](F)CCN2C(=O)OCc2ccccc2)c1F ; O=C(O)CCOc1c(F)cc(F)cc1-c1cccc(C[C@@H]2NCC[C@H](F)[C@@H]2NS(=O)(=O)C2(F)CC2)c1F]

A mixture of intermediate 5 (35 mg, 50 μmol, 98% purity, 1 eq) in ethyl acetate (0.7 mL) was added Pd/C (5.4 mg, 5.0 μmol, 10% purity, 0.1 eq) and degassed and purged with H₂ for 3 times, and then the mixture was stirred at 25° C. for 3 hours under H₂ atmosphere (15 Psi). The reaction was filtered and the filtrate was concentrated under reduced pressure to give intermediate 6, 3-[2,4-difluoro-6-[2-fluoro-3-[[(2S,3R,4S)-4-fluoro-3-[(1-fluorocyclopropyl) sulfonylamino]-2-piperidyl]methyl]phenyl]phenoxy]propanoic acid (30 mg, yield 77%) as a white solid. LCMS Method J (ESI⁺): m/z 548.14 (M+H)⁺, RT: 1.337 min

### Synthesis of Compound 120, 1-fluoro-N-((4²S, 4³R,4⁴S)-1³,1⁵,2²,4⁴-tetrafluoro-5-oxo-8-oxa-4(2,1)-piperidina-1(1,2),2(1,3)-dibenzenacyclooctaphane-4³-yl)cyclopropane-1-sulfonamide

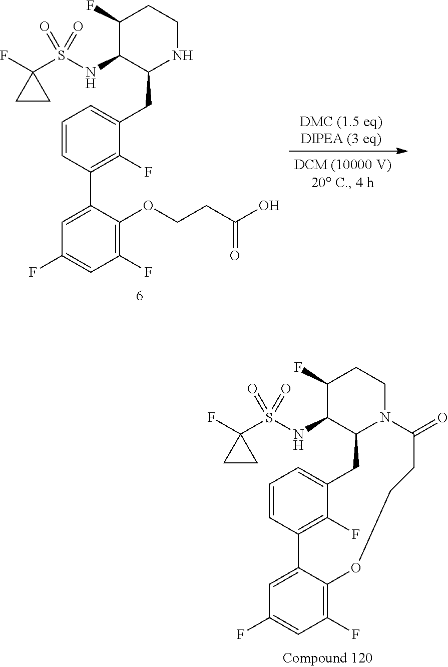 [SMILES: O=C(O)CCOc1c(F)cc(F)cc1-c1cccc(C[C@@H]2NCC[C@H](F)[C@@H]2NS(=O)(=O)C2(F)CC2)c1F ; O=C1CCOc2c(F)cc(F)cc2-c2cccc(c2F)C[C@H]2[C@@H](NS(=O)(=O)C3(F)CC3)[C@@H](F)CCN12]

To a solution of intermediate 6 (50 mg, 74 μmol, 81% purity, 1 eq) in dichloromethane (500 mL) was added DIEA (29 mg, 220 μmol, 39 μL, 3 eq) and 2-chloro-1,3-dimethyl-4,5-dihydroimidazol-1-ium chloride (19 mg, 0.11 mmol, 1.5 eq). The mixture was stirred at 20° C. for 4 hours. The reaction mixture was added to water (5 mL) and extracted with dichloromethane (10 mL×3), dried over Na₂SO₄, filtered and concentrated under reduced pressure to give a residue. The residue was purified by prep-HPLC (neutral condition) to give Compound 120, 1-fluoro-N-((4²S,4³R,4⁴S)-1³,1⁵,2²,4⁴-tetrafluoro-5-oxo-8-oxa-4(2,1)-piperidina-1(1,2),2(1,3)-dibenzenacyclooctaphane-4³-yl)cyclopropane-1-sulfonamide (30 mg, yield 77%) as white solid. Prep column: Waters Xbridge Prep OBD C18 150*40 mm*10 μm; mobile phase: [water(NH₄HCO₃)-ACN]; B %: 30%-70%, 8 min).

¹H NMR (400 MHz, DMSO-d₆) δ ppm 1.40-1.70 (m, 4H), 1.85-2.20 (m, 3H), 2.95 (br d, 1H), 3.10-3.25 (m, 2H), 3.50-3.75 (m, 2H), 3.80-3.95 (m, 2H), 4.05-4.15 (m, 1H), 4.80-5.10 (m, 2H), 7.05-7.25 (m, 4H), 7.40 (ddd, 1H), 8.80 (br s, 1H). LCMS Method I (ESI⁺): m/z 531.0 (M+H)⁺, RT: 2.692 min

### Example 44. Synthesis of Compound 119, 1-fluoro-N-((4²S,4³R,4⁴S)-1³,2²,4⁴-trifluoro-5-oxo-8-oxa-4(2,1)-piperidina-1(1,2),2(1,3)-dibenzenacyclooctaphane-4³-yl)cyclopropane-1-sulfonamide

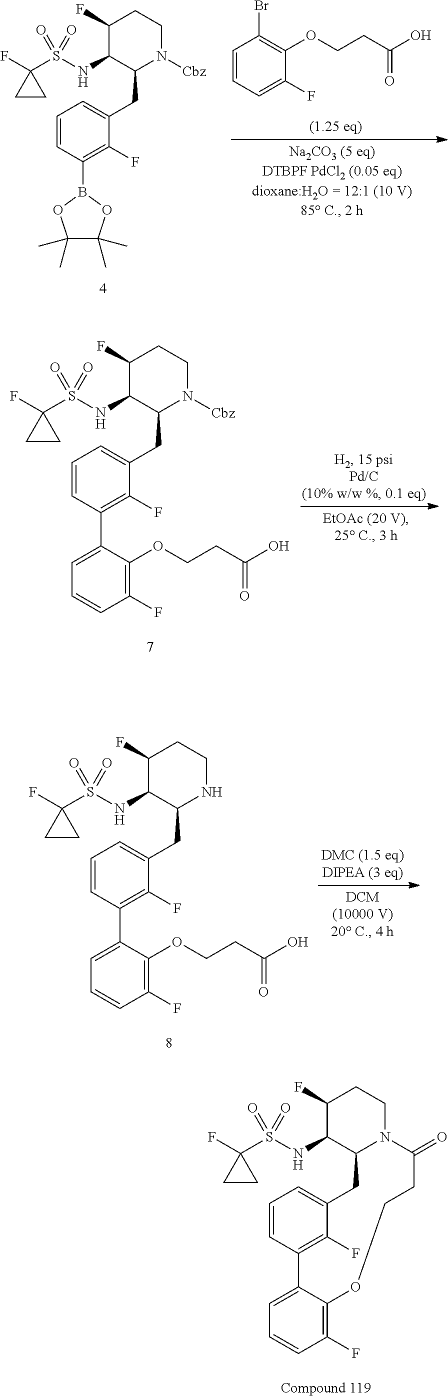

### Synthesis of intermediate 7, 3-[2-[3-[[(2S,3R,4S)-1-benzyloxycarbonyl-4-fluoro-3-[(1-fluorocyclopropyl)sulfonylamino]-2-piperidyl]methyl]-2-fluoro-phenyl]-6-fluoro-phenoxy]propanoic acid

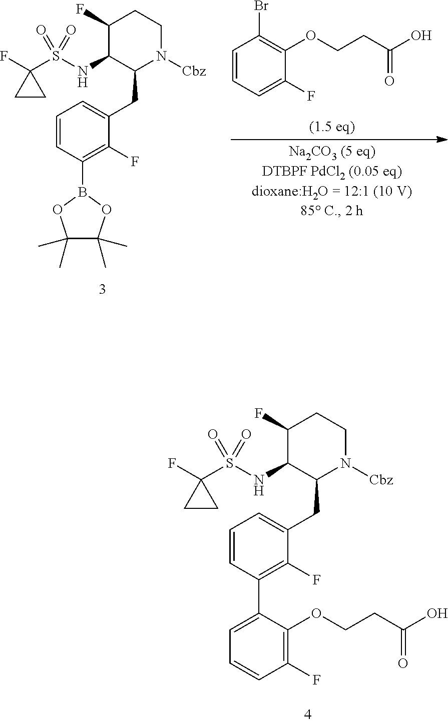 [SMILES: CC1(C)OB(c2cccc(C[C@H]3[C@@H](NS(=O)(=O)C4(F)CC4)[C@@H](F)CCN3C(=O)OCc3ccccc3)c2F)OC1(C)C ; O=C(O)CCOc1c(F)cccc1Br ; O=C(O)CCOc1c(F)cccc1-c1cccc(C[C@H]2[C@@H](NS(=O)(=O)C3(F)CC3)[C@@H](F)CCN2C(=O)OCc2ccccc2)c1F]

A mixture of intermediate 4 (50 mg, 70 μmol, 85% purity, 1 eq), 3-(2-bromo-6-fluoro-phenoxy)propanoic acid (27.8 mg, 105 μmol, 99% purity, 1.5 eq), ditert-butyl(cyclopentyl)phosphane:dichloropalladium:iron (2.28 mg, 3.49 μmol, 0.05 eq), Na₂CO₃ (37 mg, 349 μmol, 5 eq) in 1,4-dioxane (0.9 mL) and H₂O (0.08 mL) was degassed and purged with N₂ for 3 times, and then the mixture was stirred at 85° C. for 2 hours under N₂ atmosphere. The reaction mixture was adjusted to pH 3 with 1N HCl (1 mL) and extracted with ethyl acetate (1 mL×3). The combined organic layers were washed with brine (3 mL×3), dried over Na₂SO₄, filtered and concentrated under reduced pressure to give a residue. Another three identical reactions were set up and all four reactions were combined to give the residue. The residue was purified by prep-HPLC (neutral condition) to give intermediate 7, 3-[2-[3-[[(2S,3R,4S)-1-benzyloxycarbonyl-4-fluoro-3-[(1-fluorocyclopropyl) sulfonylamino]-2-piperidyl]methyl]-2-fluoro-phenyl]-6-fluoro-phenoxy]propanoic acid (120 mg, yield 64%) as a white solid (column: Phenomenex C18 75*30 mm*3 μm; mobile phase: [water(NH₄HCO₃)-ACN]; B %: 25%-55%, 8 min). LCMS Method J (ESI⁺): m/z 687.3 (M+Na)⁺, RT: 1.934 min

### Synthesis of intermediate 8, 3-[2-fluoro-6-[2-fluoro-3-[[(2S,3R,4S)-4-fluoro-3-[(1-fluorocyclopropyl) sulfonylamino]-2-piperidyl]methyl]phenyl]phenoxy]propanoic acid

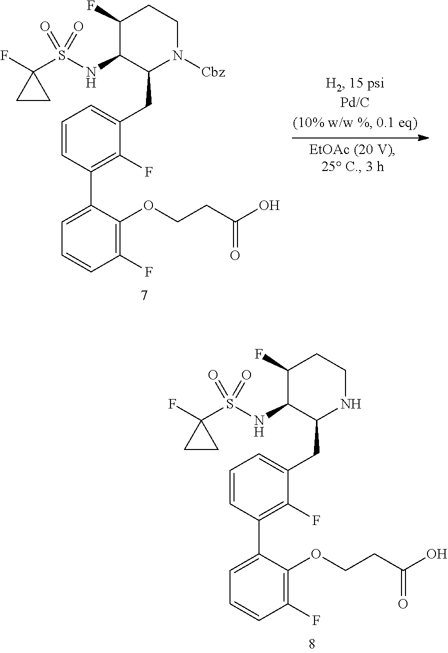 [SMILES: O=C(O)CCOc1c(F)cccc1-c1cccc(C[C@H]2[C@@H](NS(=O)(=O)C3(F)CC3)[C@@H](F)CCN2C(=O)OCc2ccccc2)c1F ; O=C(O)CCOc1c(F)cccc1-c1cccc(C[C@@H]2NCC[C@H](F)[C@@H]2NS(=O)(=O)C2(F)CC2)c1F]

A mixture of intermediate 7 (60 mg, 88 μmol, 97% purity, 1 eq) in ethyl acetate (1.2 mL) was added palladium (9.3 mg, 8.8 μmol, 10% purity, 0.1 eq) and the mixture was degassed and purged with H₂ 3 times, and then stirred at 25° C. for 3 h under H₂ atmosphere. Another identical reaction was set up and both reactions were combined to give the residue. The reaction was filtered and the filtrate was concentrated under reduced pressure to give intermediate 8, 3-[2-fluoro-6-[2-fluoro-3-[[(2S,3R,4S)-4-fluoro-3-[(1-fluorocyclopropyl) sulfonylamino]-2-piperidyl]methyl]phenyl]phenoxy]propanoic acid (80 mg, yield 70%) as a white solid. LCMS Method K (ESI⁺): m/z 531.1 (M+H)⁺, RT: 0.586 min

### Synthesis of Compound 119, 1-fluoro-N-((4²S, 4³R,4⁴S)-1³,2²,4⁴-trifluoro-5-oxo-8-oxa-4(2,1)-piperidina-1(1,2),2(1,3)-dibenzenacyclooctaphane-4³-yl)cyclopropane-1-sulfonamide

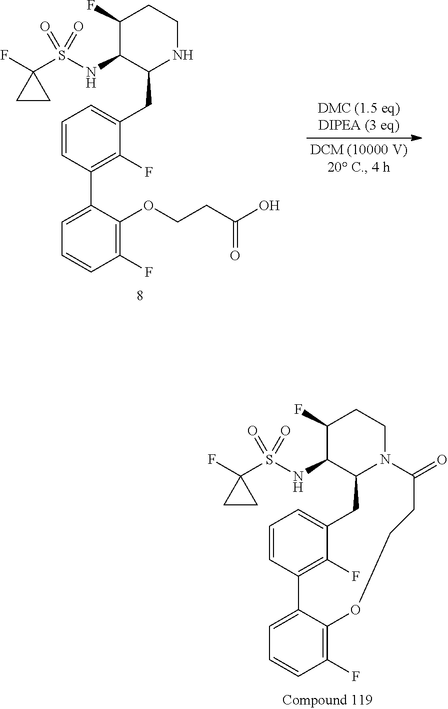 [SMILES: O=C(O)CCOc1c(F)cccc1-c1cccc(C[C@@H]2NCC[C@H](F)[C@@H]2NS(=O)(=O)C2(F)CC2)c1F ; O=C1CCOc2c(F)cccc2-c2cccc(c2F)C[C@H]2[C@@H](NS(=O)(=O)C3(F)CC3)[C@@H](F)CCN12]

To a solution of intermediate 8 (75 mg, 124 μmol, 88% purity, 1 eq) in dichloromethane (750 mL) was added DIEA (48 mg, 373 μmol, 65 μL, 3 eq) and 2-chloro-1,3-dimethyl-4,5-dihydroimidazol-1-ium chloride (32 mg, 187 μmol, 1.5 eq). The mixture was stirred at 25° C. for 4 hours. The reaction mixture was added water (5 mL) and extracted with dichloromethane (10 mL×3), dried over Na₂SO₄, filtered and concentrated under reduced pressure to give a residue. The residue was purified by prep-HPLC (neutral condition) to give Compound 119, 1-fluoro-N-((4²S,4³R,4⁴S)-1³,2²,4⁴-trifluoro-5-oxo-8-oxa-4(2,1)-piperidina-1(1,2),2(1,3)-dibenzenacyclooctaphane-4³-yl)cyclopropane-1-sulfonamide (27 mg, yield 42%) as a white solid. Prep column: Waters Xbridge Prep OBD C18 150*40 mm*10 μm; mobile phase: [water(NH₄HCO₃)-ACN]; B %: 30%-70%, 8 min).

1H NMR (400 MHz, DMSO-d₆) δ ppm 1.40-1.70 (m, 4H), 1.80-2.05 (m, 2H), 2.20 (ddd, 1H), 3.00 (br d, 1H), 3.15-3.25 (m, 2H), 3.50-3.70 (m, 2H), 3.80 (br d, 1H), 3.90-4.00 (m, 1H), 4.10-4.20 (m, 1H), 4.85-5.10 (m, 2H), 7.05-7.40 (m, 6H), 8.85 (br d, 1H). LCMS Method I (ESI⁺): m/z 513.14 (M+H)⁺, RT: 2.631 min

### Example 45. Synthesis of Compound 121, 1-fluoro-N-((4²S,4³R,4⁴S)-4⁴-fluoro-5-oxo-8-oxa-4(2,1)-piperidina-1(1,2),2(1,3)-dibenzenacyclooctaphane-4³-yl)cyclopropane-1-sulfonamide

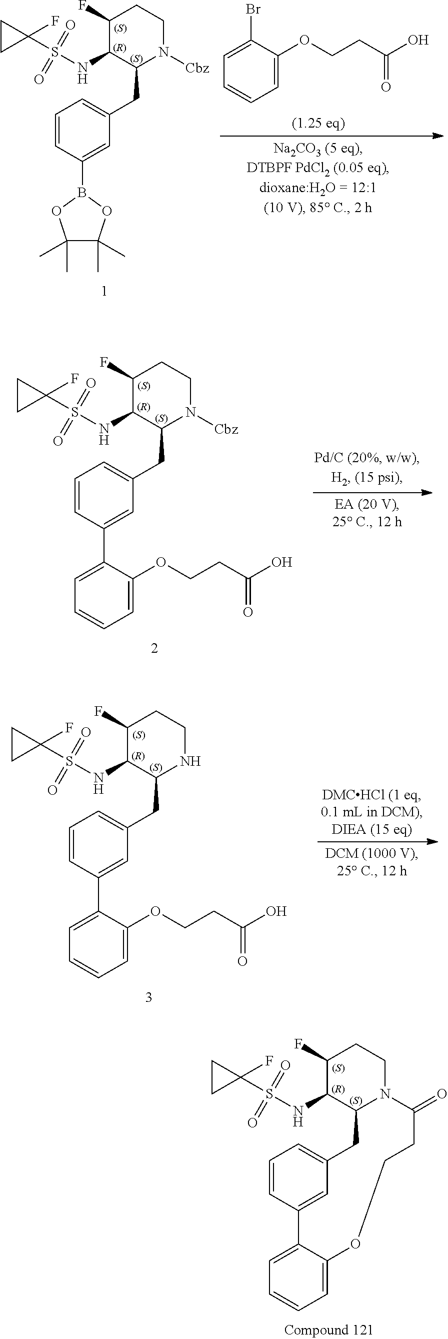

Intermediate 1 was prepared in an analogous fashion to benzyl (2S,3R,4S)-4-fluoro-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate.

### Synthesis of intermediate 2, 3-((3′-(((2S,3R,4S)-1-((benzyloxy)carbonyl)-4-fluoro-3-((1-fluorocyclopropane)-1-sulfonamido)piperidin-2-yl)methyl)-[1,1′-biphenyl]-2-yl)oxy)propanoic acid

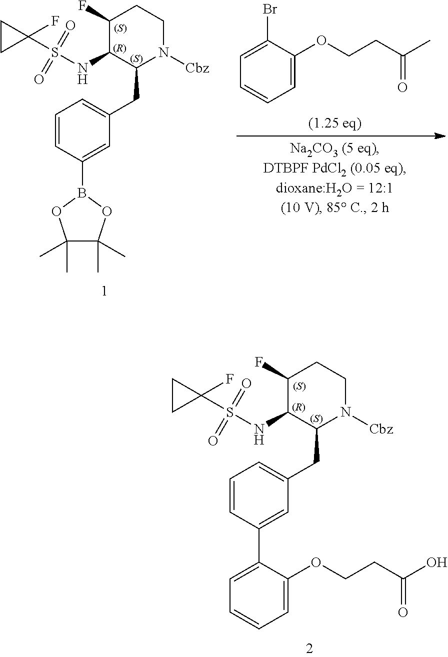

To a solution of Intermediate 1 (0.2 g, 0.340 mmol, 1 eq) and 3-(2-bromophenoxy) propanoic acid (107.91 mg, 0.44 mmol, 1.25 eq) in 1,4-dioxane (1.85 mL) and H₂O (0.15 mL) was added ditert-butyl(cyclopentyl)phosphane:dichloropalladium:iron (22 mg, 16.9 μmol, 0.05 eq) and Na₂CO₃ (180 mg, 1.69 mmol, 5 eq) at 25° C. The resulting mixture was stirred at 85° C. for 2 h. The reaction mixture was diluted with water (10 mL) and extracted with ethyl acetate (3×10 mL). The combined organic phase was washed with brine (5 mL), dried over anhydrous sodium sulfate, filtered and the filtrate was concentrated under reduced pressure to give intermediate 2, 3-((3′-(((2S,3R,4S)-1-((benzyloxy)carbonyl)-4-fluoro-3-((1-fluorocyclopropane)-1-sulfonamido)piperidin-2-yl)methyl)-[1,1′-biphenyl]-2-yl)oxy)propanoic acid (100 mg, yield 47%) as yellow solid. LCMS Method E (ESI⁺): m/z 629.3 (M+H)⁺, RT: 0.696 min

### Synthesis of intermediate 3, 3-((3′-(((2S,3R,4S)-4-fluoro-3-((1-fluorocyclopropane)-1-sulfonamido) piperidin-2-yl)methyl)-[1,1′-biphenyl]-2-yl)oxy)propanoic acid

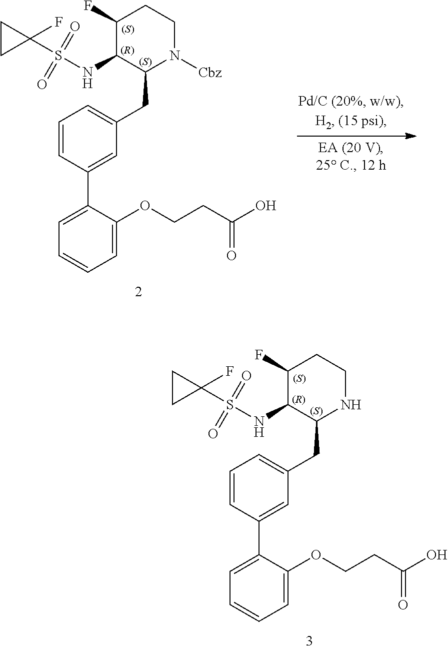

To a solution of intermediate 2 (100 mg, 0.16 mmol, 1 eq) in ethyl acetate (2 mL) was added Pd/C (8.5 mg, 20% purity) and degassed and purged with hydrogen 3 times, the mixture was stirred at 25° C. for 12 h under hydrogen atmosphere. After filtered and concentrated, the residue was purified by prep-HPLC (NH₄HCO₃ condition, column: C18 150*40 mm*10 μm; mobile phase: [water(NH₄HCO₃)-ACN]; gradient: 25%-55% B over 8 min) to give intermediate 3, 3-((3′-(((2S,3R,4S)-4-fluoro-3-((1-fluorocyclopropane)-1-sulfonamido) piperidin-2-yl)methyl)-[1,1′-biphenyl]-2-yl)oxy)propanoic acid (20 mg, yield 25%) as white solid. LCMS Method E (ESI⁺): m/z 495.2 (M+H)⁺, RT: 0.574 min

### Synthesis of Compound 121, 1-fluoro-N-((4²S, 4³R,4⁴S)-4⁴-fluoro-5-oxo-8-oxa-4(2,1)-piperidina-1(1,2),2(1,3)-dibenzenacyclooctaphane-4³-yl)cyclopropane-1-sulfonamide

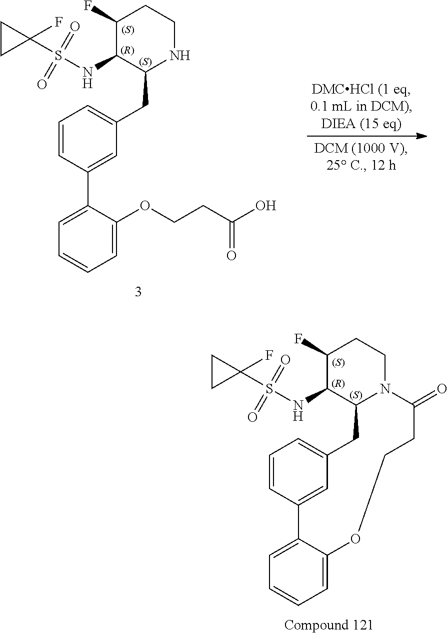

To a solution of intermediate 3 (20 mg, 40.4 μmol, 1 eq) in dichloromethane (20 mL) was added N, N-diisopropylethylamine (78.4 mg, 610 μmol, 15 eq). Then a solution of 2-chloro-1, 3-dimethyl-4, 5-dihydroimidazol-1-ium; chloride (6.84 mg, 40.4 μmol, 1 eq) in dichloromethane (0.1 mL) was added into the mixture. The mixture was stirred at 25° C. for 12 h under nitrogen atmosphere. The reaction was concentrated under reduced pressure to give a residue. The residue was purified by prep-HPLC (NH₄HCO₃ condition, column: C18 100*30 mm*10 μm; mobile phase: [water (NH₄HCO₃)-ACN]; gradient: 40%-70% B over 8 min) to give Compound 121, 1-fluoro-N-((4²S,4³R,4⁴S)-4⁴-fluoro-5-oxo-8-oxa-4(2,1)-piperidina-1(1,2),2(1,3)-dibenzenacyclooctaphane-4³-yl)cyclopropane-1-sulfonamide (4 mg, yield 21%) as white solid.

LCMS Method B (ESI⁺): m/z 477.1 (M+H)⁺, RT: 2.524 min. ¹H NMR: (400 MHz, CDCl₃) δ 1.50-1.60 (m, 1H), 1.65-1.75 (m, 3H), 1.80-2.00 (m, 1H), 2.15-2.30 (m, 2H), 3.05-3.20 (m, 3H), 3.60 (t, 1H), 3.70-3.85 (m, 2H), 3.95 (br dd, 1H), 4.05-4.15 (m, 1H), 5.00-5.20 (m, 1H), 5.30-5.45 (m, 2H), 7.10 (d, 1H), 7.15-7.20 (m, 2H), 7.25-7.50 (m, 5H).

### Example 46. Synthesis of Compound 118, N-((4²S,4³R,4⁴S)-1³,4⁴-difluoro-5-oxo-8-oxa-4(2,1)-piperidina-1(1,2),2(1,3)-dibenzenacyclooctaphane-4³-yl)-1-fluorocyclopropane-1-sulfonamide

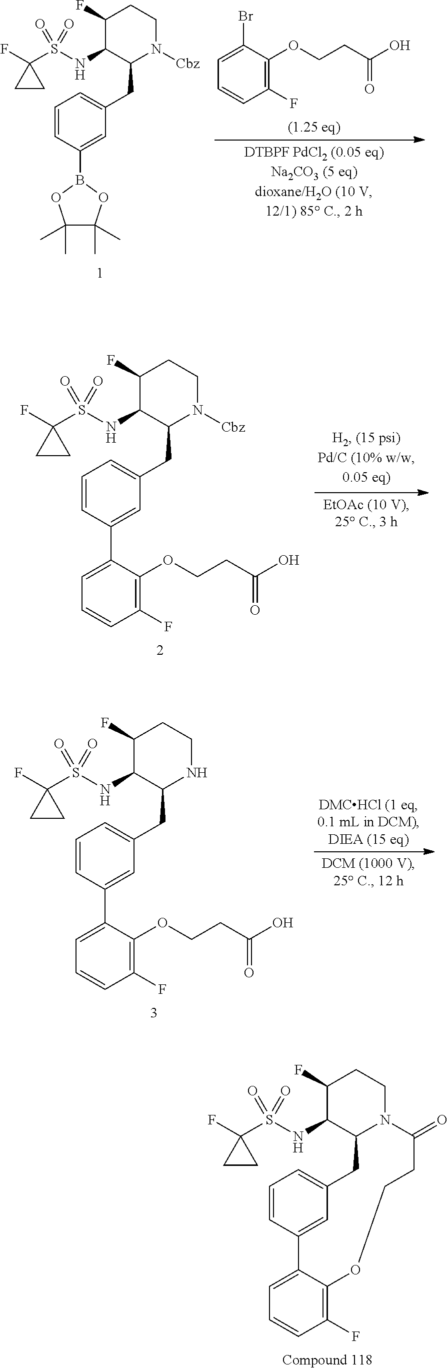

### Synthesis of intermediate 2, 3-((3′-(((2S,3R,4S)-1-((benzyloxy)carbonyl)-4-fluoro-3-((1-fluorocyclopropane)-1-sulfonamido)piperidin-2-yl)methyl)-3-fluoro-[1,1′-biphenyl]-2-yl)oxy) propanoic acid

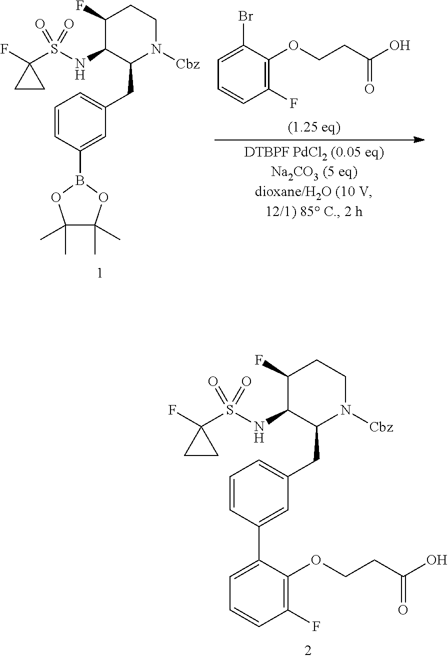

To a solution of intermediate 1 (0.8 g, 1.35 mmol, 1 eq) and 3-(2-bromo-6-fluoro-phenoxy)propanoic acid (446 mg, 1.68 mmol, 1.25 eq) in dioxane (7.4 mL) and H₂O (0.6 mL) was added ditert-butyl(cyclopentyl)phosphane:dichloropalladium:iron (88 mg, 67.5 μmol, 0.05 eq) and Na₂CO₃ (720 mg, 6.75 mmol, 5 eq) at 25° C. The resulting mixture was stirred at 85° C. for 2 h. The reaction mixture was diluted with water (10 mL) and extracted with ethyl acetate (3×15 mL). The combined organic phase was washed with brine (12 mL), dried over anhydrous sodium sulfate, filtered and the filtrate was concentrated under reduced pressure to give a residue. The residue was purified by column chromatography on silica gel eluted with petroleum ether:ethyl acetate (50:1 to 1:1) to give intermediate 2, 3-((3′-(((2S,3R,4S)-1-((benzyloxy)carbonyl)-4-fluoro-3-((1-fluorocyclopropane)-1-sulfonamido)piperidin-2-yl) methyl)-3-fluoro-[1,1′-biphenyl]-2-yl)oxy)propanoic acid (250 mg, yield 29%) as yellow solid. LCMS Method E (ESI⁺): m/z 669.3 (M+Na)⁺, RT: 0.792 min.

### Synthesis of intermediate 3, 3-((3-fluoro-3′-(((2S,3R,4S)-4-fluoro-3-((1-fluorocyclopropane)-1-sulfonamido)piperidin-2-yl)methyl)-[1,1′-biphenyl]-2-yl)oxy)propanoic acid

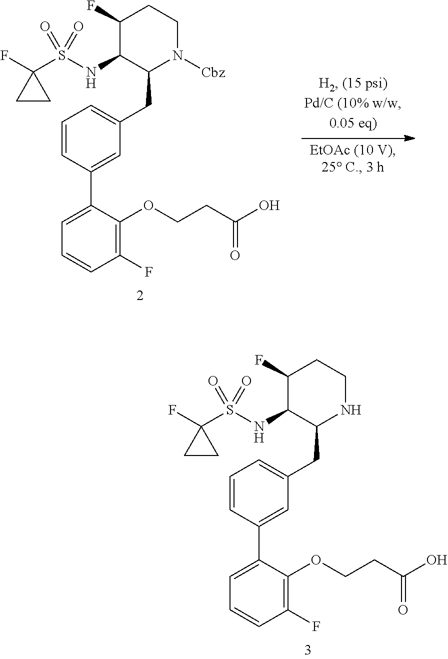 [SMILES: O=C(O)CCOc1c(F)cccc1-c1cccc(C[C@H]2[C@@H](NS(=O)(=O)C3(F)CC3)[C@@H](F)CCN2C(=O)OCc2ccccc2)c1 ; O=C(O)CCOc1c(F)cccc1-c1cccc(C[C@@H]2NCC[C@H](F)[C@@H]2NS(=O)(=O)C2(F)CC2)c1]

To a solution of intermediate 2 (250 mg, 387 μmol, 1 eq) in ethyl acetate (2.5 mL) was added Pd/C (2 mg, 19.3 μmol, 10% purity, 0.05 eq) and the mixture degassed and purged with hydrogen 3 times. The mixture was stirred at 25° C. for 3 h under hydrogen atmosphere. The reaction was filtered and the filtrate was concentrated to give intermediate 3, 3-((3-fluoro-3′-(((2S,3R,4S)-4-fluoro-3-((1-fluorocyclopropane)-1-sulfonamido)piperidin-2-yl)methyl)-[1,1′-biphenyl]-2-yl) oxy) propanoic acid (120 mg, yield 61%) as white solid. LCMS Method E (ESI⁺): m/z 513.2 (M+H)⁺, RT: 0.594 min

### Synthesis of Compound 118, N-((4²S,4³R,4⁴S)-1³,4⁴-difluoro-5-oxo-8-oxa-4(2,1)-piperidina-1(1,2),2(1,3)-dibenzenacyclooctaphane-4³-yl)-1-fluorocyclopropane-1-sulfonamide

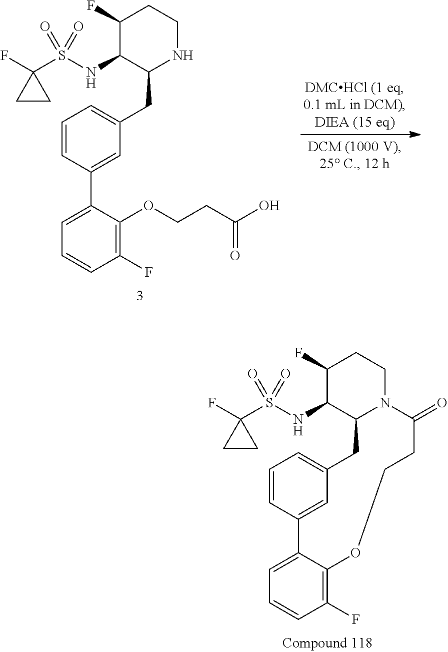 [SMILES: O=C(O)CCOc1c(F)cccc1-c1cccc(C[C@@H]2NCC[C@H](F)[C@@H]2NS(=O)(=O)C2(F)CC2)c1 ; O=C1CCOc2c(F)cccc2-c2cccc(c2)C[C@H]2[C@@H](NS(=O)(=O)C3(F)CC3)[C@@H](F)CCN12]

To a solution of intermediate 3 (120 mg, 0.234 mmol, 1 eq) in dicholoromethane (120 mL) was added N,N-diisopropylethylamine (454 mg, 3.51 mmol, 15 eq). A solution of 2-chloro-1,3-dimethyl-4,5-dihydroimidazol-1-ium:chloride (40 mg, 234 μmol, 1 eq) in dichloromethane (0.1 mL) was added into the mixture. The mixture was stirred at 25° C. for 12 h under nitrogen atmosphere. The reaction was filtered and the filtrate was concentrated to give a residue. The residue was purified by prep-HPLC (NH₄HCO₃ condition, column: C18 100*30 mm*10 μm; mobile phase: [water(NH₄HCO₃)-ACN]; gradient: 40%-70% B over 8 min) to give Compound 118, N-((4²S,4³R,4⁴S)-1³,4⁴-difluoro-5-oxo-8-oxa-4(2,1)-piperidina-1(1,2),2(1,3)-dibenzenacyclooctaphane-4³-yl)-1-fluorocyclopropane-1-sulfonamide (25.5 mg, yield 22%) as white solid.

LCMS Method B (ESI⁺): m/z 495.1 (M+H)⁺, RT: 2.648 min. ¹H NMR: (400 MHz, DMSO-d₆) δ 1.40-1.70 (m, 4H), 1.90-2.20 (m, 3H), 2.90 (t, 1H), 3.05 (br d, 1H), 3.20-3.30 (m, 1H), 3.55-3.75 (m, 3H), 4.00 (br d, 1H), 4.20 (br s, 1H), 4.80-5.05 (m, 2H), 7.10 (br d, 1H), 7.20-7.40 (m, 6H), 8.80 (br s, 1H)

### Example 47. Synthesis of Compound 67A, 1-fluoro-N-((4²S,4³S)-1³,1⁵,2²-trifluoro-5-oxo-8-oxa-4(2,1)-piperidina-1(1,2),2(1,3)-dibenzenacyclooctaphane-4³-yl)cyclopropane-1-sulfonamide

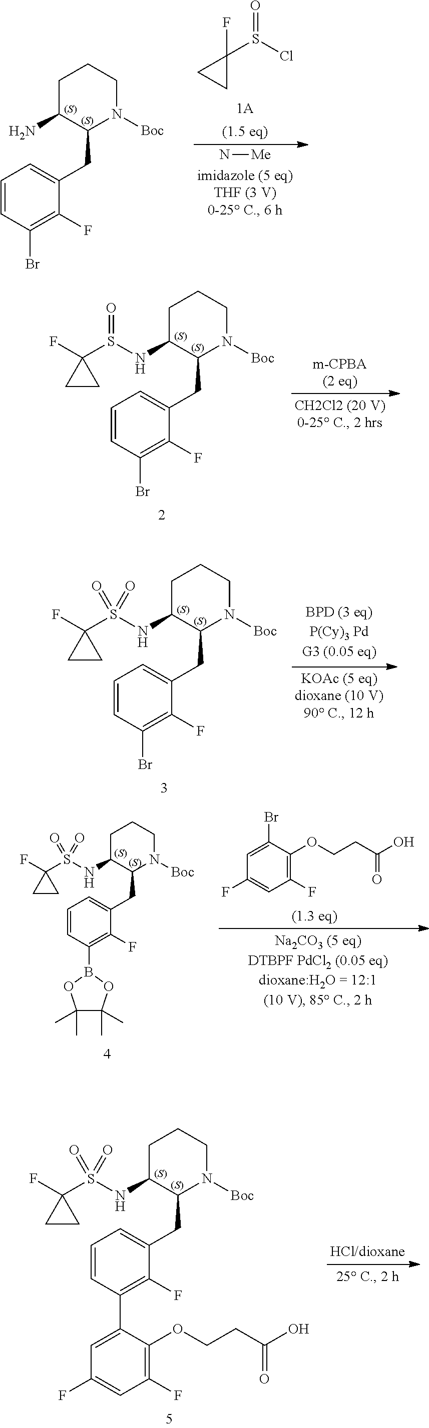

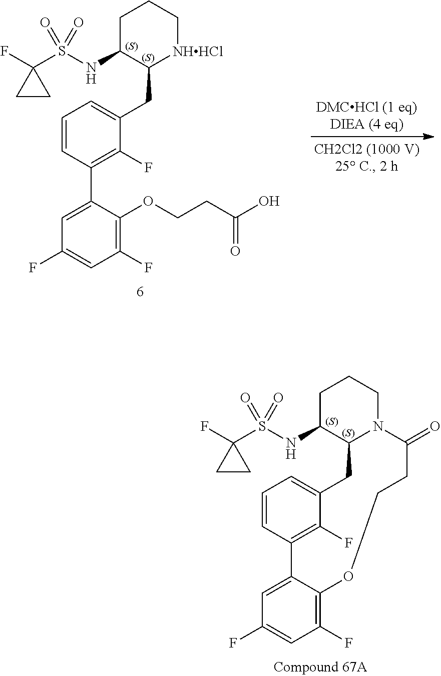

### Synthesis of intermediate 2, tert-butyl (2S,3S)-2-[(3-bromo-2-fluoro-phenyl)methyl]-3-[(1-fluorocyclopropyl)sulfinylamino]piperidine-1-carboxylate

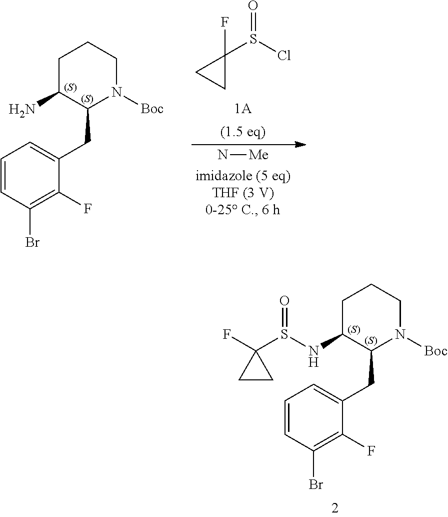

To a solution of tert-butyl (2S,3S)-3-amino-2-(3-bromo-2-fluorobenzyl)piperidine-1-carboxylate (34 g, 87.8 mmol, 1 eq) in THE (68 mL) was added 1-methylimidazole (36.04 g, 439 mmol, 35 mL, 5 eq) at 25° C. The reaction was cooled to 0° C. and 1-fluorocyclopropanesulfinyl chloride (18.78 g, 131.7 mmol, 1.5 eq) in THE (34 mL) was slowly added, with the temperature maintained at 0° C. and allowed to warm to 25° C. for 6 h. The reaction mixture was quenched with (100 mL) at 0° C., and then extracted with ethyl acetate (50 mL×3). The combined organic layers were washed with brine (50 mL), dried over Na₂SO₄, filtered and concentrated under reduced pressure to give intermediate 2, tert-butyl (2S,3S)-2-[(3-bromo-2-fluoro-phenyl)methyl]-3-[(1-fluorocyclopropyl)sulfinylamino]piperidine-1-carboxylate (32 g, purity: 90.9%, yield: 67.15%) as a white solid. HPLC: RT 4.436 min

HPLC Method: 10-80 HPLC-AB-6.0 min: HPLC (The gradient was 10-80% B in 4.50 min with a hold at 80% B for 0.9 min, 80-10% B in 0.01 min, and then held at 10% for 0.6 min (0.01-5.40 min: 0.5 mL/min flow rate; 5.41-6.00 min: 1.0 mL/min flow rate). Mobile phase A was 0.04% trifluoroacetic acid in water, mobile phase B was 0.02% trifluoroacetic acid in acetonitrile. The column used for chromatography was a Kintex-C18 2.1*50 mm column (5 μm particles). Detection methods are diode array (DAD).

### Synthesis of intermediate 3, tert-butyl (2S,3S)-2-[(3-bromo-2-fluoro-phenyl) methyl]-3-[(1-fluorocyclopropyl) sulfonylamino]piperidine-1-carboxylate

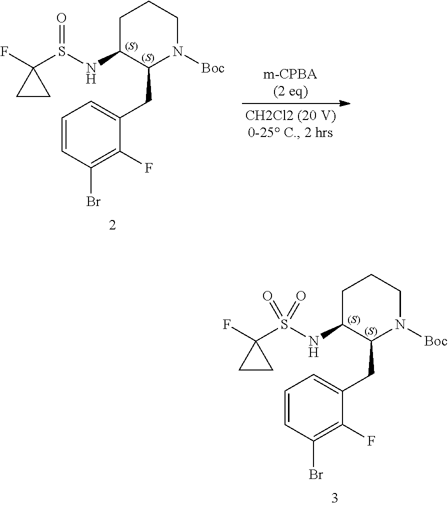

m-CPBA (23.94 g, 117.9 mmol, 85% purity, 2 eq) was added at 0° C. to a solution of intermediate 2 (32 g, 59.0 mmol, 1 eq) in dichloromethane (640 mL) at 0° C. The resulting mixture was stirred at 25° C. for 2 hrs. The reaction mixture was poured into 500 mL saturated sodium thiosulfate solution, stirred to quench the residual m-CPBA, and monitored with KI test paper. Then the organic phase was separated and washed with saturated potassium carbonate (150 mL×3). The organic layer was dried over anhydrous sodium sulfate, filtered and concentrated under reduced pressure. When ½ volume of organic layers was concentrated, the solution was tested with KI again. The crude product was purified by trituration from n-heptane/EtOAc (10/1, 5 V) at 20° C. for 2 h. Then the suspension was filtered and the filter cake was washed with n-heptane/EtOAc (10/1, 10 mL×4). The solid was dried under reduced pressure (45° C., 0.01 Mpa) to give intermediate 3, tert-butyl (2S,3S)-2-[(3-bromo-2-fluoro-phenyl) methyl]-3-[(1-fluorocyclopropyl) sulfonylamino]piperidine-1-carboxylate (26 g, 49.8 mmol, 85% yield, 97.6% purity) as a white solid. LCMS Method L (ESI⁺): m/z 409.0 (M−100+H)⁺, RT: 8.525 min

### Synthesis of intermediate 4, tert-butyl (2S,3S)-3-[(1-fluorocyclopropyl)sulfonylamino]-2-[[2-fluoro-3-(4,4,5,5-tetramethyl-1, 3,2-dioxaborolan-2-yl)phenyl]methyl]piperidine-1-carboxylate

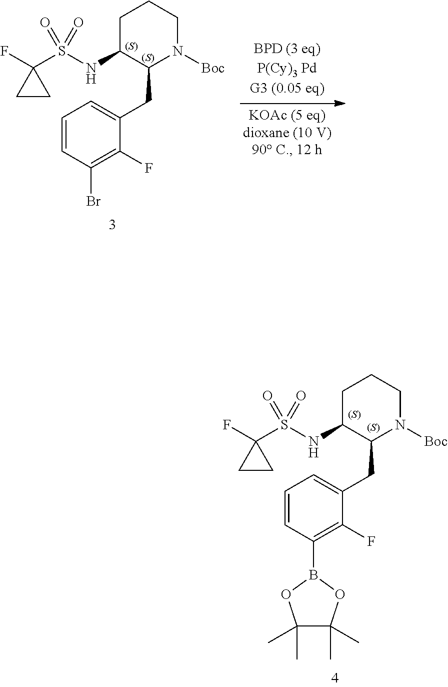

To a solution of intermediate 3 (14 g, 26.82 mmol, 1 eq)) in 1,4-dioxane (140 mL) was added KOAc (13.16 g, 134.1 mmol, 5 eq), bis-pinacolatodiborane (20.43 g, 80.6 mmol, 3 eq). The resulting suspension was degassed with argon for 30 minutes. P(Cy)₃ Pd G3 (985.58 mg, 1.34 mmol, 0.05 eq) was added, and the mixture was heated to 90° C. (internal) over 20 minutes then stirred for 12 h. The mixture was cooled to room temperature and filtered through a pad of celite (5 g). The celite pad was washed with ethyl acetate (50 mL) and the filtrate was concentrated under reduced pressure to afford a crude dark brown solid. The crude product was purified by trituration from n-heptane/EtOAc (10/1, 5 V) at 20° C. for 2 h. After being washed with n-heptane/EtOAc (10/1) and dried, intermediate 4, tert-butyl (2S,3S)-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate (10.4 g, 17.4 mmol, 64.8% yield, 93% purity) was obtained as a grey solid. LCMS Method L (ESI+): m/z 579.3 (M+23)+, R.T: 9.023 min. R.T: 6.574 was boric acid.

### Synthesis of intermediate 5, 3-[2-[3-[[(2S,3S)-1-tert-butoxycarbonyl-3-[(1-fluorocyclopropyl) sulfonyl amino]-2-piperidyl]methyl]-2-fluoro-phenyl]-4, 6-difluoro-phenoxy]propanoic acid

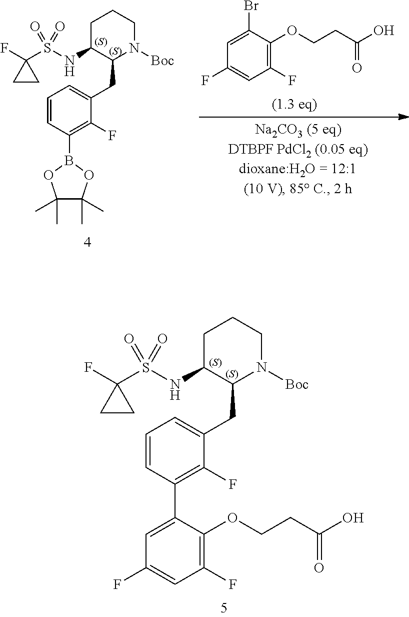

To a solution of intermediate 4 (5.2 g, 8.69 mmol, 1 eq) in 1,4-dioxane (52 mL) was added Na₂CO₃ (4.61 g, 43.5 mmol, 5 eq) and 3-(2-bromo-6-fluoro-phenoxy) propanoic acid (3.18 g, 11.3 mmol, 1.3 eq). The resulting suspension degassed with argon for 30 minutes. Ditert-butyl(cyclopentyl)phosphane; dichloropalladium; iron (283.20 mg, 435 μmol, 0.05 eq) was added, and the mixture was heated to 85° C. (internal) over 20 minutes then stirred for 2 h. The mixture was cooled to room temperature and filtered through a pad of celite (5 g). The reaction mixture was quenched by addition water (50 mL) at 0° C., and then extracted with ethyl acetate (30 mL×3). The combined organic layers were washed with brine (50 mL), dried over Na₂SO₄, filtered and concentrated under reduced pressure to give the crude. The residue was purified by column chromatography (SiO₂, Petroleum ether/Ethyl acetate=3/1 to 1/1). Intermediate 5, 3-[2-[3-[[(2S,3S)-1-tert-butoxycarbonyl-3-[(1-fluorocyclopropyl) sulfonyl amino]-2-piperidyl]methyl]-2-fluoro-phenyl]-4,6-difluoro-phenoxy]propanoic acid (4.6 g, 6.86 mmol, 79% yield, 94% purity) was obtained as a brown solid.

LCMS Method H (ESI⁺): m/z 531.1 (M−100+H)⁺, RT: 2.594 min

### Synthesis of intermediate 6, 3-[2,4-difluoro-6-[2-fluoro-3-[[(2S,3S)-3-[(1-fluorocyclopropyl)sulfonylamino]-2-piperidyl]methyl]phenyl]phenoxy]propanoic acid]

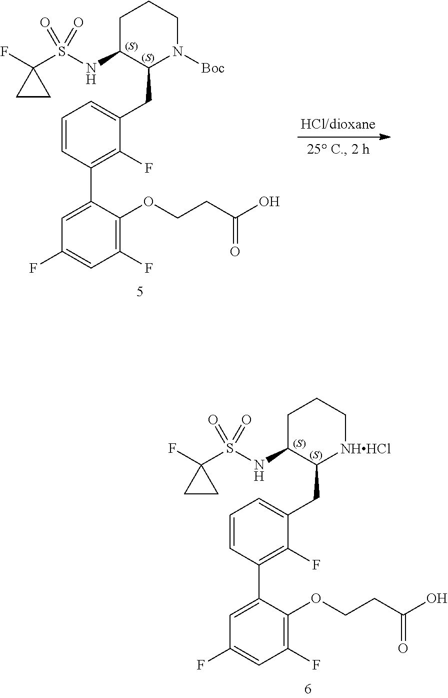

A solution of intermediate 5 (4.6 g, 6.20 mmol, 1 eq) in HCl/dioxane (20 mL) was stirred at 25° C. for 2 h. The reaction mixture was concentrated under reduced pressure to give intermediate 6, 3-[2,4-difluoro-6-[2-fluoro-3-[[(2S,3S)-3-[(1-fluorocyclopropyl)sulfonylamino]-2-piperidyl]methyl]phenyl]phenoxy]propanoic acid](4.3 g, 6.37 mmol, 93% yield, 97% purity, 13% w/w of residual dioxane) as an off-white solid.

LCMS Method H (ESI+): m/z 531.1 (M+H)⁺, RT: 2.411 min

### Synthesis of Compound 67A, 1-fluoro-N-((4²S, 4³S)-1³,1⁵,2²-trifluoro-5-oxo-8-oxa-4(2,1)-piperidina-1(1,2),2(1,3)-dibenzenacyclooctaphane-4³-yl)cyclopropane-1-sulfonamide

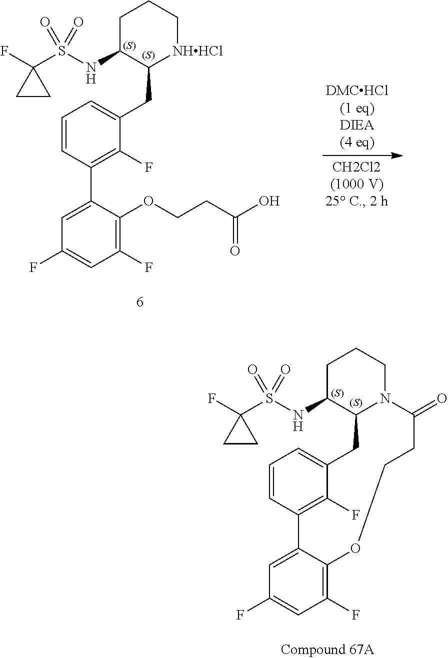

To a solution of intermediate 6 (4.3 g, 6.37 mmol, 1 eq, HCl) in dichloromethane (4.1 L) was added DIEA (3.29 g, 25.5 mmol, 4.44 mL, 4 eq) and 2-chloro-1,3-dimethyl-4,5-dihydroimidazol-1-ium chloride (1.08 g, 6.37 mmol, 1 eq). The mixture was stirred at 25° C. for 1 h. The reaction mixture was concentrated under reduced pressure and then quenched by addition of H₂O (30 mL) at 0° C., and then extracted with dichloromethane 150 mL (50 mL×3). The combined organic layers were washed with NaCl 30 mL, dried over Na₂SO₄, filtered and concentrated under reduced pressure to give a residue. The residue was purified by chromatography on silica, eluted with PE:EA=10:1 to 1:1. The solid was stirred in ethanol (30 mL) at 60° C. for 30 minutes and the resulting suspension filtered while hot. Compound 67A, 1-fluoro-N-((4²S,4³S)-1³,1⁵,2²-trifluoro-5-oxo-8-oxa-4(2,1)-piperidina-1(1,2),2(1,3)-dibenzenacyclooctaphane-4³-yl)cyclopropane-1-sulfonamide (1.6 g, 3.08 mmol, 48% yield, 98.5% purity) was obtained as a white solid.

LCMS Method I (ESI+): m/z 513.2 (M+H)⁺, RT: 2.682 min. ¹H NMR (400 MHz, DMSO-d6) δ ppm 1.37-1.62 (m, 5H) 1.65-2.08 (m, 3H) 2.05-2.10 (m, 1H), 2.80 (d, 1H), 3.05-3.20 (m, 2H), 3.25-3.45 (m, 2H), 3.75-3.85 (m, 2H), 4.05 (t, 1H), 7.05-7.25 (m, 4H), 7.40-7.50 (m, 1H), 8.45 (br s, 1H).

### Example 48. Synthesis of Compound 66A, N-((4²S,4³S)-13,22-difluoro-5-oxo-8-oxa-4(2,1)-piperidina-1(1,2),2(1,3)-dibenzenacyclooctaphane-4³-yl)-1-fluorocyclopropane-1-sulfonamide

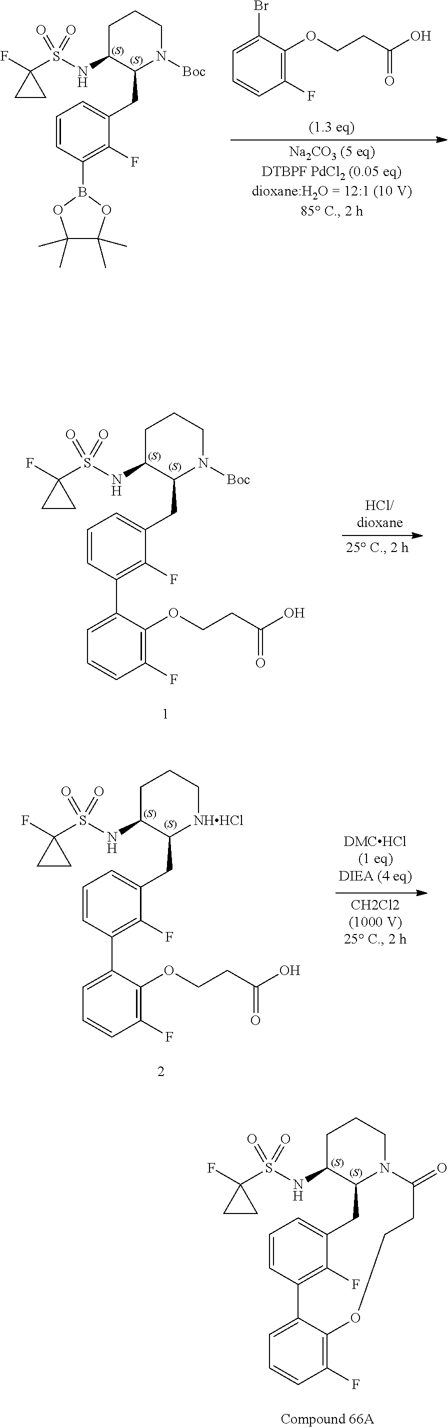

### Synthesis of intermediate 1: 3-[2-[3-[[(2S,3S)-1-tert-butoxycarbonyl-3-[(1-fluorocyclopropyl)sulfonylamino]-2-piperidyl]methyl]-2-fluoro-phenyl]-6-fluoro-phenoxy]propanoic acid]

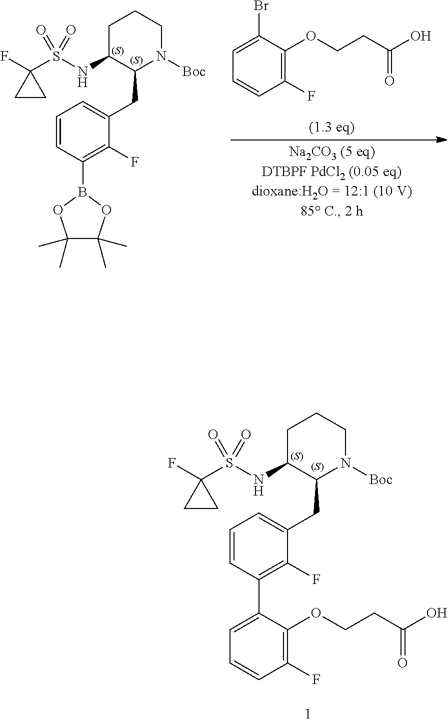

To a solution of tert-butyl (2S,3S)-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate (5.2 g, 7.94 mmol, 1 eq) in 1,4-dioxane (52 mL) was added Na₂CO₃ (4.21 g, 39.71 mmol, 5 eq) and 3-(2-bromo-6-fluoro-phenoxy)propanoic acid (2.72 g, 10.3 mmol, 1.3 eq). The resulting suspension degassed with argon for 30 minutes. Ditert-butyl(cyclopentyl)phosphane; dichloropalladium; iron (258.84 mg, 397.15 μmol, 0.05 eq) was added, and the mixture was heated to 85° C. for 20 minutes then stirred for 2 h. The mixture was cooled to room temperature and filtered through a pad of celite (5 g). The reaction mixture was quenched by addition water (50 mL) at 0° C., and then extracted with ethyl acetate (30 mL×3). The combined organic layers were washed with brine (50 mL), dried over Na₂SO₄, filtered and concentrated under reduced pressure to give the crude. The residue was purified by column chromatography (SiO₂, Petroleum ether/Ethyl acetate=3/1 to 1/1). Intermediate 1: 3-[2-[3-[[(2S,3S)-1-tert-butoxycarbonyl-3-[(1-fluorocyclopropyl)sulfonylamino]-2-piperidyl]methyl]-2-fluoro-phenyl]-6-fluoro-phenoxy]propanoic acid](4.2 g, 6.20 mmol, 78% yield, 90.5% purity) was obtained as a brown solid. LCMS Method I (ESI+): m/z 635.2 (M+23)⁺, RT: 2.652 min

### Synthesis of intermediate 2, 3-[2,4-difluoro-6-[2-fluoro-3-[[(2S,3S)-3-[(1-fluorocyclopropyl) sulfonylamino]-2-piperidyl]methyl]phenyl]phenoxy]propanoic acid; hydrochloride

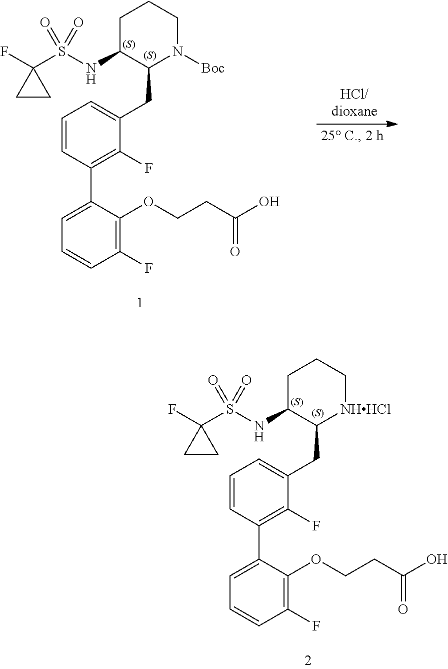

A solution of intermediate 1 (4.2 g, 6.20 mmol, 1 eq) in HCl/dioxane (20 mL) was stirred at 25° C. for 2 hr. The reaction mixture was concentrated under reduced pressure to give intermediate 2, 3-[2,4-difluoro-6-[2-fluoro-3-[[(2S,3S)-3-[(1-fluorocyclopropyl)sulfonylamino]-2-piperidyl]methyl]phenyl]phenoxy]propanoic acid; hydrochloride (4.1 g, 5.97 mmol, 96% yield, 92.7% purity, 12.7% w/w of residual dioxane) as an off-white solid. LCMS Method H (ESI+): m/z 513.1 (M+H)⁺, RT: 2.387 min

### Synthesis of Compound 66A, N-((4²S, 4³S)-13,22-difluoro-5-oxo-8-oxa-4(2,1)-piperidina-1(1,2),2(1,3)-dibenzenacyclooctaphane-4³-yl)-1-fluorocyclopropane-1-sulfonamide

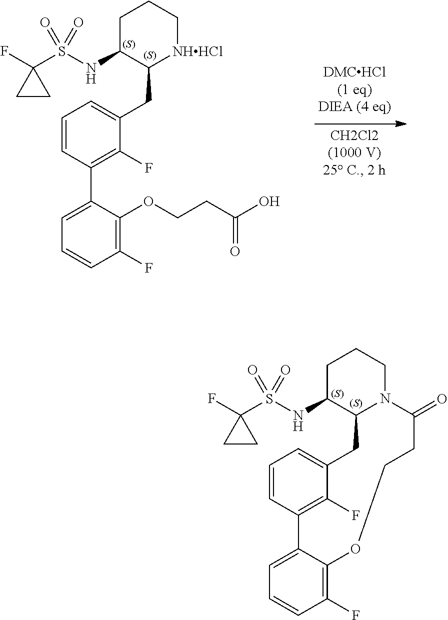

To a solution of intermediate 2 (4.1 g, 6.40 mmol, 1 eq) in dichloromethane (4.1 L) was added DIEA (3.31 g, 25.6 mmol, 4.46 mL, 4 eq) and 2-chloro-1,3-dimethyl-4,5-dihydroimidazol-1-ium chloride (1.08 g, 6.40 mmol, 1 eq). The mixture was stirred at 25° C. for 1 h. The reaction mixture was concentrated under reduced pressure and then quenched by addition of H₂O (30 mL) at 0° C., and then extracted with dichloromethane (50 mL×3). The combined organic layers were washed with NaCl 30 mL, dried over Na₂SO₄, filtered and concentrated under reduced pressure to give a residue. The residue was purified by chromatography on silica, eluted with (PE:EtOAc=10:1 to 1:1) to give product with purity less than 95%. Then, the solid was stirred in ethanol (30 mL) at 60° C. for 30 minutes and the resulting suspension was filtered to give Compound 66A, N-((4²S,4³S)-13,22-difluoro-5-oxo-8-oxa-4(2,1)-piperidina-1(1,2),2(1,3)-dibenzenacyclooctaphane-4³-yl)-1-fluorocyclopropane-1-sulfonamide (1.4 g, 2.81 mmol, 44% yield, 99.1% purity) as a white solid.

LCMS Method I (ESI+): m/z 495.15 (M+H)⁺, RT: 2.621 min. ¹H NMR (400 MHz, DMSO-d6) δ ppm 1.30-1.70 (m, 5H), 1.75-1.85 (m, 3H), 2.05-2.20 (m, 1H), 2.80 (br d, 1H), 3.05-3.20 (m, 2H), 3.35-3.45 (m, 2H), 3.80 (br d, 1H), 3.95 (br t, 1H), 4.05-4.15 (m, 1H), 4.90 (dd, 1H), 7.00-7.45 (m, 6H), 8.50 (br s, 1H).

### Example 49. Synthesis of Compound 122, 1-fluoro-N-((4²S,4³S)-1³-fluoro-5-oxo-8-oxa-4(2,1)-piperidina-1(1,2),2(1,3)-dibenzenacyclooctaphane-4³-yl)cyclopropane-1-sulfonamide

(rac)-Intermediate 1 was prepared in an analogous fashion to tert-butyl (2S,3S)-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate

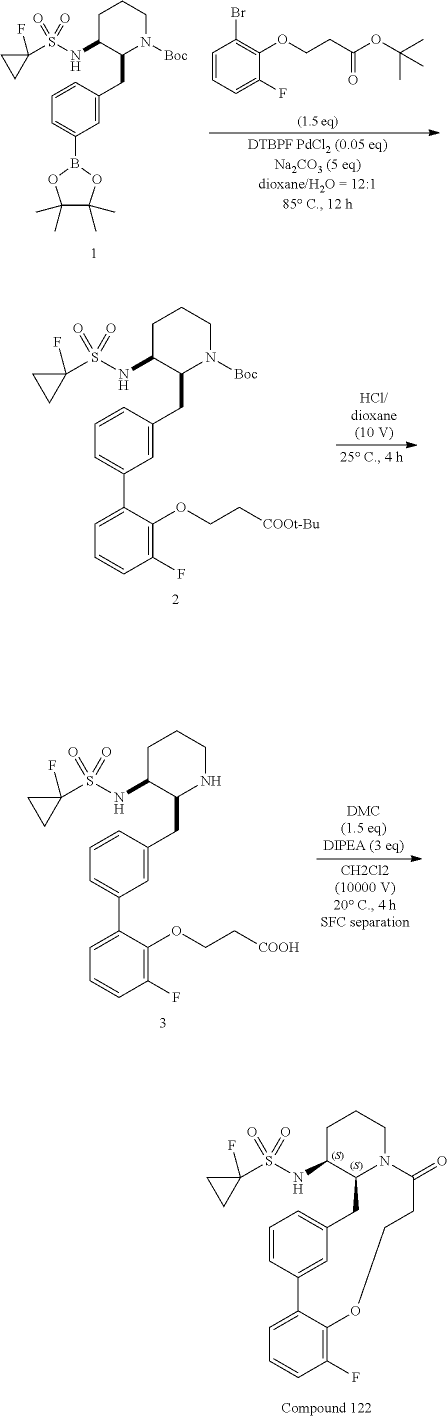

### Synthesis of intermediate 2, (rac)-(2S,3S)-tert-butyl 2-[[3-[2-(3-tert-butoxy-3-oxo-propoxy)-3-fluoro-phenyl]phenyl]methyl]-3-[(1-fluorocyclopropyl)sulfonylamino]piperidine-I-carboxylate

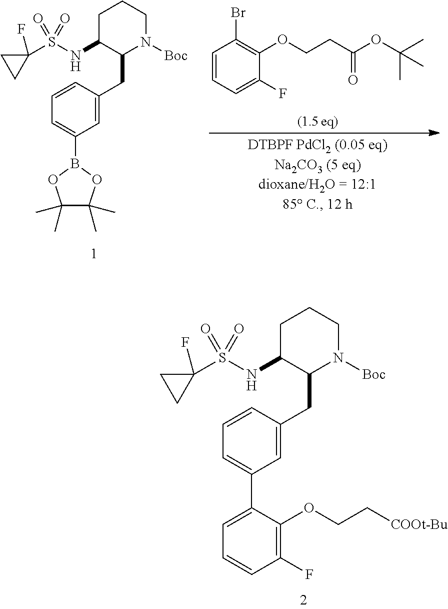

A mixture of racemic intermediate 1 (prepared according to similar methods as racemic tert-butyl (2S,3S)-3-[(1-fluorocyclopropyl)sulfonylamino]-2-[[2-fluoro-3-(4,4,5,5-tetramethyl-1,3,2-dioxaborolan-2-yl)phenyl]methyl]piperidine-1-carboxylate, 250 mg, 180 μmol, 39% purity, 1 eq), tert-butyl 3-(2-bromo-6-fluoro-phenoxy)propanoate (87 mg, 0.27 mmol, 1.5 eq), ditert-butyl(cyclopentyl) phosphane:dichloropalladium:iron (5.9 mg, 9.0 μmol, 0.05 eq) and Na₂CO₃ (96 mg, 0.91 mmol, 5 eq) in dioxane (2 mL) and H₂O (0.18 mL) was degassed and purged with N₂ for 3 times, and then the mixture was stirred at 85° C. for 2 hours under N₂ atmosphere. The reaction mixture was quenched by addition water (5 mL), and then extracted with ethyl acetate (10 mL×3). The combined organic layers were washed with brine (20 mL×3), dried over Na₂SO₄, filtered and concentrated under reduced pressure to give a residue. The residue was purified by prep-TLC (SiO₂, Petroleum ether/Ethyl acetate=3:2) to give intermediate 2, (rac)-(2S,3S)-tert-butyl 2-[[3-[2-(3-tert-butoxy-3-oxo-propoxy)-3-fluoro-phenyl]phenyl]methyl]-3-[(1-fluorocyclopropyl)sulfonylamino]piperidine-1-carboxylate (0.19 g, yield 77%) as a yellow oil. LCMS Method K (ESI+): m/z 673.3 (M+Na)⁺, RT: 1.006 min

### Synthesis of intermediate 3, (rac)-(2S,3S)-3-[2-fluoro-6-[3-[[3-[(1-fluorocyclopropyl)sulfonylamino]-2-piperidyl]methyl]phenyl]phenoxy]propanoic acid]

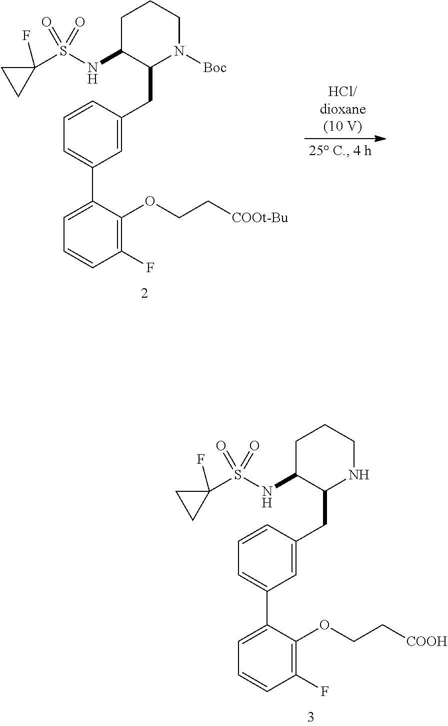

A mixture of intermediate 2 (0.14 g, 210 μmol, 96% purity, 1 eq) in HCl/dioxane (1.4 mL) was stirred at 25° C. for 4 hours. The mixture was concentrated under reduced pressure to give intermediate 3, (rac)-(2S,3S)-3-[2-fluoro-6-[3-[[3-[(1-fluorocyclopropyl)sulfonylamino]-2-piperidyl]methyl]phenyl]phenoxy]propanoic acid](100 mg, yield 92%) as a yellow oil.

LCMS Method K (ESI+): m/z 495.1 (M+H)⁺, RT: 0.554 min

### Synthesis of Compound 122, 1-fluoro-N-((4²S, 4³S)-1³-fluoro-5-oxo-8-oxa-4(2,1)-piperidina-1(1,2),2(1,3)-dibenzenacyclooctaphane-4³-yl)cyclopropane-1-sulfonamide

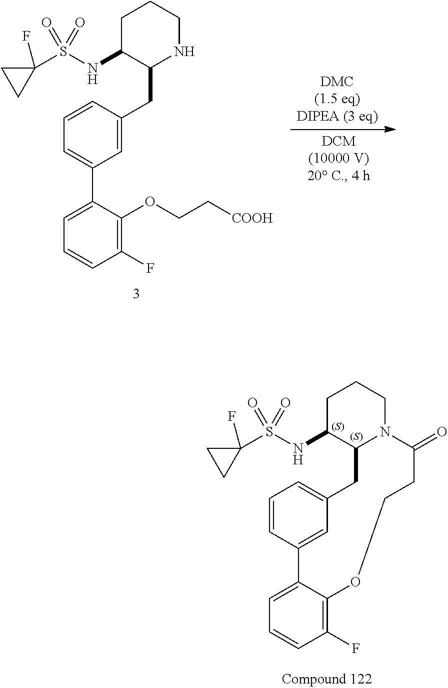

To a solution of intermediate 17 (0.1 g, 184 μmol, 91% purity, 1 eq) in dichloromethane (100 mL) was added DIEA (71 mg, 530 μmol, 96 μL, 3 eq) and 2-chloro-1,3-dimethyl-4,5-dihydroimidazol-1-ium:chloride (47 mg, 276 μmol, 1.5 eq). The mixture was stirred at 20° C. for 4 hours. The reaction mixture was added water 5 mL and extracted with dichloromethane (10 mL×3), dried over Na₂SO₄, filtered and concentrated under reduced pressure to give a residue. The residue was purified by prep-HPLC (neutral condition) to give racemic Compound 122 (65 mg, yield 71%) as white solid. Prep HPLC column: Phenomenex C18 80*40 mm*3 μm; mobile phase: [water(NH₄HCO₃)-ACN]; B %: 30%-60%,8 min). LCMS Method L (ESI+): m/z 477.2 (M+H)⁺, RT: 7.484 min

The enantiomers of Compound 122 were separated by preparative SFC.

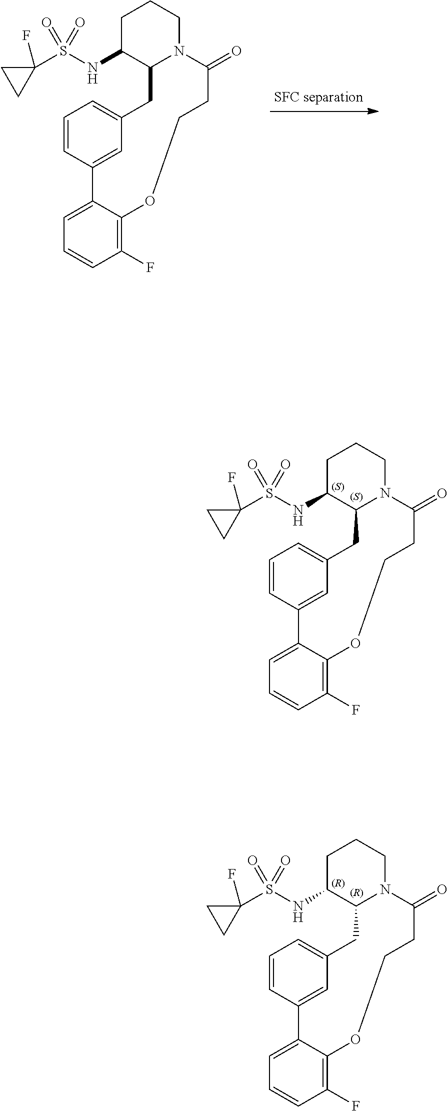

The racemic Compound 122 (60 mg, 126 μmol, 1 eq) was resolved by chiral chromatography to give 1-fluoro-N-((4²R,4³R)-1³-fluoro-5-oxo-8-oxa-4(2,1)-piperidina-1(1,2),2(1,3)-dibenzenacyclooctaphane-4³-yl)cyclopropane-1-sulfonamide (20 mg, 32% yield) and compound 122, 1-fluoro-N-((4²S,4³S)-1³-fluoro-5-oxo-8-oxa-4(2,1)-piperidina-1(1,2),2(1,3)-dibenzenacyclooctaphane-4³-yl)cyclopropane-1-sulfonamide (15 mg, 25% yield) as white solid.

SFC column: DAICEL CHIRALPAK IG (250 mm*30 mm, 10 μm); mobile phase: [0.1% NH₃H₂O ETOH]; B %: 50%-50%, 9 min). The second eluting peak was the S,S enantiomer. ¹H NMR (400 MHz, DMSO-d₆) δ ppm 1.35-1.65 (m, 5H), 1.75-1.90 (m, 3H), 2.00-2.10 (m, 1H), 2.80-3.00 (m, 2H), 3.20-3.30 (m, 1H), 3.40-3.50 (m, 2H), 3.65 (t, 1H), 3.95 (br d, 1H), 4.05-4.20 (m, 1H), 4.80-5.00 (m, 1H), 7.05-7.35 (m, 7H), 8.40 (br d, 1H). LCMS Method I (ESI+): m/z 477.2 (M+H)⁺, RT: 2.573 min.

### Example 50. Synthesis of Compound 123, 1-fluoro-N-((4²S,4³S)-5-oxo-8-oxa-4(2,1)-piperidina-1(1,2),2(1,3)-dibenzenacyclooctaphane-4³-yl)cyclopropane-1-sulfonamide

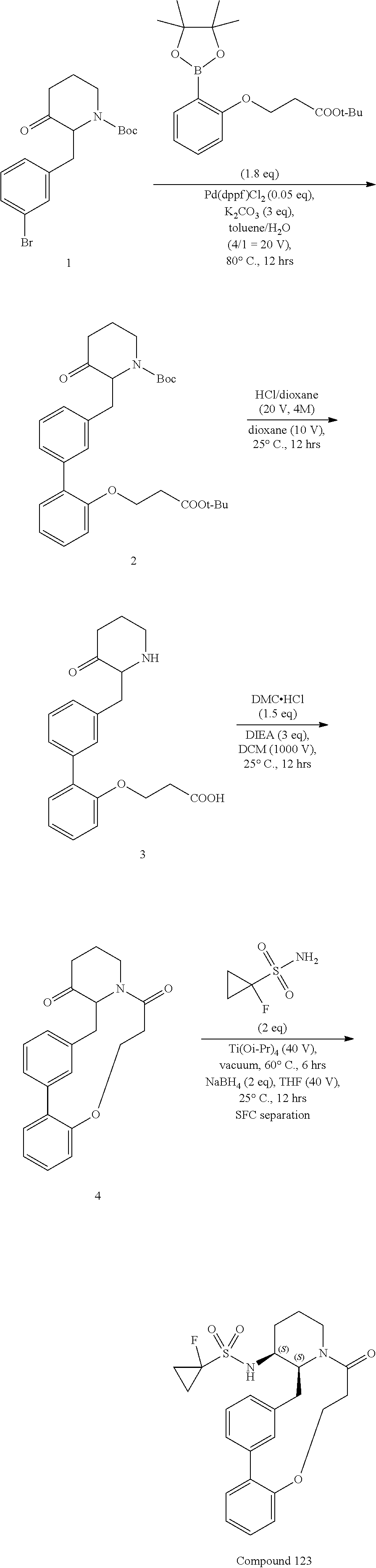

### Synthesis of intermediate 2, tertbutyl 2-[[3-[2-(3-tert-butoxy-3-oxo-propoxy)phenyl]phenyl]methyl]-3-oxo-piperidine-1-carboxylate

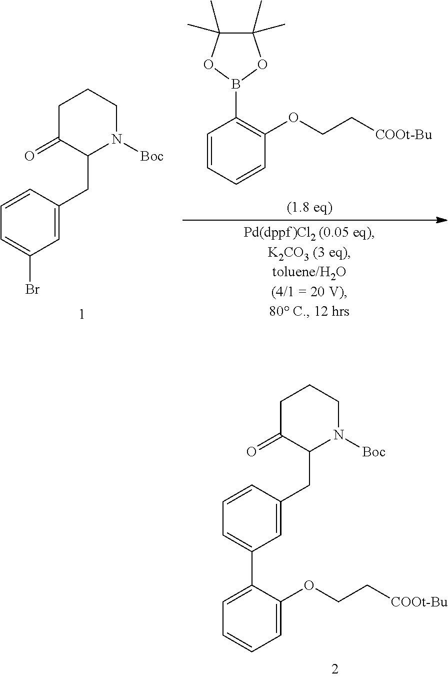

To a solution of tert-butyl 2-(3-bromobenzyl)-3-oxopiperidine-1-carboxylate (300 mg, 815 μmol, 1 eq) in toluene (4.8 mL) and H₂O (1.4 mL) were added K₂CO₃ (338 mg, 2.44 mmol, 3 eq), tert-butyl 3-[2-(4,4,5,5-tetramethyl-1,3,2-dioxaborolan-2-yl)phenoxy]propanoate (511 mg, 1.47 mmol, 1.8 eq) and Pd(dppf)Cl₂ (29.8 mg, 40.7 μmol, 0.05 eq) at 25° C. The reaction mixture was stirred at 80° C. for 12 hrs under N₂. Four additional reactions were set up as detailed above. After cooling to room temperature, all five reaction mixtures were combined. The mixture was filtered over celite and the filtrate was concentrated under reduced pressure. The residue was purified by chromatography on silica gel (eluted with petroleum ether/ethyl acetate=100/1 to 4/1) to give intermediate 2, tert-butyl 2-[[3-[2-(3-tert-butoxy-3-oxo-propoxy)phenyl]phenyl]methyl]-3-oxo-piperidine-1-carboxylate (1.9 g, yield 82%) as a yellow oil. LCMS Method E (ESI+): m/z 532.3 (M+Na)⁺, RT: 0.966 min

### Synthesis of intermediate 3, 3-[2-[3-[(3-oxo-2-piperidyl)methyl]phenyl]phenoxy]propanoic acid]

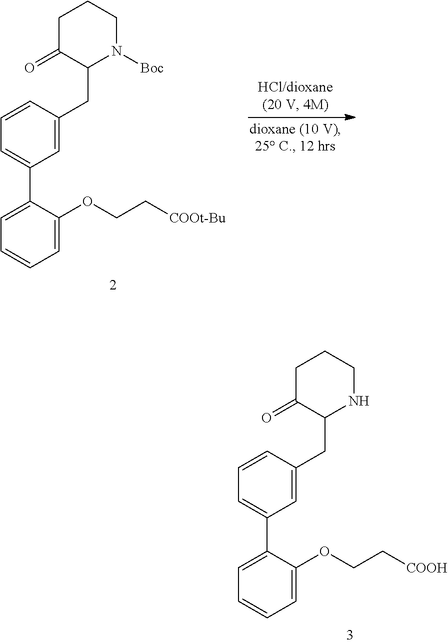

To a solution of intermediate 22 (1.5 g, 2.94 mmol, 1 eq) in 1,4-dioxane (15 mL) was added HCl/dioxane (4 M, 30 mL) at 25° C. The reaction mixture was stirred at 25° C. for 12 hrs. The reaction mixture was concentrated under reduced pressure to give intermediate 3, 3-[2-[3-[(3-oxo-2-piperidyl)methyl]phenyl]phenoxy]propanoic acid](1 g, yield 87%) as a yellow solid. ¹H NMR (400 MHz, methanol-d₄) δ ppm 1.60-2.00 (m, 3H), 2.20-2.40 (m, 1H), 2.70 (t, 2H), 2.75-2.85 (m, 1H), 2.90-3.00 (m, 1H), 3.15-3.25 (m, 1H), 3.40-3.55 (m, 2H), 4.25-4.30 (m, 2H), 7.05 (t, 1H), 7.10 (d, 1H), 7.20 (br d, 1H), 7.30-7.50 (m, 5H).

### Synthesis of intermediate 4, 8-oxa-4(2,1)-piperidina-1(1,2),2(1,3)-dibenzenacyclooctaphane-4³,5-dione

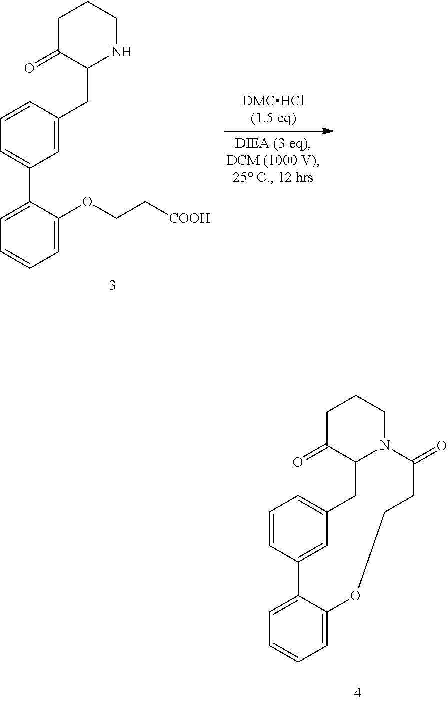

To a solution of intermediate 3 (1 g, 2.56 mmol, 1 eq, HCl) in dichloromethane (1 L) were added 2-chloro-1,3-dimethyl-4,5-dihydroimidazol-1-ium chloride (650 mg, 3.85 mmol, 1.5 eq) and DIEA (994 mg, 7.69 mmol, 1.34 mL, 3 eq) at 25° C. The reaction mixture was stirred at 25° C. for 12 hrs. The reaction mixture was concentrated under reduced pressure. The residue was purified by chromatography on silica gel (eluted with dichloromethane/THF=100/1 to 95/5) to give residue, which was triturated with dichloromethane (3 mL) and the resulting solid was collected by filtration to give intermediate 4, 8-oxa-4(2,1)-piperidina-1(1,2),2(1,3)-dibenzenacyclooctaphane-4³,5-dione (500 mg, yield 52%) as a white solid. LCMS Method H (ESI+): m/z 334.0 (M−H)⁻, RT: 1.621 min

### Synthesis of Compound 123, 1-fluoro-N-((4²S, 4³S)-5-oxo-8-oxa-4(2,1)-piperidina-1(1,2),2(1,3)-dibenzenacyclooctaphane-4³-yl)cyclopropane-1-sulfonamide

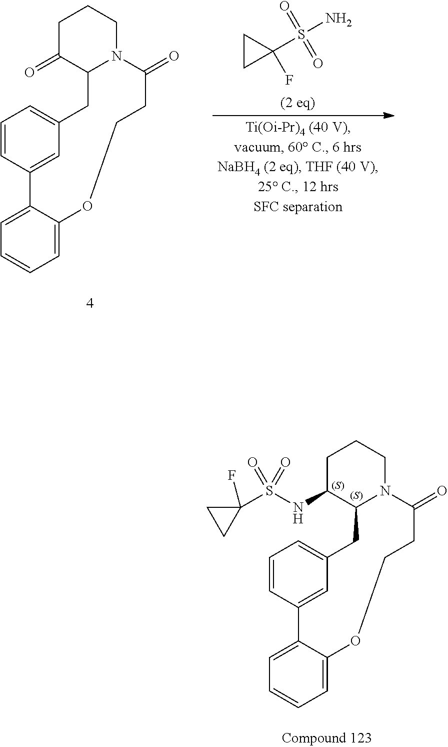

To a solution of intermediate 4 (50 mg, 0.149 mmol, 1 eq) in Ti(Oi-Pr)₄ (2 mL) was added 1-fluorocyclopropanesulfonamide (41.5 mg, 0.298 mmol, 2 eq) at 25° C. The reaction mixture was stirred at 60° C. for 6 hrs under vacuum. Then the reaction mixture was cooled to 0° C., THE (2 mL) and NaBH₄ (11 mg, 0.30 mmol, 2 eq) were added to the above solution. The reaction mixture was stirred at 25° C. for 12 hrs. Five additional reactions were set up as detailed above. All six reaction mixtures were combined. Dichloromethane (50 mL) and water (200 mL) were added. The mixture was filtered and extracted with dichloromethane (3×50 mL). The combined organic layer was dried over Na₂SO₄, filtered and concentrated under reduced pressure to give a residue. The residue was purified by chromatography on silica gel (eluted with dichloromethane/THF=100/0 to 9/1) to give 190 mg of product as racemate, which was separated by SFC (column: DAICEL CHIRALPAK IG (250 mm*30 mm, 10 μm); mobile phase: [0.1% NH₃H₂O MEOH]; B %: 30%-30%, 15 min) to give Compound 123, 1-fluoro-N-((4²S,4³S)-5-oxo-8-oxa-4(2,1)-piperidina-1(1,2),2(1,3)-dibenzenacyclooctaphane-4³-yl)cyclopropane-1-sulfonamide (30.5 mg, yield 9.9%) (RT=1.800 min in SFC) as a white solid.

LCMS Method B (ESI+): m/z 459.1 (M+H)⁺, RT: 2.416 min. 1H NMR (400 MHz, CDCl₃) δ ppm 1.45-1.70 (m, 5H), 1.75-1.95 (m, 2H), 2.00-2.10 (m, 1H), 2.20 (br d, 1H), 2.95 (dd, 1H), 3.10-3.25 (m, 3H), 3.65-3.80 (m, 1H), 3.85-4.00 (m, 2H), 4.10-4.20 (m, 1H), 4.95 (d, 1H), 5.40-5.50 (m, 1H), 7.05 (d, 1H), 7.10-7.20 (m, 2H), 7.25 (br d, 1H), 7.30-7.35 (m, 2H), 7.35-7.40 (m, 1H), 7.40 (br d, 1H). SFC: Column: Chiralpak IG-3, 50×4.6 mm I.D., 3 μm; Mobile phase: A: CO₂ B: MeOH: CH₂Cl₂=3:1 (0.1% IPAm). Column temp.: 35° C. ABPR: 1800 psi. Gradient: The gradient was 5% B in 0.20 min and 5-50% B in 1.00 min, hold on 50% B in 1.00 min, and then 95-5% B in 0.40 min, flow rate was 3.4 mL/min.

### Example 51. Orexin Type 2 Receptor Agonist Activity of Exemplary Compounds

Stable cell line generation. Obtainment of cells stably expressing either human orexin type 2 or human orexin type 1 receptor: to obtain a stable cell line the Orexin receptor cDNA was inserted into pcDNA3.1(+) plasmid vector and clones identified by G418 drug resistance selection. Clones demonstrating functional activity in response to Orexin A were selected and taken into continuous culture. A single clone for OX2R—CHO and OX1R—CHO were grown in bulk and frozen to generate a cell bank for routine screening.

Measurement of orexin type 2 receptor agonist activity. Chinese hamster ovary (CHO) cells expressing human orexin type 2 receptor (hOX2R) or human orexin type 1 receptor (hOX1R) were seeded in each well of 384 well black clear bottom plates (BD Flacon) at 10,000 cells per well and cultured for 24 hr in an Ham's F12 (Gibco) medium containing 10% fetal calf serum (Sigma Aldrich) under the conditions of 37° C., 5% CO₂. After removal of the medium, 50 μl of assay buffer 1 (0.1% bovine serum albumin (Sigma Aldrich), 20 mM HEPES (Molecular Dimensions), 250 mM probenecid (Sigma Aldrich), 1× Calcium 5 dye (Molecular Devices) in Hank's balanced salt solution (Invitrogen)) was added, and the cells were incubated for 60 min under the conditions of 37° C., 5% CO₂. A test compound was dissolved in dimethyl sulfoxide (Sigma Aldrich) to 10 mM, and then diluted with assay buffer 2 (20 mM HEPES, Hank's balanced salt solution, 0.1% bovine serum albumin). For the reaction, a test compound solution (10 μl) was added using Fluorescent Imaging Plate Reader TETRA (FLIPR TETRA: manufactured by Molecular Devices), a fluorescence value (excitation wavelength 488 nm, measurement wavelength 570 nm) of each well was measured every one second for 2 min, and the agonist activity was determined using the area of the fluorescence value as an indicator of intracellular Ca²⁺ concentration. The agonist activity of the test compound was calculated assuming that the fluorescence value of the well added with only the dilution buffer was 0% o and the fluorescence value of the well added with 10 nM human orexin A (Tocris) buffer was 100%. The agonist activity values EC₅₀ and Eₘₐₓ of each compound are shown in Table B below.

Values of hOx2 pEC₅₀ in Table B are presented in ranges, in which 6.0≤“+”<7.0, 7.0≤“++”<8.0, 8.0≤“+++”<9.0, and 9.0≤“++++”<10.4.

TABLE B

| Example No. | hOx2 pEC₅₀ |
|---|---|
| 1 | ++ |
| 2 | ++ |
| 3 | ++++ |
| 4 | +++ |
| 5 | +++ |
| 6 | +++ |
| 7 | +++ |
| 8 | ++++ |
| 9 | +++ |
| 10 | +++ |
| 11 | ++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | ++++ |
| 29 | +++ |
| 30 | ++ |
| 31 | ++ |
| 32 | ++ |
| 33 | +++ |
| 34 | +++ |
| 35 | +++ |
| 36 | +++ |
| 37 | ++++ |
| 38 | +++ |
| 39 | +++ |
| 40 | +++ |
| 41 | ++++ |
| 42 | ++++ |
| 43 | ++++ |
| 44 | ++++ |
| 45 | +++ |
| 46 | ++++ |
| 47 | ++++ |
| 48 | ++++ |
| 49 | ++++ |
| 50 | ++++ |

Wake-promoting efficacy of oral small molecules. Wake-promoting efficacy was evaluated in the B6.Cg-Tg(HCRT-MJD)1Stak/J (Atax) mouse model of NT1 and wild type (WT) colony mates. Mice were monitored in home cages using piezoelectric sensors for rapid, non-invasive classification of sleep and wakefulness by unsupervised machine learning on physiologically relevant readouts, such as body movement and breath rate. Piezoelectric detection is highly correlated with conventional time-intensive electroencephalogram/electromyography measures of sleep/wake states in both WT and NT1 mice. In a counterbalanced, repeated-measures design, mice were orally dosed at 5 h after light onset with test articles and vehicle. The increases in time spent awake over vehicle levels during the first hour post dose are indicated in Table X.

TABLE C

| Compound | Oral | | Wake % time 1 h | |
|---|---|---|---|---|
| No./ | dose | Geno- | post dose (increase | P |
| Example No. | (mg/kg) | type | from vehicle) | (vs. vehicle) |
| 125/41 | 1 | Atax | 32.4 | <0.0001 |
| | 3 | Atax | 38.2 | <0.0001 |
| | 10 | WT | 28.0 | 0.0003 |
| | 30 | WT | 31.5 | <0.0001 |
| 124/42 | 1 | Atax | 33.7 | <0.0001 |
| | 3 | Atax | 34.2 | <0.0001 |
| | 3 | WT | 17.1 | 0.009 |
| | 10 | WT | 22.8 | 0.01 |
| | 30 | WT | 37.7 | <0.0001 |
| 66A/48 | 0.3 | Atax | 44.4 | <0.0001 |
| | 1 | Atax | 40.5 | <0.0001 |
| | 3 | Atax | 48.0 | <0.0001 |
| | 3 | WT | 18.6 | 0.002 |
| | 10 | WT | 34.8 | 0.0003 |
| 67A/47 | 0.3 | Atax | 41.3 | <0.0001 |
| | 1 | Atax | 41.8 | <0.0001 |
| | 3 | Atax | 40.4 | <0.0001 |
| | 1 | WT | 20.7 | 0.006 |
| | 3 | WT | 37.6 | <0.0001 |
| | 10 | WT | 39.0 | <0.0001 |

Atax, B6.Cg-Tg(HCRT-MJD)1Stak/J;
WT, wild type

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A compound of Formula (III′): 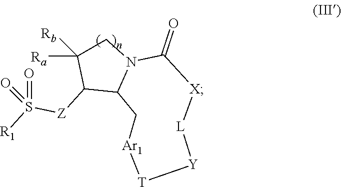
or a pharmaceutically acceptable salt thereof, wherein:
X is —N(C₁-C₆ alkyl)-, —N(C₁-C₆ haloalkyl)-, or —C₁-C₆ alkyl-;
L is absent or —C₁-C₆ alkyl-;
Y is —O—;
n is 1 or 2;
Rₐ is H or halogen;
R_{b} is halogen or C₁-C₆ alkyl;
or Rₐ and R_{b}, together with the atom they attach to, form C₃ cycloalkyl;
Z is —NH—;
R₁ is C₁-C₆ haloalkyl, C₁-C₆ alkyl, or C₃ cycloalkyl substituted with halogen;
Ar₁ is —C₆-C₁₀ aryl- optionally substituted with one or more halogen; and
T is —C₆-C₁₀ aryl- optionally substituted with one or more halogen or C₁-C₆ alkyl,
provided that when X is alkyl, L is absent.

2. The compound of claim 1, wherein R₁ is C₃ cycloalkyl substituted with halogen.

3. The compound of claim 2, wherein R₁ is C₃ cycloalkyl substituted with fluoro.

4. The compound of claim 1, wherein R₁ is C₁-C₆ haloalkyl.

5. The compound of claim 1, wherein R₁ is C₁-C₆ alkyl.

6. The compound of claim 1, wherein X is —C₁-C₆ alkyl-.

7. The compound of claim 1, wherein L is absent.

8. The compound of claim 1, wherein L is —C₁-C₆ alkyl-.

9. The compound of claim 1, wherein n is 1.

10. The compound of claim 1, wherein n is 2.

11. The compound of claim 1, wherein R_{b} is halogen.

12. The compound of claim 1, wherein Ar₁ is phenyl optionally substituted with one or more halogen.

13. The compound of claim 1, wherein T is phenyl optionally substituted with one or more halogen.

14. The compound of claim 1, wherein the compound is of Formula (II′-a): 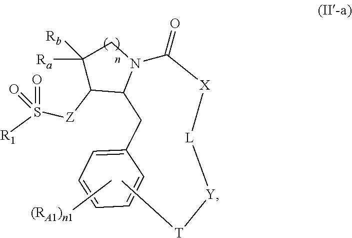
or a pharmaceutically acceptable salt thereof, wherein
R_{A1} is halogen; and
n1 is an integer from 0 to 4.

15. The compound of claim 1, wherein the compound is of Formula (IIIA′): 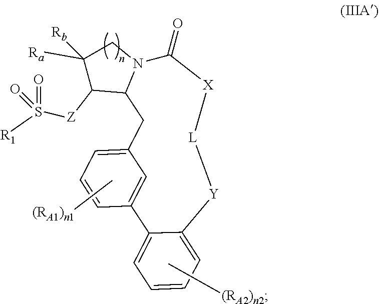
or a pharmaceutically acceptable salt thereof, wherein
R_{A1} is halogen;
R_{A2} is halogen or C₁-C₆ alkyl;
n1 is an integer from 0 to 4; and
n2 is an integer from 0 to 4.

16. The compound or salt of claim 1, wherein the compound is of Formula (IIIA′-1): 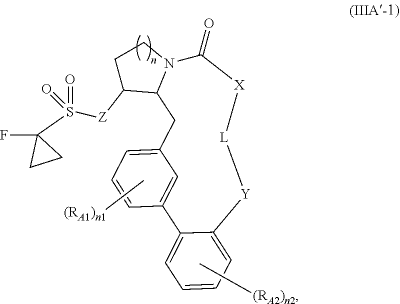
or a pharmaceutically acceptable salt thereof, wherein:
n1 is an integer ranging from 0 to 4; and
n2 is an integer ranging from 0 to 4.

17. The compound of claim 1, wherein the compound of Formula (III′) is selected from: 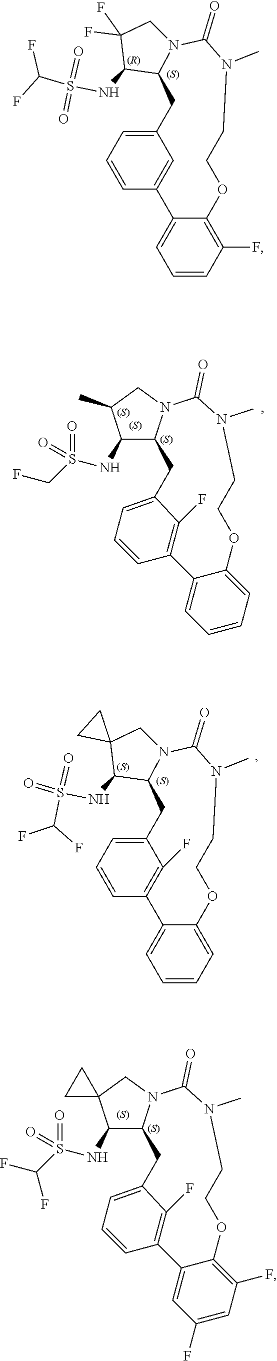 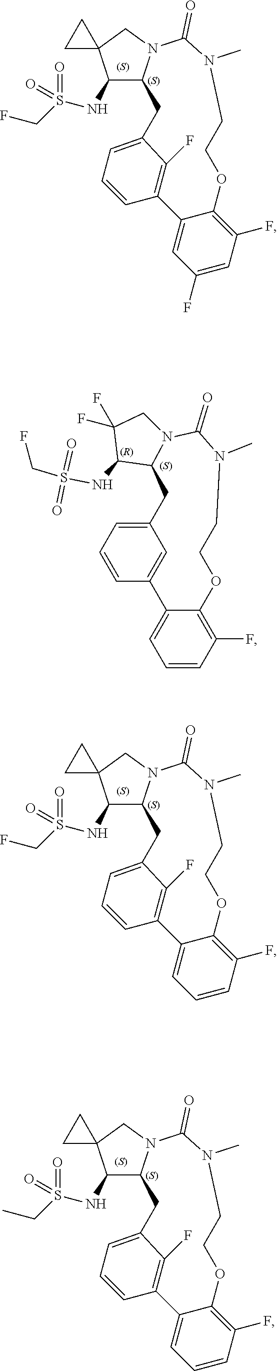 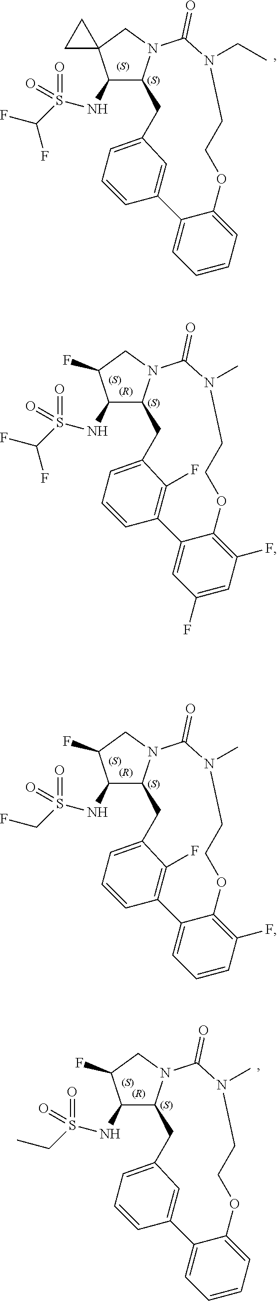 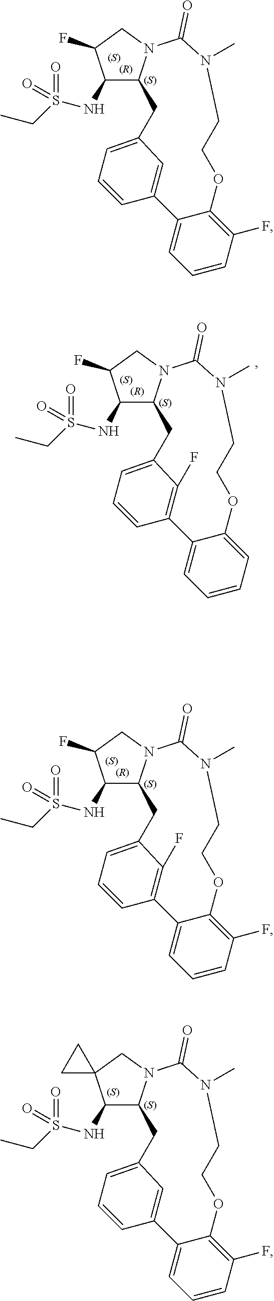 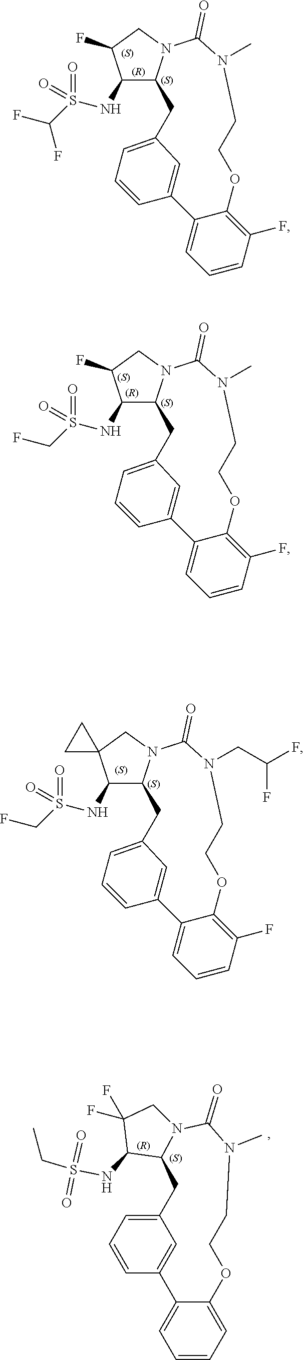 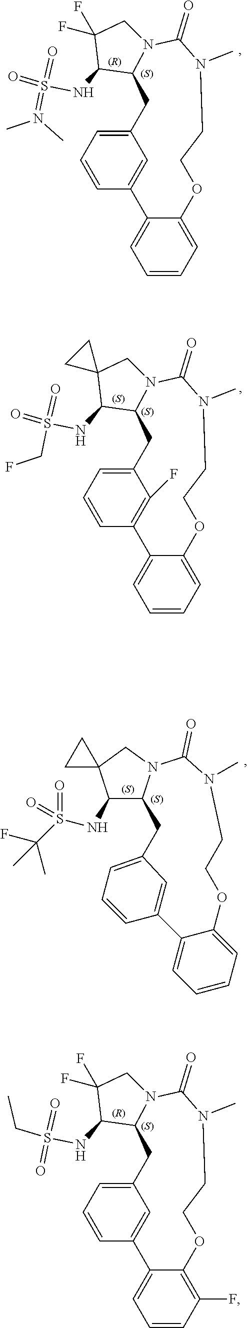 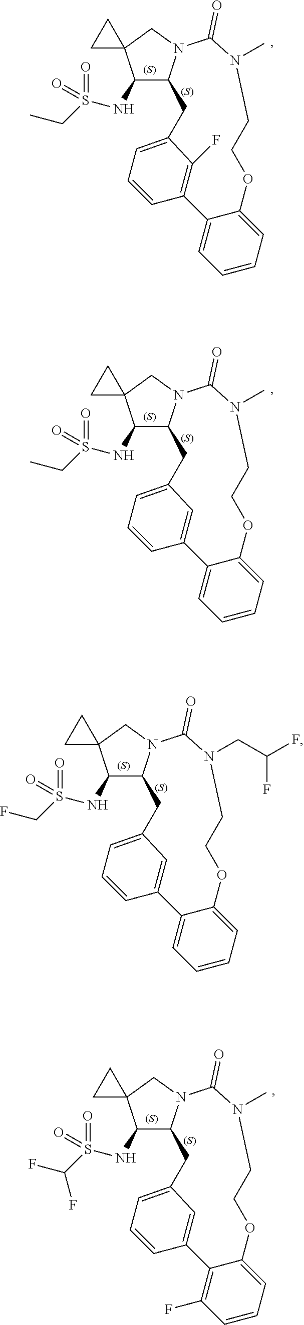 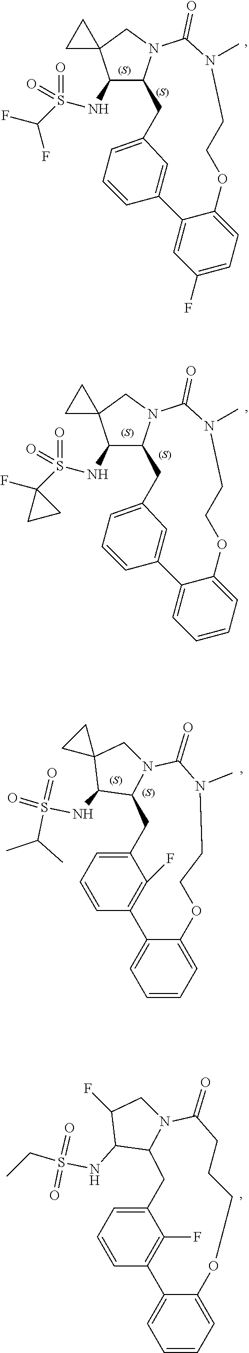 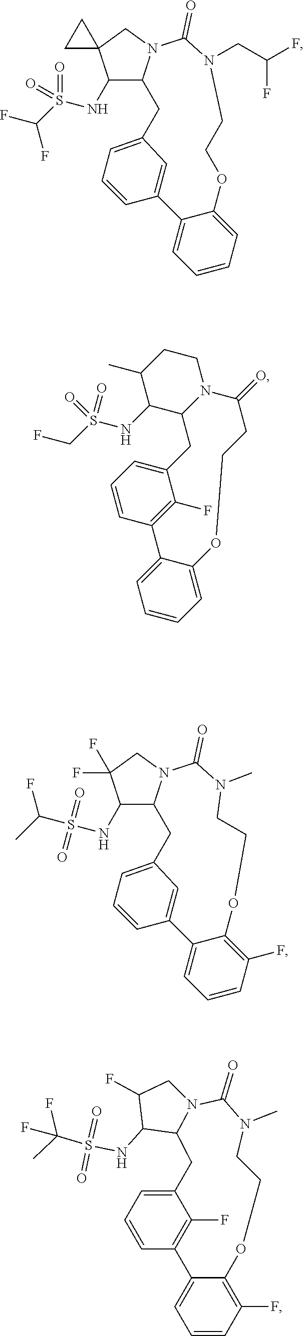 [SMILES: O=C1N(CC(F)F)CCOc2ccccc2-c2cccc(c2)CC2C(NS(=O)(=O)C(F)F)C3(CC3)CN12 ; CC1CCN2C(=O)CCOc3ccccc3-c3cccc(c3F)CC2C1NS(=O)(=O)CF ; CC(F)S(=O)(=O)NC1C2Cc3cccc(c3)-c3cccc(F)c3OCCN(C)C(=O)N2CC1(F)F ; CN1CCOc2c(F)cccc2-c2cccc(c2F)CC2C(NS(=O)(=O)C(C)(F)F)C(F)CN2C1=O] 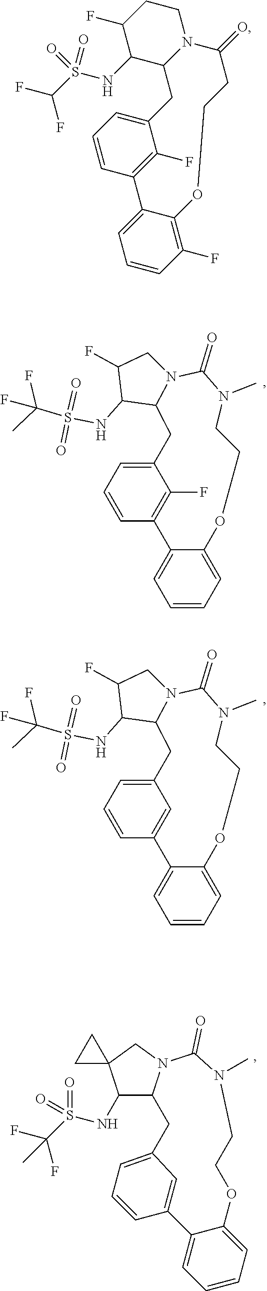 [SMILES: O=C1CCOc2c(F)cccc2-c2cccc(c2F)CC2C(NS(=O)(=O)C(F)F)C(F)CCN12 ; CN1CCOc2ccccc2-c2cccc(c2F)CC2C(NS(=O)(=O)C(C)(F)F)C(F)CN2C1=O ; CN1CCOc2ccccc2-c2cccc(c2)CC2C(NS(=O)(=O)C(C)(F)F)C(F)CN2C1=O ; CN1CCOc2ccccc2-c2cccc(c2)CC2C(NS(=O)(=O)C(C)(F)F)C3(CC3)CN2C1=O] 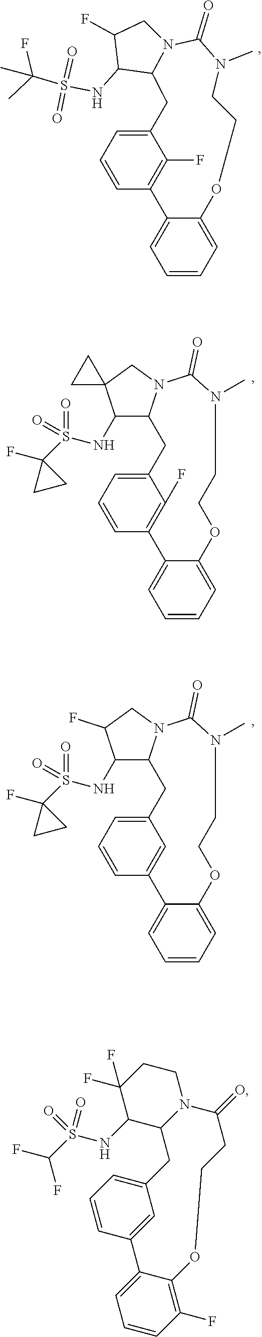 [SMILES: CN1CCOc2ccccc2-c2cccc(c2F)CC2C(NS(=O)(=O)C(C)(C)F)C(F)CN2C1=O ; CN1CCOc2ccccc2-c2cccc(c2F)CC2C(NS(=O)(=O)C3(F)CC3)C3(CC3)CN2C1=O ; CN1CCOc2ccccc2-c2cccc(c2)CC2C(NS(=O)(=O)C3(F)CC3)C(F)CN2C1=O ; O=C1CCOc2c(F)cccc2-c2cccc(c2)CC2C(NS(=O)(=O)C(F)F)C(F)(F)CCN12] 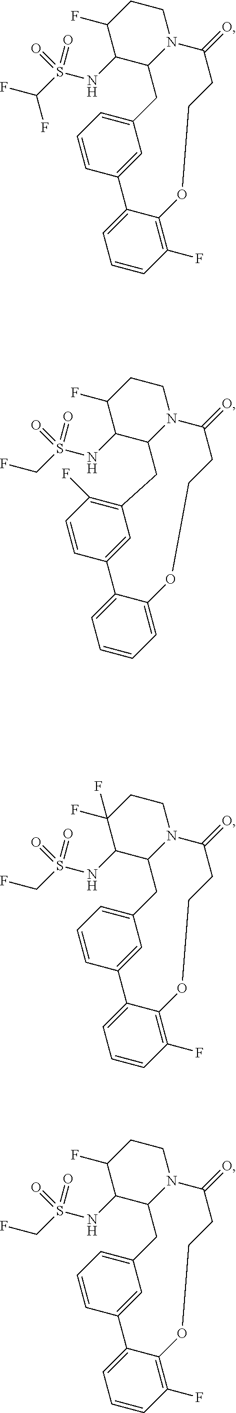 [SMILES: O=C1CCOc2c(F)cccc2-c2cccc(c2)CC2C(NS(=O)(=O)C(F)F)C(F)CCN12 ; O=C1CCOc2ccccc2-c2ccc(F)c(c2)CC2C(NS(=O)(=O)CF)C(F)CCN12 ; O=C1CCOc2c(F)cccc2-c2cccc(c2)CC2C(NS(=O)(=O)CF)C(F)(F)CCN12 ; O=C1CCOc2c(F)cccc2-c2cccc(c2)CC2C(NS(=O)(=O)CF)C(F)CCN12] 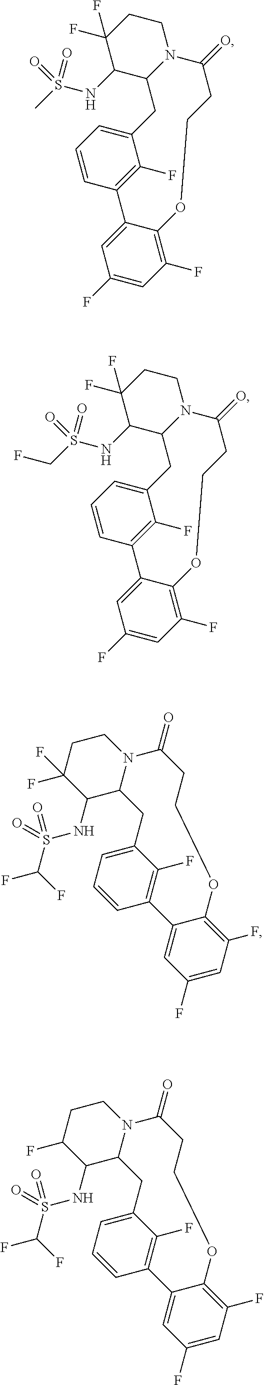 [SMILES: CS(=O)(=O)NC1C2Cc3cccc(c3F)-c3cc(F)cc(F)c3OCCC(=O)N2CCC1(F)F ; O=C1CCOc2c(F)cc(F)cc2-c2cccc(c2F)CC2C(NS(=O)(=O)CF)C(F)(F)CCN12 ; O=C1CCOc2c(F)cc(F)cc2-c2cccc(c2F)CC2C(NS(=O)(=O)C(F)F)C(F)(F)CCN12 ; O=C1CCOc2c(F)cc(F)cc2-c2cccc(c2F)CC2C(NS(=O)(=O)C(F)F)C(F)CCN12] 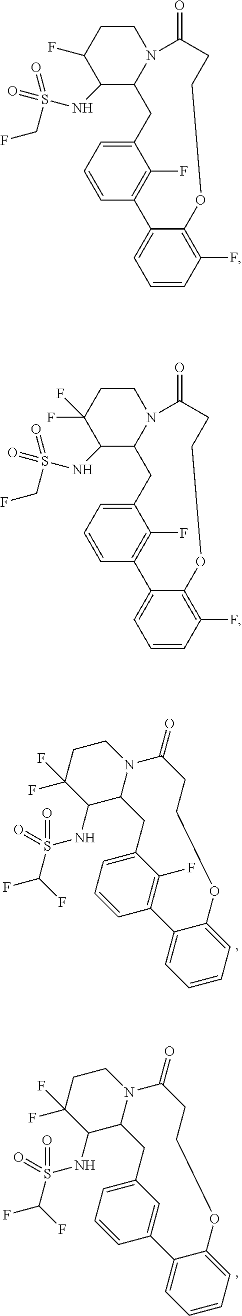 [SMILES: O=C1CCOc2c(F)cccc2-c2cccc(c2F)CC2C(NS(=O)(=O)CF)C(F)CCN12 ; O=C1CCOc2c(F)cccc2-c2cccc(c2F)CC2C(NS(=O)(=O)CF)C(F)(F)CCN12 ; O=C1CCOc2ccccc2-c2cccc(c2F)CC2C(NS(=O)(=O)C(F)F)C(F)(F)CCN12 ; O=C1CCOc2ccccc2-c2cccc(c2)CC2C(NS(=O)(=O)C(F)F)C(F)(F)CCN12] 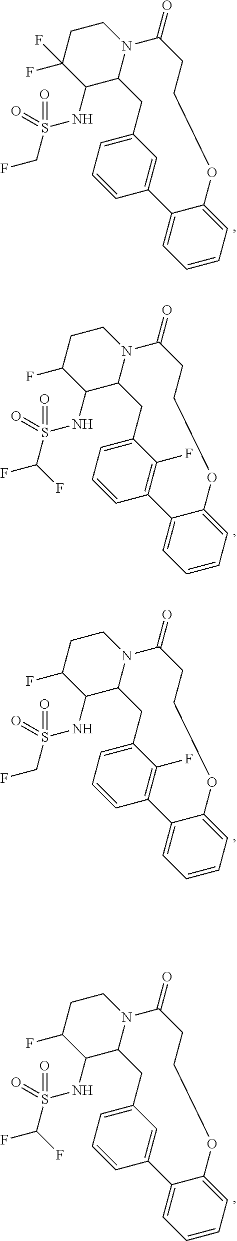 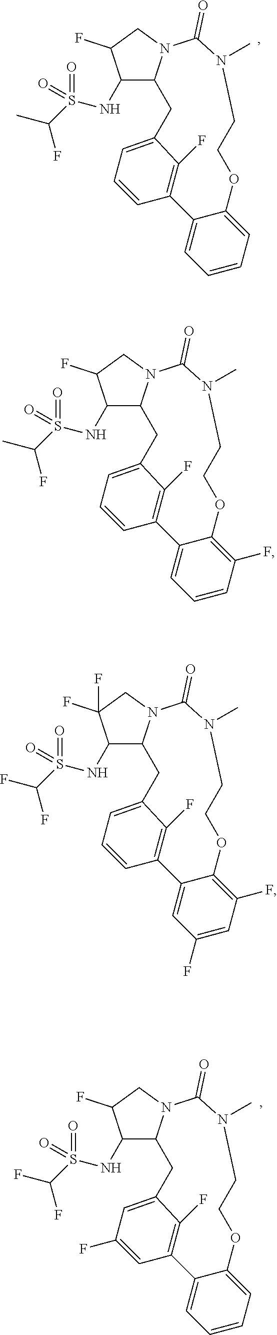 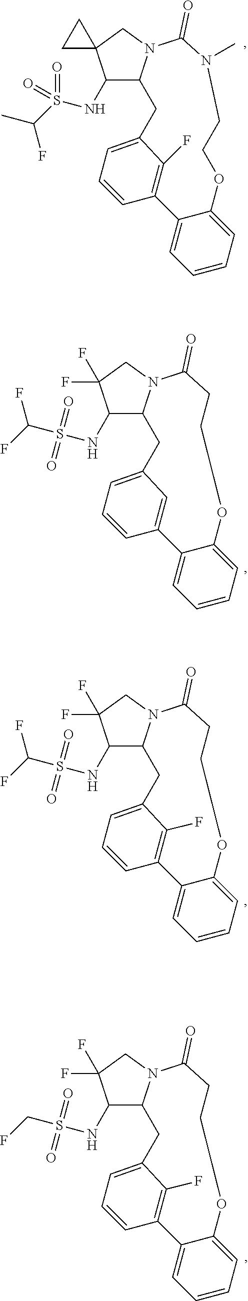 [SMILES: CC(F)S(=O)(=O)NC1C2Cc3cccc(c3F)-c3ccccc3OCCN(C)C(=O)N2CC12CC2 ; O=C1CCOc2ccccc2-c2cccc(c2)CC2C(NS(=O)(=O)C(F)F)C(F)(F)CN12 ; O=C1CCOc2ccccc2-c2cccc(c2F)CC2C(NS(=O)(=O)C(F)F)C(F)(F)CN12 ; O=C1CCOc2ccccc2-c2cccc(c2F)CC2C(NS(=O)(=O)CF)C(F)(F)CN12] 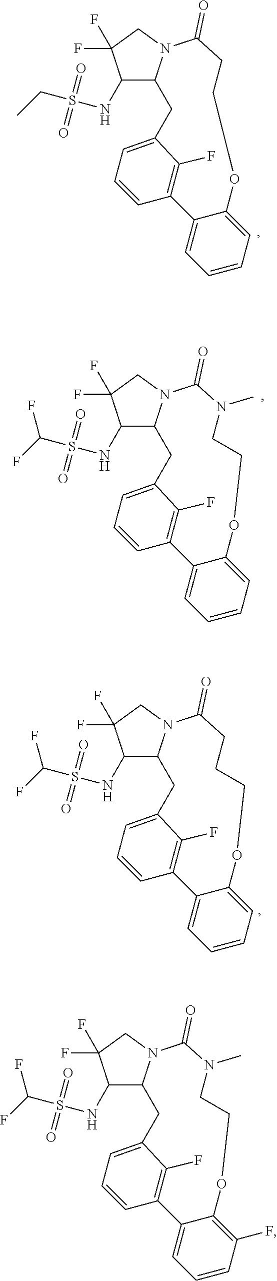 [SMILES: CCS(=O)(=O)NC1C2Cc3cccc(c3F)-c3ccccc3OCCC(=O)N2CC1(F)F ; CN1CCOc2ccccc2-c2cccc(c2F)CC2C(NS(=O)(=O)C(F)F)C(F)(F)CN2C1=O ; O=C1CCCOc2ccccc2-c2cccc(c2F)CC2C(NS(=O)(=O)C(F)F)C(F)(F)CN12 ; CN1CCOc2c(F)cccc2-c2cccc(c2F)CC2C(NS(=O)(=O)C(F)F)C(F)(F)CN2C1=O] 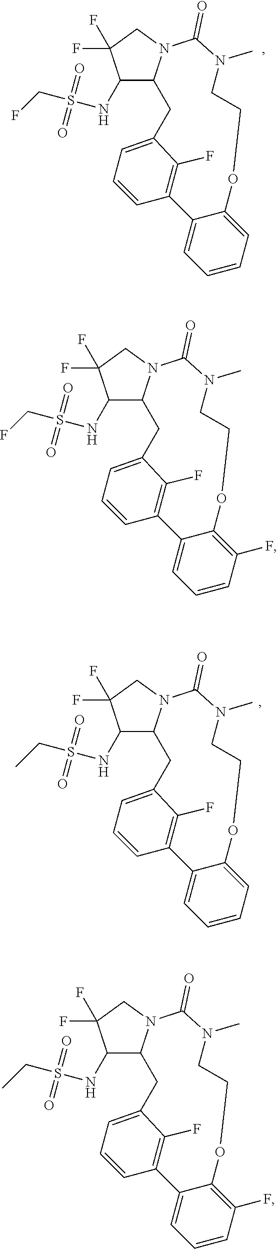 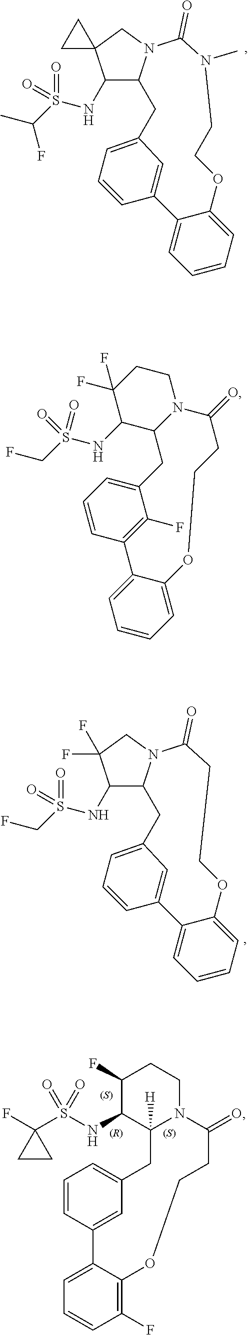 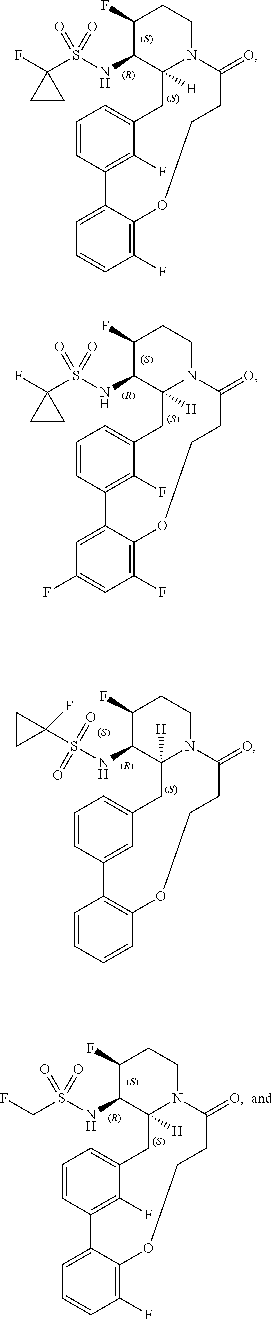 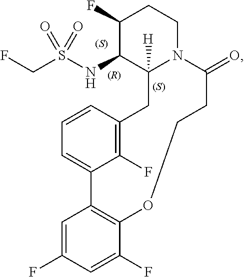
or a pharmaceutically acceptable salt thereof.

18. The compound of claim 1, wherein the compound of Formula (III′) is selected from: 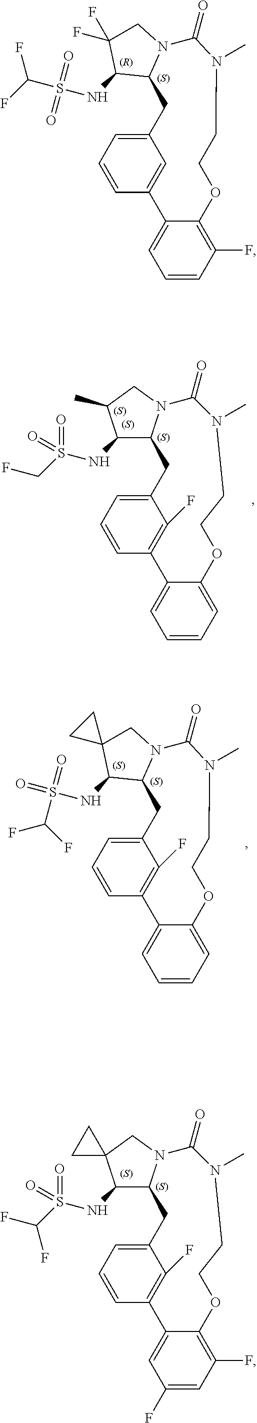 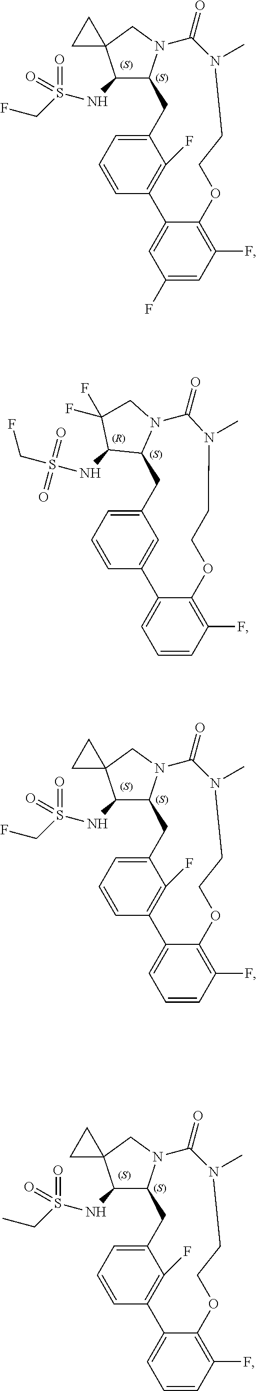 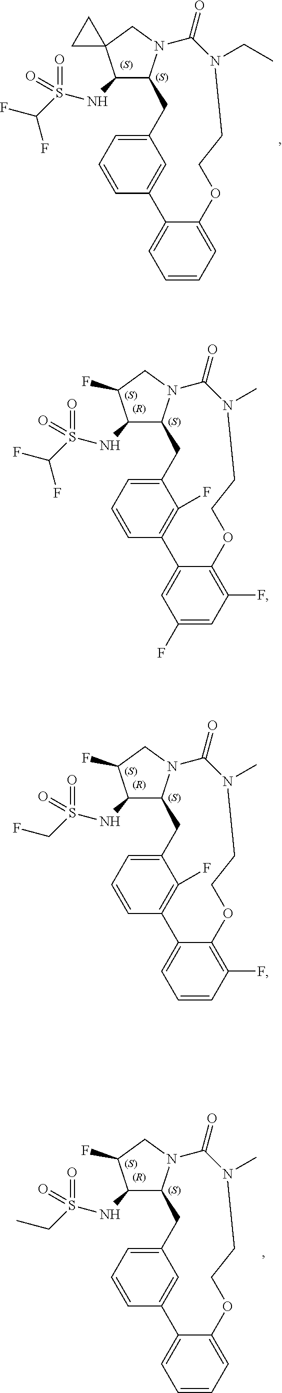 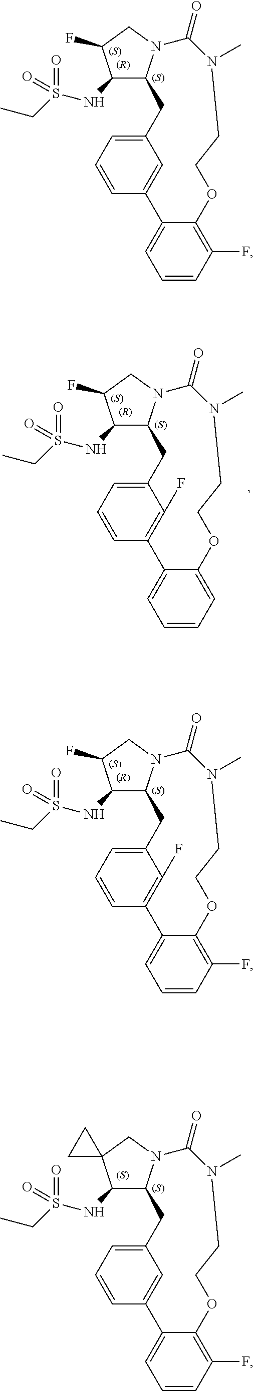 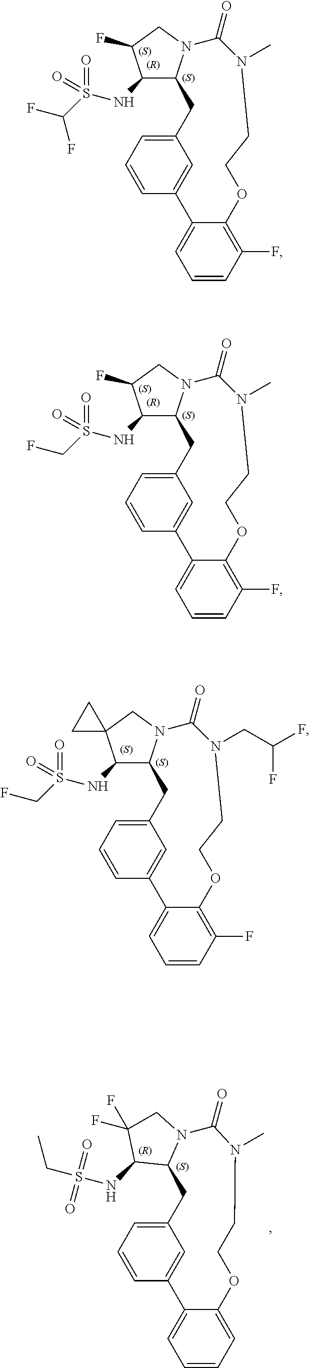 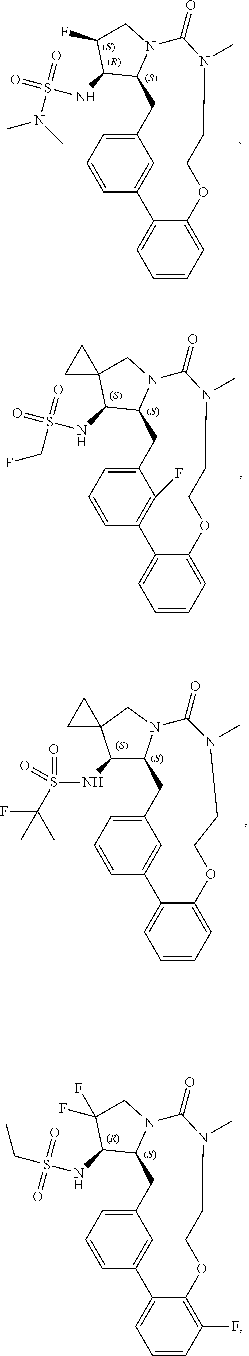 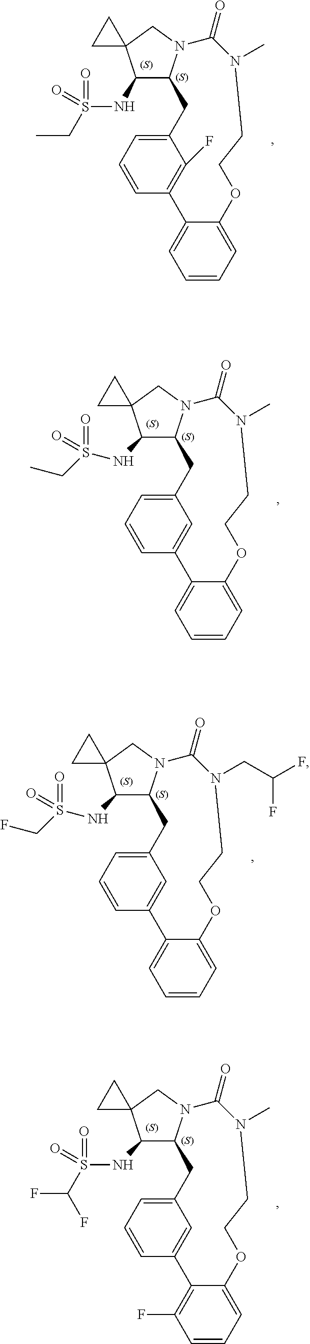 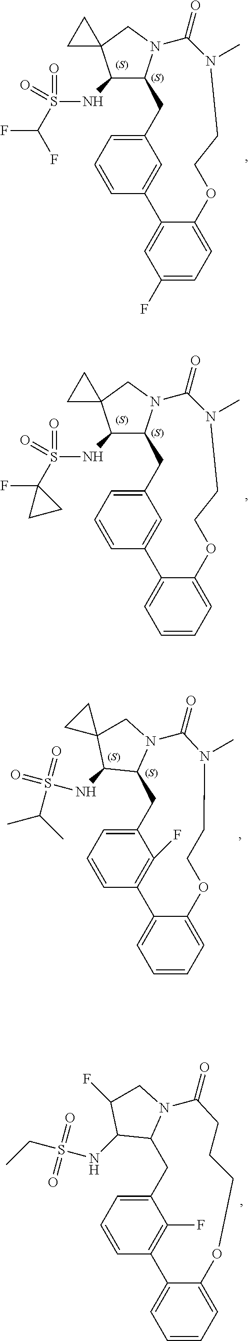 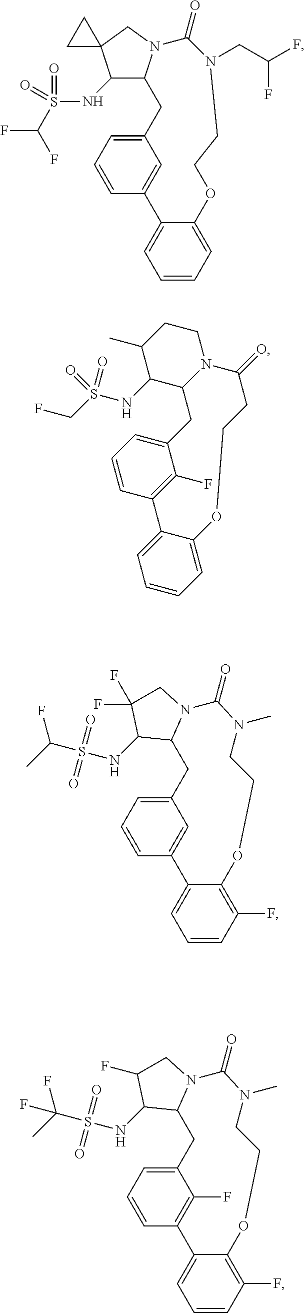 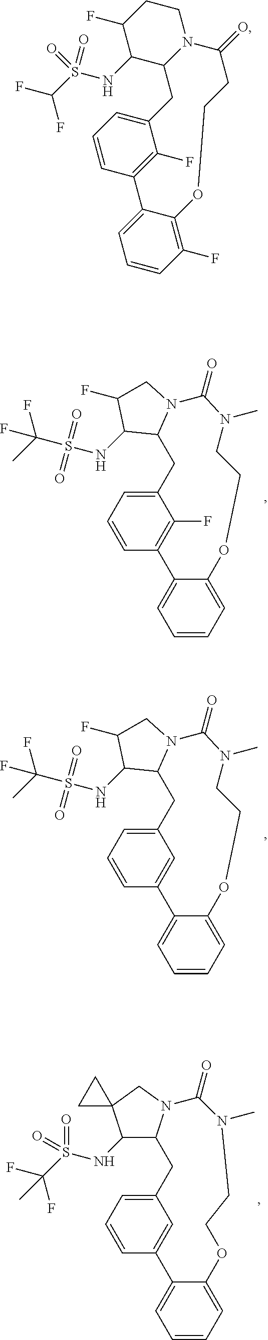 [SMILES: O=C1CCOc2c(F)cccc2-c2cccc(c2F)CC2C(NS(=O)(=O)C(F)F)C(F)CCN12 ; CN1CCOc2ccccc2-c2cccc(c2F)CC2C(NS(=O)(=O)C(C)(F)F)C(F)CN2C1=O ; CN1CCOc2ccccc2-c2cccc(c2)CC2C(NS(=O)(=O)C(C)(F)F)C(F)CN2C1=O ; CN1CCOc2ccccc2-c2cccc(c2)CC2C(NS(=O)(=O)C(C)(F)F)C3(CC3)CN2C1=O] 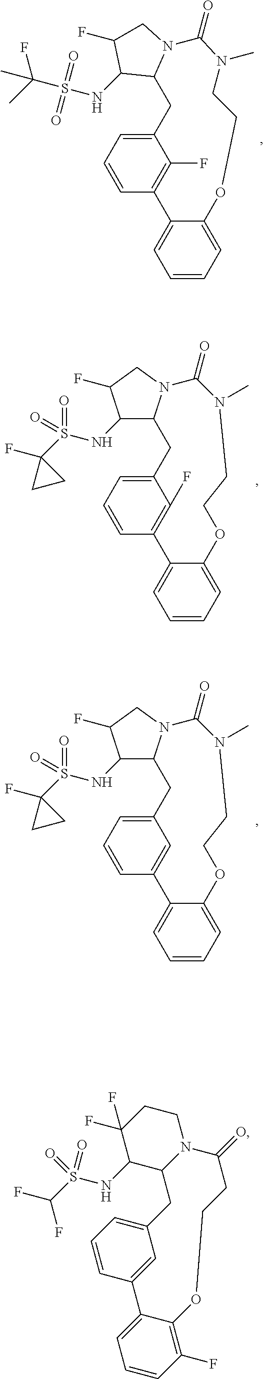 [SMILES: CN1CCOc2ccccc2-c2cccc(c2F)CC2C(NS(=O)(=O)C(C)(C)F)C(F)CN2C1=O ; CN1CCOc2ccccc2-c2cccc(c2F)CC2C(NS(=O)(=O)C3(F)CC3)C(F)CN2C1=O ; CN1CCOc2ccccc2-c2cccc(c2)CC2C(NS(=O)(=O)C3(F)CC3)C(F)CN2C1=O ; O=C1CCOc2c(F)cccc2-c2cccc(c2)CC2C(NS(=O)(=O)C(F)F)C(F)(F)CCN12] 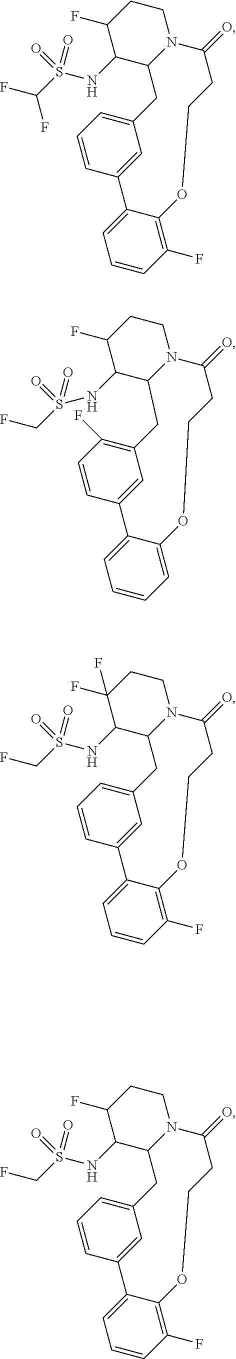 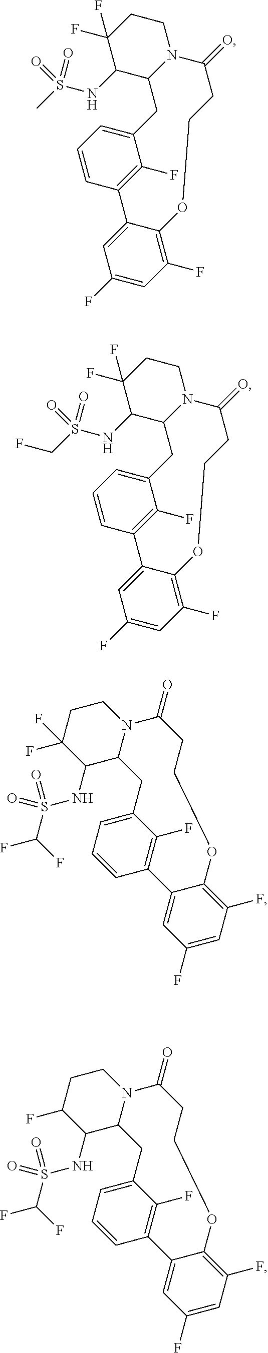 [SMILES: CS(=O)(=O)NC1C2Cc3cccc(c3F)-c3cc(F)cc(F)c3OCCC(=O)N2CCC1(F)F ; O=C1CCOc2c(F)cc(F)cc2-c2cccc(c2F)CC2C(NS(=O)(=O)CF)C(F)(F)CCN12 ; O=C1CCOc2c(F)cc(F)cc2-c2cccc(c2F)CC2C(NS(=O)(=O)C(F)F)C(F)(F)CCN12 ; O=C1CCOc2c(F)cc(F)cc2-c2cccc(c2F)CC2C(NS(=O)(=O)C(F)F)C(F)CCN12] 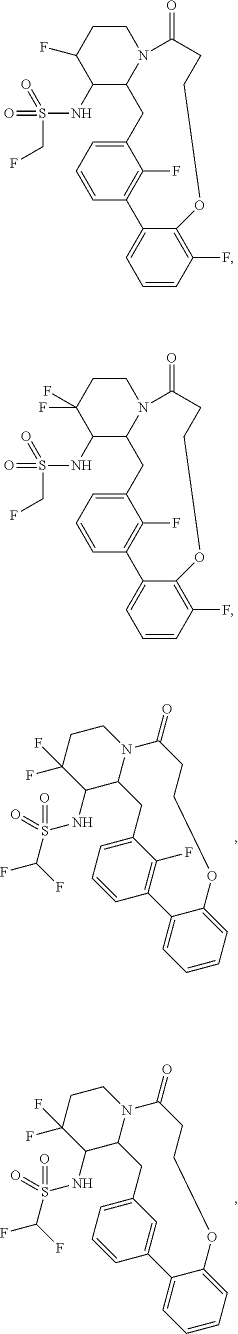 [SMILES: O=C1CCOc2c(F)cccc2-c2cccc(c2F)CC2C(NS(=O)(=O)CF)C(F)CCN12 ; O=C1CCOc2c(F)cccc2-c2cccc(c2F)CC2C(NS(=O)(=O)CF)C(F)(F)CCN12 ; O=C1CCOc2ccccc2-c2cccc(c2F)CC2C(NS(=O)(=O)C(F)F)C(F)(F)CCN12 ; O=C1CCOc2ccccc2-c2cccc(c2)CC2C(NS(=O)(=O)C(F)F)C(F)(F)CCN12] 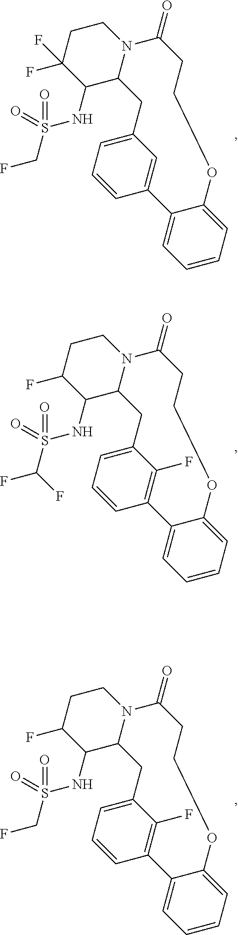 [SMILES: O=C1CCOc2ccccc2-c2cccc(c2)CC2C(NS(=O)(=O)CF)C(F)(F)CCN12 ; O=C1CCOc2ccccc2-c2cccc(c2F)CC2C(NS(=O)(=O)C(F)F)C(F)CCN12 ; O=C1CCOc2ccccc2-c2cccc(c2F)CC2C(NS(=O)(=O)CF)C(F)CCN12] 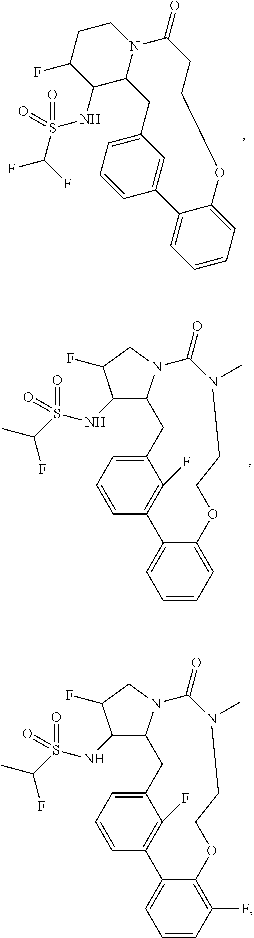 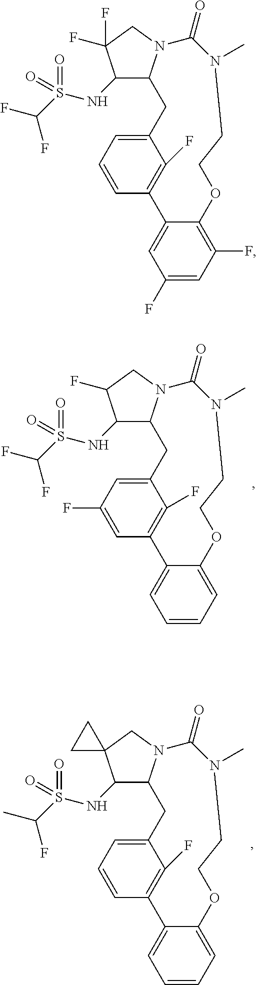 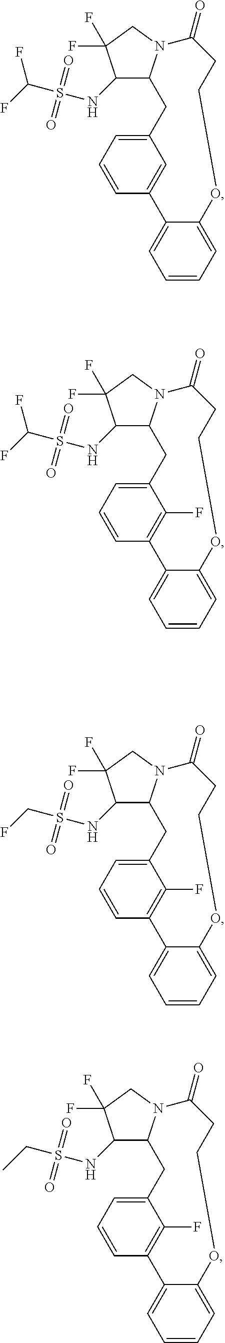 [SMILES: O=C1CCOc2ccccc2-c2cccc(c2)CC2C(NS(=O)(=O)C(F)F)C(F)(F)CN12 ; O=C1CCOc2ccccc2-c2cccc(c2F)CC2C(NS(=O)(=O)C(F)F)C(F)(F)CN12 ; O=C1CCOc2ccccc2-c2cccc(c2F)CC2C(NS(=O)(=O)CF)C(F)(F)CN12 ; CCS(=O)(=O)NC1C2Cc3cccc(c3F)-c3ccccc3OCCC(=O)N2CC1(F)F] 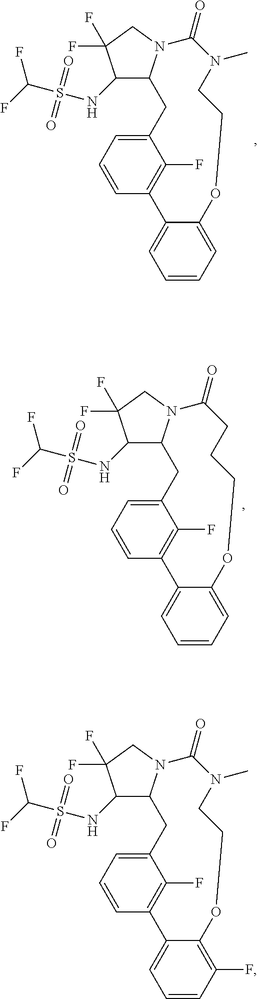 [SMILES: CN1CCOc2ccccc2-c2cccc(c2F)CC2C(NS(=O)(=O)C(F)F)C(F)(F)CN2C1=O ; O=C1CCCOc2ccccc2-c2cccc(c2F)CC2C(NS(=O)(=O)C(F)F)C(F)(F)CN12 ; CN1CCOc2c(F)cccc2-c2cccc(c2F)CC2C(NS(=O)(=O)C(F)F)C(F)(F)CN2C1=O] 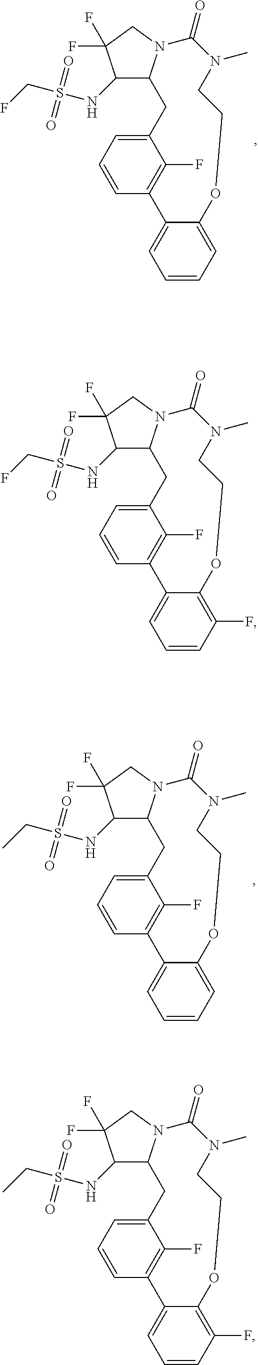 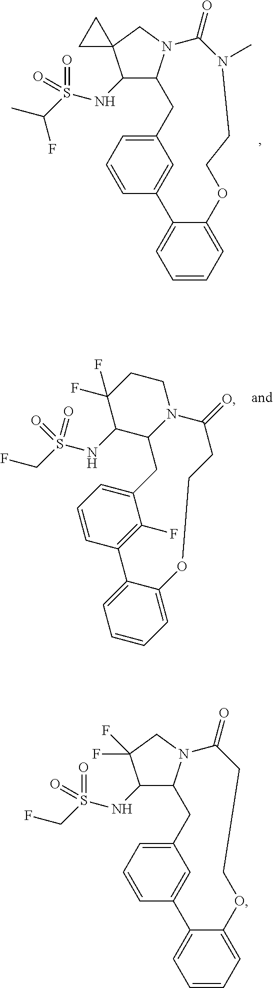 [SMILES: CC(F)S(=O)(=O)NC1C2Cc3cccc(c3)-c3ccccc3OCCN(C)C(=O)N2CC12CC2 ; O=C1CCOc2ccccc2-c2cccc(c2F)CC2C(NS(=O)(=O)CF)C(F)(F)CCN12 ; O=C1CCOc2ccccc2-c2cccc(c2)CC2C(NS(=O)(=O)CF)C(F)(F)CN12]
or a pharmaceutically acceptable salt thereof.

19. A pharmaceutical composition comprising the compound of claim 1, or a pharmaceutically acceptable salt thereof, and a pharmaceutically acceptable excipient.

20. A method of treating a disease or disorder in a subject in need thereof, comprising administering to the subject a therapeutically effective amount of the compound of claim 1, or a pharmaceutically acceptable salt thereof.
